SEQUENCE LISTING (28 sequences)

SEQ ID NO: 1 - DNA, 1983 bp, Homo sapiens
  misc_feature (1)..(1983): human MTHFR (NM_005957.4) C677T and A1298C
aggaacccagccatggtgaacgaagccagaggaaacagcagcctcaacccctgcttggagggcagtgccagcagtggcagtgagagctccaaagatagttcgagatgttccaccccgggcctggaccccgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagycgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagcgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagmaagtgtctttgaagtcttcgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatga

SEQ ID NO: 2 - DNA, 3798 bp, Homo sapiens
  misc_feature (1)..(3798): human MTR (NM_000254.2) A2756G
atgtcacccgcgctccaagacctgtcgcaacccgaaggtctgaagaaaaccctgcgggatgagatcaatgccattctgcagaagaggattatggtgctggatggagggatggggaccatgatccagcgggagaagctaaacgaagaacacttccgaggtcaggaatttaaagatcatgccaggccgctgaaaggcaacaatgacattttaagtataactcagcctgatgtcatttaccaaatccataaggaatacttgctggctggggcagatatcattgaaacaaatacttttagcagcactagtattgcccaagctgactatggccttgaacacttggcctaccggatgaacatgtgctctgcaggagtggccagaaaagctgccgaggaggtaactctccagacaggaattaagaggtttgtggcaggggctctgggtccgactaataagacactctctgtgtccccatctgtggaaaggccggattataggaacatcacatttgatgagcttgttgaagcataccaagagcaggccaaaggacttctggatggcggggttgatatcttactcattgaaactatttttgatactgccaatgccaaggcagccttgtttgcactccaaaatctttttgaggagaaatatgctccccggcctatctttatttcagggacgatcgttgataaaagtgggcggactctttccggacagacaggagagggatttgtcatcagcgtgtctcatggagaaccactctgcattggattaaattgtgctttgggtgcagctgaaatgagaccttttattgaaataattggaaaatgtacaacagcctatgtcctctgttatcccaatgcaggtcttcccaacacctttggtgactatgatgaaacgccttctatgatggccaagcacctaaaggattttgctatggatggcttggtcaatatagttggaggatgctgtgggtcaacaccagatcatatcagggaaattgctgaagctgtgaaaaattgtaagcctagagttccacctgccactgcttttgaaggacatatgttactgtctggtctagagcccttcaggattggaccgtacaccaactttgttaacattggagagcgctgtaatgttgcaggatcaaggaagtttgctaaactcatcatggcaggaaactatgaagaagccttgtgtgttgccaaagtgcaggtggaaatgggagcccaggtgttggatgtcaacatggatgatggcatgctagatggtccaagtgcaatgaccagattttgcaacttaattgcttccgagccagacatcgcaaaggtacctttgtgcatcgactcctccaattttgctgtgattgaagctgggttaaagtgctgccaagggaagtgcattgtcaatagcattagtctgaaggaaggagaggacgacttcttggagaaggccaggaagattaaaaagtatggagctgctatggtggtcatggcttttgatgaagaaggacaggcaacagaaacagacacaaaaatcagagtgtgcacccgggcctaccatctgcttgtgaaaaaactgggctttaatccaaatgacattatttttgaccctaatatcctaaccattgggactggaatggaggaacacaacttgtatgccattaattttatccatgcaacaaaagtcattaaagaaacattacctggagccagaataagtggaggtctttccaacttgtccttctccttccgaggaatggaagccattcgagaagcaatgcatggggttttcctttaccatgcaatcaagtctggcatggacatggggatagtgaatgctggaaacctccctgtgtatgatgatatccataaggaacttctgcagctctgtgaagatctcatctggaataaagaccctgaggccactgagaagctcttacgttatgcccagactcaaggcacaggagggaagaaagtcattcagactgatgagtggagaaatggccctgtcgaagaacgccttgagtatgcccttgtgaagggcattgaaaaacatattattgaggatactgaggaagccaggttaaaccaaaaaaaatatccccgacctctcaatataattgaaggacccctgatgaatggaatgaaaattgttggtgatctttttggagctggaaaaatgtttctacctcaggttataaagtcagcccgggttatgaagaaggctgttggccaccttatccctttcatggaaaaagaaagagaagaaaccagagtgcttaacggcacagtagaagaagaggacccttaccagggcaccatcgtgctggccactgttaaaggcgacgtgcacgacataggcaagaacatagttggagtagtccttggctgcaataatttccgagttattgatttaggagtcatgactccatgtgataagatactgaaagctgctcttgaccacaaagcagatataattggcctgtcaggactcatcactccttccctggatgaaatgatttttgttgccaaggaaatggagagattagctataaggattccattgttgattggaggagcaaccacttcaaaaacccacacagcagttaaaatagctccgagatacagtgcacctgtaatccatgtcctggacgcgtccaagagtgtggtggtgtgttcccagctgttagatgaaaatctaaaggatgaatactttgaggaaatcatggaagaatatgaagatattagacaggrccattatgagtctctcaaggagaggagatacttacccttaagtcaagccagaaaaagtggtttccaaatggattggctgtctgaacctcacccagtgaagcccacgtttattgggacccaggtctttgaagactatgacctgcagaagctggtggactacattgactggaagcctttctttgatgtctggcagctccggggcaagtacccgaatcgaggctttcccaagatatttaacgacaaaacagtaggtggagaggccaggaaggtctacgatgatgcccacaatatgctgaacacactgattagtcaaaagaaactccgggcccggggtgtggttgggttctggccagcacagagtatccaagacgacattcacctgtacgcagaggctgctgtgccccaggctgcagagcccatagccaccttctatgggttaaggcaacaggctgagaaggactctgccagcacggagccatactactgcctctcagacttcatcgctcccttgcattctggcatccgtgactacctgggcctgtttgccgttgcctgctttggggtagaagagctgagcaaggcctatgaggatgatggtgacgactacagcagcatcatggtcaaggcgctgggggaccggctggcagaggcctttgcagaagagctccatgaaagagttcgccgagaactgtgggcctactgtggcagtgagcagctggacgtcgcagacctgcgcaggctgcggtacaagggcatccgcccggctcctggctaccccagccagcccgaccacaccgagaagctcaccatgtggagactcgcagacatcgagcagtctacaggcattaggttaacagaatcattagcaatggcacctgcttcagcagtctcaggcctctacttctccaatttgaagtccaaatattttgctgtggggaagatttccaaggatcaggttgaggattatgcattgaggaagaacatatctgtggctgaggttgagaaatggcttggacccattttgggatatgatacagactaa

SEQ ID NO: 3 - DNA, 2097 bp, Homo sapiens
  misc_feature (1)..(2097): human MTRR (NM_002454.2) A66G
atgaggaggtttctgttactatatgctacacagcagggacaggcaaaggccatcgcagaagaaatrtgtgagcaagctgtggtacatggattttctgcagatcttcactgtattagtgaatccgataagtatgacctaaaaaccgaaacagctcctcttgttgttgtggtttctaccacgggcaccggagacccacccgacacagcccgcaagtttgttaaggaaatacagaaccaaacactgccggttgatttctttgctcacctgcggtatgggttactgggtctcggtgattcagaatacacctacttttgcaatggggggaagataattgataaacgacttcaagagcttggagcccggcatttctatgacactggacatgcagatgactgtgtaggtttagaacttgtggttgagccgtggattgctggactctggccagccctcagaaagcattttaggtcaagcagaggacaagaggagataagtggcgcactcccggtggcatcacctgcatcctcgaggacagaccttgtgaagtcagagctgctacacattgaatctcaagtcgagcttctgagattcgatgattcaggaagaaaggattctgaggttttgaagcaaaatgcagtgaacagcaaccaatccaatgttgtaattgaagactttgagtcctcacttacccgttcggtacccccactctcacaagcctctctgaatattcctggtttacccccagaatatttacaggtacatctgcaggagtctcttggccaggaggaaagccaagtatctgtgacttcagcagatccagtttttcaagtgccaatttcaaaggcagttcaacttactacgaatgatgccataaaaaccactctgctggtagaattggacatttcaaatacagacttttcctatcagcctggagatgccttcagcgtgatctgccctaacagtgattctgaggtacaaagcctactccaaagactgcagcttgaagataaaagagagcactgcgtccttttgaaaataaaggcagacacaaagaagaaaggagctaccttaccccagcatatacctgcgggatgttctctccagttcatttttacctggtgtcttgaaatccgagcaattcctaaaaaggcatttttgcgagcccttgtggactataccagtgacagtgctgaaaagcgcaggctacaggagctgtgcagtaaacaaggggcagccgattatagccgctttgtacgagatgcctgtgcctgcttgttggatctcctcctcgctttcccttcttgccagccaccactcagtctcctgctcgaacatcttcctaaacttcaacccagaccatattcgtgtgcaagctcaagtttatttcacccaggaaagctccattttgtcttcaacattgtggaatttctgtctactgccacaacagaggttctgcggaagggagtatgtacaggctggctggccttgttggttgcttcagttcttcagccaaacatacatgcatcccatgaagacagcgggaaagccctggctcctaagatatccatctctcctcgaacaacaaattctttccacttaccagatgacccctcaatccccatcataatggtgggtccaggaaccggcatagccccgtttattgggttcctacaacatagagagaaactccaagaacaacacccagatggaaattttggagcaatgtggttgttttttggctgcaggcataaggatagggattatctattcagaaaagagctcagacatttccttaagcatgggatcttaactcatctaaaggtttccttctcaagagatgctcctgttggggaggaggaagccccagcaaagtatgtgcaagacaacatccagcttcatggccagcaggtggcgagaatcctcctccaggagaacggccatatttatgtgtgtggagatgcaaagaatatggccaaggatgtacatgatgcccttgtgcaaataataagcaaagaggttggagttgaaaaactagaagcaatgaaaaccctggccactttaaaagaagaaaaacgctaccttcaggatatttggtcataa

SEQ ID NO: 4 - protein, 656 aa, Homo sapiens
  VARIANT (222)..(222): Xaa = Ala or Val
MVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGXDFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEXSVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 5 - protein, 1265 aa, Homo sapiens
  VARIANT (919)..(919): Xaa = Asp or Gly
MSPALQDLSQPEGLKKTLRDEINAILQKRIMVLDGGMGTMIQREKLNEEHFRGQEFKDHARPLKGNNDILSITQPDVIYQIHKEYLLAGADIIETNTFSSTSIAQADYGLEHLAYRMNMCSAGVARKAAEEVTLQTGIKRFVAGALGPTNKTLSVSPSVERPDYRNITFDELVEAYQEQAKGLLDGGVDILLIETIFDTANAKAALFALQNLFEEKYAPRPIFISGTIVDKSGRTLSGQTGEGFVISVSHGEPLCIGLNCALGAAEMRPFIEIIGKCTTAYVLCYPNAGLPNTFGDYDETPSMMAKHLKDFAMDGLVNIVGGCCGSTPDHIREIAEAVKNCKPRVPPATAFEGHMLLSGLEPFRIGPYTNFVNIGERCNVAGSRKFAKLIMAGNYEEALCVAKVQVEMGAQVLDVNMDDGMLDGPSAMTRFCNLIASEPDIAKVPLCIDSSNFAVIEAGLKCCQGKCIVNSISLKEGEDDFLEKARKIKKYGAAMVVMAFDEEGQATETDTKIRVCTRAYHLLVKKLGFNPNDIIFDPNILTIGTGMEEHNLYAINFIHATKVIKETLPGARISGGLSNLSFSFRGMEAIREAMHGVFLYHAIKSGMDMGIVNAGNLPVYDDIHKELLQLCEDLIWNKDPEATEKLLRYAQTQGTGGKKVIQTDEWRNGPVEERLEYALVKGIEKHIIEDTEEARLNQKKYPRPLNIIEGPLMNGMKIVGDLFGAGKMFLPQVIKSARVMKKAVGHLIPFMEKEREETRVLNGTVEEEDPYQGTIVLATVKGDVHDIGKNIVGVVLGCNNFRVIDLGVMTPCDKILKAALDHKADIIGLSGLITPSLDEMIFVAKEMERLAIRIPLLIGGATTSKTHTAVKIAPRYSAPVIHVLDASKSVVVCSQLLDENLKDEYFEEIMEEYEDIRQXHYESLKERRYLPLSQARKSGFQMDWLSEPHPVKPTFIGTQVFEDYDLQKLVDYIDWKPFFDVWQLRGKYPNRGFPKIFNDKTVGGEARKVYDDAHNMLNTLISQKKLRARGVVGFWPAQSIQDDIHLYAEAAVPQAAEPIATFYGLRQQAEKDSASTEPYYCLSDFIAPLHSGIRDYLGLFAVACFGVEELSKAYEDDGDDYSSIMVKALGDRLAEAFAEELHERVRRELWAYCGSEQLDVADLRRLRYKGIRPAPGYPSQPDHTEKLTMWRLADIEQSTGIRLTESLAMAPASAVSGLYFSNLKSKYFAVGKISKDQVEDYALRKNISVAEVEKWLGPILGYDTD

SEQ ID NO: 6 - protein, 698 aa, Homo sapiens
  VARIANT (22)..(22): Xaa = Ile or Met
MRRFLLLYATQQGQAKAIAEEXCEQAVVHGFSADLHCISESDKYDLKTETAPLVVVVSTTGTGDPPDTARKFVKEIQNQTLPVDFFAHLRYGLLGLGDSEYTYFCNGGKIIDKRLQELGARHFYDTGHADDCVGLELVVEPWIAGLWPALRKHFRSSRGQEEISGALPVASPASSRTDLVKSELLHIESQVELLRFDDSGRKDSEVLKQNAVNSNQSNVVIEDFESSLTRSVPPLSQASLNIPGLPPEYLQVHLQESLGQEESQVSVTSADPVFQVPISKAVQLTTNDAIKTTLLVELDISNTDFSYQPGDAFSVICPNSDSEVQSLLQRLQLEDKREHCVLLKIKADTKKKGATLPQHIPAGCSLQFIFTWCLEIRAIPKKAFLRALVDYTSDSAEKRRLQELCSKQGAADYSRFVRDACACLLDLLLAFPSCQPPLSLLLEHLPKLQPRPYSCASSSLFHPGKLHFVFNIVEFLSTATTEVLRKGVCTGWLALLVASVLQPNIHASHEDSGKALAPKISISPRTTNSFHLPDDPSIPIIMVGPGTGIAPFIGFLQHREKLQEQHPDGNFGAMWLFFGCRHKDRDYLFRKELRHFLKHGILTHLKVSFSRDAPVGEEEAPAKYVQDNIQLHGQQVARILLQENGHIYVCGDAKNMAKDVHDALVQIISKEVGVEKLEAMKTLATLKEEKRYLQDIWS

SEQ ID NO: 7 - DNA, 52 bp, Homo sapiens
cttgaaggagaaggtgtctgcgggagycgatttcatcatcacgcagcttttc

SEQ ID NO: 8 - DNA, 52 bp, Homo sapiens
cgaggcctttgccctgtggattgagcrgtggggaaagctgtatgaggaggag

SEQ ID NO: 9 - DNA, 52 bp, Homo sapiens
ggaagaatatgaagatattagacaggrccattatgagtctctcaaggtaagt

SEQ ID NO: 10 - DNA, 52 bp, Homo sapiens
caggcaaaggccatcgcagaagaaatrtgtgagcaagctgtggtacatggat

SEQ ID NO: 11 - DNA, 52 bp, Homo sapiens
taagttccattccatctcagcccgaartgttttcagagccggagacctcaca

SEQ ID NO: 12 - DNA, 52 bp, Homo sapiens
ctgctgctggtatcagcctggaggaartgagtgacatcagttctcagcatta

SEQ ID NO: 13 - DNA, 51 bp, Homo sapiens
accaatcacaacaaggcagataaagwaggatgagttgtcagattttgataa

SEQ ID NO: 14 - DNA, 52 bp, Homo sapiens
gcttcggagctggagcgcatgaatccyggcccaggcgggaagctgggacacg

SEQ ID NO: 15 - DNA, 52 bp, Homo sapiens
aagctgagaacatcaagaagttcttayagtaagtacatcctcgaaagtttat

SEQ ID NO: 16 - DNA, 52 bp, Homo sapiens
gggagggcacccgcagaggcctgcgcrctgacactgctgagtggctctgctc

SEQ ID NO: 17 - DNA, 52 bp, Homo sapiens
tgaccccgagctccggtcctggcggcrcctcgtgtgctacctttgcttctac

SEQ ID NO: 18 - DNA, 52 bp, Homo sapiens
caataggagcgtgtgtttgaacagtayacgccaaacttcagtcattcaagta

SEQ ID NO: 19 - DNA, 52 bp, Homo sapiens
ggcctaatcaatccttctcatcttttrtacccaccttttgcaggaaacctgt

SEQ ID NO: 20 - DNA, 52 bp, Homo sapiens
ctgactctccccgacccgtcccaccayggtctccacagcactcccgacagcc

SEQ ID NO: 21 - DNA, 52 bp, Homo sapiens
gtccctgcagtttaattatcctcaacrttactgccataccctacatttttgg

SEQ ID NO: 22 - DNA, 52 bp, Homo sapiens
ctcacagtttgtggttgagactaagtrtgacaacagtggcactttgtggtcc

SEQ ID NO: 23 - DNA, 52 bp, Homo sapiens
accaaggagcagcgcatcctgaaccaygtgctgcagcatgcggagcccggga

SEQ ID NO: 24 - DNA, 52 bp, Homo sapiens
cccagcggatggtggatttcgctggcrtgaaggacaaggtgtgcatgcctga

SEQ ID NO: 25 - DNA, 52 bp, Homo sapiens
taatatggccaccccaactttcgtatsattactgtttgtgtggtattatctt

SEQ ID NO: 26 - DNA, 52 bp, Homo sapiens
atcagccctagatgcttgaccagctcytcgggcctcacctcctggttcttcc

SEQ ID NO: 27 - DNA, 52 bp, Homo sapiens
ctgggccaacaagcttgagtgcgatcyggtctgcaatgatggaggaattgcc

SEQ ID NO: 28 - protein, 271 aa, Homo sapiens
  VARIANT (158)..(158): Xaa = Val or Met
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGXKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

(19) United States
(12) Patent Application Publication
(10) Pub. No.: US 2016/0058766 A1
(43) Pub. Date: Mar. 3, 2016

(54) ASSAYS AND METHODS FOR SELECTING A TREATMENT REGIMEN FOR A SUBJECT WITH DEPRESSION

(71) Applicant: NESTEC S.A., Vevey (CH)

(72) Inventors: MAURIZIO FAVA, Newton, MA (US); George Papakostas, Brookline, MA (US); Harold O. Koch, JR., Mandeville, LA (US); David Kronlage, Slidell, LA (US)

(21) Appl. No.: 14/835,627

(22) Filed: Aug. 25, 2015

Related U.S. Application Data
(63) Continuation of application No. PCT/US2014/023695, filed on Mar. 11, 2014.
Application No. PCT/US2014/023695 is a continuation-in-part of application No. 13/796,362, filed on Mar. 12, 2013.
Application No. 13/796,362 is a continuation-in-part of application No. PCT/US2012/065084, filed on Nov. 14, 2012.
(60) Provisional application No. 61/777,650, filed on Mar. 12, 2013.
(60) Provisional application No. 61/914,338, filed on Dec. 10, 2013.
(60) Provisional application No. 61/559,541, filed on Nov. 14, 2011.

(51) Int. Cl. A61K 31/519; C12Q 1/68; A61K 45/06

(52) U.S. Cl. CPC A61K 31/519; A61K 45/06; C12Q 1/6883; C12Q 2600/156; C12Q 2600/158; C12Q 2600/118; C12Q 2600/106

(57) ABSTRACT

The present invention provides assays, methods and compositions for selecting a treatment regimen for a patient having depression or at risk for depression and/or treating at least one symptom of depression in the subject, based on the recognition that specific combinations of single nucleotide polymorphisms (SNPs) are associated with a therapeutic response to a folate-comprising compound. Provided herein are also methods for improving the effectiveness of an antidepressant drug administered to a subject with depression or at risk for depression by administering an adjunctive therapy of a folate-comprising compound to the subject if the subject carries a specific combination of SNPs that are predictive of a therapeutic response. Furthermore, provided herein are compositions of the folate-comprising compound.

### CROSS-REFERENCES TO RELATED APPLICATIONS

This application is a continuation of PCT/US2014/023695, filed Mar. 11, 2014, which application claims priority to U.S. Provisional Application No. 61/777,650, filed Mar. 12, 2013 and U.S. Provisional Application No. 61/914,338, filed Dec. 10, 2013. This application is also a continuation-in-part of U.S. application Ser. No. 13/796,362, filed Mar. 12, 2013, which application is a continuation-in-part of PCT/US2012/065084, filed Nov. 14, 2012, which application claims priority to U.S. Provisional Application No. 61/559,541, filed Nov. 14, 2011. The disclosures of all of the above-referenced applications are hereby incorporated by reference in their entireties for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file SEQ_LISTING_{—}95768-902165.TXT, created on Mar. 11, 2014, 37,509 bytes, machine format IBM-PC, MS-Windows operating system, is hereby incorporated by reference in its entirety for all purposes.

### BACKGROUND OF THE INVENTION

Recent estimates indicate that more than 19 million Americans over the age of 18 experience a depressive illness each year. It has been generally believed that there is an association between folate-deficiency states and depression.

The role of folate in central-nervous-system function has been investigated, including the role of folate in the one-carbon cycle that furnishes SAMe, the principal methyl donor for a broad range of reactions involving the synthesis of neuroactive substances, the formation of membrane phospholipids, and the metabolism of nucleic acids. The evidence suggests that folate can affect depression. When administered in parenteral and certain oral forms, SAM has been reported in European studies to have antidepressant efficacy greater than placebo and comparable to that of tricyclic antidepressants. Folate also appears to influence the rate of synthesis of tetrahydrobiopterin, a cofactor in the hydroxylation of phenylalanine and tryptophan, rate-limiting steps in the biosynthesis of dopamine, norepinephrine, and serotonin, neurotransmitters postulated to play a role in the pathogenesis of depression. In addition, methyltetrahydrofolate (MTHF) has been shown to bind to presynaptic glutamate receptors, where it may potentially modulate the release of other neurotransmitters, including the monoamines Elevated levels of homocysteine, resulting from folate deficiency, can play a role in mediating some of its neuropsychiatric complications by both generating elevated levels of S-adenosyl-homocysteine (SAH), which broadly inhibits methylation reactions, and also possibly exerting direct excitoxic effects via activity at the N-methyl-aspartate glutamate receptors.

Neuropsychiatric and depressive symptoms, including apathy, fatigue, insomnia, irritability, and impaired concentration, have been discussed in clinical descriptions of folate-deficiency states associated with malabsorption, anticonvulsant-treated epilepsy, megaloblastic anemia, and dietary folate restriction. Previous studies have reported that as many as one-third of patients among psychiatric cohorts, mainly from the United Kingdom, exhibited low or deficient serum folate values, with generally comparable findings in the few studies that have assessed red blood cell (RBC) folate as a more accurate reflection of tissue folate stores. In the subset of the studies in which depressed patients were compared with psychiatric or non-psychiatric control subjects, depressed patients were reported to have serum folate, RBC folate[21], or serum methyltetrahydrofolate (MTHF) levels that were lower than levels in all other groups except for patients with alcoholism who had a similar prevalence of low folate. Furthermore, low serum or RBC folate and serum MTHF were often associated with greater symptom severity among depressed patients.

While significant advances in the treatment of depression have been made in the past decade, as many as 29% to 46% of patients with depression taking an antidepressant are still partially or totally resistant to the treatment. In addition, those who suffer from treatment-resistant depression have almost no alternatives. Approximately 50% of MDD patients receiving antidepressant drug have no response or partial response. The presence of residual symptoms is also associated with a higher risk of recurrence, more chronic depressive episodes and a shorter duration between episodes. Guidelines for treatment recommend four possible strategies for managing non-response or partial response including: increasing the dose of the antidepressant drug; replacing the drug with a different antidepressant drug; augmenting the antidepressent therapy with a non-antidepressant agent; or combining the initial antidepressant with a second antidepressant. As not every treatment regimen is effective for each individual, there is a strong need to identify predictive markers that can facilitate selection of an appropriate and effective treatment regimen for a subject with depression.

### BRIEF SUMMARY OF THE INVENTION

A significant portion of patients with depression, such as major depressive disorders, show only partial or no response to conventional antidepressant drugs, e.g., selective serotonin reuptake inhibitors. Thus, there is a strong need to develop effective antidepressant therapies and/or to stratify patients with depression such that they can receive appropriate antidepressant therapies. Aspects of various embodiments described herein stem from the discovery of single nucleotide polymorphisms (SNPs), peripheral biomarkers and/or clinical features that are associated with an efficacy response to the use of a folate-containing compound for treatment of depression, e.g., major depressive disorders, as a monotherapy or as an adjunct to an antidepressant drug. In some embodiments, the inventors have also shown that these markers or conditions described herein can also be used to select a more effective treatment for subjects with treatment-resistant depression (TRD), e.g., resistant to at least one selective serotonin reuptake inhibitor (SSRI).

Particularly, one or a combination of biomarkers that can be indicative of a patient (e.g., with major depressive disorders and/or TRD) suitable for a treatment regimen comprising a folate-containing compound include, but are not limited to, at least one or more SNPs identified by rs numbers as follows: rs1801133 present in methylenetetrahydrofolate reductase (MTHFR); rs2274976 present in MTHFR; rs1805087 present in methionine synthase (MTR); rs1801394 present in methionine synthase reductase (MTRR); rs1006737 present in calcium channel, voltage-dependent, L-type, alpha 1C subunit (CACNA1C); rs1883729 present in DNA (cytosine-5)-methyltransferase 3 beta (DNMT3B); rs7163862 present in GTP cyclohydrolase 1 feedback regulatory protein (GCHFR); rs12659 present in reduced folate carrier protein (RCF2); rs202676 present in folate hydrolase (prostate-specific membrane antigen) (FOLH1); rs2297291 present in reduced folate carrier protein (RCF1); rs1051266 present in reduced folate carrier protein 1 (RCF1); rs8007267 present in GTP cyclohydrolase 1 (GCH1); rs7639752 present in choline-phosphate cytidylytransferase A (PCYT1A); rs6275 present in dopamine receptor D2 (DRD2); rs1079596 present in DRD2; rs11240594 present in DRD2; rs4633 present in catechol-O-methyltransferase (COMT); rs4680 present in COMT; rs250682 present in dopamine active transporter (DAT, or SLC6A3); rs2277820 present in formiminotransferase cyclodeaminase (FTCD); rs2236225 present in methylenetetrahydrofolate dehydrogenase (NADP+ dependent) 1 (MTHFD1); and any combinations thereof; and/or expression of at least one of s-adenosyl methionine (SAM), s-adenosyl homocysteine (SAH), 4-hydroxynonenal (4-HNE), high sensitivity c-reactive protein (hsCRP), and any combinations thereof. Additionally or alternatively, determination of whether a human subject is obese or not (e.g., by measurement of a BMI value) can also be used as a biomarker to select an appropriate treatment regimen (e.g., comprising a folate-containing compound or not) for a patient with depression or a risk for depression. Any individual or combinations of such biomarkers disclosed herein can be used to identify patients, who are diagnosed as having depression or having a risk for depression, for receiving a treatment regimen comprising a folate-containing compound. In some embodiments, a folate-containing compound can be used in the absence of an anti-depressant drug for treatment of depression (e.g., major depressive disorders) in subjects selected for carrying at least one or more biomarkers described herein. In alternative embodiments, a folate-containing compound can be used alone or in combination (e.g., as an adjunct) with an anti-depressant drug for treatment of depression (e.g., major depressive disorders) in subjects selected for carrying at least one or more biomarkers described herein. n one embodiment, the anti-depressant drug can include a selective serotonin reuptake inhibitor (SSRI).

Examples of the SSRI include, but are not limited to, fluoxetine, citalopram, paroxetine, escitalopram, sertraline, and any combinations thereof.

Accordingly, provided herein relate to assays, methods, systems, and kits for selecting a treatment regimen for a subject with depression or a risk for depression, treating a subject with depression or a risk for depression, and/or improving the effectiveness of a treatment regimen recommended for or administered to a subject with depression or a risk for depression. Provided herein also relate to folate-comprising compositions for use in treatment of depression in a subject (e.g., a human subject) selected to carry at least one (e.g., at least two or more) or any combinations of the biomarkers or conditions described herein.

In one aspect, provided herein is a method for treating at least one symptom of depression in a human subject. The method comprises administering a composition comprising an effective amount of a folate-comprising compound to a human subject, who is diagnosed to have depression or have a risk for depression, and is further determined to carry a combination of at least two of the following biomarkers:
i. a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR);
ii. a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR),
iii. a SNP at position 1793 of SEQ ID NO: 1 or position 27 of SEQ ID NO: 8 (identified by rs2274976) comprising at least one adenine “A” allele or the complement thereof, wherein the SEQ ID NO: 1 and SEQ ID NO: 8 are each independently a portion of a genomic nucleic acid sequence of methylenetetrahydrofolate reductase (MTHFR);
iv. a SNP at position 66 of SEQ ID NO: 3 or position 27 of SEQ ID NO: 10 (identified by rs1801394) comprising at least one guanine “G” allele or the complement thereof, wherein the SEQ ID NO: 3 and SEQ ID NO: 10 are each independently a portion of a genomic nucleic acid sequence of methionine synthase reductase (MTRR);
v. a SNP at position 27 of SEQ ID NO: 11 (identified by rs1006737) comprising at least one adenine “A” allele or the complement thereof, wherein the SEQ ID NO: 11 is a portion of a genomic nucleic acid sequence of calcium channel, voltage-dependent, L type, alpha 1C subunit (CACNA1C);
vi. a SNP at position 27 of SEQ ID NO: 12 (identified by rs1883729) comprising at least one adenine “A” allele or the complement thereof, wherein the SEQ ID NO: 12 is a portion of a genomic nucleic acid sequence of DNA (cytosine-5)-methyltransferase 3 beta (DNMT3B);
vii. a SNP at position 27 of SEQ ID NO: 13 (identified by rs7163862) comprising at least one thymine “T” allele or the complement thereof, wherein the SEQ ID NO: 13 is a portion of a genomic nucleic acid sequence of GTP cyclohydrolase 1 feedback regulatory protein (GCHFR);
viii. a SNP at position 27 of SEQ ID NO: 14 (identified by rs12659) comprising two thymine “T” allele or the complement thereof, wherein the SEQ ID NO: 14 is a portion of a genomic nucleic acid sequence of reduced folate carrier protein (RCF2);
ix. a SNP at position 27 of SEQ ID NO: 15 (identified by rs202676) comprising at least one guanine “G” allele or the complement thereof, wherein the SEQ ID NO: 15 is a portion of a genomic nucleic acid sequence of folate hydrolase (prostate-specific membrane antigen) 1 (FOLH1);
x. a SNP at position 27 of SEQ ID NO: 16 (identified by rs2297291) comprising two adenine “A” alleles or the complement thereof, wherein the SEQ ID NO: 16 is a portion of a genomic nucleic acid sequence of reduced folate carrier protein (RCF1);
xi. a SNP at position 27 of SEQ ID NO: 17 (identified by rs1051266) comprising two adenine “A” alleles or the complement thereof, wherein the SEQ ID NO: 17 is a portion of a genomic nucleic acid sequence of reduced folate carrier protein (RCF1);
xii. a SNP at position 27 of SEQ ID NO: 18 (identified by rs8007267) comprising at least one thymine “T” allele or the complement thereof, wherein the SEQ ID NO: 18 is a portion of a genomic nucleic acid sequence of GTP cyclohydrolase 1 (GCH1);
xiii. a SNP at position 27 of SEQ ID NO: 19 (identified by rs7639752) comprising at least one adenine “A” allele or the complement thereof, wherein the SEQ ID NO: 19 is a portion of a genomic nucleic acid sequence of choline-phosphate cytidylyltransferase A (PCYT1A);
xiv. a SNP at position 27 of SEQ ID NO: 20 (identified by rs6275) comprising two thymine “T” alleles or the complement thereof, wherein the SEQ ID NO: 20 is a portion of a genomic nucleic acid sequence of dopamine receptor D2 (DRD2);
xv. a SNP at position 27 of SEQ ID NO: 21 (identified by rs1079596) comprising at least one thymine “T” allele or the complement thereof, wherein the SEQ ID NO: 21 is a portion of a genomic nucleic acid sequence of dopamine receptor D2 (DRD2);
xvi. a SNP at position 27 of SEQ ID NO: 22 (identified by rs11240594) comprising at least one adenine “A” allele or the complement thereof, wherein the SEQ ID NO: 22 is a portion of a genomic nucleic acid sequence of dopamine receptor D2 (DRD2);
xvii. a SNP at position 27 of SEQ ID NO: 23 (identified by rs4633) comprising two cytosine “C” alleles or the complement thereof, wherein the SEQ ID NO: 23 is a portion of a genomic nucleic acid sequence of catechol-O-methyltransferase (COMT);
xviii. a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT);
xix. a SNP at position 27 of SEQ ID NO: 25 (identified by rs250682) comprising at least one cytosine “C” allele or the complement thereof, wherein the SEQ ID NO: 25 is a portion of a genomic nucleic acid sequence of solute carrier family 6 (neurotransmitted transported, dopamine), member 3 (SLC6A3);
xx. a SNP at position 27 of SEQ ID NO: 26 (identified by rs2277820) comprising at least one thymine “T” allele or the complement thereof, wherein the SEQ ID NO: 26 is a portion of a genomic nucleic acid sequence of formiminotransferase cyclodeaminase (FTCD);
xxi. a SNP at position 27 of SEQ ID NO: 27 (identified by rs2236225) comprising at least one adenine “A” allele or the complement thereof, wherein the SEQ ID NO: 27 is a portion of a genomic nucleic acid sequence of methylenetetrahydrofolate dehydrogenase (NADP+ dependent) 1 (MTHFD1);
xxii. obesity;
xxiii. an expression ratio of SAM to SAH smaller than a pre-determined reference ratio;
xxiv. an expression of 4-HNE greater than a first pre-determined reference value; and
xxv. an expression of hsCRP greater than a second pre-determined reference value,
   based on the recognition that the combination of said at least two of the biomarkers is associated with positive-symptom-reducing response to the folate-comprising compound.

In some embodiments, the combination of said at least two biomarkers comprises the following:
i. a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR); and
ii. a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR).

In some embodiments, the combination of said at least two biomarkers further comprises at least one of the biomarkers (iii)-(xxv) described herein. In other embodiments, the obesity is characterized by at least one of the following obesity indicators:
a. a BMI value greater than 30 kg/m2;
b. a waist circumference greater than 40 inches (or greater than 120 cm) in men, or greater than 35 inches (or greater than 88 cm) in women;
c. a waist-hip ratio above 0.95 for men or above 0.80 for women; and
d. a body fat percentage of at least about 25% in men or at least about 32% in women.

In some embodiments, the method further comprises assaying a biological sample obtained from the subject for determination of the presence of said at least two biomarkers.

In some embodiments, the biological sample comprises a sample selected from a blood sample, a urine sample, a buccal sample, a saliva sample or a cerebrospinal fluid sample.

In some embodiments, the assaying comprises amplifying the biological sample with at least one set of primers flanking any one of the SNPs. In some instances, at least two sets of primers amplifying at least two of the SNPs are used in a multiplex amplification assay. In other embodiments, the assaying comprises separating and/or detecting the presence of SAM, SAH, 4-HNE, hsCRP or any combinations thereof in the biological sample with gas chromatography, mass spectrometry, high performance liquid chromatography, nuclear magnetic resonance (NMR) spectroscopy, an enzyme-coupled-assay, or any combinations thereof.

In some embodiments, the pre-determined reference ratio of SAM/SAH is a control ratio of SAM/SAH as measured in a biological sample of normal healthy subjects. In some embodiments, the control ratio of SAM/SAH as measured in a serum sample of the normal healthy subjects ranges from about 4 to about 12. In certain embodiments, the pre-determined reference ratio of SAM/SAH is about 3.0 as measured in a plasma sample. In some embodiments, the first pre-determined reference value of 4-HNE is a control value of 4-HNE as measured in a biological sample of normal healthy subjects. In some embodiments, the control value of 4-HNE as measured in a serum sample of the normal healthy subjects is about 0.24 μmol per liter of serum (or about 0.04 mg per liter of serum). In other embodiments, the first pre-determined reference value of 4-HNE is about 3 mg per liter of plasma as measured in a plasma sample. In some embodiments, the second pre-determined reference value of hsCRP is a control value of hsCRP as measured in a biological sample of normal healthy subjects. In other embodiments, the control value of hsCRP as measured in a serum sample of the normal healthy subjects ranges from about 0.5 mg per liter of serum to about 4.5 mg per liter of serum. In some embodiments, the second pre-determined reference value of hsCRP is about 2.3 mg per liter of plasma as measured in a plasma sample.

In some embodiments, the method further comprises determining a body measurement of the subject. In some instances, the body measurement comprises weight, height, waist circumference, hip circumference, body fat percentage, or any combinations thereof.

In some embodiments, the effective amount of the folate-comprising compound is about 7.5 mg/day to about 50 mg/day.

In some embodiments, the effective amount of the folate-comprising compound is administered as a single daily dose. In other embodiments, the effective amount of the folate-comprising compound is administered in more than one divided doses per day.

In some embodiments, the administration is oral.

In some embodiments, the composition is formulated to release at least a portion of the folate-comprising compound over a period of at least about 3-6 hours, upon the administration of the composition. In some instances, the release is a steady-state release.

In some embodiments, the method further comprises administering to the subject an anti-depressant drug. In some instances, the administration of the anti-depressant drug in combination with the folate-comprising compound increases the effectiveness of the anti-depressant drug. In some embodiments, the anti-depressant drug comprises a selective serotonin reuptake inhibitor. In some instances, the selective serotonin reuptake inhibitor is selected from the group consisting of fluoxetine, citalopram, paroxetine, escitalopram, sertraline, and any combinations thereof.

In some embodiments, the method further selecting for the subject a treatment comprising the folate-comprising compound, optionally administered in combination with the anti-depressant drug.

In some embodiments, the depression is major depressive disorder.

In some embodiments, the subject who is diagnosed as having depression is resistant to at least one antidepressant monotherapy. In some embodiments, the subject is an adult subject.

In some embodiments, the at least one symptom of depression is selected from low or depressed mood, anhedonia, low energy levels, guilt, decreased work and interests, psychomotor retardation, agitation, psychic anxiety, somatic anxiety, general somatic symptoms, reduced cognition or any combinations thereof.

In another aspect, provided herein is a method of improving the effectiveness of an anti-depressant drug administered to a human subject. The method comprises administering a composition comprising an effective amount of a folate-comprising compound, in combination with the anti-depressant drug, to the human subject who is diagnosed to have depression and is further determined to carry a combination of at least two of the following biomarkers:
i. a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR);
ii. a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR),
iii. a SNP at position 1793 of SEQ ID NO: 1 or position 27 of SEQ ID NO: 8 (identified by rs2274976) comprising at least one adenine “A” allele or the complement thereof, wherein the SEQ ID NO: 1 and SEQ ID NO: 8 are each independently a portion of a genomic nucleic acid sequence of methylenetetrahydrofolate reductase (MTHFR);
iv. a SNP at position 66 of SEQ ID NO: 3 or position 27 of SEQ ID NO: 10 (identified by rs1801394) comprising at least one guanine “G” allele or the complement thereof, wherein the SEQ ID NO: 3 and SEQ ID NO: 10 are each independently a portion of a genomic nucleic acid sequence of methionine synthase reductase (MTRR);
v. a SNP at position 27 of SEQ ID NO: 11 (identified by rs1006737) comprising at least one adenine “A” allele or the complement thereof, wherein the SEQ ID NO: 11 is a portion of a genomic nucleic acid sequence of calcium channel, voltage-dependent, L type, alpha 1C subunit (CACNA1C);
vi. a SNP at position 27 of SEQ ID NO: 12 (identified by rs1883729) comprising at least one adenine “A” allele or the complement thereof, wherein the SEQ ID NO: 12 is a portion of a genomic nucleic acid sequence of DNA (cytosine-5)-methyltransferase 3 beta (DNMT3B);
vii. a SNP at position 27 of SEQ ID NO: 13 (identified by rs7163862) comprising at least one thymine “T” allele or the complement thereof, wherein the SEQ ID NO: 13 is a portion of a genomic nucleic acid sequence of GTP cyclohydrolase 1 feedback regulatory protein (GCHFR);
viii. a SNP at position 27 of SEQ ID NO: 14 (identified by rs12659) comprising two thymine “T” allele or the complement thereof, wherein the SEQ ID NO: 14 is a portion of a genomic nucleic acid sequence of reduced folate carrier protein (RCF2);
ix. a SNP at position 27 of SEQ ID NO: 15 (identified by rs202676) comprising at least one guanine “G” allele or the complement thereof, wherein the SEQ ID NO: 15 is a portion of a genomic nucleic acid sequence of folate hydrolase (prostate-specific membrane antigen) 1 (FOLH1);
x. a SNP at position 27 of SEQ ID NO: 16 (identified by rs2297291) comprising two adenine “A” alleles or the complement thereof, wherein the SEQ ID NO: 16 is a portion of a genomic nucleic acid sequence of reduced folate carrier protein (RCF1);
xi. a SNP at position 27 of SEQ ID NO: 17 (identified by rs1051266) comprising two adenine “A” alleles or the complement thereof, wherein the SEQ ID NO: 17 is a portion of a genomic nucleic acid sequence of reduced folate carrier protein (RCF1);
xii. a SNP at position 27 of SEQ ID NO: 18 (identified by rs8007267) comprising at least one thymine “T” allele or the complement thereof, wherein the SEQ ID NO: 18 is a portion of a genomic nucleic acid sequence of GTP cyclohydrolase 1 (GCH1);
xiii. a SNP at position 27 of SEQ ID NO: 19 (identified by rs7639752) comprising at least one adenine “A” allele or the complement thereof, wherein the SEQ ID NO: 19 is a portion of a genomic nucleic acid sequence of choline-phosphate cytidylyltransferase A (PCYT1A);
xiv. a SNP at position 27 of SEQ ID NO: 20 (identified by rs6275) comprising two thymine “T” alleles or the complement thereof, wherein the SEQ ID NO: 20 is a portion of a genomic nucleic acid sequence of dopamine receptor D2 (DRD2);
xv. a SNP at position 27 of SEQ ID NO: 21 (identified by rs1079596) comprising at least one thymine “T” allele or the complement thereof, wherein the SEQ ID NO: 21 is a portion of a genomic nucleic acid sequence of dopamine receptor D2 (DRD2);
xvi. a SNP at position 27 of SEQ ID NO: 22 (identified by rs11240594) comprising at least one adenine “A” allele or the complement thereof, wherein the SEQ ID NO: 22 is a portion of a genomic nucleic acid sequence of dopamine receptor D2 (DRD2);
xvii. a SNP at position 27 of SEQ ID NO: 23 (identified by rs4633) comprising two cytosine “C” alleles or the complement thereof, wherein the SEQ ID NO: 23 is a portion of a genomic nucleic acid sequence of catechol-O-methyltransferase (COMT);
xviii. a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT);
xix. a SNP at position 27 of SEQ ID NO: 25 (identified by rs250682) comprising at least one cytosine “C” allele or the complement thereof, wherein the SEQ ID NO: 25 is a portion of a genomic nucleic acid sequence of solute carrier family 6 (neurotransmitted transported, dopamine), member 3 (SLC6A3);
xx. a SNP at position 27 of SEQ ID NO: 26 (identified by rs2277820) comprising at least one thymine “T” allele or the complement thereof, wherein the SEQ ID NO: 26 is a portion of a genomic nucleic acid sequence of formiminotransferase cyclodeaminase (FTCD);
xxi. a SNP at position 27 of SEQ ID NO: 27 (identified by rs2236225) comprising at least one adenine “A” allele or the complement thereof, wherein the SEQ ID NO: 27 is a portion of a genomic nucleic acid sequence of methylenetetrahydrofolate dehydrogenase (NADP+ dependent) 1 (MTHFD1);
xxii. obesity;
xxiii. an expression ratio of SAM to SAH smaller than a pre-determined reference ratio;
xxiv. an expression of 4-HNE greater than a first pre-determined reference value; and
xxv. an expression of hsCRP greater than a second pre-determined reference value,
   based on the recognition that the combination of said at least two of the biomarkers is associated with increasing the effectiveness of the anti-depressant drug when administered in combination with the folate-comprising compound.

In some embodiments, the combination of said at least two biomarkers comprises the following:
i. a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR); and
ii. a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR).

In some embodiments, the combination of said at least two biomarkers further comprises at least one of the biomarkers (iii)-(xxv) described herein.

In some embodiments, the obesity is characterized by at least one of the following obesity indicators:
a) a BMI value greater than 30 kg/m²;
b) a waist circumference greater than 40 inches (or greater than 120 cm) in men, or greater than 35 inches (or greater than 88 cm) in women;
c) a waist-hip ratio above 0.95 for men or above 0.80 for women; and
d) a body fat percentage of at least about 25% in men or at least about 32% in women.

In some embodiments, the method further comprises assaying a biological sample obtained from the subject for determination of the presence of said at least two biomarkers.

In some embodiments, the biological sample comprises a sample selected from a blood sample, a urine sample, a buccal sample, a saliva sample or a cerebrospinal fluid sample.

In some embodiments, the assaying comprises amplifying the biological sample with at least one set of primers flanking any one of the SNPs. In some instances, at least two sets of primers amplifying at least two of the SNPs are used in a multiplex amplification assay. In other embodiments, the assaying comprises separating and/or detecting the presence of SAM, SAH, 4-HNE, hsCRP or any combinations thereof in the biological sample with gas chromatography, mass spectrometry, high performance liquid chromatography, nuclear magnetic resonance (NMR) spectroscopy, an enzyme-coupled-assay, or any combinations thereof.

In some embodiments, the pre-determined reference ratio of SAM/SAH is a control ratio of SAM/SAH as measured in a biological sample of normal healthy subjects. In some instances, the control ratio of SAM/SAH as measured in a serum sample of the normal healthy subjects ranges from about 4 to about 12. In some embodiments, the pre-determined reference ratio of SAM/SAH is about 3.0 as measured in a plasma sample.

In some embodiments, the first pre-determined reference value of 4-HNE is a control value of 4-HNE as measured in a biological sample of normal healthy subjects. In some instances, the control value of 4-HNE as measured in a serum sample of the normal healthy subjects is about 0.24 μmol per liter of serum (or about 0.04 mg per liter of serum). In other embodiments, the first pre-determined reference value of 4-HNE is about 3 mg per liter of plasma as measured in a plasma sample.

In some embodiments, the second pre-determined reference value of hsCRP is a control value of hsCRP as measured in a biological sample of normal healthy subjects. In some instances, the control value of hsCRP as measured in a serum sample of the normal healthy subjects ranges from about 0.5 mg per liter of serum to about 4.5 mg per liter of serum. In some embodiments, the second pre-determined reference value of hsCRP is about 2.3 mg per liter of plasma as measured in a plasma sample.

In some embodiments, the method comprises determining a body measurement of the subject. In some instances, the body measurement comprises weight, height, waist circumference, hip circumference, body fat percentage, or any combinations thereof.

In some embodiments, the effective amount of the folate-comprising compound is about 7.5 mg/day to about 50 mg/day. In some instances, the effective amount of the folate-comprising compound is administered as a single daily dose. In other instances, the effective amount of the folate-comprising compound is administered in more than one divided doses per day. In some embodiments, the administration is oral.

In some embodiments, the composition is formulated to release at least a portion of the folate-comprising compound over a period of at least about 3-6 hours, upon the administration of the composition. In some instances, the release is a steady-state release.

In some embodiments, the anti-depressant drug comprises a selective serotonin reuptake inhibitor. In some instances, the selective serotonin reuptake inhibitor is selected from the group consisting of fluoxetine, citalopram, paroxetine, escitalopram, sertraline, and any combinations thereof.

In some embodiments, the method further comprises selecting for the subject a treatment comprising the folate-comprising compound administered in combination with the anti-depressant drug.

In some embodiments, the depression is major depressive disorder.

In some embodiments, the subject who is diagnosed as having depression is resistant to at least one antidepressant monotherapy. In some embodiments, the subject is an adult subject.

In some embodiments, the method of improving the effectiveness of an anti-depressant drug administered to a human subject results in improvement of at least one symptom of depression selected from low or depressed mood, anhedonia, low energy levels, guilt, decreased work and interests, psychomotor retardation, agitation, psychic anxiety, somatic anxiety, general somatic symptoms, reduced cognition or any combinations thereof.

In another aspect, provided herein is a method of treating at least one symptom of depression in a subject comprising administering a composition comprising an effective amount of a folate-comprising compound to a subject, who is diagnosed to have, or have a risk for depression, and is further determined to carry a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR), based on the recognition that the presence of the SNP allele(s) is associated with positive-symptom-reducing response to the folate-comprising compound.

In yet another aspect, provided herein is a method for selecting a treatment regimen for a subject diagnosed with depression. The method comprising:
assaying a test sample from the subject for the presence of one of the following SNPs:
   (i) a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR);
   (ii) a SNP at position 27 of SEQ ID NO: 23 (identified by rs4633) comprising two cytosine “C” alleles or the complement thereof, wherein the SEQ ID NO: 23 is a portion of a genomic nucleic acid sequence of catechol-O-methyltransferase (COMT); or
   (iii) a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT); and
optionally administering to the subject a folate-comprising compound (optionally in combination with an antidepressant drug), when the subject is determined to carry one of the MTR, COMT (rs4633) and COMT (rs4680) SNP biomarkers.

In another aspect, provided herein is an assay for selecting a treatment regimen for a human subject diagnosed as having depression or having a risk for depression. The assay comprising:
(a) analyzing a sample from the subject to determine the genotype of at least two genetic biomarkers selected from the group of methylenetetrahydrofolate reductase (MTHFR), methionine synthase (MTR), GTP cyclohydrolase 1 (GCH1), catechol-O-methyltransferase (COMT), and a combination thereof:
(b) detecting by genotyping for the presence or absence of a single nucleotide polymorphism (SNP) in each of the at least two genetic biomarkers, wherein the presence of the SNP is set forth as the following:
(i) a SNP at position 677 of SEQ ID NO: 1 or position 27 of SEQ ID NO: 7 as identified as rs1801133 comprising at least one thymine “T” allele for MTHFR;
(ii) a SNP at position 2756 of SEQ ID NO: 2 or position 27 of SEQ ID NO: 9 as identified as rs1805087 comprising at least one guanine “G” allele for MTR;
(iii) a SNP at position 27 of SEQ ID NO: 18 as identified as rs8007267 comprising at least one thymine “T” allele for GCH1;
(iv) a SNP at position 27 of SEQ ID NO: 24 as identified as rs4680 comprising two guanine “G” alleles for COMT; and
(c) selecting the treatment regimen comprising an effective amount of a folate-comprising compound based on the presence of the SNPs.

In some embodiments, the assay further comprises administering the treatment regimen.

In some embodiments, the at least two genetic biomarkers are the MTHFR and MTR pair. In other embodiments, the at least two genetic biomarkers are the GCH1 and COMT pair.

In some embodiments, step (a) further comprises determining at least one additional condition selected from the group consisting of obesity, SAM/SAH ratio, level of 4-HNE, level of hsCRP, and a combination thereof.

In some embodiments, step (b) further comprises detecting at least one of the following conditions:
(i) an expression level ratio of SAM to SAH smaller than a pre-determined reference ratio;
(ii) an expression level of 4-HNE greater than a first pre-determined reference value; and
(iii) an expression level of hsCRP greater than a second pre-determined reference value.

In some instances, obesity is determined, when any of the following conditions are present in the subject: a BMI value is 30 kg/m² or greater, a waist circumference is greater than 40 inches in men or greater than 35 inches in women, a waist-hip ratio is about 0.95 for men or above 0.8 for women, or a body fat percentage of at least about 25% in men or at least about 32% in women.

In some instances, the pre-determined reference ratio of SAM/SAH is from about 4 to about 12 if measured in a serum sample from a normal, healthy subject. In other instances, the pre-determined reference ratio of SAM/SAH is about 3.0 if measured in a plasma sample from a normal, healthy subject.

In some instances, the first pre-determined reference value of 4-HNE is about 0.24 mole per liter or about 0.04 mg per liter if measured in a serum sample from a normal, healthy subject. In other instances, the first pre-determined reference value of 4-HNE is about 3.0 mg per liter if measured in a plasma sample from a normal, healthy subject.

In some instances, the second pre-determined reference value of hsCRP is from about 0.5 mg per liter to about 4.5 mg per liter if measured in a serum sample from a normal, healthy subject. In other instances, the second pre-determined reference value of hsCRP is about 2.3 mg per liter if measured in a plasma sample from a normal, healthy subject.

In some embodiments, the sample is selected from the group of a blood sample, a serum sample, a plasma sample, a urine sample, a buccal sample, and a saliva sample.

In some embodiments, depression is major depressive disorder.

In some embodiments of the assay, the effective amount of the folate-comprising compound is about 15 mg/day to about 50 mg/day. In other embodiments, the effective amount of the folate-comprising compound is about 20 mg/day. In yet other embodiments, the effective amount of the folate-comprising compound is about 40 mg/day. In some instances, the folate-comprising compound is administered at about a 20 mg dose twice per day.

In some embodiments, the treatment regimen further comprises an antidepressant drug. In some instances, the antidepressant drug is a selective serotonin reuptake inhibitor (SSRI). In some embodiments, the selective serotonin reuptake inhibitor is selected from the group consisting of fluoxetine, citalopram, paroxetine, escitalopram, sertraline, or a combination thereof.

In some embodiments, the subject has an inadequate response or is resistant to an antidepressant monotherapy.

In some embodiments, the step of detecting the presence or absence of the SNP comprises a hybridization assay, an amplification assay, a primer extension assay, an oligonucleotide ligation assay, a sequencing assay or a combination thereof.

In some embodiments, the step of detecting the condition comprises an immunoassay, immunohistochemistry (IHC), gas chromatography (GC), mass spectrometry (MS), high performance liquid chromatography (HPLC), nuclear magnetic resonance (NMR) spectrometry, or flow cytometry.

In another aspect, provided herein is a method for treating at least one symptom of depression in a human subject diagnosed as having depression or having a risk for depression. The method comprising:
(a) analyzing a sample from the subject to determine the presence or absence of at least one combination of at least two single nucleotide polymorphisms (SNPs) selected from the group consisting of:
(i) a SNP at position 677 of SEQ ID NO: 1 or position 27 of SEQ ID NO: 7 as identified as rs1801133 comprising at least one thymine “T” allele for MTHFR and a SNP at position 2756 of SEQ ID NO: 2 or position 27 of SEQ ID NO: 9 as identified as rs1805087 comprising at least one guanine “G” allele for MTR; and
(ii) a SNP at position 27 of SEQ ID NO: 18 as identified by rs8007267 comprising at least one thymine “T” allele for GCH1 and a SNP at position 27 of SEQ ID NO: 24 as identified as rs4680 comprising two guanine “G” alleles for COMT, wherein the presence of at least one combination is associated with a symptom-reducing response to a folate-comprising compound; and
(b) administering a treatment regimen comprising an effective amount of the folate-comprising compound to the subject to treat at least one symptom of depression.

In some embodiments, the treatment regimen further comprises an antidepressant drug. In some instances, the antidepressant drug is a selective serotonin reuptake inhibitor (SSRI). In some embodiments, the selective serotonin reuptake inhibitor is selected from the group consisting of fluoxetine, citalopram, paroxetine, escitalopram, sertraline, or a combination thereof.

In some embodiments, the presence of at least one thymine “T” allele or the complement thereof at rs1801133 and the presence of at least one guanine “G” alleles or the complement thereof at rs1805087 are associated with a symptom-reducing response to the folate-comprising compound. In other embodiments, the presence of at least one thymine “T” allele or the complement thereof at rs8007267 and the presence of two guanine “G” alleles or the complement thereof at rs4860 are associated with a symptom-reducing response to the folate-comprising compound.

In some embodiments, depression is major depressive disorder. In some embodiments, the at least one symptom of depression is selected from the group consisting of depressed mood, guilt, reduced work or interests, psychomotor retardation, agitation, psychic anxiety, somatic anxiety, general somatic symptoms, cognition impairment, and a combination thereof.

In some embodiments, the subject is obese. In some instances, obesity is characterized by at least one of the following conditions present in the subject: a BMI value is 30 kg/m² or greater, a waist circumference is greater than 40 inches in men or greater than 35 inches in women, a waist-hip ratio is about 0.95 for men or above 0.8 for women, or a body fat percentage of at least about 25% in men or at least about 32% in women.

In some embodiments, the sample is selected from the group consisting of a blood sample, a serum sample, a plasma sample, a urine sample, a buccal sample, and a saliva sample.

In some embodiments of the method, the effective amount of the folate-comprising compound is about 15 mg/day to about 50 mg/day. In other embodiments, the effective amount of the folate-comprising compound is about 20 mg/day. In yet other embodiments, the effective amount of the folate-comprising compound is about 40 mg/day. In some instances, the folate-comprising compound is administered at about a 20 mg dose twice per day.

In some embodiments, the folate-comprising compound is administered orally.

In some embodiments, the folate-comprising compound is L-methylfolate.

In some embodiments, the subject has an inadequate response or is resistant to antidepressant monotherapy such as a SSRI.

In some embodiments, the method further comprises measuring the expression level of at least one biomarker and determining whether the level of the biomarker(s) is associated with a symptom-reducing response to a folate-comprising compound. In some instances, the additional biomarker is selected from the group consisting of SAM, SAH, 4-HNE, hsCRP and a combination thereof.

In some embodiments, the subject is likely to have a symptom-reducing response to the folate-comprising compound if one or more of the following conditions are met:
(a) an expression level ratio of SAM to SAH smaller than a pre-determined reference ratio;
(b) an expression level of 4-HNE greater than a first pre-determined reference value; and
(c) an expression level of hsCRP greater than a second pre-determined reference value.

In some instances, the pre-determined reference ratio of SAM/SAH is from about 4 to about 12 if measured in a serum sample from a normal, healthy subject. In other instances, the pre-determined reference ratio of SAM/SAH is about 3.0 if measured in a plasma sample from a normal, healthy subject.

In some instances, the first pre-determined reference value of 4-HNE is about 0.24 mole per liter or about 0.04 mg per liter if measured in a serum sample from a normal, healthy subject. In other instances, the first pre-determined reference value of 4-HNE is about 3.0 mg per liter if measured in a plasma sample from a normal, healthy subject.

In some instances, the second pre-determined reference value of hsCRP is from about 0.5 mg per liter to about 4.5 mg per liter if measured in a serum sample from a normal, healthy subject. In other instances, the second pre-determined reference value of hsCRP is about 2.3 mg per liter if measured in a plasma sample from a normal, healthy subject.

In yet another aspect, provided herein is a method for improving the effectiveness of an antidepressant drug administered to a human subject who is diagnosed as having depression or having a risk for depression. The method comprising:
administering a therapeutic composition comprising an effective amount of a folate-comprising compound in combination with the antidepressant drug if the subject is carrying at least one of the following combinations of single nucleotide polymorphisms (SNPs)
(i) a SNP at position 677 of SEQ ID NO: 1 or position 27 of SEQ ID NO: 7 as identified as rs1801133 comprising at least one thymine “T” allele for MTHFR and a SNP at position 2756 of SEQ ID NO: 2 or position 27 of SEQ ID NO: 9 as identified as rs1805087 comprising at least one guanine “G” allele for MTR; or
(ii) a SNP at position 27 of SEQ ID NO: 18 as identified by rs8007267 comprising at least one thymine “T” allele for GCH1 and a SNP at position 27 of SEQ ID NO: 24 as identified as rs4680 comprising two guanine “G” alleles for COMT.

In some embodiments, the subject has received antidepressant monotherapy. In some embodiments, the subject had an inadequate response to antidepressant monotherapy. In some instances, the inadequate response is based on a clinical assessment (e.g., neuropsychological test).

In some embodiments, the antidepressant drug is a selective serotonin reuptake inhibitor (SSRI). In some embodiments, the selective serotonin reuptake inhibitor is selected from the group consisting of fluoxetine, citalopram, paroxetine, escitalopram, sertraline, or a combination thereof.

In some embodiments of the method, the effective amount of the folate-comprising compound is about 15 mg/day to about 50 mg/day. In other embodiments, the effective amount of the folate-comprising compound is about 20 mg/day. In yet other embodiments, the effective amount of the folate-comprising compound is about 40 mg/day. In some instances, the folate-comprising compound is administered at about a 20 mg dose twice per day.

In some embodiments, the effective amount of the folate-comprising compound is administered as a single daily dose. In other embodiments, the effective amount of the folate-comprising compound is administered in at least two divided doses per day.

In some embodiments, the folate-comprising compound is administered orally.

In some embodiments, the folate-comprising compound is L-methylfolate.

In some embodiments, depression is major depressive disorder.

In another aspect, provided herein is a folate-comprising composition for use in the treatment of depression in a human subject who is diagnosed as having depression or having a risk for depression and carries at least two of the following SNPs selected from the group consisting of:
(i) the SNP at position 677 of SEQ ID NO: 1 or position 27 of SEQ ID NO: 7 as identified as rs1801133 comprising at least one thymine “T” allele for MTHFR;
(ii) the SNP at position 2756 of SEQ ID NO: 2 or position 27 of SEQ ID NO: 9 as identified as rs1805087 comprising at least one guanine “G” allele for MTR; (iii) the SNP at position 27 of SEQ ID NO: 18 as identified as rs8007267 comprising at least one thymine “T” allele for GCH1; and
(iv) the SNP at position 27 of SEQ ID NO: 24 as identified as rs4680 comprising two guanine “G” alleles for COMT.

In some embodiments, the at least two SNPs are the SNP at position 677 of SEQ ID NO: 1 or position 27 of SEQ ID NO: 7 as identified as rs1801133 comprising at least one thymine “T” allele for MTHFR and the SNP at position 2756 of SEQ ID NO: 2 or position 27 of SEQ ID NO: 9 as identified as rs1805087 comprising at least one guanine “G” allele for MTR. In other embodiments, the at least two SNPs are the SNP at position 27 of SEQ ID NO: 18 as identified as rs8007267 comprising at least one thymine “T” allele for GCH1 and the SNP at position 27 of SEQ ID NO: 24 as identified as rs4680 comprising two guanine “G” alleles for COMT.

In some embodiments, depression is major depressive disorder.

In some embodiments, the subject is receiving at least one antidepressant drug.

In some embodiments, the subject who carries the at least two of the SNPs is administered an adjunctive therapy comprising the folate-comprising composition and an antidepressant drug.

In some embodiments, the antidepressant drug is a selective serotonin reuptake inhibitor (SSRI).

In some embodiments, the folate-comprising compound comprises about 15 mg to about 50 mg of L-methylfolate. In some embodiments, the folate-comprising compound comprises about 20 mg of L-methylfolate.

In some embodiments, the folate-comprising compound has a pre-determined release profile. In some instances, the pre-determined release profile is a sustained release. In other instances, the pre-determined release profile is a pulsatile release. In yet other instances, the pre-determined release profile is a chrono-controlled release.

In some embodiments, the folate-comprising composition is formulated to release at least 30% of the folate-comprising compound over a period of at least 3 to 6 hours upon the administration of the composition.

In yet another aspect, provided herein is a kit for use in selecting a treatment regimen for a human subject diagnosed as having depression or having a risk for depression. The kit comprises at least one reagent for determining the presence or absence of at least two (e.g., 2, 3 or 4) of the following single nucleotide polymorphisms (SNPs) in a sample taken from the subject:
(i) a SNP at position 677 of SEQ ID NO: 1 or position 27 of SEQ ID NO: 7 as identified as rs1801133 comprising at least one thymine “T” allele for MTHFR;
(ii) a SNP at position 2756 of SEQ ID NO: 2 or position 27 of SEQ ID NO: 9 as identified as rs1805087 comprising at least one guanine “G” allele for MTR;
(iii) a SNP at position 27 of SEQ ID NO: 18 as identified as rs8007267 comprising at least one thymine “T” allele for GCH1;
(iv) a SNP at position 27 of SEQ ID NO: 24 as identified as rs4680 comprising two guanine “G” alleles for COMT; and instructions for use of the kit.

In some embodiments, the at least one reagent is selected from the group consisting of a restriction enzyme, an oligonucleotide, a nucleic acid probe, a polymerase and a combination thereof.

Other objects, features, and advantages of the present invention will be apparent to one of skill in the art from the following detailed description and figures.

### BRIEF DESCRIPTION OF THE DRAWINGS

FIG. 1A-B are tables summarizing mean changes in HAMD-28, HAM7, and CPFQ in MDD patients carrying one SNP marker (e.g., a rare variant on the indicated gene), or a combination of 2 indicated SNP markers, as compared to the MDD patients carrying fully normal on the respective gene(s), after both groups were treated with a folate-comprising compound, e.g., as an adjunct to an SSRI. FIG. 1A is a set of tables summarizing mean changes in HAMD-28, HAM7, and CPFQ in MDD patients carrying one SNP marker (e.g., a rare variant on the indicated gene), as compared to the MDD patients carrying fully normal on the respective gene, after both groups were treated with a folate-comprising compound, e.g., as an adjunct to an SSRI. FIG. 1B is a set of tables summarizing mean changes in HAMD-28, HAM7, and CPFQ in MDD patients carrying a combination of 2 indicated SNP markers, as compared to the MDD patients carrying fully normal on the respective gene(s), after both groups were treated with a folate-comprising compound, e.g., as an adjunct to an SSRI.

FIG. 2 is a set of result tables showing effects of the presence or absence of an indicated condition (a combination of 2 SNP markers, or a combination of 1 SNP marker and obesity indicator, e.g., BMI>30 kg/m²), in MDD patients on HAMD-28 or HAMD-7 value, when the patients were treated with a treatment regimen comprising a folate-comprising compound, e.g., as an adjunct to an SSRI.

FIG. 3 is a set of result tables showing effects of the presence or absence of an indicated condition (a single SNP marker), in MDD patients on HAMD-7 value, when the patients were treated with a treatment regimen comprising a folate-comprising compound, e.g., as an adjunct to an SSRI.

FIG. 4 is a set of result tables showing effects of the presence or absence of an indicated condition (a combination of 1 SNP marker and obesity indicator, e.g., BMI>30 kg/m²), in MDD patients on HAMD-28 or HAMD-7 value, when the patients were treated with a treatment regimen comprising a folate-comprising compound, e.g., as an adjunct to an SSRI.

FIG. 5 is a set of result tables showing effects of the presence or absence of an indicated condition (a combination of 2 SNP markers, or a combination of 1 SNP marker and obesity indicator, e.g., BMI>30 kg/m²), in MDD patients on CPFQ value, when the patients were treated with a treatment regimen comprising a folate-comprising compound, e.g., as an adjunct to an SSRI.

FIG. 6 is a set of result tables showing effects of the presence or absence of an indicated condition (a single SNP marker), in MDD patients on CPFQ value, when the patients were treated with a treatment regimen comprising a folate-comprising compound, e.g., as an adjunct to an SSRI.

FIG. 7A-B are a set of result tables showing the statistical analysis on the effects of genetic moderators (e.g., race, age, sex, and BMI) comparing biomarker positive versus biomarker negative subjects within the placebo (an antidepressant administered without a folate-comprising compound) or folate-comprising treatment (a folate-comprising compound administered as an adjuvant to the antidepressant) arms of clinical studies. FIG. 7A represents one set of patients. FIG. 7B represents a second set of patients. Results show statistically significant treatment effect within all of the indicated genetic modifiers (e.g., race, age, sex, and BMI) for subjects positive for the MTR 2756 AG or GG genotype (rs1805087) compared to subjects negative for this SNP. Subjects positive for the COMT CC (rs4633) or COMT GG (rs4680) SNP also show statistically significant treatment effect in at least one genetic moderator category.

FIG. 8A-B is a set of result tables showing the response rates of biomarker positive subjects within the placebo and folate-comprising treatment arms of clinical studies. A responder is indicated by a reduction of at least about 50% in HAMD-28 over the evaluation period. FIG. 8A shows the response rate of individual biomarkers. FIG. 8B shows the response rate of dual-marker combinations.

FIG. 9A-B show the HDRS-28 response rate (treatment minus placebo) with L-methylfolate stratified by markers involved with methylation (top) and markers involved with L-methylfolate metabolism (bottom) that were normal and putative positive. FIG. 9A shows the response rate for total, MTHFR CC, MTHFR CT/TT, FOLH1 AA, FOLH1 AG/GG, GCHFR AA, GCHFR TA/TT, RFC2 AA (e.g., RFC1 815 AA), RFC2 815 TT (e.g., RFC1 815 TT), RFC1 GG, RFC1 AA, RFC1 80 GG, RFC1 80 AA, GCH1 CC and GCH1 TC/TT. FIG. 9B shows the response rate for total, CACNA1C GG, CACNA1C AG/AA, DNMT3B GG, DNMT3B AG/AA, DRD2 129 CC, DRD2 129 TT CC, MTR 2756 AA, MTR 2756 AG/GG, COMT TT, COMT CC, COMT AA and COMT GG.

FIG. 10 shows the mean change from baseline for L-methylfolate vs. placebo on the HDRS-28 according to the presence of the individual markers, e.g., COMT A/GG, COMT GG, GCH1 CC, GCH1 TC/TT, MTR 2756 AA, MTR2756 AA/GG, MTHFR 677 CC, MTHFR 677 TC/TT, BMI<30 kg/m² and BMI≧30 kg/m². P-value is for L-methylfolate vs. placebo comparison.

FIG. 11 shows the pooled effect size for L-methylfolate vs. placebo according to the presence of the individual markers.

FIG. 12 shows the pooled mean change for L-methylfolate vs. placebo on the CGI, HDRS-7, and CPFQ scores according to the presence of dual combinations of biomarkers. NA indicates data not available due to small sample size. * p<0.05 and ** for p<0.001 for L-methylfolate vs. placebo.

### DETAILED DESCRIPTION OF THE INVENTION

### I. Introduction

The present invention provides in-part an assay for selecting a treatment regimen, e.g., adjunctive (add-on) therapy of a folate-comprising compound such as L-methylfolate, for a patient with depression or a risk for depression. Also provided is a method for treating at least one symptom of depression in a patient with depression or at risk for depression. In addition, a method for improving the effective amount of an antidepressant drug administered to a patient with depression or at risk for depression is also provided. The assays and/or methods include identifying whether the patient carries a synergistic dual-marker combination, such as the methylenetetrahydrofolate reductase (MTHFR 677 CT/TT; rs1801133) and methionine synthase (MTR 2756 AG/GG; rs1805087) SNP pair and/or the GTP cyclohydrolase 1 (GCH1 TC/TT; rs8007267) and catechol-O-methyltransferase (COMT Val158Met GG; rs4680) pair of markers. Also provided herein is a folate-comprising composition for use in treating a patient diagnosed with depression or at risk for depression who also carries a synergistic dual-marker combination.

The assay, methods and compositions provided herein are related to the descriptions found in, e.g., U.S. Patent App. Nos. US 2013/0172361 and US 2013/0267523 and International Patent. App. Pub. NO: WO 2013/074676, the disclosures of which are herein incorporated by reference in their entirety for all purposes.

### II. Definitions

As used herein, the following terms have the meanings ascribed to them unless specified otherwise.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

The term “biomarker” or “marker” includes any genetic marker, biochemical marker, serological marker, or other clinical characteristic that can be used in predicting, identifying, evaluating, assessing, determining, monitoring, and/or optimizing folate-comprising compound response, efficacy, toxicity, and/or resistance according to the assays and/or methods provided herein

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G” a thymine “T” or a cytosine “C”) or RNA (e.g. an A, a G. an uracil “U” or a “C”). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length

The term “complementary” or “complement” as used herein refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an anti-parallel fashion, at least one nucleotide residue of the first region is capable of base pairing with a residue of the second region. Preferably, the first region comprises a first portion and the second region comprises a second portion, whereby, when the first and second portions are arranged in an anti-parallel fashion, such that at least about 50%, and preferably at least about 75%, at least about 90%, or at least about 95% or at least 100% of the nucleotide residues of the first portion are capable of base pairing with nucleotide residues in the second portion. More preferably, all nucleotide residues of the first portion are capable of base pairing with nucleotide residues in the second portion.

The terms “variant”, “variance”, “mutation” or “polymorphism” are used interchangeably herein, and refer to a difference in nucleic acid sequence among members if a population of individuals. Polymorphisms can sometimes be referred to as “single nucleotide polymorphism” or “SNP” when they vary at a single nucleotide. In some embodiments, polymorphisms can be synonymous or non-synonymous. Synonymous polymorphisms when present in the coding region or non-coding region typically do not result in an amino acid change, but can result in altered mRNA stability or altered alternative splice sites. Non-synonymous polymorphism, when present in the coding region, can result in the alteration of one or more codons resulting in an amino acid replacement in the amino acid chain. Such mutations and polymorphisms may be either heterozygous or homozygous within an individual. Homozygous individuals have identical alleles at one or more corresponding loci on homologous chromosomes, while heterozygous individuals have two different alleles at one or more corresponding loci on homologous chromosomes. A polymorphism is thus said to be “allelic,” in that, due to the existence of the polymorphism, some members of a species carry a gene with one sequence (e.g., the normal or wild-type “allele”), whereas other members may have an altered sequence (e.g., the variant or, mutant “allele”).

In defining a SNP position, SNP allele, or nucleotide sequence, reference to an adenine “A”, a thymine “T” (uridine “U”), a cytosine “C”, or a guanine “G” at a particular site on one strand of a nucleic acid molecule also defines the thymine “T” (uridine “U”), adenine “A”, guanine “G”, or cytosine “C” (respectively) at the corresponding site on a complementary strand of the nucleic acid molecule. Thus, reference can be made to either strand in order to refer to a particular SNP position, SNP allele, or nucleotide sequence.

The term “genotype” refers to the specific allelic composition of an entire cell or a certain gene, whereas the term “phenotype” refers to the detectable outward manifestations of a specific genotype.

The term “allele”, as used herein, refers to one member of a pair of different forms of a gene. As used herein alleles refer to coding and to non-coding sequences. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The term “SAM” refers to S-adenosyl methionine, commonly known as SAM, or SAM-e, or AdoMet, which is a natural compound found in all living cells. It is one of the most used enzymatic substrates in biochemical reactions. S-Adenosyl methionine is a common cosubstrate involved in methyl group transfers. It is made from adenosine triphosphate (ATP) and methionine by methionine adenosyltransferase. SAM is used in metabolic pathways such as transmethylation, transsulfuration, and aminopropylation.

The term “SAH” refers to S-adenosylhomocysteine which is formed by the demethylation of S-adenosyl-L-methionine. The relative levels of S-adenosylmethionine (SAM) and S-adenosylhomocysteine (SAH) in blood, plasma or serum can be used to predict metabolic changes associated with nutritional and chronic disease states, such as cardiovascular disease, some cancers and neuropsychiatric disease.

The term “SAM/”SAH ratio” refers to the relative levels of S-adenosyl methionine to S-adenosylhomocysteine. In some instances, a decrease in the SAM/SAH ratio is associated with increased homocysteine in serum and an increase in SAH.

The term “4-HNE” refers to 4-hydroxynonenal, or 4-hydroxy-2-nonenal which is an α,β-unsaturated hydroxyalkenal that is produced by lipid peroxidation in cells. 4-HNE is the primary α,β-unsaturated hydroxyalkenal formed in this process. It is found throughout body tissue, and in higher quantities during oxidative stress due to the increase in the lipid peroxidation chain reaction in stress events. It appears to play a key role in cell signal transduction in a variety of pathways from cell cycle events to cellular adhesion. 4-HNE is also considered as possible causal agents of numerous diseases, such as chronic inflammation, neurodegenerative diseases, adult respiratory distress syndrome, atherogenesis, diabetes and different types of cancer.

The term “hsCRP” refers to the high-sensitivity c-reactive protein which is found in, e.g., blood, serum, and plasma. Elevated levels of hsCRP in serum and plasma have been detected in patients with symptoms of depression, as well as patients with an inflammatory disease. For example, it has been reported that major depression is associated with increased levels of hsCRP and inflammatory markers such as IL-6 and TNF-α (see, e.g., Dinan T G. Current Opinion Psychia., 2009, 22(1):32-6).

The term “depression” refers to a mental state of depressed mood characterized by feelings of sadness, despair and discouragement. In some instances, depression is a clinical symptom, and can include, but not limited to, major depressive disorder (including single episode and recurrent), unipolar depression, treatment-refractory depression, resistant depression, anxious depression and dysthymia (also referred to as dysthymic disorder). Further, the term “depression” can encompass any major depressive disorder, dysthymic disorder, mood disorders due to medical conditions with depressive features, mood disorders due to medical conditions with major depressive-like episodes, substance-induced mood disorders with depressive features and depressive disorder not otherwise specific as defined by their diagnostic criteria, as listed in the American Psychiatric Association's Diagnostic and Statistical Manual of Mental Disorders, 4th Edition (DSM-IV) or any later edition thereof, or the World Health Organization's International Statistical Classification of Diseases and Related Health Problems (ICD-10).

The terms “treatment” and “treating” as used herein, with respect to treatment of a disease, means preventing the progression of the disease, or altering the course of the disorder (for example, but are not limited to, slowing the progression of the disorder), or partially reversing a symptom of the disorder or reducing one or more symptoms and/or one or more biochemical markers in a subject, preventing one or more symptoms from worsening or progressing, promoting recovery or improving prognosis.

The term “treatment regimen” refers to a clinically relevant alleviation of at least one symptom associated with a disease or disorder, e.g., depression.

The term “negative response” includes a worsening of a disorder condition in a patient receiving therapy, such that the patient experiences increased or additional signs or symptoms of the disorder.

The term “positive response” includes an improvement in a patient with a disorder condition, such that the therapy alleviates signs or symptoms of the disorder.

The term “antidepressant” or “antidepressant drug” refers to any pharmaceutical agent which treats depression. In some embodiments, the antidepressant drug administered to the subject in accordance with the methods described herein can be any conventional pharmaceutical agent which is commonly indicated for treating depression.

The term “folate-comprising compound” or “folate-comprising drug” refers to a compound containing an effective amount of at least one folate for use in the methods described herein. Folate is a form of the water-soluble vitamin B9. The term “folate” encompasses the naturally-occurring form of folate, folic acid (also known as vitamin B9 or folacin) and metabolites or derivatives thereof such as methylfolate, tetrahydrofolate, and methyltetrahydrofolate. The term “folate” can also refer to both pteroic acid monoglutamate (folic acid) and reduced forms such as dihydrofolates and tetrahydrofolates, e.g. 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid and tetrahydrofolic acid, polyglutamates thereof, optical isomers thereof (e.g., optically pure natural isomers thereof, and also mixtures of optical isomers such as racemic mixtures), derivatives thereof, pharmaceutically acceptable salts and esters thereof, glucosamine salts thereof, and galactosamine salts thereof.

The term “pharmaceutically acceptable salts and esters” refers to pharmacologically acceptable and pharmaceutically acceptable salts and esters. Pharmacologically and pharmaceutically acceptable salts can include, but are not limited to, alkali metal or alkaline earth metal salts, e.g., sodium, potassium, magnesium or calcium salts. Pharmacologically and pharmaceutically acceptable esters can include, but are not limited to, C₁-C₄ alkyl, C₅ cycloalkyl or C₆ cycloalkyl, phenyl, C₁-C₄ alkylphenyl, benzyl or C₁-C₄-alkylbenzyl esters. The esters can be monoesters or diesters. Diesters can be homogeneous or heterogeneous. In some embodiments, pharmacologically and pharmaceutically acceptable esters can be homogeneous diesters such as C₁-C₄ dialkylesters, for example dimethyl- or diethylesters. The term “adjuvant,” “adjunctive agent,” “adjunctive drug,” or “adjunctive therapy” as used herein generally refers to any agent or entity which increases the effect of another agent or entity. In certain embodiments, the term “adjuvant” is used herein in reference to a folate-comprising compound as an add-on agent (drug) to increase or enhance the effect (e.g., efficacy and/or therapeutic effect) of an antidepressant drug.

As used herein, the term “administer” or “administration” refers to the placement of a composition into a subject by a method or route which results in at least partial localization of the composition at a desired site such that desired effect is produced. Routes of administration suitable for the methods described herein can include both local and systemic administration. Generally, local administration results in a higher amount of an antidepressant (e.g., SSRI) and/or a folate-comprising compound being delivered to a specific location (e.g., serotonin receptors in the central and/or peripheral nervous systems) as compared to the entire body of the subject, whereas, systemic administration results in delivery of an antidepressant (e.g., SSRI) and/or a folate-comprising compound to essentially the entire body of the subject. In some embodiments, the compositions described herein are administered to subjects with depression orally. In other embodiments, the compositions described herein can be administered to subjects with depression by injection.

The term “an inadequate response” in the context of a therapy refers to a drug response that has failed to reduce or minimize a symptom(s) of a disease/disorder and thus, does not provide a therapeutic benefit to the patient. In some instances, an inadequate response for a patient with depression includes no or little decrease in the severity of a symptom of depression according to a neuropsychological assessment scale.

The term “symptom-reducing response” or “positive symptom-reducing response” refers to a drug response that reduces or minimizing a symptom of a disease/disorder, thereby providing a therapeutic benefit to the subject. In some instances, a symptom-reducing response for a subject with depression is characterized as the reduction of at least one symptom of depression, by a clinically-relevant amount of a drug as determined by a skilled practitioner, without a significant adverse effect on the subject.

The term “subject” or “patient” or “individual” typically includes humans, but can also include other animals, such as, e.g., other primates, rodents, canines, felines, equines, ovines, porcines, and the like.

The term “normal healthy subject” refers to a subject who has no symptoms of any diseases or disorders, or who is not identified with any diseases or disorders, or who is not on any medication treatment, or a subject who is identified as healthy by physicians based on medical examinations.

### III. Detailed Description of Embodiments

The assays, methods and compositions described herein directed to individual who are diagnosed with depression, e.g., major depressive disorder, or are at risk for depression. Depression can be diagnosed using standard clinical criteria, e.g., the DSM-IV-TR system. For example, the DSM-IV system for diagnosing MDD requires the presence of at least five out of the ten depressive symptoms including depressed mood or irritable, decreased interest or pleasure, significant weight change (5%) or change in appetite, change in sleep (e.g., insomnia or hypersomnia), change in activity, fatigue or loss of energy, guilt or worthlessness, diminished concentration and suicidiality. In addition, the symptoms should be represent for at least two weeks and each symptom should be at sufficient severity for nearly every day.

Generally, depression evaluated by a clinician using, e.g., the criteria list in the DSM-IV or efficacy measures (neuropsychological assessments) such as the Hamilton Depression Rating Scale (HAMD-28 or HAMD-7), the Clinical Global Impression (CGI) Scale, the Montgomery-Asberg Depression Rating Scale (MADRS), the Beck Depression Inventory (BDI), the Zung Self-Rating Depression Scale, the Wechsler Depression Rating Scale, the Raskin Depression Rating Scale, the Inventory of Depressive Symptomatology (IDS), and the Quick Inventory of Depressive Symptomatology (QIDS). For example, measurable lessening of depression (improvement of depression) includes any clinically significant decline in a measurable marker or symptom, such as measuring markers for depression in the blood, e.g., red blood cell folate, serum folate, serum MTHF, or assessing the degree of depression, e.g., using a neuropsychological assessment.

For example, a score of 0-7 on HAMD is typically considered to be normal. Scores of 20 or higher indicate moderate, severe, or very severe depression. Questions 18-21 may be recorded to give further information about the depression (such as whether diurnal 1 variation or paranoid symptoms are present), but are not necessary part of the scale. Thus, a reduction of symptoms can be considered clinically relevant if, e.g., the HAMD score is decreased to under, e.g., 20.

In one aspect, some embodiments provided herein relate to assays or assay methods for selecting a treatment regimen for a patient with depression or at risk for depression by genotyping the patient to determine if the presence of a synergistic pair of folate-responsive markers. In some embodiments, the genotype that indicates folate responsiveness includes a SNP in MTHFR (identified by rs1801133) comprising at least one thymine “T” allele or the complement thereof, and a SNP in MTR (identified by rs1805087) comprising at least one guanine “G” allele or the complement thereof (e.g., MTHFR 677 CT/TT and MTR 2756 AG/GG). In other embodiments, the genotype that indicates folate responsiveness includes a SNP in GCH1 (identified by rs8007267) comprising at least one thymine “T” allele or the complement thereof, and a SNP in COMT (identified by rs4680) comprising two guanine “G” alleles or the complement thereof (e.g., GCH1 TC/TT and COMT Val158Met GG). In yet other embodiments, the genotype that indicates folate responsiveness includes any synergistic dual marker described in Table 4. A treatment regimen comprising an effective amount of a folate-comprising compound is selected for the patient if it is determined that the patient carries the folate-responsive SNP pairs.

In some instances, the adjunctive therapy of folate-comprising compound is prescribed if the patient carries the SNP pair of MTHFR 677 CT/TT and MTR 2756 AG/GG, and is obese. In some instances, the adjunctive therapy is prescribed if the patient carries the SNP pair of MTHFR 677 CT/TT and MTR 2756 AG/GG pair, and has a SAM/SAH ratio less than a pre-determined reference ratio. In other instances, the adjunctive therapy is prescribed if the patient carries the SNP pair of MTHFR 677 CT/TT and MTR 2756 AG/GG, and has an expression level of 4-HNE greater than a pre-determined reference value. In yet some instances, the adjunctive therapy of folate-comprising compound is prescribed if the patient carries the SNP pair of MTHFR 677 CT/TT and MTR 2756 AG/GG, and has an expression level of hsCRP level is greater than a pre-determined reference value.

In some instances, the adjunctive therapy of folate-comprising compound is prescribed if the patient carries the SNP pair of GCH1 TC/TT and COMT Val158Met GG, and is obese. In some instances, the adjunctive therapy is prescribed if the patient carries the SNP pair of GCH1 TC/TT and COMT Val158Met GG, and has a SAM/SAH ratio less than a pre-determined reference ratio. In other instances, the adjunctive therapy is prescribed if the patient carries the SNP pair of GCH1 TC/TT and COMT Val158Met GG, and has an expression level of 4-HNE greater than a pre-determined reference value. In yet some instances, the adjunctive therapy is prescribed if the patient carries the SNP pair of GCH1 TC/TT and COMT Val158Met GG, and has an expression level of hsCRP level is greater than a pre-determined reference value.

In another aspect, some embodiments provided herein relate to methods of treating a human subject with depression comprises administering to the subject a folate-comprising compound (and optionally in combination with an antidepressant drug). In one embodiment, the method of treating a depressed subject by administering a folate-comprising compound (and optionally in combination with an antidepressant drug) is based on the determination that the subject carries both folate-responsive markers: a SNP in MTHFR (identified by rs1801133) comprising at least one thymine “T” allele or the complement thereof, and a SNP in MTR (identified by rs1805087) comprising at least one guanine “G” allele or the complement thereof. In another embodiment, the method of treating a human subject with depression by administering to the subject a folate-comprising compound (and optionally in combination with an antidepressant drug) is based on the determination that the subject carries both folate-responsive markers: a SNP in GCH1 (identified by rs8007267) comprising at least one thymine “T” allele or the complement thereof, and a SNP in COMT (identified by rs4680) comprising two guanine “G” alleles or the complement thereof.

In some instances, the administration of a folate-comprising compound is selected for a patient who carries the SNP pair of MTHFR 677 CT/TT and MTR 2756 AG/GG, and is obese. In some instances, the administration of a folate-comprising compound is selected for a patient who carries the SNP pair of MTHFR 677 CT/TT and MTR 2756 AG/GG, and has a SAM/SAH ratio less than a pre-determined reference ratio. In other instances, the administration of a folate-comprising compound is selected for a patient who carries the SNP pair of MTHFR 677 CT/TT and MTR 2756 AG/GG, and has an expression level of 4-HNE greater than a pre-determined reference value. In yet some instances, the administration of a folate-comprising compound is selected for a patient who carries the SNP pair of MTHFR 677 CT/TT and MTR 2756 AG/GG, and has an expression level of hsCRP level is greater than a pre-determined reference value.

In some instances, the administration of a folate-comprising compound is selected for a patient who carries the SNP pair of GCH1 TC/TT and COMT Val158Met GG, and is obese. In some instances, the administration of a folate-comprising compound is selected for a patient who carries the SNP pair of GCH1 TC/TT and COMT Val158Met GG, and has a SAM/SAH ratio less than a pre-determined reference ratio. In other instances, the administration of a folate-comprising compound is selected for a patient who carries the SNP pair of GCH1 TC/TT and COMT Val158Met GG, and has an expression level of 4-HNE greater than a pre-determined reference value. In yet some instances, the administration of a folate-comprising compound is selected for a patient who carries the SNP pair of GCH1 TC/TT and COMT Val158Met GG, and has an expression level of hsCRP level is greater than a pre-determined reference value.

The subject with depression being treated with the methods described herein can be a subject currently taking an antidepressant. Accordingly, the methods of treating a human subject with depression described herein can also be used to select a human subject to be treated with the combination of a folate-comprising compound and an antidepressant to improve the effectiveness of an antidepressant drug currently taken by a subject. Accordingly, if the human subject currently taking an antidepressant is determined to carry one or more of the synergistic dual-biomarker combinations, the subject can be further administered or prescribed with a folate-comprising compound as an adjuvant to the antidepressant.

In another aspect, some embodiments provided herein relate to methods for treating at least one symptom of depression in a subject with depression or at risk for depression. The symptom includes depressed mood, guilt, reduced work or interests, psychomotor retardation, agitation, psychic anxiety, somatic anxiety, general somatic symptoms, cognition impairment, or any combinations thereof. In some embodiments, the method for treating at least one symptom of depression in a subject comprises genotyping the subject to determine if the subject carries a synergistic pair of SNPs such as (i) a SNP in MTHFR (identified by rs1801133) comprising at least one thymine “T” allele or the complement thereof, and a SNP in MTR (identified by rs1805087) comprising at least one guanine “G” allele or the complement thereof, or (ii) a SNP in GCH1 (identified by rs8007267) comprising at least one thymine “T” allele or the complement thereof and a SNP in COMT (identified by rs4680) comprising two guanine “G” alleles or the complement thereof. A treatment regimen comprising an effective amount of a folate-comprising compound is administered to the subject if the presence of the synergistic SNPs pairs are determined.

In some instances, the treatment regimen comprising an effective amount of a folate-comprising compound is selected for a patient who carries the SNP pair of MTHFR 677 CT/TT and MTR 2756 AG/GG, and is obese. In some instances, the treatment regimen is selected for a patient who carries the SNP pair of MTHFR 677 CT/TT and MTR 2756 AG/GG, and has a SAM/SAH ratio less than a pre-determined reference ratio. In other instances, the treatment regimen is selected for a patient who carries the SNP of MTHFR 677 CT/TT and MTR 2756 AG/GG, and has an expression level of 4-HNE greater than a pre-determined reference value. In yet some instances, the treatment regimen is selected for a patient who carries the SNP pair of MTHFR 677 CT/TT and MTR 2756 AG/GG, and has an expression level of hsCRP level is greater than a pre-determined reference value.

In some instances, the treatment regimen comprising an effective amount of a folate-comprising compound is selected for a patient who carries the SNP pair of GCH1 TC/TT and COMT Val158Met GG, and is obese. In some instances, the treatment regimen is selected for a patient who carries the SNP pair of GCH1 TC/TT and COMT Val158Met GG, and has a SAM/SAH ratio less than a pre-determined reference ratio. In other instances, the treatment regimen is selected for a patient who carries the SNP pair of GCH1 TC/TT and COMT Val158Met GG, and has an expression level of 4-HNE greater than a pre-determined reference value. In yet some instances, the treatment regimen is selected for a patient who carries the SNP pair of GCH1 TC/TT and COMT Val158Met GG, and has an expression level of hsCRP level is greater than a pre-determined reference value.

In some embodiments, at least one symptom of depression is alleviated by a “clinically relevant amount” or “effective amount” as evaluated by a physician or a psychologist, as compared to a control (e.g., a subject having the same or similar degree of depression as the treated subject is administered without a folate-comprising compound, or a subject who has met none of the conditions described herein is administered with treatment regimen comprising a folate-comprising compound).

For example, in some embodiments, at least one neuropsychological test is improved (e.g., HAMD-17 rating is decreased) by at least about 10%, at least about 15%, at least about 20%, at least about 30%, at least about 40%, or at least about 50%, after the subject is treated with an treatment regimen. In another embodiment, at least one neuropsychological test is improved (e.g., HAMD-17 rating is decreased) by more than 50%, e.g., at least about 60%, or at least about 70%. In one embodiment, at least one neuropsychological test is improved (e.g., HAMD-17 rating is decreased) by at least about 80%, at least about 90% or greater, as compared to a control (e.g., a subject having the same or similar degree of depression as the treated subject is administered without a folate-comprising compound, or a subject who has met none of the conditions described herein is administered with treatment regimen comprising a folate-comprising compound). In some embodiments, at least one symptom of depression can be alleviated by a clinically relevant amount as evaluated by a physician or a psychologist within a treatment period of at least about 10 days, including, e.g., at least about 20 days, at least about 30 days, at least about 40 days, or longer. In some embodiments, at least one neuropsychological test is improved (e.g., HAMD-17 rating is decreased) by at least about 10%, at least about 15%, at least about 20%, at least about 30%, at least about 40%, or at least about 50% or higher within a treatment period of at least about 10 days, including, e.g., at least about 20 days, at least about 30 days, at least about 40 days, or longer.

In another aspect, some embodiments provided herein relate to methods of increasing the effectiveness of an antidepressant drug administered to a human subject with depression, which comprises administering to the subject a folate-comprising compound in combination with the antidepressant drug, based on the determination that the subject carries at least two folate-responsive markers, the combination of which yields a synergistic effect. In some embodiments, the method of increasing the effectiveness of an antidepressant drug administered to a human subject comprises administering to a subject, who is diagnosed to have depression, and is further determined to carry one or more (e.g., 1, 2, 3 or more) of the synergistic dual-biomarker combinations, based on the recognition that the combination of the indicated biomarkers is associated with increasing the effectiveness of the antidepressant drug when administered in combination with the folate-comprising compound. In some embodiments, the method of increasing the effectiveness of an antidepressant drug comprises administering to the subject a folate-comprising compound in combination with the antidepressant drug, based on the determination that the subject carries both folate-responsive markers: a SNP in MTHFR (identified by rs1801133) comprising at least one thymine “T” allele or the complement thereof, and a SNP in MTR (identified by rs1805087) comprising at least one guanine “G” allele or the complement thereof. In other embodiments, the method of increasing the effectiveness of an antidepressant drug comprises administering to the subject a folate-comprising compound in combination with the antidepressant drug, based on the determination that the subject carries both folate-responsive markers: a SNP in GCH1 (identified by rs8007267) comprising at least one thymine “T” allele or the complement thereof, and a SNP in COMT (identified by rs4680) comprising two guanine “G” alleles or the complement thereof.

In some embodiments, the method of determining and/or improving the effectiveness of an antidepressant drug administered to a human subject, e.g., by determining if the human subject is amenable to folate or a derivative thereof as an adjuvant, e.g., using the assay described herein.

In some instances, the subject has an inadequate response to the antidepressant drug. An adequate response to an antidepressant drug may be a 50-percent decrease in symptom severity. For instance, a patient responding to an antidepressant has a 50% or greater reduction in neuropsychological test score compared to baseline. Remission from depression may be defined as being free or nearly free of symptoms for the episode of depression.

In some instances, the adjunctive therapy of folate-comprising compound is prescribed if the patient carries the SNP pair, MTHFR 677 CT/TT and MTR 2756 AG/GG pair, and is obese. In some instances, the adjunctive therapy is prescribed if the patient carries the SNP pair, MTHFR 677 CT/TT and MTR 2756 AG/GG pair, and has a SAM/SAH ratio less than a pre-determined reference ratio. In other instances, the adjunctive therapy is prescribed if the patient carries the SNP pair, MTHFR 677 CT/TT and MTR 2756 AG/GG pair, and has an expression level of 4-HNE greater than a pre-determined reference value. In yet some instances, the adjunctive therapy is prescribed if the patient carries the SNP pair, MTHFR 677 CT/TT and MTR 2756 AG/GG pair, and has an expression level of hsCRP level is greater than a pre-determined reference value.

In some instances, the adjunctive therapy of folate-comprising compound is prescribed if the patient carries the SNP pair, GCH1 TC/TT and COMT Val158Met GG pair, and is obese. In some instances, the adjunctive therapy is prescribed if the patient carries the SNP pair, GCH1 TC/TT and COMT Val158Met GG pair, and has a SAM/SAH ratio less than a pre-determined reference ratio. In other instances, the adjunctive therapy is prescribed if the patient carries the SNP pair, GCH1 TC/TT and COMT Val158Met GG pair, and has an expression level of 4-HNE greater than a pre-determined reference value. In yet some instances, the adjunctive therapy is prescribed if the patient carries the SNP pair, GCH1 TC/TT and COMT Val158Met GG pair, and has an expression level of hsCRP level is greater than a pre-determined reference value.

In yet another aspect, some embodiments provided herein relate to folate-comprising compounds for use to treat a subject with depression or at risk for depression if the subject has a genotype indicative of folate-responsiveness. In some embodiments, the subject is likely to respond to a folate-comprising compound if carrying a SNP in MTHFR (identified by rs1801133) comprising at least one thymine “T” allele or the complement thereof, and a SNP in MTR (identified by rs1805087) comprising at least one guanine “G” allele or the complement thereof. In other embodiments, the subject is likely to respond to a folate-comprising compound if carrying a SNP in GCH1 (identified by rs8007267) comprising at least one thymine “T” allele or the complement thereof, and a SNP in COMT (identified by rs4680) comprising two guanine “G” alleles or the complement thereof.

In some instances, the folate-comprising compound is administered to a patient who carries the SNP pair, MTHFR 677 CT/TT and MTR 2756 AG/GG pair, and is obese. In some instances, the folate-comprising compound is administered to a patient who carries the SNP pair, MTHFR 677 CT/TT and MTR 2756 AG/GG pair, and has a SAM/SAH ratio less than a pre-determined reference ratio. In other instances, the folate-comprising compound is administered to a patient who carries the SNP pair, MTHFR 677 CT/TT and MTR 2756 AG/GG pair, and has an expression level of 4-HNE greater than a pre-determined reference value. In yet some instances, the folate-comprising compound is administered to a patient who carries the SNP pair, MTHFR 677 CT/TT and MTR 2756 AG/GG pair, and has an expression level of hsCRP level is greater than a pre-determined reference value.

In some instances, the folate-comprising compound is administered to a patient who carries the SNP pair, GCH1 TC/TT and COMT Val158Met GG pair, and is obese. In some instances, the folate-comprising compound is administered to a patient who carries the SNP pair, GCH1 TC/TT and COMT Val158Met GG pair, and has a SAM/SAH ratio less than a pre-determined reference ratio. In other instances, the folate-comprising compound is administered to a patient who carries the SNP pair, GCH1 TC/TT and COMT Val158Met GG pair, and has an expression level of 4-HNE greater than a pre-determined reference value. In yet some instances, the folate-comprising compound is administered to a patient who carries the SNP pair, GCH1 TC/TT and COMT Val158Met GG pair, and has an expression level of hsCRP level is greater than a pre-determined reference value.

In some embodiments, the folate-comprising compound can be administered in an amount effective to reduce at least one symptom (e.g., but not limited to, low mood, anhedonia, low energy, insomnia, agitation, anxiety and/or weight loss) associated with depression, e.g., major depressive disorders. In some embodiments, the effective amount of a folate-comprising compound can provide at least about 0.1 to about 1 mg/kg body weight per day administration to the human subject. In some embodiments, the effective amount of the folate-comprising compound is about 7.5 mg/day to about 50 mg/day. In some embodiments, the effective amount of a folate-comprising compound can provide at least about 15 mg/day to about 50 mg/day administration to the human subject. In one embodiment, the effective amount of a folate-comprising compound can provide at least about 15 mg/day of folate administration to the human subject. In other embodiments, the effective amount of a folate-comprising compound can provide at least about 20 mg/day of folate administration to the human subject. In other embodiments, the effective amount of a folate-comprising compound can provide at least about 40 mg/day of folate administration to the human subject. In some instance, the folate-comprising compound is administered once a day as a single daily dose or twice a day as a divided dose via any suitable administration route, e.g., oral administration. For example, a single dose of 20 mg can be administered per day. Alternatively, two doses of 20 mg can be administered a day. In other instances, the folate-comprising compound is administered more than twice a day.

In some embodiments, the folate-comprising compound is administered in combination with an antidepressant drug. In some instances, the therapeutic effect (e.g., reducing at least one of core symptoms associated with depression) can be synergistic when a human subject, who is determined to carry at least two of the folate-responsive biomarkers, is administered with an adjunctive therapy of the folate-comprising compound. The term “synergy” or “synergistic” as used herein in the context of a therapeutic effect refers to the combined effect of at least two or more agents being greater than the sum of their individual effects. In particular, the term “synergy” or “synergistic” as used herein refers to the combined therapeutic effect associated with a human subject carrying two or more folate-responsive biomarkers that, when the human subject is administered with a treatment comprising a folate-comprising compound, is greater than the sum of the therapeutic effect associated with the individual folate-responsive biomarkers (additive effect). In some embodiments, the synergistic effect can be greater than the additive effect by at least about 10%, at least about 20%, at least about 30%, at least about 40%, at least about 50%, at least about 60%, at least about 70%, at least about 80%, at least about 90%, at least about 95% or more (e.g., as determined by HAMD-28, or any other equivalent measures for evaluating depression symptom). In some embodiments, the synergistic effect can be greater than the additive effect by at least about 1.5-fold, at least about 2-fold, at least about 3-fold or higher, e.g., as determined by HAMD-28, or any other equivalent measures as described elsewhere in this application, for evaluating depression symptoms.

In some embodiments, the therapeutic effect can be determined using a neuropsychological test such as HAMD-28, CGI-S and CPFQ. Accordingly, in some embodiments, the synergistic therapeutic effect refers to a total reduction in HAMD-28 score that, when a human subject carrying at least two or more folate-responsive biomarkers is administered with a folate-comprising compound, is greater than the sum of individual reduction in HAMD-28 associated with each respective biomarker. In some embodiments, the synergistic effect on mean change in HAMD-28 can be greater than the additive effect by at least about 10%, at least about 20%, at least about 30%, at least about 40%, at least about 50%, at least about 60%, at least about 70%, at least about 80%, at least about 90%, at least about 95% or more. In some embodiments, the synergistic effect on the mean change in HAMD-28 can be greater than the additive effect by at least about 1.5-fold, at least about 2-fold, at least about 3-fold or higher.

For example, patients who were determined to carry a single folate-responsive biomarker (i) (i.e., at least one “T” variant in the MTHFR) showed a mean change in HAMD-28 of about −3.0 to about −5.0 after administration with a folate-comprising compound (e.g., optionally in combination with an antidepressant), and also shows that patients who were determined to carry a single folate-responsive biomarker (iii) (i.e., at least one “G” variant in the MTR gene) showed a mean change in HAMD-28 of about −8.2 after administration with a folate-comprising compound (e.g., optionally in combination with an antidepressant). When patients carry both of the gene mutations, the patients' mean change in HAMD-28 in response to the treatment comprising a folate-comprising compound (e.g., optionally in combination with an antidepressant drug) was about −23.3, which is not only greater than the effect associated with each biomarker individually, but is also surprisingly greater than the sum of the effects associated with each individual biomarkers (i.e., the additive effect would yield a mean change in HAMD-28 of about −11.2 to about −13.2) by at least about 2-fold.

A. Folate-Responsive Genetic Biomarkers

The SNPs that can predict the therapeutic efficacy of a folate-comprising compound (e.g., in a combination therapy to increase the efficacy of an antidepressant) administered to a subject for the treatment of depression include at least two of the SNPs as follows: rs1801133 present in methylenetetrahydrofolate reductase (MTHFR); rs2274976 present in MTHFR; rs1805087 present in methionine synthase (MTR); rs1801394 present in methionine synthase reductase (MTRR); rs1006737 present in calcium channel, voltage-dependent, L-type, alpha 1C subunit (CACNA1C); rs1883729 present in DNA (cytosine-5)-methyltransferase 3 beta (DNMT3B); rs7163862 present in GTP cyclohydrolase 1 feedback regulatory protein (GCHFR); rs12659 present in reduced folate carrier protein (RFC1); rs202676 present in folate hydrolase (prostate-specific membrane antigen) (FOLH1); rs2297291 present in reduced folate carrier protein (RFC1); rs1051266 present in reduced folate carrier protein 1 (RFC1); rs8007267 present in GTP cyclohydrolase 1 (GCH1); rs7639752 present in choline-phosphate cytidylytransferase A (PCYT1A); rs6275 present in dopamine receptor D2 (DRD2); rs1079596 present in DRD2; rs11240594 present in DRD2; rs4633 present in catechol-O-methyltransferase (COMT); rs4680 present in COMT; rs250682 present in dopamine active transporter (DAT, or SLC6A3); rs2277820 present in formiminotransferase cyclodeaminase (FTCD); rs2236225 present in methylenetetrahydrofolate dehydrogenase (NADP+ dependent) 1 (MTHFD1); and any combination thereof (see, Table 1 and 2).

TABLE 1
Single nucleotide polymorphisms of folate responsive genes

| Folate-responslve SNP Blomarkers | | | | |
|---|---|---|---|---|
| SEQ ID | | | Chromosome | Gene |
| NO | Sequence | rs number | locus | name |
| 7 | CTTGAAGGAGAAGGTGTCTGCGGGAG[C/T]CGATTTCATCATCACGCAGCTTTTC | rs1801133 | 1p36.3 | MTHFR |
| 8 | CGAGGCCTTTGCCCTGTGGATTGAGC[A/G]GTGGGGAAAGCTGTATGAGGAGGAG | rs2274976 | 1p36.3 | MTHFR |
| 9 | GGAAGAATATGAAGATATTAGACAGG[A/G]CCATTATGAGTCTCTCAAGGTAAGT | rs1805087 | 1q43 | MTR |
| 10 | CAGGCAAAGGCCATCGCAGAAGAAAT[A/G]TGTGAGCAAGCTGTGGTACATGGAT | rs1801394 | 5p15.31 | MTRR |
| 11 | TAAGTTCCATTCCATCTCAGCCCGAA[A/G]TGTTTTCAGAGCCGGAGACCTCACA | rs1006737 | 12p13.33 | CACNA1C |
| | | | | [Ca ion] |
| 12 | CTGCTGCTGGTATCAGCCTGGAGGAA[A/G]TGAGTGACATCAGTTCTCAGCATTA | rs1883729 | 20q11.2 | DNMT3B |
| 13 | AACCAATCACAACAAGGCAGATAAAG[A/T]AGGATGAGTTGTCAGATTTTGATAA | rs7163862 | 15q15.1 | GCHFR |
| | | | | [BH4] |
| 14 | GCTTCGGAGCTGGAGCGCATGAATCC[C/T]GGCCCAGGCGGGAAGCTGGGACACG | rs12659 | 21q22.3 | RCF2 |
| 15 | AAGCTGAGAACATCAAGAAGTTCTTA[C/T]AGTAAGTACATCCTCGAAAGTTTAT | rs202676 | 11p11.2 | FOLH1 |
| | | | | (GCPII) |
| 16 | GGGAGGGCACCCGCAGAGGCCTGCGC[A/G]CTGACACTGCTGAGTGGCTCTGCTC | rs2297291 | 21q22.3 | RCF1 |
| 17 | TGACCCCGAGCTCCGGTCCTGGCGGC[A/G]CCTCGTGTGCTACCTTTGCTTCTAC | rs1051266 | 21q22.3 | RCF1 |
| 18 | CAATAGGAGCGTGTGTTTGAACAGTA[C/T]ACGCCAAACTTCAGTCATTCAAGTA | rs8007267 | 14q22.1- | GCH1 |
| | | | q22.2 | [BH4] |
| 19 | GGCCTAATCAATCCTTCTCATCTTTT[A/G]TACCCACCTTTTGCAGGAAACCTGT | rs7639752 | 3q29 | PCYT1A |
| 20 | CTGACTCTCCCCGACCCGTCCCACCA[C/T]GGTCTCCACAGCACTCCCGACAGCC | rs6275 | 11q23.2 | DRD2 |
| 21 | GTCCCTGCAGTTTAATTATCCTCAAC[A/G]TTACTGCCATACCCTACATTTTTGG | rs1079596 | 11q23.2 | DRD2 |
| 22 | CTCACAGTTTGTGGTTGAGACTAAGT[A/G]TGACAACAGTGGCACTTTGTGGTCC | rs11240594 | | DRD2 |
| 23 | ACCAAGGAGCAGCGCATCCTGAACCA[C/T]GTGCTGCAGCATGCGGAGCCCGGGA | rs4633 | 22q11.21- | COMT |
| | | | q11.23\| | |
| | | | 22q11.21 | |
| 24 | CCCAGCGGATGGTGGATTTCGCTGGC[A/G]TGAAGGACAAGGTGTGCATGCCTGA | rs4680 | 22q11.21- | COMT |
| | | | q11.23\| | |
| | | | 22q11.21 | |
| 25 | TAATATGGCCACCCCAACTTTCGTAT[C/G]ATTACTGTTTGTGTGGTATTATCTT | rs250682 | 5p15.3 | SLC6A3 |
| 26 | ATCAGCCCTAGATGCTTGACCAGCTC[C/T]TCGGGCCTCACCTCCTGGTTCTTCC | rs2277820 | 21q22.3 | FTCD |
| 27 | CTGGGCCAACAAGCTTGAGTGCGATC[C/T]GGTCTGCAATGATGGAGGAATTGCC | rs2236225 | 14q24 | MTHFD1 |

TABLE 2
Folate responsive alleles for marker genes

| | Folate-responsive Conditions | | | |
|---|---|---|---|---|
| | | Folate- | Folate-responsive | Pos. No of |
| | Condition | responsive | complementary | SNP in the |
| Gene name | Identifier | allele | allele | Sequence |
| MTHFR | A | T | A | 27 |
| MTHFR | D | A | I | 27 |
| MTH | C | G | C | 27 |
| MTRH | D | C | C | 27 |
| CACNA1C | E | A | I | 27 |
| (Ca Ion) | | | | |
| DNMT3B | F | A | T | 27 |
| GCHFR (SHA) | G | T | A | 27 |
| BCF2 | H | T | A | 27 |
| FOLH1 | I | G | C | 27 |
| (GCH1) | | | | |
| RCF1 | J | A | F | 27 |
| RCF1 | K | A | T | 27 |
| GCH1 (BH4) | L | T | A | 27 |
| PCYT1A | M | A | I | 27 |
| GRD2 | N | T | A | 27 |
| DRD2 | O | T | A | 27 |
| DRD2 | P | A | T | 27 |
| COMT | Q | C | G | 27 |
| COMT | R | G | C | 27 |
| SLC6A3 | D | C | G | 27 |
| FFCD | I | I | A | 27 |
| MTHFD1 | G | A | T | 27 |

In some embodiments, a pair of the SNPs described herein is used to determine whether a subject with depression or at risk for depression is likely to have a positive response to a folate-comprising compound. In particular, the presence of at least two of the following SNPs is associated with a response to a folate-comprising compound:
i. a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR);
ii. a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR);
iii. a SNP at position 1793 of SEQ ID NO: 1 or position 27 of SEQ ID NO: 8 (identified by rs2274976) comprising at least one adenine “A” allele or the complement thereof, wherein the SEQ ID NO: 1 and SEQ ID NO: 8 are each independently a portion of a genomic nucleic acid sequence of methylenetetrahydrofolate reductase (MTHFR);
iv. a SNP at position 66 of SEQ ID NO: 3 or position 27 of SEQ ID NO: 10 (identified by rs1801394) comprising at least one guanine “G” allele or the complement thereof, wherein the SEQ ID NO: 3 and SEQ ID NO: 10 are each independently a portion of a genomic nucleic acid sequence of methionine synthase reductase (MTRR);
v. a SNP at position 27 of SEQ ID NO: 11 (identified by rs1006737) comprising at least one adenine “A” allele or the complement thereof, wherein the SEQ ID NO: 11 is a portion of a genomic nucleic acid sequence of calcium channel, voltage-dependent, L type, alpha 1C subunit (CACNA1C);
vi. a SNP at position 27 of SEQ ID NO: 12 (identified by rs1883729) comprising at least one adenine “A” allele or the complement thereof, wherein the SEQ ID NO: 12 is a portion of a genomic nucleic acid sequence of DNA (cytosine-5)-methyltransferase 3 beta (DNMT3B);
vii. a SNP at position 27 of SEQ ID NO: 13 (identified by rs7163862) comprising at least one thymine “T” allele or the complement thereof, wherein the SEQ ID NO: 13 is a portion of a genomic nucleic acid sequence of GTP cyclohydrolase 1 feedback regulatory protein (GCHFR);
viii. a SNP at position 27 of SEQ ID NO: 14 (identified by rs12659) comprising two thymine “T” allele or the complement thereof, wherein the SEQ ID NO: 14 is a portion of a genomic nucleic acid sequence of reduced folate carrier protein (RCF2);
ix. a SNP at position 27 of SEQ ID NO: 15 (identified by rs202676) comprising at least one guanine “G” allele or the complement thereof, wherein the SEQ ID NO: 15 is a portion of a genomic nucleic acid sequence of folate hydrolase (prostate-specific membrane antigen) 1 (FOLH1); x. a SNP at position 27 of SEQ ID NO: 16 (identified by rs2297291) comprising two adenine “A” alleles or the complement thereof, wherein the SEQ ID NO: 16 is a portion of a genomic nucleic acid sequence of reduced folate carrier protein (RCF1);
xi. a SNP at position 27 of SEQ ID NO: 17 (identified by rs1051266) comprising two adenine “A” alleles or the complement thereof, wherein the SEQ ID NO: 17 is a portion of a genomic nucleic acid sequence of reduced folate carrier protein (RCF1);
xii. a SNP at position 27 of SEQ ID NO: 18 (identified by rs8007267) comprising at least one thymine “T” allele or the complement thereof, wherein the SEQ ID NO: 18 is a portion of a genomic nucleic acid sequence of GTP cyclohydrolase 1 (GCH1);
xiii. a SNP at position 27 of SEQ ID NO: 19 (identified by rs7639752) comprising at least one adenine “A” allele or the complement thereof, wherein the SEQ ID NO: 19 is a portion of a genomic nucleic acid sequence of choline-phosphate cytidylyltransferase A (PCYT1A);
xiv. a SNP at position 27 of SEQ ID NO: 20 (identified by rs6275) comprising two thymine “T” alleles or the complement thereof, wherein the SEQ ID NO: 20 is a portion of a genomic nucleic acid sequence of dopamine receptor D2 (DRD2);
xv. a SNP at position 27 of SEQ ID NO: 21 (identified by rs1079596) comprising at least one thymine “T” allele or the complement thereof, wherein the SEQ ID NO: 21 is a portion of a genomic nucleic acid sequence of dopamine receptor D2 (DRD2);
xvi. a SNP at position 27 of SEQ ID NO: 22 (identified by rs11240594) comprising at least one adenine “A” allele or the complement thereof, wherein the SEQ ID NO: 22 is a portion of a genomic nucleic acid sequence of dopamine receptor D2 (DRD2);
xvii. a SNP at position 27 of SEQ ID NO: 23 (identified by rs4633) comprising two cytosine “C” alleles or the complement thereof, wherein the SEQ ID NO: 23 is a portion of a genomic nucleic acid sequence of catechol-O-methyltransferase (COMT);
xviii. a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT);
xix. a SNP at position 27 of SEQ ID NO: 25 (identified by rs250682) comprising at least one cytosine “C” allele or the complement thereof, wherein the SEQ ID NO: 25 is a portion of a genomic nucleic acid sequence of solute carrier family 6 (neurotransmitted transported, dopamine), member 3 (SLC6A3);
xx. a SNP at position 27 of SEQ ID NO: 26 (identified by rs2277820) comprising at least one thymine “T” allele or the complement thereof, wherein the SEQ ID NO: 26 is a portion of a genomic nucleic acid sequence of formiminotransferase cyclodeaminase (FTCD); and
xxi. a SNP at position 27 of SEQ ID NO: 27 (identified by rs2236225) comprising at least one adenine “A” allele or the complement thereof, wherein the SEQ ID NO: 27 is a portion of a genomic nucleic acid sequence of methylenetetrahydrofolate dehydrogenase (NADP+ dependent) 1 (MTHFD1).

The combinations of two biomarkers that show a therapeutic response greater than the response produced by either marker alone are presented in Table 3.

TABLE 3
Dual-biomarker combinations indicative of folate response

| Combination | Biomarkers and associated folate-responsive conditions |
|---|---|
| 1 | (i) a SNP at position 677 of SEQ ID NO: 1 or position 27 of SEQ ID NO: 7 |
| | (identified by rs1801133) comprising at least one thymine “T” allele or the |
| | complement thereof, wherein the SEQ ID NO: 1 and SEQ ID NO: 7 are each |
| | independently a portion of a genomic nucleic acid sequence of |
| | methylenetetrahydrofolate reductase (MTHFR); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 2 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1); |
| | and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 3 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1); |
| | and |
| | (ii) a SNP at position 27 of SEQ ID NO: 24 (identified by rs4680) comprising two |
| | guanine “G” alleles or the complement thereof, wherein the SEQ ID NO: 24 is a |
| | portion of a genomic nucleic acid sequence of catechol-O-methyltransferase |
| | (COMT) |
| 4 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 24 (identified by rs4680) comprising two |
| | guanine “G” alleles or the complement thereof, wherein the SEQ ID NO: 24 is a |
| | portion of a genomic nucleic acid sequence of catechol-O-methyltransferase |
| | (COMT) |
| 5 | (i) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR); and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 6 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 7 | (i) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B); and |
| | (ii) a SNP at position 27 of SEQ ID NO: 24 (identified by rs4680) comprising two |
| | guanine “G” alleles or the complement thereof, wherein the SEQ ID NO: 24 is a |
| | portion of a genomic nucleic acid sequence of catechol-O-methyltransferase |
| | (COMT) |
| 8 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 9 | (i) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 10 | (i) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 24 (identified by rs4680) comprising two |
| | guanine “G” alleles or the complement thereof, wherein the SEQ ID NO: 24 is a |
| | portion of a genomic nucleic acid sequence of catechol-O-methyltransferase |
| | (COMT) |
| 11 | (i) a SNP at position 677 of SEQ ID NO: 1 or position 27 of SEQ ID NO: 7 |
| | (identified by rs1801133) comprising at least one thymine “T” allele or the |
| | complement thereof, wherein the SEQ ID NO: 1 and SEQ ID NO: 7 are each |
| | independently a portion of a genomic nucleic acid sequence of |
| | methylenetetrahydrofolate reductase (MTHFR); and |
| | (ii) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1) |
| 12 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1); |
| | and |
| | (ii) a SNP at position 27 of SEQ ID NO: 17 (identified by rs1051266) comprising |
| | two adenine “A” alleles or the complement thereof, wherein the SEQ ID NO: 17 is a |
| | portion of a genomic nucleic acid sequence of reduced folate carrier protein (RFC1) |
| 13 | (i) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B); and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 14 | (i) a SNP at position 27 of SEQ ID NO: 16 (identified by rs2297291) comprising two |
| | adenine “A” alleles or the complement thereof, wherein the SEQ ID NO: 16 is a |
| | portion of a genomic nucleic acid sequence of reduced folate carrier protein (RFC1); |
| | and |
| | (ii) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1) |
| 15 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1) |
| 16 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1), |
| | and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 17 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 20 (identified by at rs6275) comprising two |
| | thymine “T” alleles or the complement thereof, wherein the SEQ ID NO: 20 is a |
| | portion of a genomic nucleic acid sequence of dopamine receptor D2 (DRD2) |
| 18 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 21(identified by rs1079596) comprising at |
| | least one thymine “T” allele or the complement thereof, wherein the SEQ ID NO: 21 |
| | is a portion of a genomic nucleic acid sequence of dopamine receptor D2 (DRD2) |
| 19 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 17 (identified by rs1051266) comprising |
| | two adenine “A” alleles or the complement thereof, wherein the SEQ ID NO: 17 is a |
| | portion of a genomic nucleic acid sequence of reduced folate carrier protein (RFC1) |
| 20 | (i) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 21 | (i) a SNP at position 27 of SEQ ID NO: 12 (identified by rs1883729) comprising two |
| | adenine “A” alleles or the complement thereof, wherein the SEQ ID NO: 12 is a |
| | portion of a genomic nucleic acid sequence of DNA (cytosine-5)-methyltransferase 3 |
| | beta (DNMT3B); and |
| | (ii) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1) |
| 22 | (i) a SNP at position 27 of SEQ ID NO: 21(identified by rs1079596) comprising at |
| | least one thymine “T” allele or the complement thereof, wherein the SEQ ID NO: 21 |
| | is a portion of a genomic nucleic acid sequence of dopamine receptor D2 (DRD2); |
| | and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 23 | (i) a SNP at position 27 of SEQ ID NO: 12 (identified by rs1883729) comprising two |
| | adenine “A” alleles or the complement thereof, wherein the SEQ ID NO: 12 is a |
| | portion of a genomic nucleic acid sequence of DNA (cytosine-5)-methyltransferase 3 |
| | beta (DNMT3B); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 24 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 23 (identified by rs4633) comprising two |
| | cytosine “C” alleles or the complement thereof, wherein the SEQ ID NO: 23 is a |
| | portion of a genomic nucleic acid sequence of catechol-O-methyltransferase |
| | (COMT) |
| 25 | (i) a SNP at position 677 of SEQ ID NO: 1 or position 27 of SEQ ID NO: 7 |
| | (identified by rs1801133) comprising at least one thymine “T” allele or the |
| | complement thereof, wherein the SEQ ID NO: 1 and SEQ ID NO: 7 are each |
| | independently a portion of a genomic nucleic acid sequence of |
| | methylenetetrahydrofolate reductase (MTHFR); and |
| | (ii) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C) |
| 26 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 20 (identified by at rs6275) comprising two |
| | thymine “T” alleles or the complement thereof, wherein the SEQ ID NO: 20 is a |
| | portion of a genomic nucleic acid sequence of dopamine receptor D2 (DRD2) |
| 27 | (i) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1) |
| 28 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 29 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 21 (identified by rs1079596) comprising at |
| | least one thymine “T” allele or the complement thereof, wherein the SEQ ID NO: 21 |
| | is a portion of a genomic nucleic acid sequence of dopamine receptor D2 (DRD2) |
| 30 | (i) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR) |
| 31 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B) |
| 32 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 16 (identified by rs2297291) comprising |
| | two adenine “A” alleles or the complement thereof, wherein the SEQ ID NO: 16 is a |
| | portion of a genomic nucleic acid sequence of reduced folate carrier protein (RFC1) |
| 33 | (i) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 1793 of SEQ ID NO: 1 or position 27 of SEQ ID NO: 8 |
| | (identified by rs2274976) comprising at least one adenine “A” allele or the |
| | complement thereof, wherein the SEQ ID NO: 1 and SEQ ID NO: 8 are each |
| | independently a portion of a genomic nucleic acid sequence of |
| | methylenetetrahydrofolate reductase (MTHFR) |
| 34 | (i) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 17 (identified by rs1051266) comprising |
| | two adenine “A” alleles or the complement thereof, wherein the SEQ ID NO: 17 is a |
| | portion of a genomic nucleic acid sequence of reduced folate carrier protein (RFC1), |
| 35 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 27 of SEQ ID NO: 12 (identified by rs1883729) comprising |
| | two adenine “A” alleles or the complement thereof, wherein the SEQ ID NO: 12 is a |
| | portion of a genomic nucleic acid sequence of DNA (cytosine-5)-methyltransferase 3 |
| | beta (DNMT3B) |
| 36 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) a SNP at position 1793 of SEQ ID NO: 1 or position 27 of SEQ ID NO: 8 |
| | (identified by rs2274976) comprising at least one adenine “A” allele or the |
| | complement thereof, wherein the SEQ ID NO: 1 and SEQ ID NO: 8 are each |
| | independently a portion of a genomic nucleic acid sequence of |
| | methylenetetrahydrofolate reductase (MTHFR) |
| 37 | (i) a SNP at position 27 of SEQ ID NO: 15(identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B) |
| 38 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 14 (identified by rs12659) comprising two |
| | thymine “T” alleles or the complement thereof, wherein the SEQ ID NO: 14 is a |
| | portion of a genomic nucleic acid sequence of reduced folate carrier protein (RFC1) |
| 39 | (i) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 21 (identified by rs1079596) comprising at |
| | least one thymine “T” allele or the complement thereof, wherein the SEQ ID NO: 21 |
| | is a portion of a genomic nucleic acid sequence of dopamine receptor D2 (DRD2) |
| 40 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1); |
| | and |
| | (ii) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B) |
| 41 | (i) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1); and |
| | (ii) a SNP at position 27 of SEQ ID NO: 16 (identified by rs2297291) comprising |
| | two adenine “A” alleles or the complement thereof, wherein the SEQ ID NO: 16 is a |
| | portion of a genomic nucleic acid sequence of reduced folate carrier protein (RFC1) |
| 42 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 1793 of SEQ ID NO: 1 or position 27 of SEQ ID NO: 8 |
| | (identified by rs2274976) comprising at least one adenine “A” allele or the |
| | complement thereof, wherein the SEQ ID NO: 1 and SEQ ID NO: 8 are each |
| | independently a portion of a genomic nucleic acid sequence of |
| | methylenetetrahydrofolate reductase (MTHFR) |
| 43 | (i) a SNP at position 27 of SEQ ID NO: 14 (identified by rs12659) comprising two |
| | thymine “T” alleles or the complement thereof, wherein the SEQ ID NO: 14 is a |
| | portion of a genomic nucleic acid sequence of reduced folate carrier protein (RFC1); |
| | and |
| | (ii) a SNP at position 27 of SEQ ID NO: 15 (identified by rs202676) comprising at |
| | least one guanine “G” allele or the complement thereof, or one adenine “A” allele or |
| | the complement thereof, wherein the SEQ ID NO: 15 is a portion of a genomic |
| | nucleic acid sequence of folate hydrolase (prostate-specific membrane antigen) 1 |
| | (FOLH1) |
| 44 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B) |
| 45 | (i) a SNP at position 2756 of SEQ ID NO: 2 or position 27 of SEQ ID NO: 9 |
| | (identified by rs1805087) comprising at least one guanine “G” allele or the |
| | complement thereof, wherein the SEQ ID NO: 2 and SEQ ID NO: 9 are each |
| | independently a portion of a genomic nucleic acid sequence of methionine synthase |
| | (MTR); and |
| | (ii) obese |
| 46 | (i) a SNP at position 27 of SEQ ID NO: 18 (identified by rs8007267) comprising at |
| | least one thymine “T” allele or the complement thereof, wherein the SEQ ID NO: 18 |
| | is a portion of a genomic nucleic acid sequence of GTP cyclohydrolase 1 (GCH1); |
| | and |
| | (ii) obese |
| 47 | (i) a SNP at position 27 of SEQ ID NO: 12 (identified by rs1883729) comprising at |
| | least one adenine “A” allele or the complement thereof, wherein the SEQ ID NO: 12 |
| | is a portion of a genomic nucleic acid sequence of DNA (cytosine-5)- |
| | methyltransferase 3 beta (DNMT3B); and |
| | (ii) obese |
| 48 | (i) a SNP at position 677 of SEQ ID NO: 1 or position 27 of SEQ ID NO: 7 |
| | (identified by rs1801133) comprising at least one thymine “T” allele or the |
| | complement thereof, wherein the SEQ ID NO: 1 and SEQ ID NO: 7 are each |
| | independently a portion of a genomic nucleic acid sequence of |
| | methylenetetrahydrofolate reductase (MTHFR); and |
| | (ii) obese |
| 49 | (i) a SNP at position 27 of SEQ ID NO: 24 (identified by rs4680) comprising two |
| | guanine “G” alleles or the complement thereof, wherein the SEQ ID NO: 24 is a |
| | portion of a genomic nucleic acid sequence of catechol-O-methyltransferase |
| | (COMT); and |
| | (ii) obese |
| 50 | (i) a SNP at position 27 of SEQ ID NO: 11 (identified by rs1006737) comprising at |
| | least one adenine “A” allele or the complement thereof, wherein the SEQ ID NO: 11 |
| | is a portion of a genomic nucleic acid sequence of calcium channel, voltage- |
| | dependent, L type, alpha 1C subunit (CACNA1C); and |
| | (ii) obese |
| 51 | (i) a SNP at position 27 of SEQ ID NO: 13 (identified by rs7163862) comprising at |
| | least one thymine “T” allele or the complement thereof, or one adenine “A” allele or |
| | the complement thereof, wherein the SEQ ID NO: 13 is a portion of a genomic |
| | nucleic acid sequence of GTP cyclohydrolase 1 feedback regulatory protein |
| | (GCHFR); and |
| | (ii) obese |
| 52 | (i) a SNP at position 27 of SEQ ID NO: 21(identified by rs1079596) comprising at |
| | least one thymine “T” allele or the complement thereof, wherein the SEQ ID NO: 21 |
| | is a portion of a genomic nucleic acid sequence of dopamine receptor D2 (DRD2); |
| | and |
| | (ii) obese |

In some embodiments, the dual-biomarker combinations set forth in Table 3 can be used in the assays and/or methods described herein to identify a patient who will positively respond to a treatment comprising a folate-containing compound. In other embodiments, the folate-comprising compound described herein can be used to treat a patient carrying a dual-biomarker combination set forth in Table 3.

The synergistic dual-biomarker combinations described herein are indicative of a therapeutic response to a folate-comprising treatment that is greater than the sum of responses produced by two individual biomarkers alone, or is greater than a response produced by either biomarker alone (Table 4).

TABLE 4
Synergistic dual-biomarker combinations that are indicative of folate response

| | | | Theoretical | |
|---|---|---|---|---|
| | | | HAMD-28 | |
| Synergistic | HAMD-28 | HAMD-28 (single | change for | Measured |
| combinations | (single marker) | marker) | additive effect | HAMD-28 |
| MTHFR+MTR | MTHFR | MTR | -12 | -23.3 |
| | (SEQ ID NO: 7) | (SEQ ID NO: 9) | | |
| | -3.8 | -8.2 | | |
| GCH1 + COMT (rs4633) | GCH1 | COMT rs4633 | -15.9 | -20.7 |
| | (SEQ ID NO: 18) | (SEQ ID NO: 23) | | |
| | -6.7 | -9.2 | | |
| GCH1 + COMT (rs4680) | GCH1 | COMT rs4680 | -17.6 | -20.7 |
| | (SEQ ID NO: 18) | (SEQ ID NO: 24) | | |
| | -6.7 | -10.9 | | |
| CACNA1C + COMT | CACNA1C | COMT rs4680 | -15.5 | -16.2 |
| (rs4680) | (SEQ ID NO: 11) | (SEQ ID NO: 24) | | |
| | -4.6 | -10.9 | | |
| BMI > 30 + MTR | BMI > 30 | MTR (SEQ ID | -12.9 | -14.4 |
| | | NO: 9) | | |
| | -4.7 | -8.2 | | |
| BMI > 30 + GCH1 | BMI > 30 | GCH1 (SEQ ID | -11.4 | -14.3 |
| | | NO: 18) | | |
| | -4.7 | -6.7 | | |
| CACNA1C + MTR | CACNA1C | MTR | -12.8 | -13.5 |
| | (SEQ ID NO: 11) | (SEQ ID NO: 9) | | |
| | -4.6 | -8.2 | | |
| BMI > 30 + DNMT3B | BMI > 30 | DNMT3B | -10.1 | -11.4 |
| | | (SEQ ID NO: 12) | | |
| | -4.7 | -5.4 | | |
| MTHFR + FOLH1 | MTHFR | FOLH1 | -9.2 | -11.4 |
| | (SEQ ID NO: 7) | (SEQ ID NO: 15) | | |
| | -3.8 | -5.4 | | |
| MTHFR + BMI > 30 | MTHFR | BMI > 30 | -8.5 | -9.9 |
| | (SEQ ID NO: 7) | | | |
| | -3.8 | -4.7 | | |

In some embodiments, the synergistic dual-biomarker combinations set forth in Table 2 can be used in the assays and/or methods described herein to identify a patient who will positively respond to a treatment comprising a folate-containing compound. In other embodiments, the folate-comprising compound described herein can be used to treat a patient carrying a synergistic dual-biomarker combination set forth in Table 2.

In some embodiments, the synergistic dual genetic pair that is predictive of responsiveness to a folate-comprising compound are 1) a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR) and 2) a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR).

In other embodiments, the synergistic dual genetic pair that is predictive of responsiveness to a folate-comprising compound are 1) a SNP at position 27 of SEQ ID NO:18 (identified by rs8007267) comprising at least one thymine “T” allele or the complement thereof, wherein the SEQ ID NO:18 is a portion of a genomic nucleic acid sequence of GTP cyclohydrolase 1 (GCH1) and 2) a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT).

In yet other embodiments, the synergistic dual genetic pair that is predictive of responsiveness to a folate-comprising compound are 1) a SNP at position 27 of SEQ ID NO:18 (identified by rs8007267) comprising at least one thymine “T” allele or the complement thereof, wherein the SEQ ID NO:18 is a portion of a genomic nucleic acid sequence of GTP cyclohydrolase 1 (GCH1) and 2) a SNP at position 27 of SEQ ID NO: 23 (identified by rs4633) comprising two cytosine “C” alleles or the complement thereof, wherein the SEQ ID NO:23 is a portion of a genomic nucleic acid sequence of catechol-O-methyltransferase (COMT).

Other synergistic dual-marker combinations include:
(i) a SNP at position 27 of SEQ ID NO: 11 (identified by rs1006737) comprising at least one adenine “A” allele or the complement thereof, wherein the SEQ ID NO: 11 is a portion of a genomic nucleic acid sequence of calcium channel, voltage-dependent, L type, alpha 1C subunit (CACNA1C); and (ii) a SNP at position 27 of SEQ ID NO: 24 (identified by rs4680) comprising two guanine “G” alleles or the complement thereof, wherein the SEQ ID NO: 24 is a portion of a genomic nucleic acid sequence of catechol-O-methyltransferase (COMT);
(i) a SNP at position 27 of SEQ ID NO: 11 (identified by rs1006737) comprising at least one adenine “A” allele or the complement thereof, wherein the SEQ ID NO: 11 is a portion of a genomic nucleic acid sequence of calcium channel, voltage-dependent, L type, alpha 1C subunit (CACNA1C); and (ii) a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR);
(i) a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR); and (ii) a SNP at position 27 of SEQ ID NO: 15 (identified by rs202676) comprising at least one guanine “G” allele or the complement thereof, or one adenine “A” allele or the complement thereof, wherein the SEQ ID NO: 15 is a portion of a genomic nucleic acid sequence of folate hydrolase (prostate-specific membrane antigen) 1 (FOLH1);
(i) a SNP at position 2756 of SEQ ID NO: 2 or position 27 of SEQ ID NO: 9 (identified by rs1805087) comprising at least one guanine “G” allele or the complement thereof, wherein the SEQ ID NO: 2 and SEQ ID NO: 9 are each independently a portion of a genomic nucleic acid sequence of methionine synthase (MTR); and (ii) obesity;
(i) a SNP at position 27 of SEQ ID NO: 18 (identified by rs8007267) comprising at least one thymine “T” allele or the complement thereof, wherein the SEQ ID NO: 18 is a portion of a genomic nucleic acid sequence of GTP cyclohydrolase 1 (GCH1); and (ii) obesity
(i) a SNP at position 27 of SEQ ID NO: 12 (identified by rs1883729) comprising at least one adenine “A” allele or the complement thereof, wherein the SEQ ID NO: 12 is a portion of a genomic nucleic acid sequence of DNA (cytosine-5)-methyltransferase 3 beta (DNMT3B); and (ii) obesity; and
(i) a SNP at position 677 of SEQ ID NO: 1 or position 27 of SEQ ID NO: 7 (identified by rs1801133) comprising at least one thymine “T” allele or the complement thereof, wherein the SEQ ID NO: 1 and SEQ ID NO: 7 are each independently a portion of a genomic nucleic acid sequence of methylenetetrahydrofolate reductase (MTHFR); and (ii) obesity.

Further, peripheral biomarker conditions that can predict efficacy of administering to a human subject a folate-comprising compound (e.g., in a combination therapy to increase the efficacy of an antidepressant) for treatment of depression include relative expression levels between s-adenosyl methionine (SAM) and s-adenosyl homocysteine (SAH), expression of 4-hydroxynonenal (4-HNE), expression of high-sensitivity c-reactive protein (hsCRP), and any combinations thereof. Additionally, obesity has also been discovered to be predicative of effectiveness of a treatment regimen comprising a folate-comprising compound (e.g., as a monotherapy or a combination therapy with an antidepressant).

In some embodiments of the assays and/or methods provided herein, at least one synergistic biomaker combination is measured, and optionally at least one peripheral biomarker, such as obesity, the SAM/SAH ratio, the level of 4-HNE, the level of hsCRP or a combination thereof, are measured to determine whether the subject will have a therapeutic response to adjunctive therapy of a folate-comprising compound, e.g., L-methylfolate.

B. Other Folate-Responsive Biomarkers

In some embodiments, when the expression ratio of s-adenosyl methionine (SAM) to s-adenosyl homocysteine (SAH) [SAM/SAH ratio] is smaller than the pre-determined reference ratio, e.g., smaller than a control ratio of SAM/SAH as measured in a biological sample of normal healthy subjects, the subject can be recommended for and/or optionally administered with a treatment regimen comprising a folate-comprising compound. In one embodiment, the pre-determined reference ratio of SAM to SAH can be the control ratio of SAM to SAH as measured in a serum sample of normal healthy subjects, wherein the control ratio of SAM to SAH can range from about 4 to about 12 such as 4, 5, 6, 7, 8, 9, 10, 11, or 12. In other embodiments, the control ratio of SAM to SAH as measured in a serum sample of normal healthy subjects can be about 7. In some embodiments, the pre-determined reference ratio of SAM to SAH can be about 3 as measured in plasma sample. In other embodiments, the pre-determined reference ratio of SAM to SAH can be about 2.8, as measured in a plasma sample. In another embodiment, the pre-determined reference ratio of SAM to SAH can be about 2.71, as measured in a plasma sample. In some embodiments, if the expression ratio of SAM to SAH is at least or greater than the pre-determined reference ratio (e.g., at least or greater than 2.71 as measured in a plasma sample), the subject is not recommended for nor administered with a treatment regimen comprising a folate-comprising compound. Depending on the test sample source, e.g., a serum sample vs. a urine sample, the pre-determined reference ratio of SAM to SAH for a blood plasma sample can be different from that for, e.g., a urine sample. See, e.g., Stabler S P and Allen R H. 2004 Clinical Chemistry 50: 365-372. Methods for detecting SAM and SAH are described below.

In some embodiments, when the expression of 4-HNE in the subject is greater than the first pre-determined reference value, e.g., greater than a control value of 4-HNE as measured in a biological sample of normal healthy subjects, the subject can be recommended for and/or optionally administered with a treatment regimen comprising a folate-comprising compound. In one embodiment, the first pre-determined reference value of 4-HNE can be the control value of 4-HNE as measured in a serum sample of the normal healthy subjects, wherein the control value of 4-HNE can be about 0.24 mol per liter of serum, or about 0.04 mg per liter of serum. See, e.g., Gocmen A Y et al. 2008 Clinical Biohemistry 41: 836-840. In one embodiment, the first pre-determined reference value of 4-HNE can be about 3 mg per liter of plasma as measured in a plasma sample. In one embodiment, the first pre-determined reference value of 4-HNE can be about 3.2 mg per liter of plasma as measured in a plasma sample. In one embodiment, the first pre-determined reference value of 4-HNE can be about 3.28 mg per liter of plasma as measured in a plasma sample. In some embodiments, if the expression of 4-HNE is less than the first pre-determined reference value (e.g., less than 3.28 mg per liter of plasma as measured in a plasma sample), the subject is not recommended for nor administered with a treatment regimen comprising a folate-comprising compound. Depending on the test sample source, e.g., a blood sample vs. a cerebrospinal fluid sample, the pre-determined reference value for a plasma sample can be different from that for, e.g., a cerebrospinal fluid sample. Methods for measuring the level of 4-HNE in a sample taken from a patient are described below.

In some embodiments, the assay and methods described herein can further comprise determining expression of high-sensitivity c-reactive protein (hsCRP), wherein the hsCRP expression greater than a second pre-determined reference value, e.g., greater than a control value of hsCRP as measured in a biological sample of normal healthy subjects, is indicative of the subject recommended for and optionally administered with a treatment regimen comprising a folate-comprising compound. In some embodiments, the second pre-determined reference value of hsCRP can be the control value of hsCRP as measured in a serum sample of normal healthy subjects, wherein the control value of hsCRP can range from about 0.5 mg per liter of serum to about 4.5 mg per liter of serum. See, e.g., Guven S F et al., 2012 Sleep Breath 16: 217-221. In other embodiments, the second pre-determined reference value of hsCRP can be about 2.3 mg per liter of plasma, as measured in a plasma sample. In some embodiments, if the expression of hsCRP is lower than the second pre-determined reference value (e.g., lower than 2.3 mg per liter of plasma, as measured in a plasma sample), then the subject is not recommended for nor administered with a treatment regimen comprising a folate-comprising compound. Depending on the test sample source, e.g., a blood sample vs. a cerebrospinal fluid sample, the hsCRP expression a plasma sample can be different from that in, e.g., a cerebrospinal fluid (CSF) sample. Methods for measuring the level of hsCRP in a sample taken from a patient are described below.

In some embodiments, a physical biomarker, e.g., obesity indicator, can also be measured, wherein obesity (e.g., defined by a BMI value of at least about 30 kg/m² or greater; a waist circumference greater than 40 inches (or greater than 120 cm) in men, or greater than 35 inches (or greater than 88 cm) in women; a waist-hip ratio above 0.95 for men or above 0.80 for women; and/or a body fat percentage of at least about 25% in men or at least about 32% in women) indicates a treatment regimen comprising a folate-comprising compound be recommended for and/or administered to the human subject.

In some embodiments, the assay/method provided herein can comprise determining if the human subject is obese or not. If the human subject is determined to be obese, then the human subject is selected for and optionally administered with a treatment regimen comprising an effective amount of a folate-comprising compound. Methods of determining obesity in a human subject are known in the art and can include, but are not limited to, body mass index (BMI) measurement, measurement of abdominal fat (e.g., by waist circumference or waist-hip ratio), measurement of body fat, skinfold thickness, underwater weighing (densitometry), air-displacement plethysmography, computerized tomography (CT) and magnetic resonance imaging (MRI), and dual energy X-ray absorptiometry (DEXA), and any combinations thereof.

C. Methods for Detecting or Measuring Biomarkers

In some embodiments, a test sample subjected to analysis performed in the assays and/or methods described herein are derived from a sample (e.g., biological sample of a subject. The term “biological sample” as used herein denotes a sample taken or isolated from a biological organism, e.g., cell lysate, a homogenate of a tissue sample from a subject or a fluid sample from a subject. The biological sample includes untreated or pre-treated (or pre-processed) biological samples. A non-limiting example of a biological sample includes a biological fluid, such as, blood (including whole blood, plasma, cord blood and serum), lactation products (e.g., milk), amniotic fluids, sputum, saliva, urine, semen, cerebrospinal fluid, bronchial aspirate, perspiration, mucus, liquefied feces, synovial fluid, lymphatic fluid, tears, tracheal aspirate, and fractions thereof. In other embodiments, the biological sample can include cell lysate and fractions thereof. For example, cells (such as red blood cells, platelets, white blood cells and any cells circulating in the biological fluid described herein) can be harvested and lysed to obtain a cell lysate. In some embodiments, the biological sample is a blood sample. In some embodiments, the biological sample is a plasma sample. In other embodiments, the biological sample is a saliva sample. In another embodiment, the biological sample is a buccal sample. In yet other embodiments, the biological sample is a urine sample. In other embodiments, the biological sample is a cerebrospinal fluid sample.

In some embodiments, the sample contains cells from the subject and/or non-cellular biological material, such as non-cellular fractions of blood, saliva, or urine, that can be used to measure plasma/serum biomarker expression levels or determine SNPs. In some embodiments, the sample is from a resection, biopsy, or core needle biopsy. In addition, fine needle aspirate samples can be used. Samples can be either paraffin-embedded or frozen tissue.

The sample can be obtained by removing a sample of cells from a subject, but can also be accomplished by using previously isolated cells (e.g. isolated by another person). In addition, the biological sample can be freshly collected or a previously collected sample. In some embodiments, the biological sample can be a frozen biological sample, e.g., a frozen tissue or fluid sample such as urine, blood, serum or plasma. The frozen sample can be thawed before employing methods, assays and systems described herein. After thawing, a frozen sample can be centrifuged before being subjected to methods, assays and systems described herein.

In some embodiments, the sample can be a nucleic acid product amplified after polymerase chain reaction (PCR). The nucleic acid product can include DNA, RNA and mRNA and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found in, e.g., Molecular Cloning: A Laboratory Manual, 3rd Ed., Sambrook and Russell, Cold Spring Harbor Laboratory Press, 2001.

The skilled artisan recognizes methods and processes appropriate for pre-processing of test or biological samples, e.g., blood, required for determination of SNPs or expression levels of serum/plasma biomarkers as described herein.

Identification method of SNPs can be of either a positive-type (inclusion of an allele) or a negative-type (exclusion of an allele). Positive-type methods determine the identity of a nucleotide contained in a polymorphic site, whereas negative-type methods determine the identity of a nucleotide not present in a polymorphic site. Thus, a wild-type site can be identified either as wild-type or not mutant. For example, at a biallelic polymorphic site where the wild-type allele contains thymine and the mutant allele contains cytosine, a site can be positively determined to be either thymine or cytosine or negatively determined to be not thymine (and thus cytosine) or not cytosine (and thus thymine).

In some aspects, provided herein is a method for determining whether a subject is homozygous for a polymorphism, heterozygous for a polymorphism, or lacking the polymorphism altogether (i.e. homozygous wildtype) is encompassed. As an exemplary embodiment only, a method to detect the C>T variance at position 677 of SEQ ID NO: 1, a method for determining the allele, heterozygous for the C- and T-alleles, or homozygous for the C-allele or the T-allele at the SNP loci are provided. Substantially any method of detecting any allele of the SNPs described herein, such as allelic discrimination, restriction enzyme digestion, restriction fragment length polymorphism analysis, allele-specific probe hybridization, allele-specific primer extension, allele specific amplification, sequencing (e.g., Sanger sequencing, Pyrosequencing™ and next-generation sequencing), 5′ nuclease digestion, molecular beacon assay, oligonucleotide ligation assay, single-base extension or minisequencing, size analysis, homogenous assay (e.g., TagMan® assay), melting-curve FRET hybridization, fluorescent polarization, INVADER® assay, SNP microarrays, and single-stranded conformational polymorphism, can be used.

Any approach that detects mutations or polymorphisms in a gene can be used to detect the presence or absence of SNP biomarkers described herein, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

SNP genotyping methods are available from, e.g., Sequenom (San Diego, Calif.), Illumina (San Diego, Calif.), Life Technologies (Carlsbad, Calif.), and Affymetrix (Santa Clara, Calif.).

In some embodiments, the assay and/or methods provided herein include measuring the level of metabolites, such as SAM, SAH and/or 4-HNE, in a sample, e.g., serum, plasma, or CSF sample, from a subject having or at risk for depression. Levels of metabolites (e.g., SAM, SAH, and/or 4-HNE) can be detected by any known methods in the art. For example, mass spectrometry (MS) can be used to identify and to quantify metabolites after separation by GC, HPLC (LC-MS), and/or CE. In some embodiments, MS can be used as a stand-alone technology, e.g., the biological sample is infused directly into the mass spectrometer which provides both separation and detection of metabolites (e.g., SAM, SAH, and/or 4-HNE). In some instances, the target metabolite (e.g., SAM, SAH and/or 4-HNE) can be optionally separated (e.g., prior to detection) from a biological sample by gas chromatography (GC), e.g., when interfaced with mass spectrometry (GC-MS), and/or high performance liquid chromatography (HPLC), and/or capillary electrophoresis (CE). Further details about detecting SAM and SAH, including immunoassays for determining SAM, SAH and/or ratios thereof are described in U.S. Pat. App. NO: US 2009/0263879, which is incorporated herein by reference.

Other useful methods for detecting a metabolite include nanostructure-initiator mass spectrometry, laser-desorption/ionization mass spectrometry, e.g., matrix assisted laser desorption/ionization (MALDI) mass spectrometry, surface-enhanced laser desorption/ionization (SELDI) mass spectrometry, secondary ion mass spectrometry (SIMS), desorption electrospray ionization (DESI) mass spectrometry, nuclear magnetic resonance (NMR) spectroscopy, on-mobility spectrometry, electrochemical detection (coupled to HPLC) and radiolabel (when combined with thin-layer chromatography), mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, or tandem mass spectrometry (e.g., MS/MS, MS/MS/MS, ESI-MS/MS, etc.).

In other embodiments, an enzyme-coupled assay can be used to determine metabolite level (e.g., SAM, SAH, and/or 4-HNE) in a biological sample. By way of example only, a stereospecific colorimetric assay of SAM based on an enzyme-coupled reaction, thiopurine methyltransferase-catalyzed thiol methylation (see, e.g., Cannon, L. M, et al, Analytical Biochemistry, 308 (2) 358-363, 2002), can detect SAM levels in a sample. Additional metabolite analysis methods, e.g., as described in U.S. Pat. No. 8,344,115, and U.S. Pat. App. Pub. Nos. US 2012/0130212, and US 2008/0081375, can be also used for measurement of metabolites (e.g., SAM, SAH, and/or 4-HNE) in a biological sample. Further details about detecting SAM and SAH, including immunoassays for determining SAM, SAH and/or ratios thereof are described in U.S. Pat. App. NO: US 2009/0263879, which is incorporated herein by reference. The levels of 4-HNE can be determined by measuring expression levels of 4-HNE adducts, e.g., 4-HNE-His. Commercial ELISA kits for measuring 4-HNE adducts, e.g., OxiSelect™ HNE-His Adduct ELISA Kit are available, e.g., from CellBioLabs.

In some embodiments, expression level of hsCRP in a sample taken from a subject can be determined by measuring the protein level or mRNA level.

Without limitations, levels of the hsCRP protein can be detected by immunoassays, such as enzyme linked immunoabsorbant assay (ELISA), radioimmunoassay (RIA), Immunoradiometric assay (IRMA), Western blotting, immunocytochemistry or immunohistochemistry. Suitable ELISA kits for determining the presence or level of hsCRP in a serum, plasma, blood, CSF sample are available from, e.g., MP Biomedicals, Abcam and Calbiotech.

One skilled in the art recognizes that an antibody, antibody fragment, immunoconjugate and the like that can specifically bind to (e.g., recognizes) the biomarker are useful to detect the level of protein expression of the analytes described herein. Any known method in the art for measuring the levels of protein expression in a sample can be used in the assays and/or methods described herein.

In other embodiments, the expression of hsCRP is detected at the level of mRNA expression with an assay such as, for example, a hybridization assay (e.g., microarray) or an amplification-based assay. In preferred embodiments, the levels of mRNA expression of the analytes provided herein are performed by Northern blotting, Reverse Transcriptase Polymerase Chain Reaction (RT-PCR), quantitative RT-PCR (qRT-PCR), TaqMan® assay, or microarray. Any known method in the art for measuring the levels of RNA expression in a sample can be used in the assays and/or methods described herein.

D. Subjects with Depression or at Risk of Depression

In some embodiments, subjects amenable to assays, methods and compositions described herein are subjects that have been diagnosed with or suspected of having or developing depression. Accordingly, the subjects that have been diagnosed or suspected of having or developing with depression can be selected prior to subjecting them to the assays, methods and/or compositions described herein. In other embodiments, the subjects described herein are individuals that presents one or more symptoms indicative of a depression including major depressive disorder (e.g., unexplained insomnia, fatigue, irritability, etc.) or are being screened for depression including major depressive disorder (e.g., during a routine physical), for example, in accordance with the criteria listed in DSM-IV or ICD-10.

The DSM-IV and ICD-10 provides a common language and standard criteria for the classification of mental disorders, and have been commonly used by a suitably trained general practitioner, or by a psychiatrist or psychologist for diagnosis of depression including major depressive disorders. Symptoms of depression can include, but are not limited to, problems concentrating, remembering, and/or making decisions, changes in eating and/or sleeping habits, a loss of interest in enjoyable activities, difficulty going to work or taking care of daily responsibilities, feelings of guilt and/or hopelessness, slowed thoughts and/or speech, and preoccupation with thoughts of death or suicide. One of skill in the art can determine the score or rating of depression based on DSM-IV or ICD-10.

Other scales or criteria for classification of mental disorders known in the art, e.g., Maier or HAMD-7 scale, or social functioning questionnaire (SFQ), visual analogue scale (VAS), and/or cognitive and physical function questionnaire (CPFQ) can also be used to determine the degree of depression.

During diagnosis for depression, the practitioner can also assess the patient's medical history, discuss the subject's current ways of regulating their mood (healthy or otherwise) such as alcohol and drug use, and/or perform a mental state examination, which is an assessment of the person's current mood and thought content, in particular the presence of themes of hopelessness or pessimism, self-harm or suicide, and an absence of positive thoughts or plans. Additionally, a practitioner can generally perform a medical examination to rule out other non-cognitive causes of depressive symptoms. For example, blood tests measuring TSH and thyroxine can be used to exclude hypothyroidism; basic electrolytes and serum calcium to rule out a metabolic disturbance; and a full blood count including ESR to rule out a systemic infection or chronic disease. Testosterone levels can also be evaluated to diagnose hypogonadism, a cause of depression in men.

Any genetic or biomarker methods known in the art can also be used for diagnosis of depression. For example, U.S. Pat. App. NO: US 2010/0273153 describes that the presence of TG7AT haplotype can be indicative of predisposition to major depressive disorder. Additional genetic markers for depression such as ATP2A2, SCYAS, STIP1, EEF1A1, GRB10, CASP6, TSSC1, RAB9, NFATC3, TPR, and any others listed in, for example, U.S. Pat. App. NO: US 2005/0239110 can also be used for diagnosing depression.

In some embodiments, subjects amenable to assays, methods and compositions described herein are subjects that have been diagnosed with or suspected of having or developing major depressive disorder. A major depressive episode is characterized by the presence of a severely depressed mood that persists for at least two weeks. Episodes can be isolated or recurrent and can be categorized by a skilled practitioner as mild (few symptoms in excess of minimum criteria), moderate, or severe (marked impact on social or occupational functioning).

In some embodiments, subjects amenable to assays, methods and compositions described herein are subjects that have been diagnosed with depression (e.g., major depressive disorder (MDD)) and are resistant to antidepressant monotherapy, i.e., a treatment for depression with a single antidepressant only. In some instances, the subject with depression is resistant to at least one antidepressant in one or more classes, e.g., at least 2, 3, 4, 5, or more antidepressants in one or more classes. In some embodiments, the subjects described herein have been diagnosed with major depressive disorder (MDD) and are resistant to at least one serotonin reuptake inhibitors (SRI), including at least 1, 2, 3, 4, 5 or more SRIs. In other embodiments, subjects described herein have been diagnosed with major depressive disorder (MDD) and are resistant to at least one selective serotonin reuptake inhibitor (SSRI), including at least 1, 2, 3, 4, 5 or more SSRIs.

In some embodiments, subjects who are resistant to antidepressant monotherapy do not show a clinically-relevant reduction (e.g., as evaluated by a physician or a psychologist) in at least one symptom of depression from which they are suffering, after they have been administered with the antidepressant monotherapy for at least about 3 weeks or more, or up to about 3 weeks. Non-limiting examples of symptoms of depression include, but are not limited to, low or depressed mood, anhedonia, low energy levels, guilt, decreased work and interests, psychomotor retardation, agitation, psychic anxiety, somatic anxiety, general somatic symptoms, reduced cognition or any combinations thereof.

In some embodiments, subjects who are resistant to antidepressant monotherapy do not show a clinically relevant reduction in at least one symptom of depression (e.g., 1, 2, 3, or more symptoms), after they have been administered with the antidepressant monotherapy for at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks or more. In some embodiments, subjects are determined to be treatment resistant if they do not show a clinically relevant reduction in at least one symptom of depression (e.g., 1, 2, 3, or more symptoms), after they have been administered with the antidepressant monotherapy for at least or up to about 3 weeks, at least or up to about 4 weeks, at least or up to about 5 weeks, at least or up to about 6 weeks, at least or up to about 7 weeks, at least or up to about 8, at least or up to about 9 weeks, at least or up to about 10 weeks, at least or up to about 11 weeks, or at least or up to about 12 weeks. The clinically relevant reduction in symptoms of depression can be evaluated by a physician or a psychologist.

In some embodiments, the subjects described herein are diagnosed with treatment-resistant depression (TRD) or treatment-refractory depression. For instance, the subjects exhibit a kind of depression that does not respond or is resistant to at least two or more antidepressant drugs, e.g., at least three or more, or at least four or more antidepressant drugs. Treatment-resistant depression can include failing to achieve remission after two treatments or two antidepressants within 4-12 weeks of time.

In some embodiments, a subject is diagnosed with a treatment-resistant depression if the subject does not show a clinically relevant reduction in at least one symptom of depression described herein, after being administered with at least two or more antidepressant drugs (either individually or in combination) for at least about 3 weeks or more, e.g., at least about 4 weeks, at least about 5 weeks, at least about 6 weeks, at least about 7 weeks, at least about 8 weeks, at least about 9 weeks, at least about 10 weeks, at least about 11 weeks, at least about 12 weeks or more. In some embodiments, a subject is diagnosed with a treatment-resistant depression if the subject does not show a clinically relevant reduction in at least one symptom of depression described herein, after he/she has been administered with at least two or more antidepressant drugs (either individually or in combination) for up to about 12 weeks, including, e.g., up to about 11 weeks, up to about 10 weeks, up to about 9 weeks, up to about 8 weeks, up to about 7 weeks, up to about 6 weeks, up to about 5 weeks, up to about 4 weeks, or up to about 3 weeks. In some embodiments, a subject is diagnosed with a treatment-resistant depression if the subject does not show a clinically relevant reduction in at least one symptom of depression described herein, after being administered with at least two or more antidepressant drugs (either individually or in combination) for at least or up to about 6 weeks, at least or up to about 7 weeks, at least or up to about 8 weeks, at least or up to about 9 weeks, at least or up to about 10 weeks, at least or up to about 11 weeks, or at least or up to about 12 weeks. In some embodiments, the treatment-resistant depression is diagnosed if the subject does not experience clinically relevant improvement in the symptoms of depression after at least or up to about 12 weeks on an antidepressant medication.

In some embodiments, the subjectsdescribed herein are diagnosed with treatment-resistant depression (TRD) or treatment-refractory depression and are currently taking non-medicine treatment for TRD, e.g., but not limited to, electroconvulsive therapy, vagus nerve stimulation, transcranial magnetic stimulation, and/or “talk” therapy. These subjects can be recommended for, or administered with, a treatment regimen comprising a folate-comprising compound, alone or in combination with a non-medicine treatment for TRD as described herein. In some embodiments, these subjects can be recommended for, or administered with, a treatment regimen comprising a folate-comprising compound, in combination with at least one antidepressant drug, and optionally non-medicine treatment for TRD. In these embodiments, the antidepressant drug that is recommended for, or administered to, the subjects with TRD, in combination with a folate-comprising compound, can be an antidepressant drug to which the subjects have previously shown resistance, or an antidepressant drug that the subject has never tried.

In some embodiments, the subject selected for the assays, methods and compositions described herein have been in remission from depression and is now diagnosed with a relapse or a predisposition to a relapse. In other embodiments, the subject selected for the assays, methods and compositions described herein have been diagnosed with depression and is currently taking at least an antidepressant.

E. Adjunctive Therapy of Folate-Comprising Compounds

The assays and/or methods described herein can be used as a screen to identify and select for particular patients with depression, where a treatment regimen comprising an antidepressant and a folate-comprising compound will be beneficial to enhance the therapeutic effect of the antidepressant drug. In some embodiments, the selected patient, e.g., a patient with depression or at risk for depression and carrying a synergistic dual SNP combination (COMT Val158Met GG and GCH1 TC/TT pair and MTHFR 677 CT/TT and MTR 2756 AG/GG pair) is currently receiving at least one antidepressant drug. In some instance, the selected patient is or has experienced an inadequate response to antidepressant monotherapy.

A folate-comprising compound included in a treatment regimen can be administered together via a single dosage form or by separate administration. In certain embodiments, the folate-comprising compound can be administered in a single dosage form. For example, the single dosage form can be administered as a single tablet, pill, capsule for oral administration or a solution for parenteral administration. Alternatively, the folate-comprising compound can be administered as separate compositions, e.g., as separate tablets or solutions. The length of time between administrations of a sub-dose of a folate-comprising compound can be adjusted to achieve the desired therapeutic effect.

In some embodiments, a treatment regimen comprising a folate-comprising compound further comprise at least one antidepressant (e.g., 1, 2, 3 or more antidepressants). Non-limiting examples of antidepressants include tricyclic antidepressants, monoamine oxidase inhibitors, selective serotonin reuptake inhibitors (SSRIs), serotonin-norepinephrine reuptake inhibitors, norepinephrine and dopamine reuptake inhibitors, and atypical antidepressants.

In some embodiments, the treatment regimen can include the folate-comprising compound in combination with an antidepressant drug. In some embodiments, the antidepressant drug includes a selective serotonin reuptake inhibitor, such as, but not limited to, fluoxetine, fluvoxamine, citalopram, paroxetine, escitalopram, sertraline, and any combinations thereof.

A treatment regimen comprising a folate-comprising compound and at least one antidepressant can be administered together via a single dosage form or by separate administration. In certain embodiments, the antidepressant and the folate-comprising compound are administered together in a single dosage form. For example, the single dosage form can be administered as a single tablet, pill, capsule for oral administration or a solution for parenteral administration. Alternatively, the antidepressant and the folate-comprising compound can be administered as separate compositions, e.g., as separate tablets or solutions. The antidepressant can be administered at the same time as the folate-comprising compound, or the antidepressant can be administered intermittently with the folate-comprising compound. The length of time between administration of the antidepressant and the folate-comprising compound can be adjusted to achieve the desired therapeutic effect. In particular, the folate-comprising compound can be administered at any frequency or administration protocol to enhance the efficacy of the antidepressant drug, as compared to efficacy of the antidepressant drug alone (e.g., in the absence of the folate-comprising compound).

In some embodiments, the folate-comprising compound can be administered only a few minutes (e.g., 1, 2, 5, 10, 30, or 60 min) before or after administration of the antidepressant. Alternatively, the folate-comprising compound can be administered several hours (e.g., 2, 4, 6, 10, 12, 24, or 36 hr) before or after administration of the antidepressant. Depending on the half-lives of the antidepressant and the folate-comprising compound, in certain embodiments, it can be advantageous to administer more than one dosage of the folate-comprising compound between administrations of the antidepressant. For example, the folate-comprising compound can be administered at 3 hours and then again at 6 hours following administration of the antidepressant. Alternatively, it can be advantageous to administer more than one dosage of the antidepressant between administrations of the folate-comprising compound. Importantly, in some embodiments, the therapeutic effect of each antidepressant and folate-comprising compound can overlap for at least a portion of the duration of each therapeutic agent so that the overall therapeutic effect of the combination therapy is attributable in part to the combined of the combination therapy. In some embodiments, the folate-comprising compound and the antidepressant can be administered in a pulse administration. In other embodiments, they can be administered as a pulse-chase administration, e.g., where a folate-comprising compound is administered for a brief period of time (pulse), followed by administration of the antidepressant for a longer period of time (e.g., chase)

In some embodiments where the antidepressant and the folate-comprising compound are administered in separate compositions, the antidepressant and the folate-comprising compound can be administrated by the same or different routes. For example, the antidepressant can be administered by intravenous injection while the folate-comprising compound can be administered orally, or vice versa. Alternatively, for example, both the antidepressant and folate-comprising compound can be administered together by intravenous injection or by oral administration.

The adjuvant effect of the folate-comprising compound administered in combination with an antidepressant can be additive. The term “additive” as used herein in the context of one agent has an additive effect on a second agent, refers to an increase in effectiveness of a first agent in the presence of a second agent as compared to the use of the first agent alone. Stated in another way, the second agent can function as an agent which enhances the physiological response of an organ or organism to the presence of a first agent. Thus, a second agent will increase the effectiveness of the first agent by increasing an individual's response to the presence of the first agent.

In some instances, the adjuvant effect of the folate-comprising compound administered in combination with an antidepressant can be synergistic, wherein the interaction of two or more agents produces a combined effect that is greater than each of their individual effects at the same dose alone.

In some embodiments, the treatment regimen can further comprise cognitive-behavioral therapy (CBT), interpersonal therapy (IPT), life-style advice, including, e.g., prescribing an exercise regime, dietary advice, and/or administering another pharmaceutical agent (e.g., antipsychotics, lithium, L-triiodonthyronine and stimulants) effective in treatment of depression.

The subject with depression being treated with the methods described herein can be a subject currently taking an antidepressant. Accordingly, the methods of treating a human subject with depression described herein can also be used to identify a human subject who may benefit from adjunctive therapy of a folate-comprising compound. The methods also provide a means for improving the effectiveness of an antidepressant drug currently taken by a subject who exhibits an inadequate (unsatisfactory) response or resistance to the drug.

F. Folate-Comprising Compounds

Any art-recognized folate-comprising compound can be selected and/or optionally administered to a human subject selected to carry at least one dual-marker combination (e.g., at least one synergistic dual-marker combination).

In some embodiments, the folate-comprising compound can include at least one (including at least two, at least three or more) alkaline metal or alkaline earth metal salt of folate, e.g., but not limited to, a calcium salt of folate.

In some embodiments, the folate-comprising compound can include at least one (including at least two, at least three or more) glucosamine salt and/or galactosamine salt of folate (including, e.g., folic acid and reduced folate, e.g., but not limited to, tetrahydrofolate, and derivatives thereof). Examples of glucosamine-folate and/or galactosamine-folate and derivatives thereof, e.g., disclosed in U.S. Pat. No. 7,947,662, can be administered to a human subject in the methods or included in the compositions described herein. In one embodiment, QUATREFOLIC® (Gnosis S.p.A, Milan, IT) or N-[4-[[[(6S)-2-amino-1,4,5,6,7,8-hexahydro-5-methyl-4-oxo-6-pteridinyl]methyl]amino]benzoyl]-L-glutamic acid, glucosamine salt can be administered to a human subject in the methods or included in the compositions described herein.

In some embodiments, for a subject with depression who is deficient in the enzyme dihydrofolate reductase, methyl folate, also known as Me-THF, N5-Methyl-THF, MTHF, 5-MTHF, L-methylfolate, and Levomefolic acid, or a pharmaceutically acceptable salt thereof (e.g., sodium salt, potassium salt, magnesium salt, calcium salt, glucosamine salt, or galactosamine salt), is more desirable for use as a folate-comprising compound. For example, methyl folate calcium salt is available by prescription in the United States as DEPLIN® (L-methylfolate calcium salt). Methyl folate calcium salt is also available outside of the United States as METAFOLIN®, BODYFOLIN®, and NUTRIFOLIN®.

Additional examples of folates or folate-comprising compounds that can be administered to a subject in the methods or included in the compositions described herein can include, but not limited to, the ones described in the U.S. Pat. Nos. 4,336,185; 6,921,754; and 7,947,662; and U.S. Pat. App. Publication NO: US 2008/0064702, the disclosures of which are incorporated are herein incorporated by reference for all purposes.

In accordance with the assays and/or methods described herein, subjects with depression who have been determined to have the presence of at least one of the conditions described herein (e.g., dual SNP combinations and/or plasma/serum biomarkers described herein) can benefit from the therapeutic effect of an antidepressant administered in combination with an effective amount of a folate-comprising compound. In some embodiments, the folate-comprising compound can comprise L-methylfolate. In some embodiments, the folate-comprising compound can comprise 6(S)-5-methyltetrahydrofolate (also known as 6(S)-5-MTHF).

The effective amount of folate for use in the treatment methods described herein can vary, depending upon the types and/or dosage of the antidepressant (if any), types of folate, severity of depression, physical conditions of a subject (e.g., ages, genders, weights). The term “effective amount” refers to an amount of folate or a folate-comprising compound that, when administered to a selected subject, can reduce at least one symptom associated with depression, e.g., by at least about 5%, at least about 10%, at least about 20%, at least about 30%, at least about 40%, at least about 50%, at least about 60%, at least about 70%, at least about 80%, at least about 90% or more, as compared to the treatment in the absence of a folate-comprising compound.

In some embodiments, the term “effective amount” as used herein refers to an amount of folate or a folate-comprising compound, when administered to a selected subject in combination with an antidepressant, can increase the effect (e.g., efficacy or therapeutic effect) of the antidepressant, e.g., by at least about 5%, at least about 10%, at least about 20%, at least about 30%, at least about 40%, at least about 50%, at least about 60%, at least about 70%, at least about 80%, at least about 90% or more, as compared to the treatment with antidepressant alone. Stated another way, the term “effective amount” as used herein refers to an amount of folate or a folate-comprising compound, when administered to a selected subject in combination with an antidepressant, can reduce at least one symptom associated with depression as described later, e.g., by at least about 5%, at least about 10%, at least about 20%, at least about 30%, at least about 40%, at least about 50%, at least about 60%, at least about 70%, at least about 80%, at least about 90% or more, as compared to the treatment with antidepressant alone.

In some embodiments, the effective amount of folate in the treatment regimen as described herein can range from about 1 mg/day to about 70 mg/day, from about 1 mg/day to about 50 mg/day, from about 2.5 mg/day to about 40 mg/day, from about 5 mg/day to about 40 mg/day, from about 5 mg/day to about 30 mg/day or from about 7 mg/day to about 15 mg/day. In some embodiments, the effective amount of folate in the treatment regimen as described herein can range from about 15 mg/day to about 50 mg/day. In other embodiments, the effective amount of folate in the treatment regimen as described herein can range from about 20 mg/day to about 40 mg/day. In some embodiments, the effective amount of folate in a treatment regimen can be about 20 mg/day. In other embodiments, the effective amount of folate in a treatment regimen can be about 40 mg/day.

G. Antidepressants

For some patients with depression or at risk for depression, a treatment regimen comprising an antidepressant and a folate-comprising compound will be beneficial to enhance the therapeutic effect of the antidepressant drug. In some instances, the antidepressant and folate-comprising compound have a synergistic therapeutic effect.

Examples of antidepressants or antidepressant drugs can include, but are not limited to, mono-amine oxidase inhibitors such as phenelzine, tranylcypromine, and moclobemide; tricyclics such as imipramine, amitriptyline, desipramine, nortriptyline, doxepin, protriptyline, trimipramine, chlomipramine, and amoxapine; tetracyclics such as maprotiline; non-cyclics such as nomifensine; triazolopyridines such as trazodone; serotonin reuptake inhibitors such as fluoxetine, sertraline, paroxetine, citalopram, and fluvoxamine; serotonin receptor antagonists such as nefazadone; serotonin noradrenergic reuptake inhibitors such as venlafaxine, and milnacipran; noradrenergic and specific serotonergic agents such as mirtazapine; noradrenaline reuptake inhibitors such as reboxetine. Additional antidepressants that can be used in the invention described herein can include, but are not limited to, bupropion; natural products such as Kava-Kava, and St. John's Wort; dietary supplements such as s-adenosylmethionine; neuropeptides such as thyrotropin-releasing hormone; compounds targeting neuropeptide receptors such as neurokinin receptor antagonists; and hormones such as triiodothyronine.

In some embodiments, the antidepressant or the antidepressant drug can be a serotonin reuptake inhibitor (SRI) or selective serotonin reuptake inhibitor (SSRI). Examples of SRIs and/or SSRIs include, without limitations, citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214TA, OPC 14523, alaproclate, cyanodothepine, trimipramine, quinupramine, dothiepin, amoxapine, nitroxazepine, McN 5652, McN 5707, Ol 77, Org 6582, Org 6997, Org 6906, amitriptyline, amitriptyline N-oxide, nortriptyline, CL 255. 663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine and clovoxamine. The SRIs can be used in the form of the base or a pharmaceutically acceptable acid addition salt thereof.

In other embodiments, other therapeutic compounds that can cause an elevation in the extracellular level of 5-HT in the synatic cleft, e.g., tianeptine, can be used as an antidepressant.

A selective serotonin reuptake inhibitor (SSRI) is an inhibitor of the monoamine transporters, which has stronger inhibitory effect at the serotonin transporter than the dopamine and the noradrenaline transporters. Examples of selective serotonin reuptake inhibitors (SSRIs) can include, without limitations, fluoxetine, citalopram, paroxetine, escitalopram, sertraline, and any combinations thereof.

Additional SRIs and/or SSRIs that can be administered to a subject with depression in combination with a folate-comprising compound can include, for example, the ones described in the U.S. Pat. App. Pub. Nos.: 2005/0054688, and 2008/0138411; and U.S. Pat. Nos. 6,720,003; 6,787,560; 7,893,261; and 7,148,238.

One skilled in the art would be able to readily determine recommended dosage levels for known and/or marketed antidepressant drugs by consulting appropriate references such as drug package inserts, FDA guidelines, and the Physician's Desk Reference. In some embodiments, the antidepressant drug dose can range from 0.1 mg/day to about 1000 mg/day, from about 0.5 mg/day to about 500 mg/day, from about 1 mg/day to about 400 mg/day, from about 5 mg/day to about 300 mg/day, or from about 10 mg/day to about 200 mg/day. One of skill in the art can readily adjust dosage for each different antidepressant drug, depending on a number of factors such as types and/or potency of antidepressants, severity of depression, physical condition of a subject (e.g., ages, genders, and weights), administration routes, other medications taken by a subject, and any combinations thereof.

H. Pharmaceutical Compositions

Provided herein are pharmaceutical compositions comprising a therapeutically effective amount of a folate-comprising compound, and a pharmaceutically acceptable carrier for in vivo administration to subjects who carry at least one synergistic dual-biomarker combination.

In some embodiments, the therapeutically effective amount of a folate-comprising compound or folate, administered with an antidepressant or a pharmaceutically salt thereof is sufficient to increase the degree of improvement in at least one neuropsychological test, e.g., as measured by HAMD-17, HAMD-28 or other efficacy measures as described in the Examples, by at least about 5%, at least about 10%, at least about 20%, at least about 30%, at least about 40%, at least about 50%, at least about 60%, at least about 70%, at least about 80%, or at least about 90%, as compared to the degree of improvement obtained in the absence of the folate-comprising compound (e.g., with or without the antidepressant monotherapy). In some embodiments, the therapeutically effective amount of a folate-comprising compound or folate administered with an antidepressant or a pharmaceutically salt thereof is sufficient to increase the degree of improvement in at least one neuropsychological test, e.g., as measured by HAMD-17, HAMD-28 or other efficacy measures as described in the Examples, by at least about 1-fold, at least about 2-fold, at least about 3-fold, at least about 4-fold, at least about 5-fold or more, as compared to the degree of improvement obtained in the absence of the folate-comprising compound (e.g., with or without the antidepressant monotherapy).

In some embodiments, a dose of a folate-comprising compound or folate for administration to a human can be in the range of about 0.01 to about 50 mg per kilogram body weight of the recipient per day, in the range of about 0.05 to about 5 mg per kilogram body weight per day, or in the range of about 0.1 to about 1 mg per kilogram body weight per day. In certain embodiments, the desired dose can be presented as one single unit dosage form, e.g., containing about 0.5 mg to about 500 mg, about 5 mg to about 250 mg, about 10 mg to about 100 mg, or about 10 mg to about 50 mg. In some embodiments, one single unit dosage form can provide about 1 mg to about 70 mg folate, about 5 mg to about 60 mg folate, or from about 7 mg to about 50 mg folate. In other embodiments, one single unit dosage form can provide about 15 mg to about 50 mg folate. In yet other embodiments, one single unit dosage form can provide about 20 mg folate. In other embodiments, the desired dose can be presented in two, three, four, five or more sub-doses administered at appropriate intervals throughout the day. These sub-doses can be administered in unit dosage forms, for example, containing about 0.1 mg to about 250 mg, about 1 mg to about 100 mg, about 2 mg to about 20 mg, or about 2 mg to about 10 mg.

In some embodiments, the pharmaceutical composition can further comprise at least one antidepressant drug. In general, a dose of an antidepressant or a pharmaceutically acceptable salt thereof suitable for administration to a human is in the range of about 0.01 to 50 mg per kilogram body weight of the recipient per day, or in the range of 0.1 to 5 mg per kilogram body weight per day. In certain embodiments, the desired dose can be presented as one single unit dosage form, e.g., containing about 1 mg to about 500 mg, or about 5 mg to about 300 mg. In other embodiments, the desired dose can be presented in two, three, four, five or more sub-doses administered at appropriate intervals throughout the day. These sub-doses can be administered in unit dosage forms, for example, containing about 0.1 mg to about 100 mg or about 1 mg to about 50 mg.

A pharmaceutically acceptable carrier includes a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alchols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutically acceptable carriers can vary in a composition described herein, depending on the administration route and formulation. For example, the pharmaceutically acceptable composition described herein can be delivered via injection. These routes for administration (delivery) include, but are not limited to, subcutaneous or parenteral including intravenous, intraarterial, intramuscular, intraperitoneal, intramyocardial, and infusion techniques. In one embodiment, the pharmaceutical acceptable composition is in a form that is suitable for injection. In another embodiment, the pharmaceutical composition is formulated for delivery by a catheter.

When administering a pharmaceutical composition parenterally, it can be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS). In some embodiments, the pharmaceutical composition can be formulated in an emulsion or a gel.

In some embodiments, the pharmaceutical compositions described herein can be formulated for oral administration or for inhalation. For oral administration, suitable dosage forms can include tablets, troches, cachets, caplets, and capsules, including hard and soft gelatin capsules.

In some embodiments, both an antidepressant and a folate-comprising compound can be formulated in a single pharmaceutical composition. For example, both an antidepressant and a folate-comprising compound can be formulated in a single tablet for oral administration.

In some embodiments where the antidepressant and folate-comprising compound are formulated in a single composition, they can be released from the composition at the same time or at different times. By way of example only, if the folate-comprising compound is formulated in an outer layer of a composition (e.g., a tablet or drug-delivery particle) while the antidepressant is formulated in an inner layer of the composition, the folate-comprising compound could be released from the composition first with a faster rate, while the antidepressant could be released from the composition later with a slower rate. On the other hand, if the antidepressant and the folate-comprising compound are mixed homogenously within the composition, both can be released simultaneously from the composition.

In other embodiments, an antidepressant and a folate-comprising compound can be formulated in separate pharmaceutical compositions for the same or different routes of administration during a therapy course. For example, an antidepressant can be formulated for inhalation administration while a folate-comprising compound can be formulated for oral administration. In other embodiments, both the antidepressant and folate-comprising compound can be formulated for oral administration, e.g., in separate tablets.

The effective amount of folate administered to a selected human subject for treatment of depression as described herein is significantly higher than the typical amount taken as a dietary supplement (between 50-600 μg/day). In some embodiments, the effective amount of folate administered to a selected human subject is at least about 2-fold, at least about 5-fold, at least about 10-fold, at least about 25-fold, at least about 50-fold, at least about 100-fold, at least about 250-fold, at least about 500-fold, at least about 1000-fold or more than the typical amount taken as a dietary supplement. Accordingly, in some embodiments, the folate-comprising compound is desirable to be formulated in slow-release or sustained release composition.

Accordingly, in some embodiments, the pharmaceutical compositions comprising a folate-comprising compound (with or without an antidepressant) can be formulated for sustained release or sustained delivery. In some embodiments, the pharmaceutical compositions can be formulated in controlled-release drug-delivery systems, e.g., to provide sustained release of a folate-comprising compound (and optionally an antidepressant). As used herein, the term “sustained release” or “sustained delivery” refers to continual delivery of a therapeutic agent in vivo over a period of time following administration. For example, sustained release can occur over a period of at least about 1 hour, at least about 2 hours, at least about 3 hours, at least about 4 hours, at least about 5 hours, at least about 6 hours, at least about 9 hours, at least about 12 hours, at least about 16 hours, at least about 24 hours following administration. In some embodiments, sustained release can occur over a period of at least about 1 day, at least about 2 days, at least about 3 days, at least about 4 days, at least about 5 days, at least about 6 days, at least about 7 days following administration. In some embodiments, the release of a folate-comprising compound from a drug-delivery system can be steady state (zero-order kinetics) with at least about 30% (e.g., including at least about 40%, at least about 50%, at least about 60%, at least about 70%, at least about 80%, at least about 90%, at least about 95% or more) of the folate-comprising compound (and optionally an antidepressant) released between about 3-6 hours post administration, or between about 4-5 hours post administration. In one embodiment, the release of a folate-comprising compound (and optionally an antidepressant) from a drug-delivery system can be steady state (zero-order kinetics) with substantially full release (e.g., ˜100%) of the folate-comprising compound released between about 3-6 hours post administration, or between about 4-5 hours post administration. In some embodiments, the folate-comprising compound can be released from a drug-delivery system at a rate that is slow enough not to overload the intestinal absorption capacity of a patient's duodenum (first ⅓ of the small intestines where ˜90% of the absorption occurs for a folate-comprising compound, e.g., L-MTHF). In some embodiments, the folate-comprising compound and an antidepressant (if any) can be released from a drug-delivery system concurrently or separately, with the same or a different release rate.

Any drug delivery system (e.g., but not limited to polymer-based) that provides a sustained release of a folate-comprising compound (and optionally an antidepressant) over a pre-determined period of time can be used for administration of a folate-comprising compound (and optionally an antidepressant). In one embodiment, the drug-delivery system can be a caplet design large enough to be blocked by a pyloric valve between the stomach and the duodenum, thus allowing the caplet to slowly and partially dissolve over a desirable period of time, e.g., over a period of about 2-3 hours, during which the folate-comprising compound is steadily released from the caplet. As the caplet dissolves to a size that can get through the pyloric valve at which time it completes its steady state release (e.g., an additional period of time, e.g., an additional 2 hours), the caplet can continue to travel into the jejunum (the second third of the small intestines) where absorption is minimal.

In some embodiments, a drug delivery system can use a blend of hydrophilic and hydrophobic polymers to control release of a folate-comprising compound (and optionally an antidepressant) via diffusion through, and erosion of, a polymer matrix.

In some embodiments, a drug delivery system can comprise a folate-comprising compound (and optionally an antidepressant) encapsulated in polymer-based particles. These folate-containing polymer-based particles can be filled into capsules or single-dose sachets for additional control of release.

Controlled-release (e.g., sustained release) drug delivery systems for different administration methods (e.g., oral administration, injection, implantation, and inhalation) are known in the art and can be adopted to deliver a folate-comprising compound (and optionally an antidepressant) for the treatment methods described herein. See, e.g., International Pat. App. Nos. WO 2012/111961 (oral formulation), WO 2012/131678 (injectable formulation); U.S. Pat. App. Nos. US 2012/0258161 (implantable formulation), US 2001/0038854, US 2001/0033866; and U.S. Pat. No. 8,268,347 (inhalation formulation), the disclosures of which are hereby incorporated by reference in their entirety for all purposes, for various types of drug-delivery systems to deliver an active agent via various administration routes.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, binders, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to compositions described herein, however, any vehicle, diluent, or additive used should have to be biocompatible with the antidepressant or a pharmaceutically acceptable salt thereof and/or a folate-comprising compound.

The pharmaceutical compositions can be isotonic, i.e., they can have the same osmotic pressure as blood and lacrimal fluid. The desired isotonicity of the compositions of the composition described herein can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Typically, any additives (in addition to the antidepressant and/or folate-comprising compound) can be present in an amount of 0.001 to 50 wt % solution in phosphate buffered saline, and the active ingredient is present in the order of micrograms to milligrams to grams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject with compression, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

The compositions described herein can be prepared by mixing the ingredients following generally-accepted procedures. For example, the ingredients can be mixed in an appropriate pharmaceutically acceptable carrier and the mixture can be adjusted to the final concentration and viscosity by the addition of water or thickening agent and possibly a buffer to control pH or an additional solute to control tonicity. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid).

I. Kits

In some embodiments, provided herein is a kit for use in selecting a treatment regimen for a human subject diagnosed as having depression or having a risk for depression. The kit comprises at least one reagent for determining the presence or absence of at least two of the following single nucleotide polymorphisms (SNPs) in a sample taken from the subject:
(i) a SNP at position 677 of SEQ ID NO: 1 or position 27 of SEQ ID NO: 7 as identified as rs1801133 comprising at least one thymine “T” allele for MTHFR;
(ii) a SNP at position 2756 of SEQ ID NO: 2 or position 27 of SEQ ID NO: 9 as identified as rs1805087 comprising at least one guanine “G” allele for MTR;
(iii) a SNP at position 27 of SEQ ID NO: 18 as identified as rs8007267 comprising at least one thymine “T” allele for GCH1;
(iv) a SNP at position 27 of SEQ ID NO: 24 as identified as rs4680 comprising two guanine “G” alleles for COMT; and instructions for use of the kit.

In some embodiments, the at least one reagent is selected from the group consisting of a restriction enzyme, an oligonucleotide, a nucleic acid probe, a polymerase and a combination thereof. The kit can be used in the methods described herein for genotyping the folate-responsive genetic biomarkers. In some embodiments, the kit comprises at least one set of primers flanking any one of the SNPs. In some instances, the at least two sets of primers may amplify at least two of the SNPs in a multiplex amplification assay.

Embodiments of the various aspects described herein can also be described by any one of the following numbered paragraphs.

1. An assay for selecting a treatment regimen for a human subject diagnosed as having depression or having a risk for depression, the assay comprising:

(a) analyzing a sample from the subject to determine the genotype of at least two genetic biomarkers selected from the group consisting of methylenetetrahydrofolate reductase (MTHFR), methionine synthase (MTR), GTP cyclohydrolase 1 (GCH1), catechol-O-methyltransferase (COMT), and a combination thereof:

(b) detecting by genotyping for the presence or absence of a single nucleotide polymorphism (SNP) in each of the at least two genetic biomarkers, wherein the presence of the SNP is set forth as the following:

(i) a SNP at position 677 of SEQ ID NO: 1 or position 27 of SEQ ID NO: 7 as identified as rs1801133 comprising at least one thymine “T” allele for MTHFR;

(ii) a SNP at position 2756 of SEQ ID NO: 2 or position 27 of SEQ ID NO: 9 as identified as rs1805087 comprising at least one guanine “G” allele for MTR;

(iii) a SNP at position 27 of SEQ ID NO: 18 as identified as rs8007267 comprising at least one thymine “T” allele for GCH1;

(iv) a SNP at position 27 of SEQ ID NO: 24 as identified as rs4680 comprising two guanine “G” alleles for COMT; and

(c) selecting the treatment regimen comprising an effective amount of a folate-comprising compound based on the presence of said SNPs.

2. The assay of paragraph 1, further comprising administering said treatment regimen.

3. The assay of paragraph 1 or 2, wherein the at least two genetic biomarkers are the MTHFR and MTR pair.

4. The assay of any one of paragraphs 1-3, wherein the at least two genetic biomarkers are the GCH1 and COMT pair.

5. The assay of any one of paragraphs 1-4, wherein step (a) further comprises determining at least one additional condition selected from the group consisting of obesity, SAM/SAH ratio, level of 4-HNE, level of hsCRP, and a combination thereof.

6. The assay of paragraph 5, wherein step (b) further comprises detecting at least one of the following conditions:

(i) an expression level ratio of SAM to SAH smaller than a pre-determined reference ratio;

(ii) an expression level of 4-HNE greater than a first pre-determined reference value; and

(iii) an expression level of hsCRP greater than a second pre-determined reference value.

7. The assay of paragraph 5, wherein obesity is determined, if any of the following conditions are present in the subject: a BMI value is 30 kg/m² or greater, a waist circumference is greater than 40 inches in men or greater than 35 inches in women, a waist-hip ratio is about 0.95 for men or above 0.8 for women, or a body fat percentage of at least about 25% in men or at least about 32% in women.

8. The assay of paragraph 6, wherein the pre-determined reference ratio of SAM/SAH is about 3.0 if measured in a plasma sample from a normal, healthy subject.

9. The assay of paragraph 6, wherein the first pre-determined reference value of 4-HNE is about 0.24 mole per liter or about 0.04 mg per liter if measured in a serum sample from a normal, healthy subject.

10. The assay of paragraph 6, wherein the first pre-determined reference value of 4-HNE is about 3.0 mg per liter if measured in a plasma sample from a normal, healthy subject.

11. The assay of paragraph 6, wherein the second pre-determined reference value of hsCRP is from about 0.5 mg per liter to about 4.5 mg per liter if measured in a serum sample from a normal, healthy subject.

12. The assay of paragraph 6, wherein the second pre-determined reference value of hsCRP is about 2.3 mg per liter if measured in a plasma sample from a normal, healthy subject.

13. The assay of any of the preceding paragraphs, wherein the sample is selected from the group consisting of a blood sample, a serum sample, a plasma sample, a urine sample, a buccal sample, and a saliva sample.

14. The assay of any of the preceding paragraphs, wherein depression is major depressive disorder.

15. The assay of any of the preceding paragraphs, wherein the effective amount of the folate-comprising compound is about 15 mg/day to about 50 mg/day.

16. The assay of paragraph 15, wherein the effective amount of the folate-comprising compound is about 20 mg/day.

17. The assay of paragraph 15, wherein the effective amount of the folate-comprising compound is about 40 mg/day.

18. The assay of paragraph 17, wherein the folate-comprising compound is administered at about a 20 mg dose twice per day.

19. The assay of any of the preceding paragraphs, wherein said treatment regimen further comprises an antidepressant drug.

20. The assay of paragraph 19, wherein the antidepressant drug is a selective serotonin reuptake inhibitor (SSRI).

21. The assay of paragraph 20, wherein the selective serotonin reuptake inhibitor is selected from the group consisting of fluoxetine, citalopram, paroxetine, escitalopram, sertraline, or a combination thereof.

22. The assay of any of the preceding paragraphs, wherein the subject has an inadequate response or is resistant to an antidepressant monotherapy.

23. The assay of any of the preceding paragraphs, wherein detecting the presence or absence of the SNP comprises a hybridization assay, an amplification assay, a primer extension assay, an oligonucleotide ligation assay, a sequencing assay or a combination thereof.

24. The assay of any one of paragraphs 6-22, wherein detecting the condition comprises an immunoassay, immunohistochemistry (IHC), gas chromatography (GC), mass spectrometry (MS), high performance liquid chromatography (HPLC), nuclear magnetic resonance (NMR) spectrometry, or flow cytometry.

25. A method for treating at least one symptom of depression in a human subject diagnosed as having depression or having a risk for depression, the method comprising:

a) analyzing a sample from the subject to determine the presence or absence of at least one combination of at least two single nucleotide polymorphisms (SNPs) selected from the group consisting of:

(i) a SNP at position 677 of SEQ ID NO: 1 or position 27 of SEQ ID NO: 7 as identified as rs1801133 comprising at least one thymine “T” allele for MTHFR and a SNP at position 2756 of SEQ ID NO: 2 or position 27 of SEQ ID NO: 9 as identified as rs1805087 comprising at least one guanine “G” allele for MTR; and

(ii) a SNP at position 27 of SEQ ID NO: 18 as identified by rs8007267 comprising at least one thymine “T” allele for GCH1 and a SNP at position 27 of SEQ ID NO: 24 as identified as rs4680 comprising two guanine “G” alleles for COMT, wherein the presence of at least one combination is associated with a symptom-reducing response to a folate-comprising compound; and

b) administering a treatment regimen comprising an effective amount of the folate-comprising compound to the subject to treat at least one symptom of depression.

26. The method of paragraph 25, wherein the treatment regimen further comprises an antidepressant drug.

27. The method of paragraph 26, wherein the antidepressant drug is a selective serotonin reuptake inhibitor (SSRI).

28. The method of paragraph 27, wherein the selective serotonin reuptake inhibitor is selected from the group consisting of fluoxetine, citalopram, paroxetine, escitalopram, sertraline and a combination thereof.

29. The method of any one of paragraphs 25-28, wherein the presence of at least one thymine “T” allele or the complement thereof at rs1801133 and the presence of at least one guanine “G” alleles or the complement thereof at rs1805087 are associated with a symptom-reducing response to the folate-comprising compound.

30. The method of any one of paragraphs 25-28, wherein the presence of at least one thymine “T” allele or the complement thereof at rs8007267 and the presence of two guanine “G” alleles or the complement thereof at rs4860 are associated with a symptom-reducing response to the folate-comprising compound.

31. The method of any one of paragraphs 25-30, wherein depression is major depressive disorder.

32. The method of any one of paragraphs 25-31, wherein the at least one symptom of depression is selected from the group consisting of depressed mood, guilt, reduced work or interests, psychomotor retardation, agitation, psychic anxiety, somatic anxiety, general somatic symptoms, cognition impairment, and a combination thereof.

33. The method of any one of paragraphs 25-32, wherein the subject is obese.

34. The method of paragraph 33, wherein the obesity is characterized by at least one of the following conditions present in the subject:

a) a BMI value greater than 30 kg/m²;

b) a waist circumference greater than 40 inches in men, or greater than 35 inches in women;

c) a waist-hip ratio above 0.95 for men or above 0.80 for women; and

d) a body fat percentage of at least about 25% in men or at least about 32% in women.

35. The method of any one of paragraphs 25-34, wherein the sample is selected from the group consisting of a blood sample, a serum sample, a plasma sample, a urine sample, a buccal sample and a saliva sample.

36. The method of any one of paragraphs 25-35, wherein the effective amount of the folate-comprising compound is about 15 mg/day to about 50 mg/day.

37. The method of paragraph 36, wherein the effective amount of the folate-comprising compound is about 20 mg/day.

38. The method of paragraph 36, wherein the effective amount of the folate-comprising compound is about 40 mg/day.

39. The method of any one of paragraphs 35-38, wherein the effective amount of the folate-comprising compound is administered as a single daily dose.

40. The method of any one of paragraphs 25-38, wherein the effective amount of the folate-comprising compound is administered in at least two divided doses per day.

41. The method of any one of paragraphs 25-36, 38 or 40, wherein the folate-comprising compound is administered in about 20 mg per dose twice per day.

42. The method of any one of paragraphs 25-41, wherein the folate-comprising compound is administered orally.

43. The method of any one of paragraphs 25-42, wherein the folate-comprising compound is L-methylfolate.

44. The method of any one of paragraphs 25-43, wherein the subject has an inadequate response or is resistant to antidepressant monotherapy.

45. The method of any one of paragraphs 25-44, further comprising measuring the expression level of at least one biomarker and determining whether the level of the biomarker(s) is associated with a symptom-reducing response to a folate-comprising compound.

46. The method of paragraph 45, wherein the additional biomarker is selected from the group consisting of SAM, SAH, 4-HNE, hsCRP and a combination thereof.

47. The method of paragraph 45, wherein the subject is likely to have a symptom-reducing response to the folate-comprising compound if one or more of the following conditions are met:

a) the expression ratio of SAM to SAH (SAM/SAH) is smaller than a pre-determined reference ratio;

b) the expression level of 4-HNE greater than a first pre-determined reference value; or

c) the expression level of hsCRP greater than a second pre-determined reference value.

48. The method of paragraph 47, wherein the pre-determined reference ratio of SAM/SAH is from about 4 to about 12 if measured in a serum sample from a normal, healthy subject.

49. The method of paragraph 47, wherein the pre-determined reference ratio of SAM/SAH is about 3.0 if measured in a plasma sample from a normal, healthy subject.

50. The method of paragraph 47, wherein the first pre-determined reference value of 4-HNE is about 0.24 mole per liter or about 0.04 mg per liter if measured in a serum sample from a normal, healthy subject.

51. The method of paragraph 47, wherein the first pre-determined reference value of 4-HNE is about 3.0 mg per liter if measured in a plasma sample from a normal, healthy subject.

52. The method of paragraph 47, wherein the second pre-determined reference value of hsCRP is from about 0.5 mg per liter to about 4.5 mg per liter if measured in a serum sample from a normal, healthy subject.

53. The method of paragraph 47, wherein the second pre-determined reference value of hsCRP is about 2.3 mg per liter if measured in a plasma sample from a normal, healthy subject.

54. A method for improving the effectiveness of an antidepressant drug administered to a human subject who is diagnosed as having depression or having a risk for depression, the method comprising:

administering a therapeutic composition comprising an effective amount of a folate-comprising compound in combination with the antidepressant drug if the subject is carrying at least one of the following combinations of single nucleotide polymorphisms (SNPs):

(i) a SNP at position 677 of SEQ ID NO: 1 or position 27 of SEQ ID NO: 7 as identified as rs1801133 comprising at least one thymine “T” allele for MTHFR and a SNP at position 2756 of SEQ ID NO: 2 or position 27 of SEQ ID NO: 9 as identified as rs1805087 comprising at least one guanine “G” allele for MTR; or

(ii) a SNP at position 27 of SEQ ID NO: 18 as identified by rs8007267 comprising at least one thymine “T” allele for GCH1 and a SNP at position 27 of SEQ ID NO: 24 as identified as rs4680 comprising two guanine “G” alleles for COMT.

55. The method of paragraph 54, wherein the subject has received antidepressant monotherapy.

56. The method of paragraphs 54 or 55, wherein the subject had an inadequate response to antidepressant monotherapy.

57. The method of paragraph 56, wherein the inadequate response is based on a clinical assessment.

58. The method of any one of paragraphs 54-57, wherein the antidepressant drug is a selective serotonin reuptake inhibitor (SSRI).

59. The method of paragraph 58, wherein the selective serotonin reuptake inhibitor is a member selected from the group consisting of fluoxetine, citalopram, paroxetine, escitalopram, sertraline, and a combination thereof.

60. The method of any one of paragraphs 54-59, wherein the effective amount of the folate-comprising compound is about 15 mg/day to about 50 mg/day.

61. The method of paragraph 60, wherein the effective amount of the folate-comprising compound is about 20 mg/day.

62. The method of paragraph 61, wherein the effective amount of the folate-comprising compound is about 40 mg/day.

63. The method of any one of paragraphs 54-62, wherein the effective amount of the folate-comprising compound is administered as a single daily dose.

64. The method of any one of paragraphs 54-62, wherein the effective amount of the folate-comprising compound is administered in at least two divided doses per day.

65. The method of any one of paragraphs 54-60, 62 or 64, wherein the folate-comprising compound is administered at about 20 mg, twice a day.

66. The method of any one of paragraphs 54-65, wherein the folate-comprising compound is administered orally.

67. The method of any one of paragraphs 54-66, wherein the folate-comprising compound is L-methylfolate.

68. The method of any one of paragraphs 54-67, wherein the depression is major depressive disorder.

69. A folate-comprising composition for use in the treatment of depression in a human subject who is diagnosed as having depression or having a risk for depression and carries at least two of the following SNPs selected from the group consisting of:

(i) the SNP at position 677 of SEQ ID NO: 1 or position 27 of SEQ ID NO: 7 as identified as rs1801133 comprising at least one thymine “T” allele for MTHFR;

(ii) the SNP at position 2756 of SEQ ID NO: 2 or position 27 of SEQ ID NO: 9 as identified as rs1805087 comprising at least one guanine “G” allele for MTR;

(iii) the SNP at position 27 of SEQ ID NO: 18 as identified as rs8007267 comprising at least one thymine “T” allele for GCH1; and

(iv) the SNP at position 27 of SEQ ID NO: 24 as identified as rs4680 comprising two guanine “G” alleles for COMT.

70. The composition of paragraph 69, wherein the at least two SNPs are the SNP at position 677 of SEQ ID NO: 1 or position 27 of SEQ ID NO: 7 as identified as rs1801133 comprising at least one thymine “T” allele for MTHFR and the SNP at position 2756 of SEQ ID NO: 2 or position 27 of SEQ ID NO: 9 as identified as rs1805087 comprising at least one guanine “G” allele for MTR.

71. The composition of paragraph 69, wherein the at least two SNPs are the SNP at position 27 of SEQ ID NO: 18 as identified as rs8007267 comprising at least one thymine “T” allele for GCH1 and the SNP at position 27 of SEQ ID NO: 24 as identified as rs4680 comprising two guanine “G” alleles for COMT.

72. The composition of any one of paragraphs 69-71, wherein depression is major depressive disorder.

73. The composition of any one of paragraphs 69-72, wherein the subject is receiving at least one antidepressant drug.

74. The composition of any one of paragraphs 69-73, wherein the subject who carries the at least two of the SNPs is administered an adjunctive therapy comprising the folate-comprising composition and an antidepressant drug.

75. The composition of paragraph 74, wherein the antidepressant drug is a selective serotonin reuptake inhibitor (SSRI).

76. The composition of any one of paragraphs 69-74, wherein the folate-comprising compound comprises about 15 mg to about 50 mg of L-methylfolate.

77. The composition of paragraph 76, wherein the folate-comprising compound comprises about 20 mg of L-methylfolate.

78. The composition of any one of paragraphs 69-77, wherein the folate-comprising compound has a pre-determined release profile.

79. The composition of any one of paragraphs 69-78, wherein the pre-determined release profile is a sustained release.

80. The composition of paragraph 79, wherein the pre-determined release profile is a steady-state release.

81. The composition of any one of paragraphs 69-78, wherein the pre-determined release profile is a pulsatile release.

82. The composition of any one of paragraphs 69-78, wherein the pre-determined release profile is a chrono-controlled release.

83. The composition of any one of paragraphs 69-82, wherein the folate-comprising composition is formulated to release at least 30% of the folate-comprising compound over a period of at least 3 to 6 hours upon the administration of the composition.

84. A kit for use in selecting a treatment regimen for a human subject diagnosed as having depression or having a risk for depression comprising:

at least one reagent for determining the presence or absence of at least two of the following single nucleotide polymorphisms (SNPs) in a sample taken from the subject:

(i) a SNP at position 677 of SEQ ID NO: 1 or position 27 of SEQ ID NO: 7 as identified as rs1801133 comprising at least one thymine “T” allele for MTHFR;

(ii) a SNP at position 2756 of SEQ ID NO: 2 or position 27 of SEQ ID NO: 9 as identified as rs1805087 comprising at least one guanine “G” allele for MTR;

(iii) a SNP at position 27 of SEQ ID NO: 18 as identified as rs8007267 comprising at least one thymine “T” allele for GCH1;

(iv) a SNP at position 27 of SEQ ID NO: 24 as identified as rs4680 comprising two guanine “G” alleles for COMT; and

instructions for use of said kit.

85. The kit of paragraph 84, wherein the at least one reagent is selected from the group consisting of a restriction enzyme, an oligonucleotide, a nucleic acid probe, a polymerase and a combination thereof.

### IV. Examples

The following examples are offered to illustrate, but not to limit the claimed invention.

### Example 1

### Identification of Biomarkers for Selecting Patients with Depression for a Treatment Comprising a Folate-Comprising Compound in Combination with an SSRI

A double-blind, placebo-controlled study of 6(S)-5-MTHF among SSRI resistant outpatients with major depressive disorder (MDD) is performed was performed to identify specific combinations of biomarkers that are associated with a greater efficacy response (e.g., a response greater than an additive response produced by both SNPs, or a response greater than a response produced by either SNP alone) when a patient is administered with a folate-comprising compound (e.g., 6(S)-5-MTHF) in addition to an antidepressant drug, e.g., an SSRI. See, e.g., Papakostas G I et al., Am J Psychiatry, 169:1267-74 (2012)

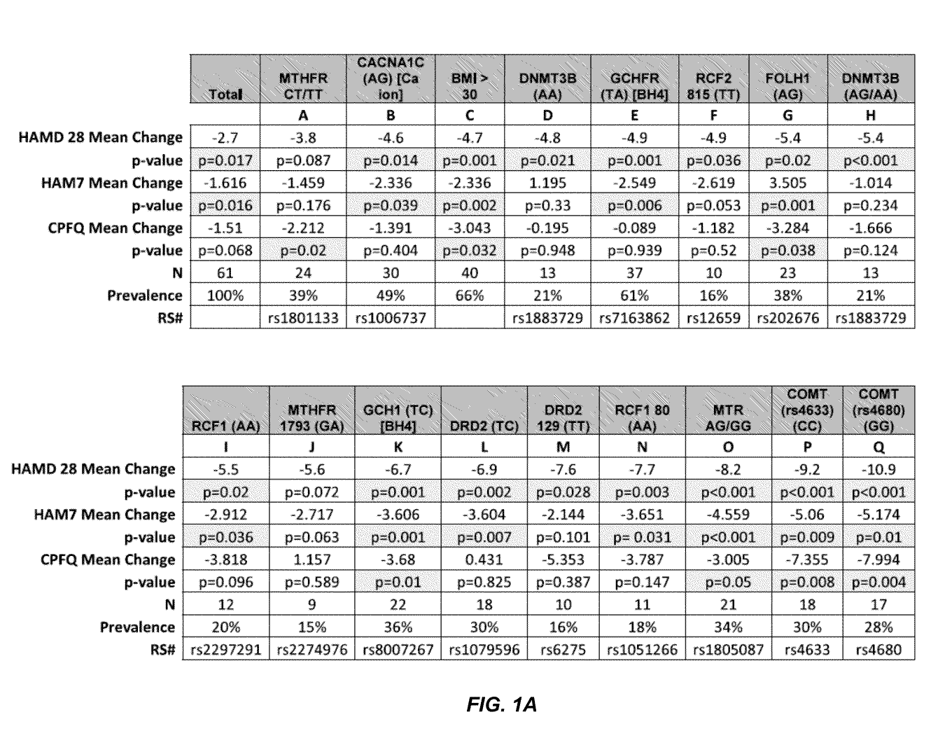

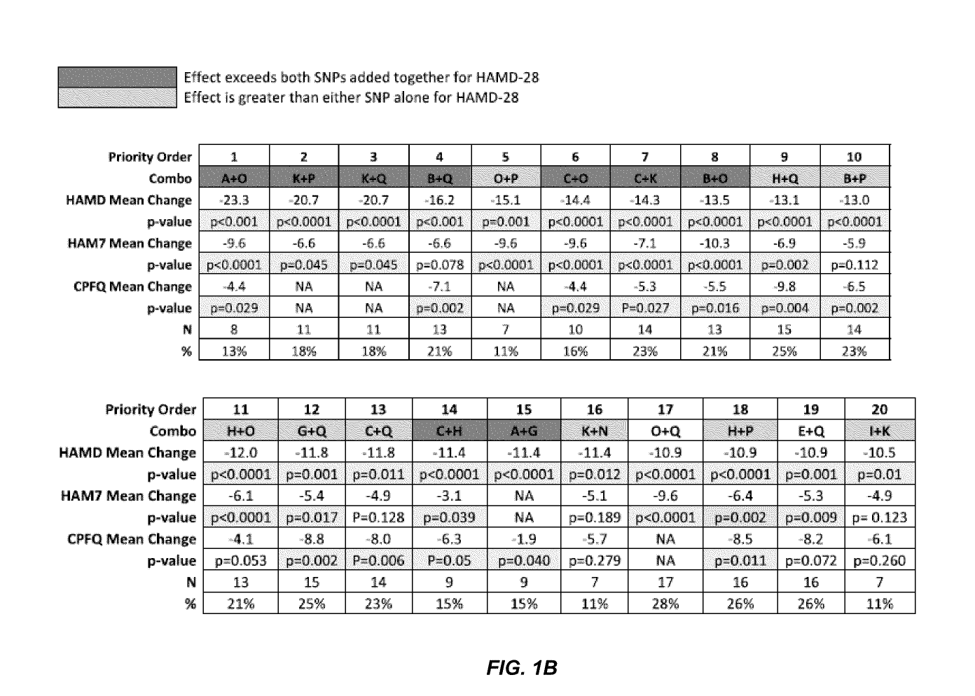

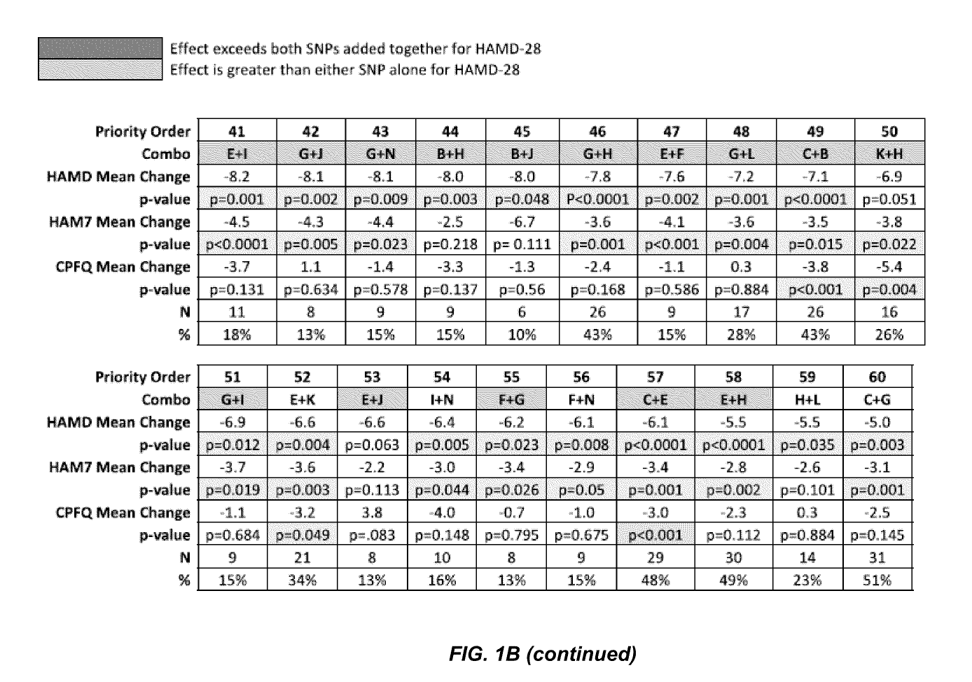

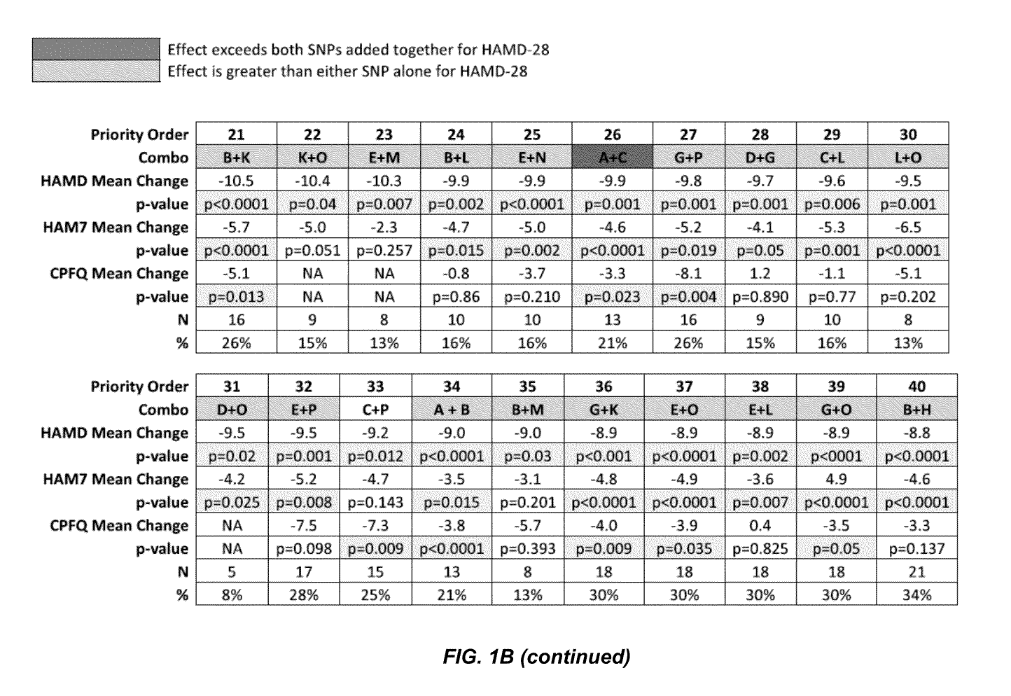

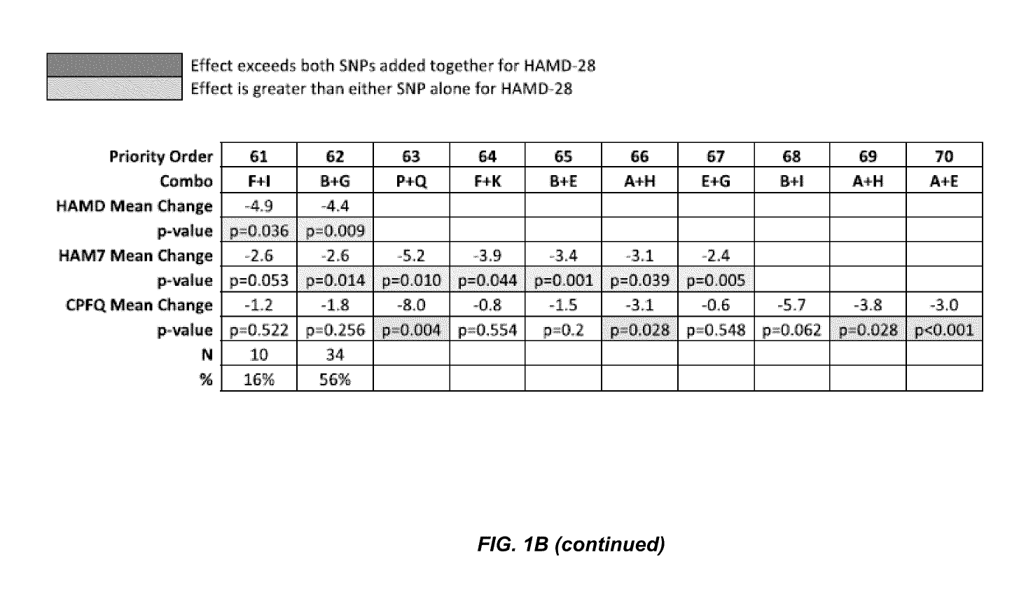

Various combinations of two genetic biomarkers, one genetic biomarker and a clinical feature were assessed for their effects on the efficacy of the treatment comprising a folate-comprising compound (e.g., 6(S)-5-MTHF) and an SSRI in patients with depression. Particularly, specific combinations that were assessed are shown in FIGS. 1B, 2, 4, and 5.

The results of how various single marker and dual-biomarker combinations affect the degree of improvement, as measured by HAMD-17 score, HAMD-28 score, or cognitive and physical function questionnaire (CPFQ) score, when patients are treated with a folate-comprising compound (e.g., 6(S)-5-MTHF) and an SSRI, are shown in FIGS. 1-6. The efficacy effect is determined by measuring the mean change in the HAMD-17 score, HAMD-28 score, or HAMD-28 by the end of Phase I and Phase II, as compared to the baseline (e.g., subjects without treatment).

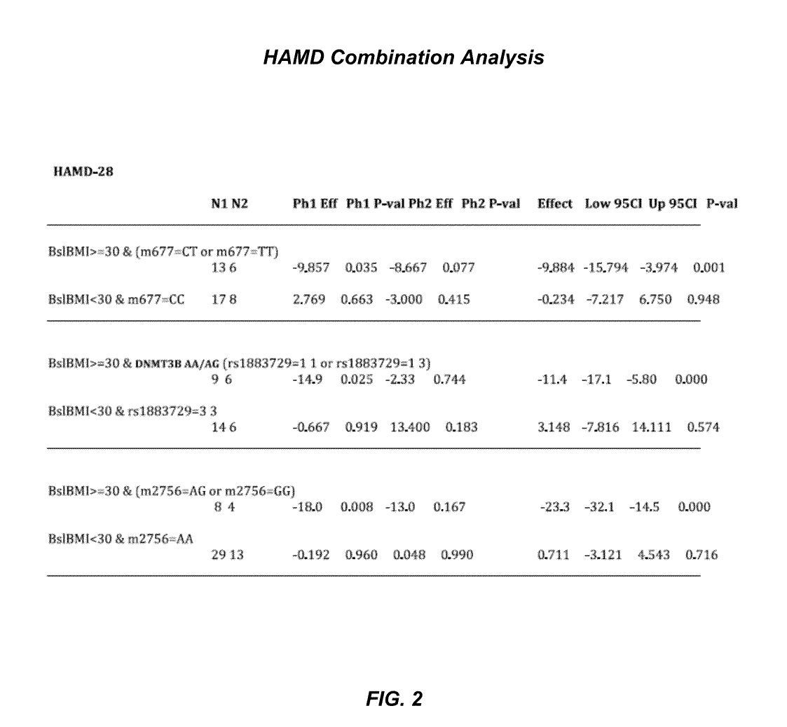

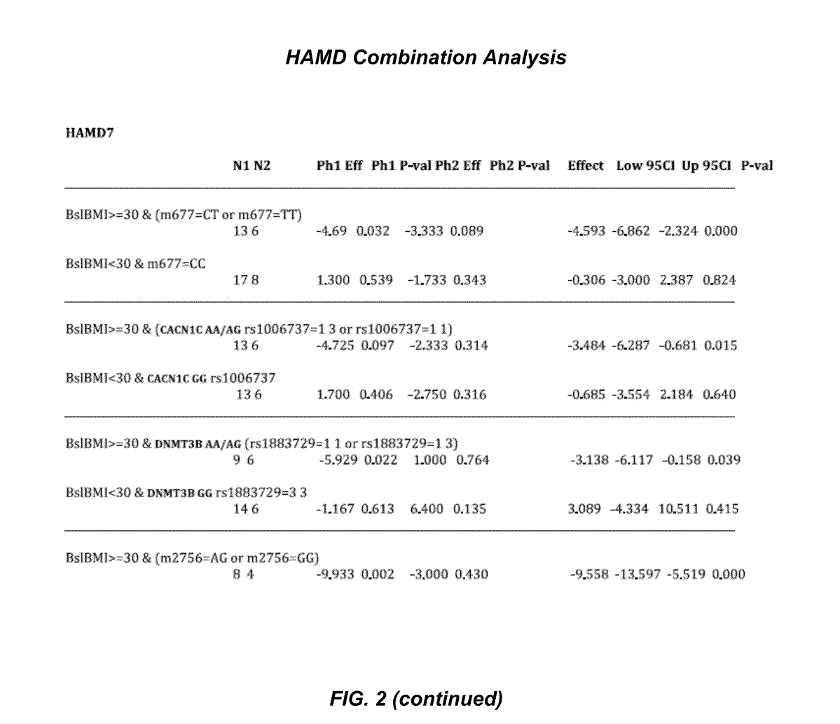

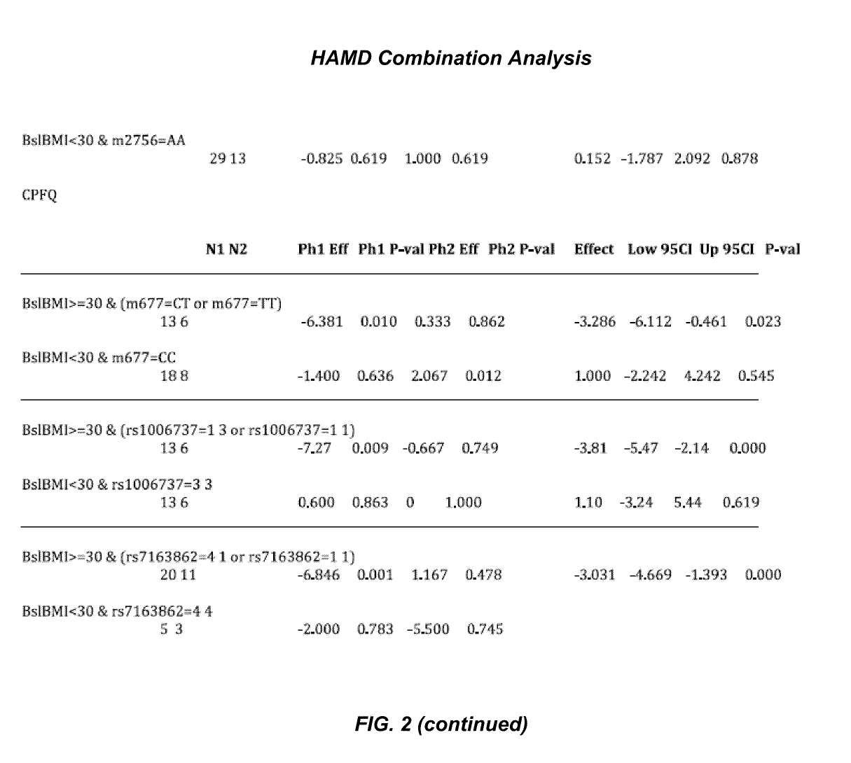

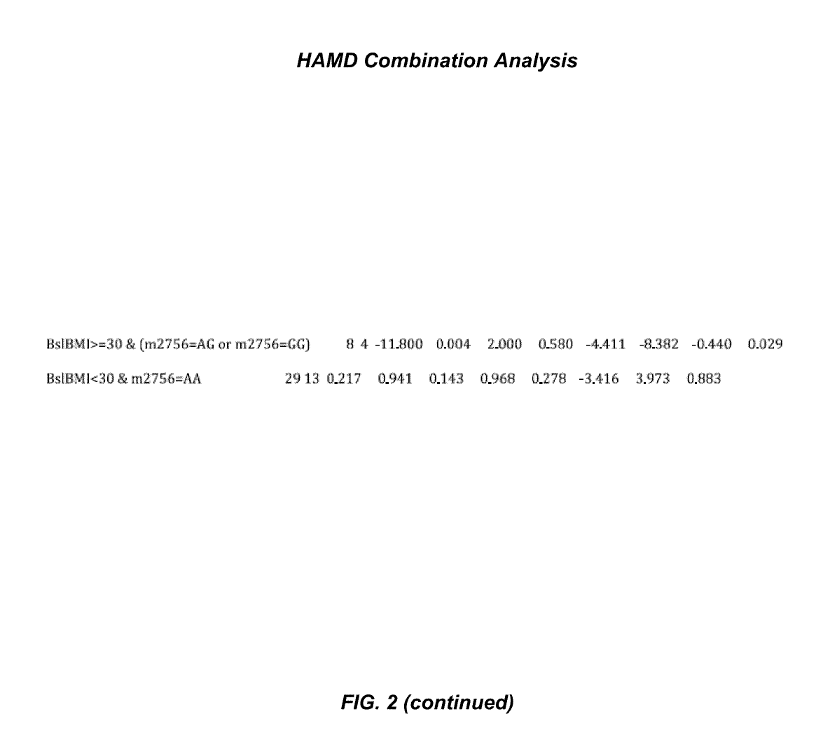

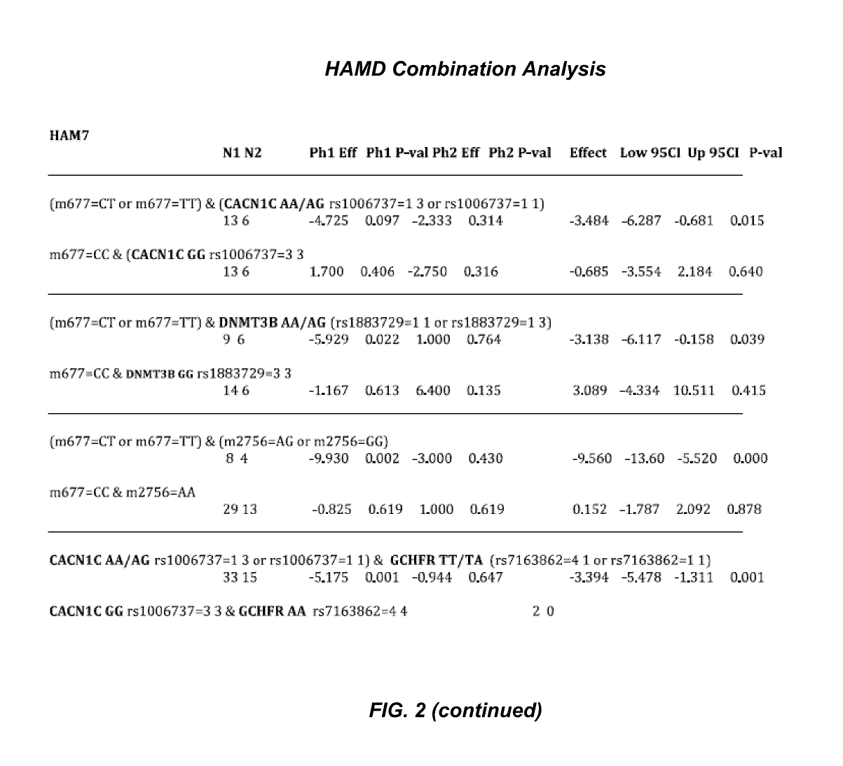

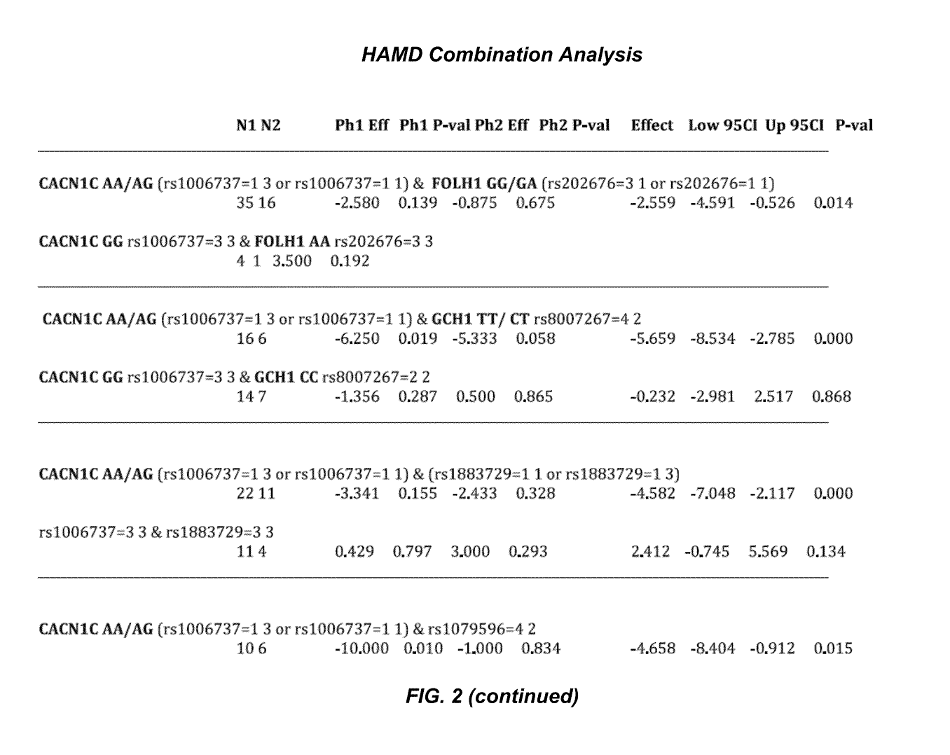

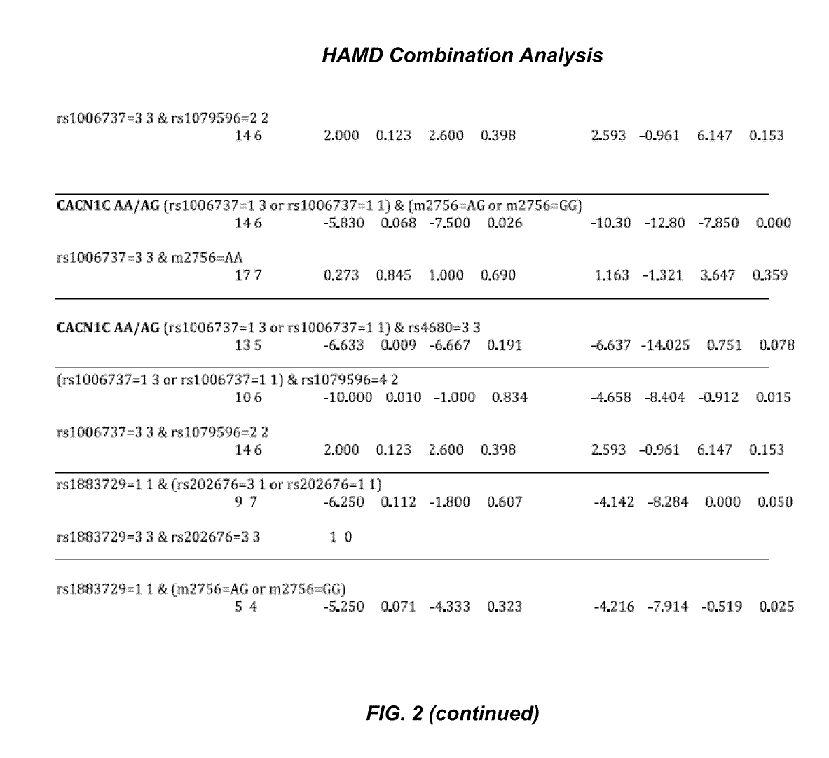

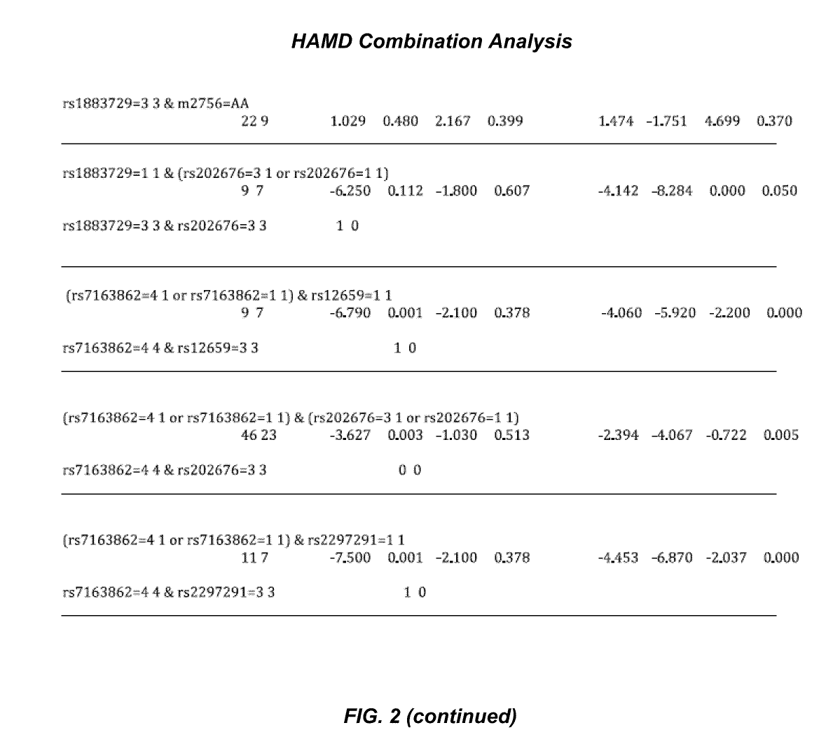

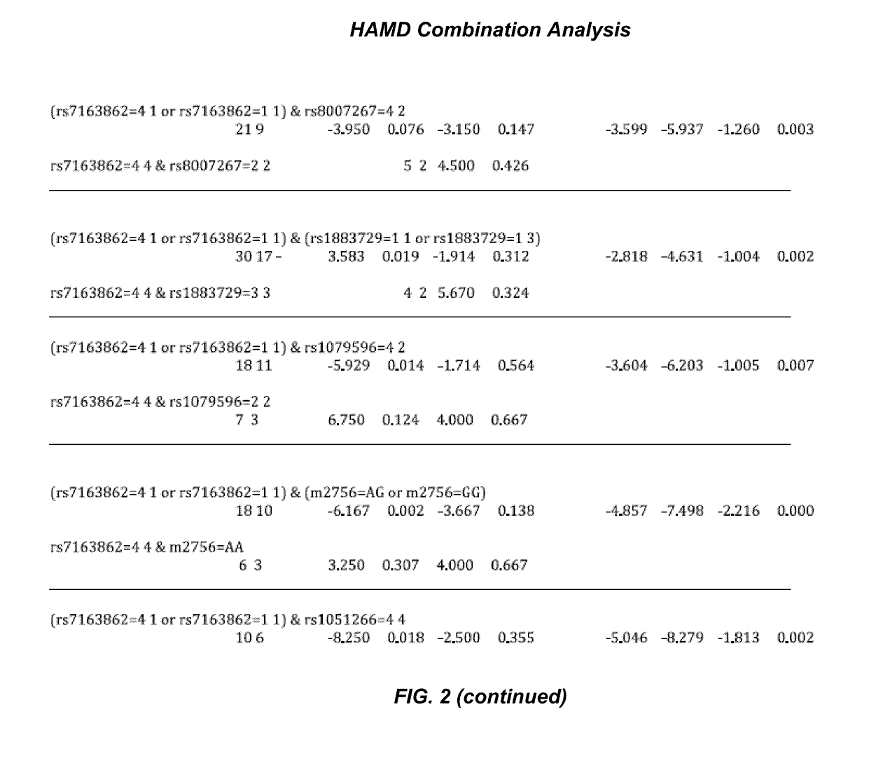

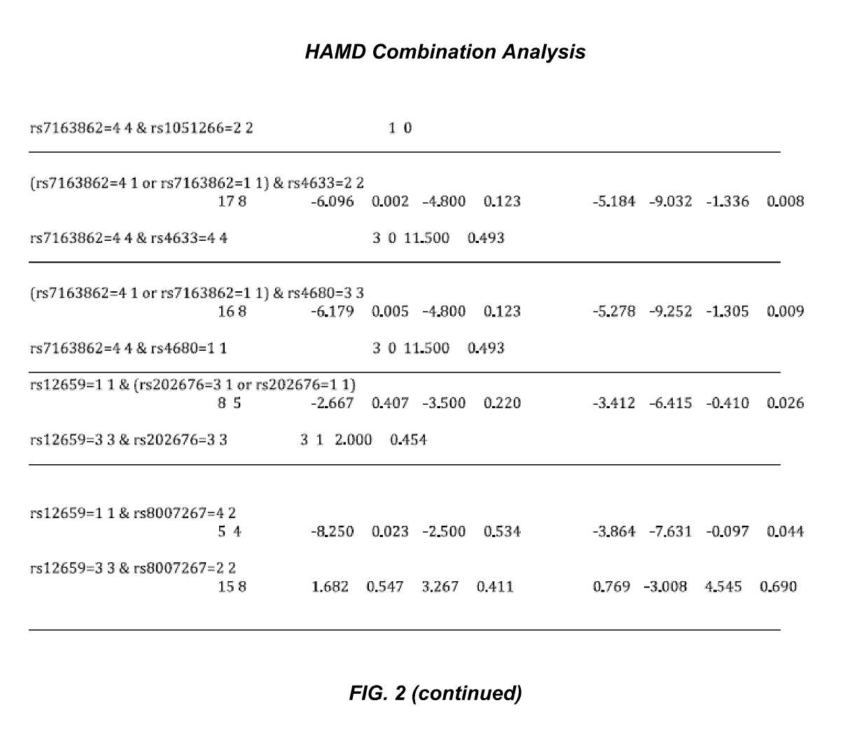

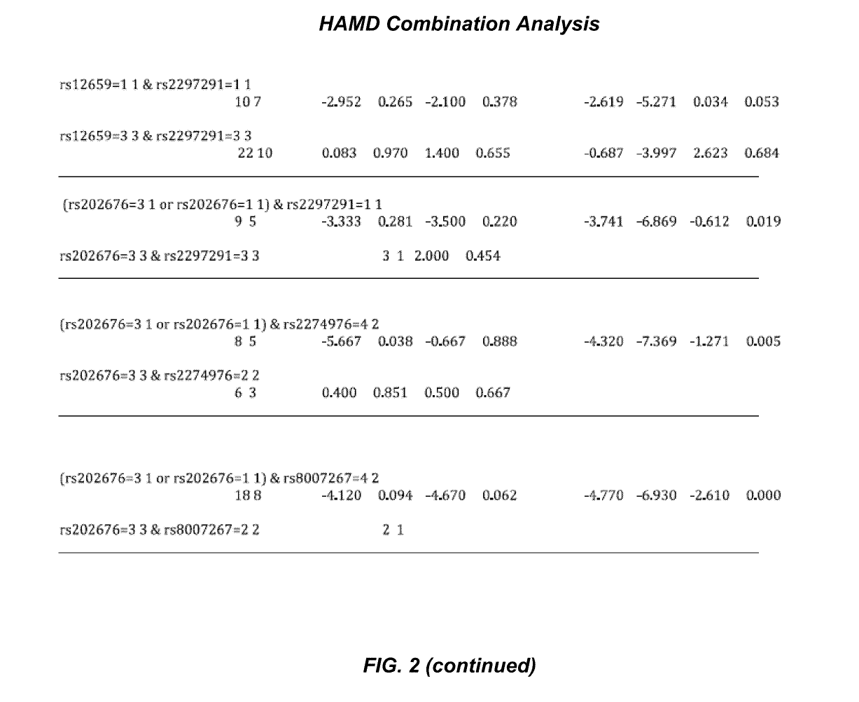

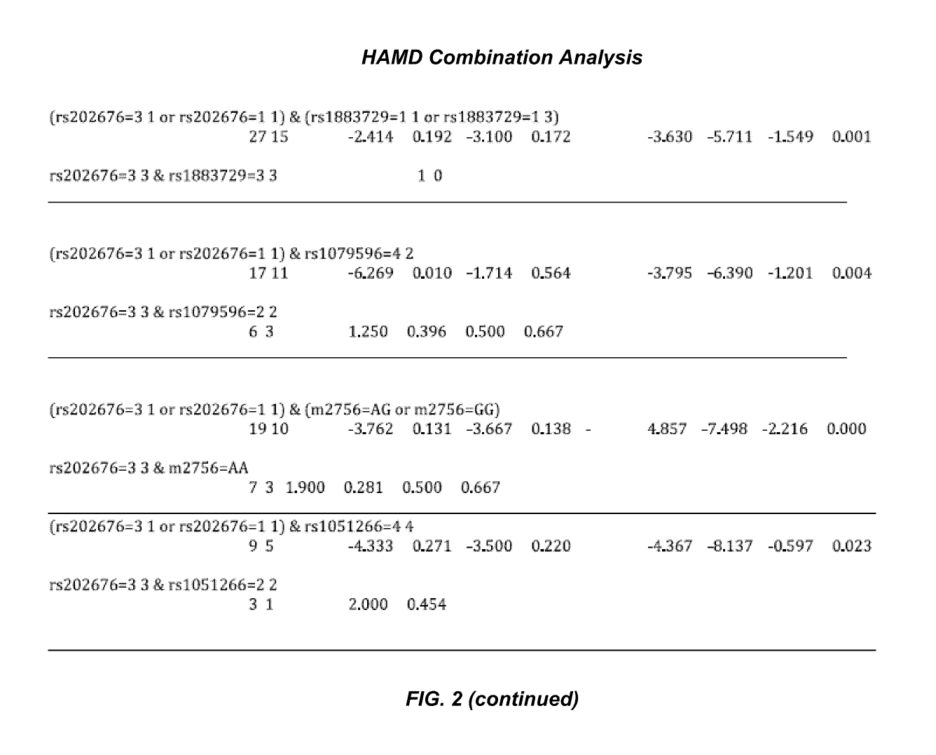

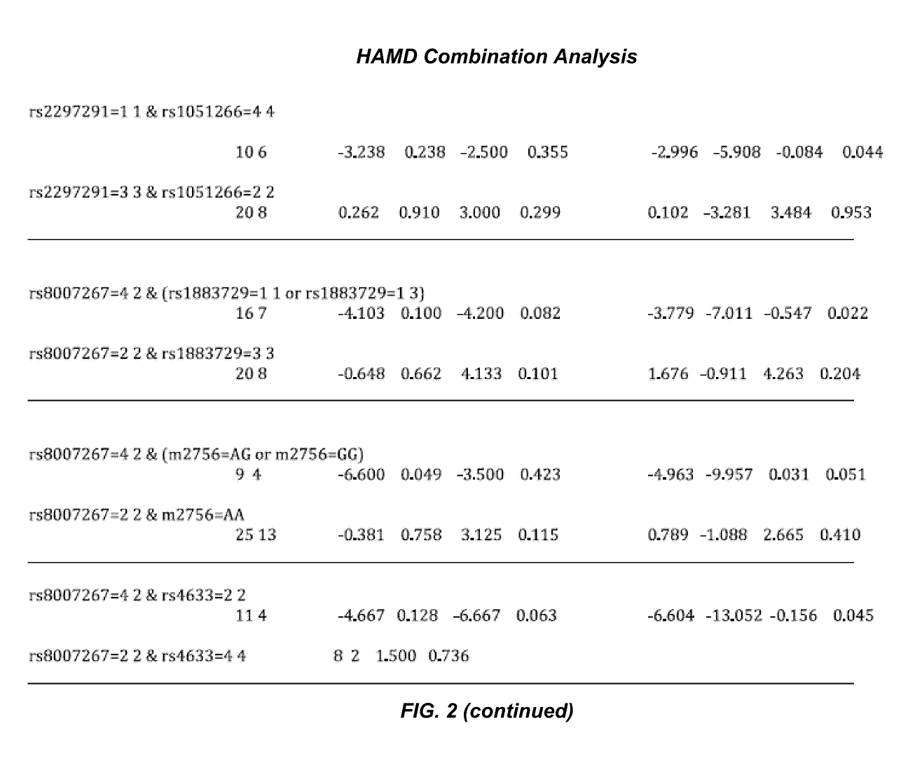

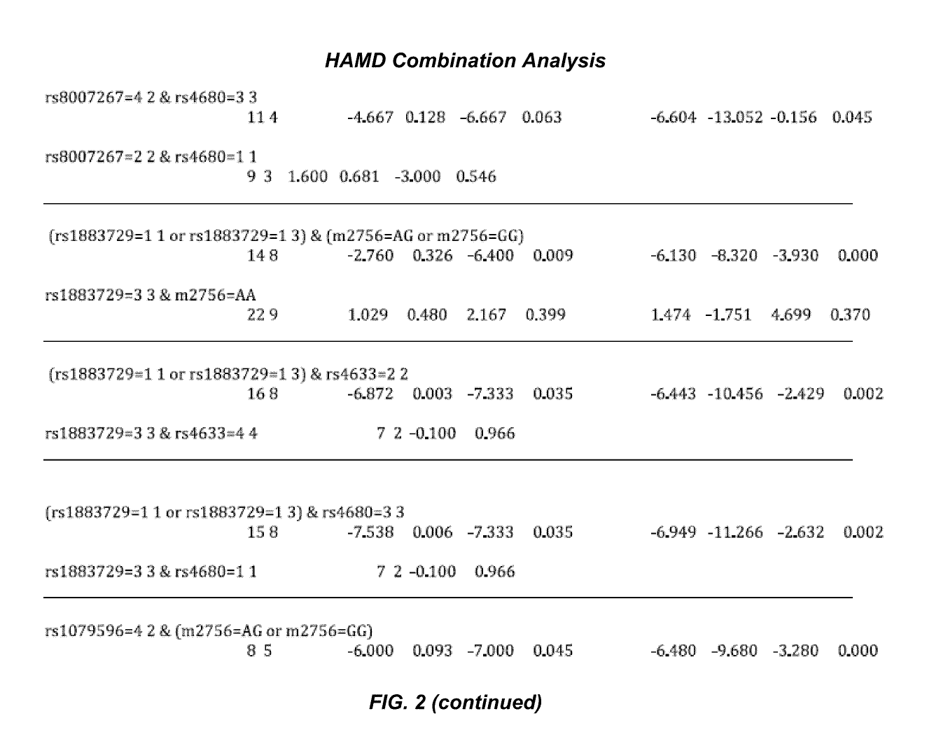

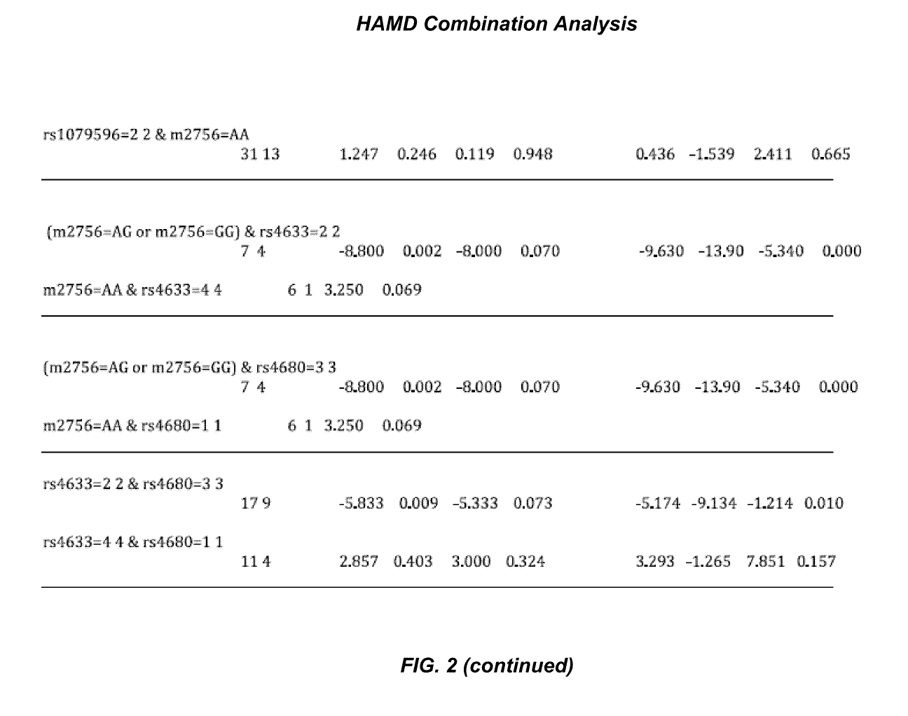

FIG. 2 is a set of result tables showing effects of the presence or absence of an indicated condition (a combination of 2 SNP markers, or a combination of 1 SNP marker and obesity indicator, e.g., BMI>30 kg/m²), in MDD patients on HAMD-28 or HAMD-7 value, when the patients were treated with a treatment regimen comprising a folate-comprising compound, e.g., as an adjunct to an SSRI.

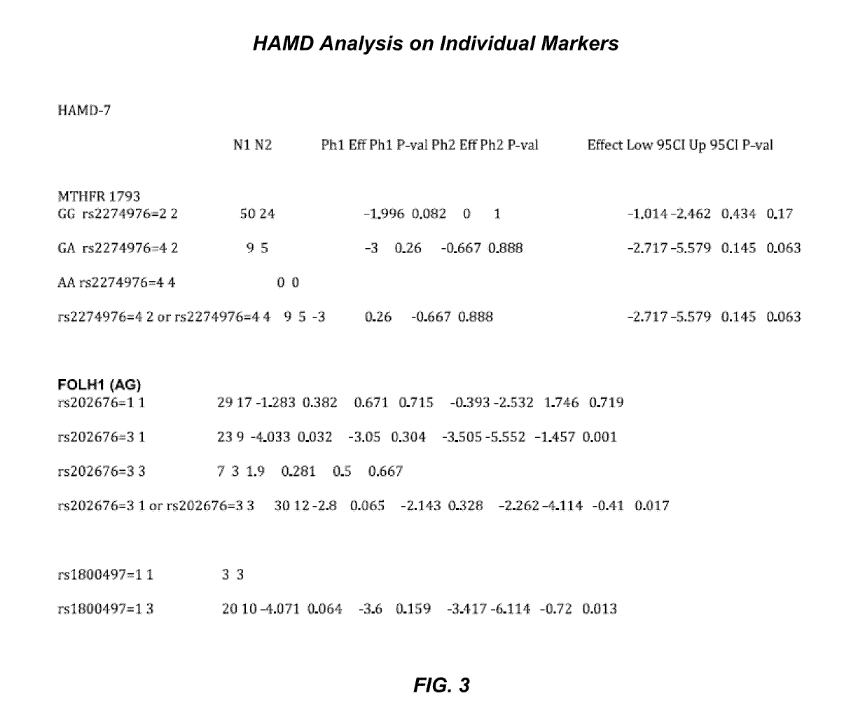

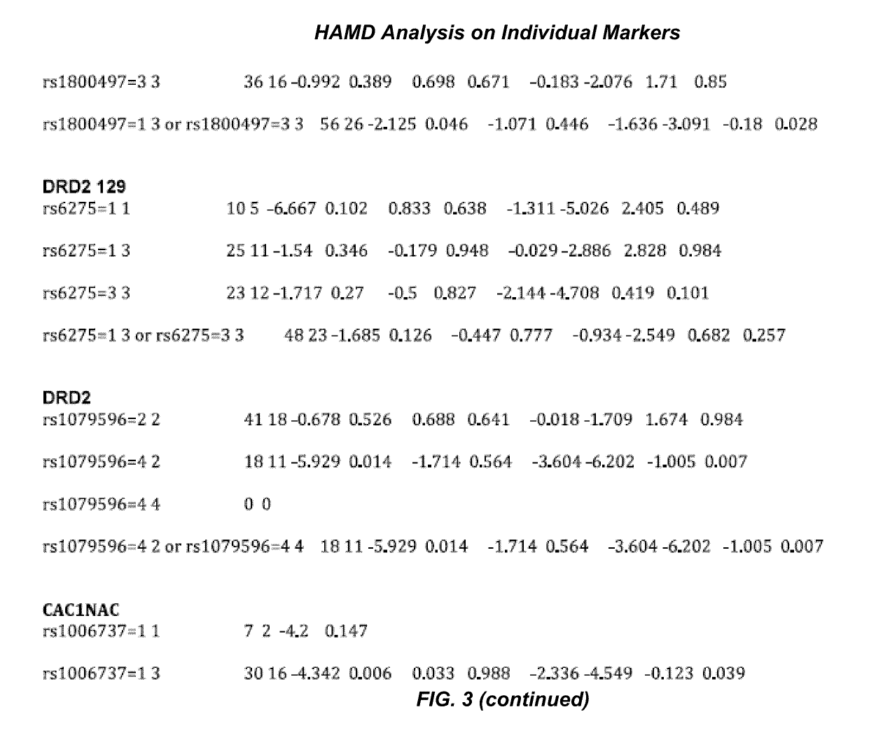

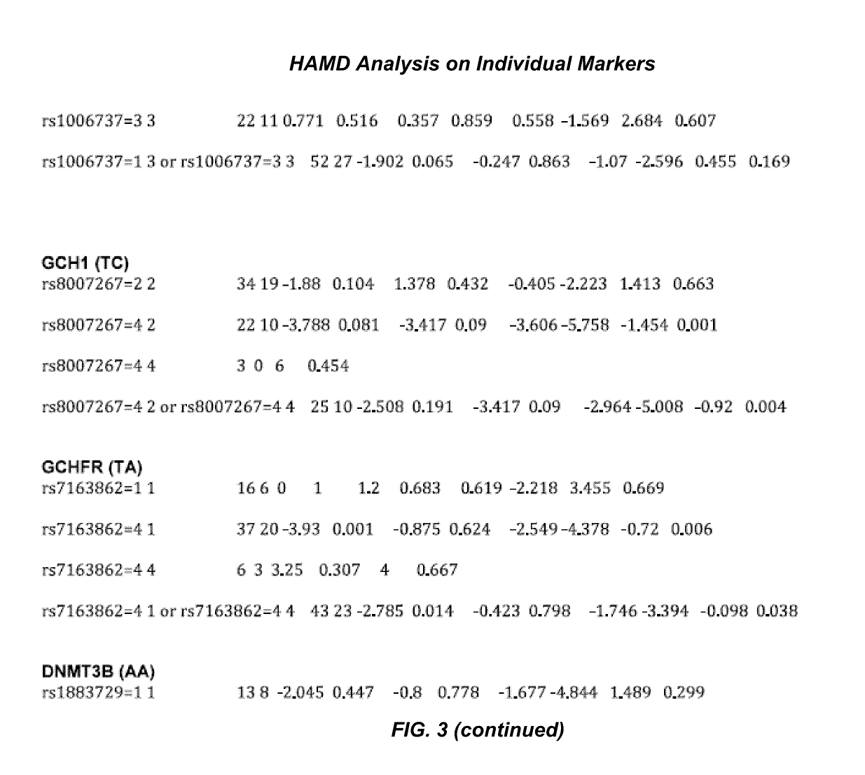

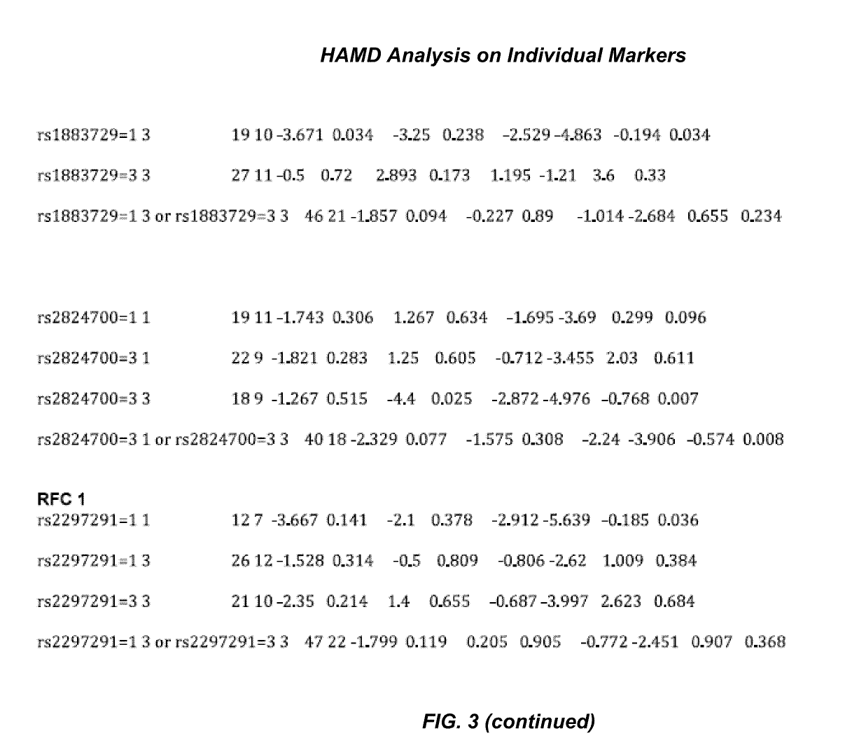

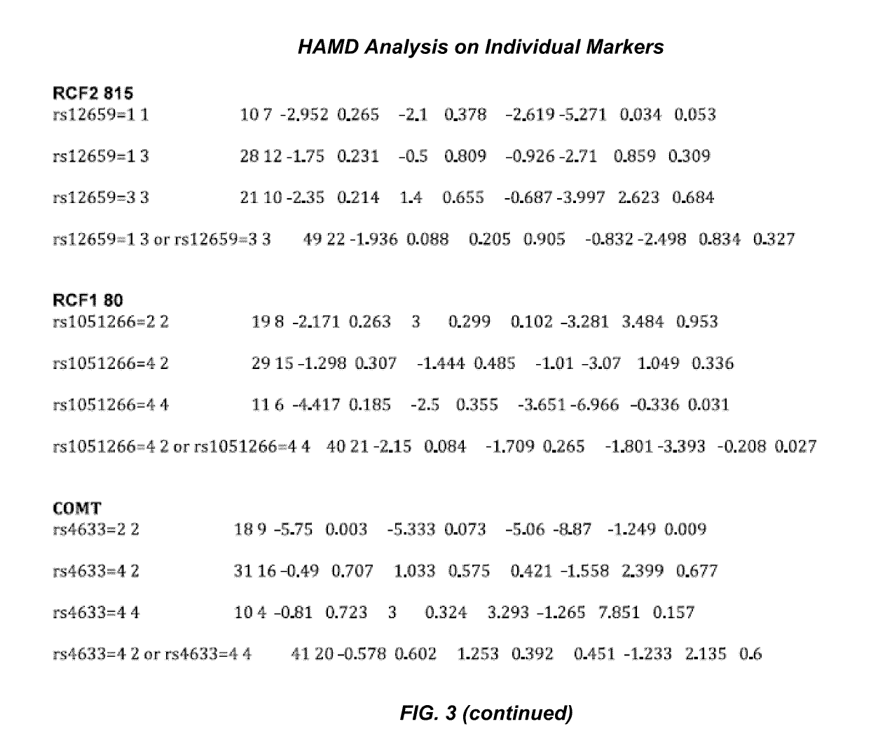

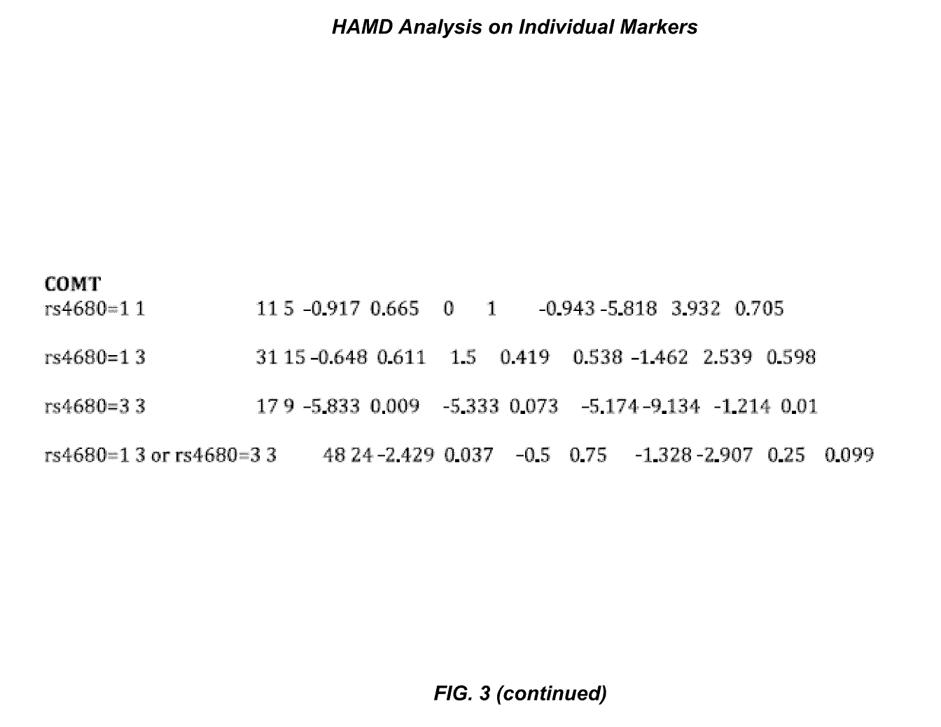

FIG. 3 is a set of result tables showing effects of the presence or absence of an indicated condition (a single SNP marker), in MDD patients on HAMD-7 value, when the patients were treated with a treatment regimen comprising a folate-comprising compound, e.g., as an adjunct to an SSRI.

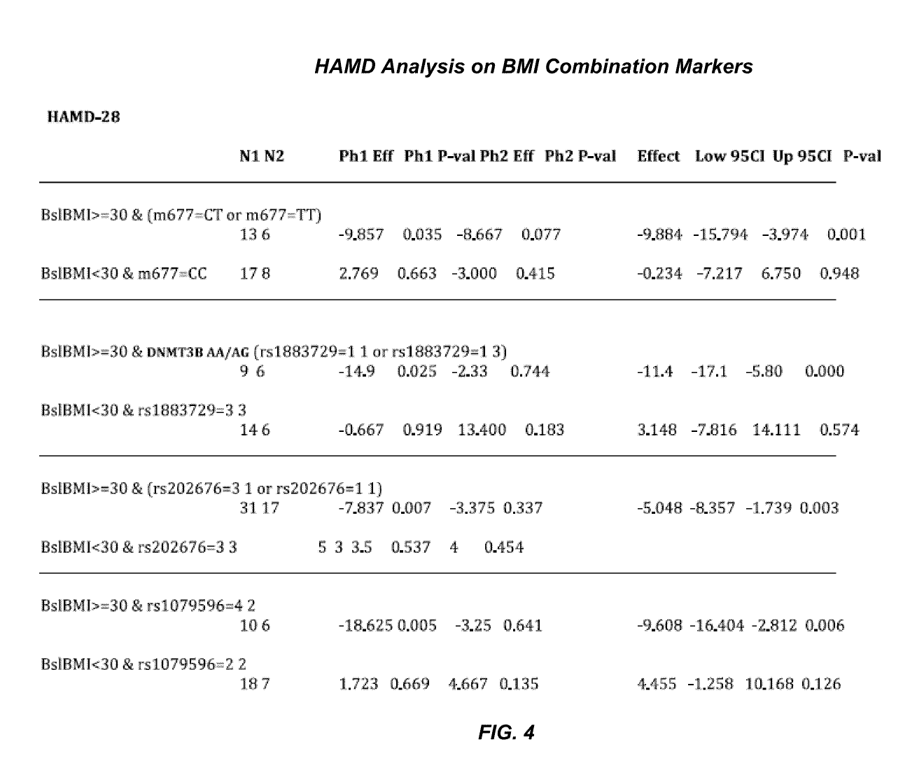

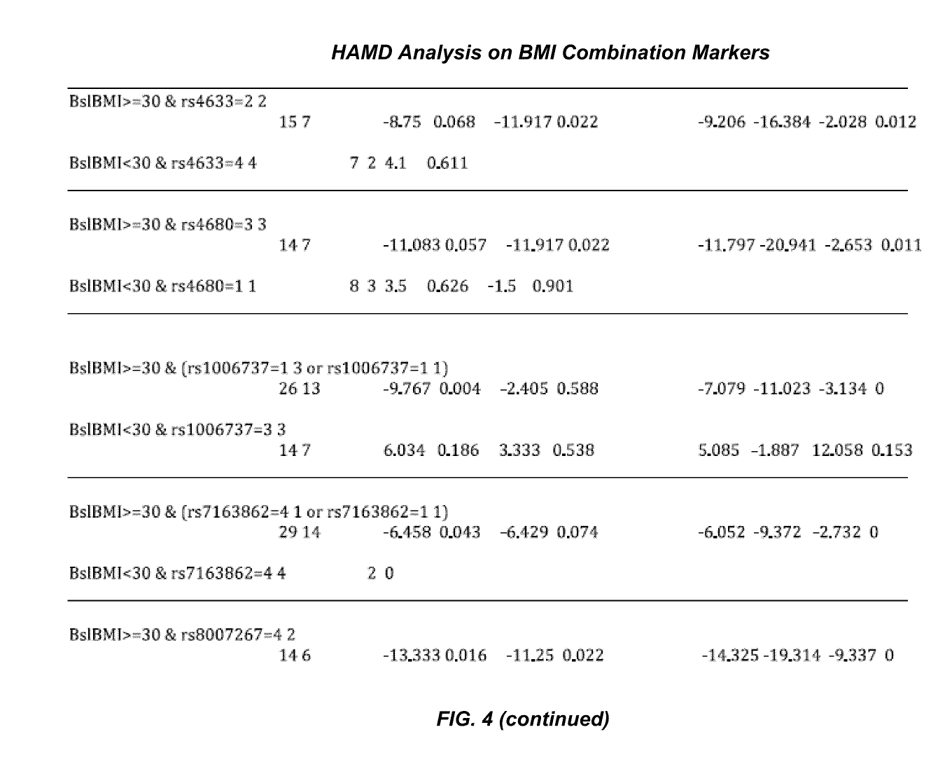

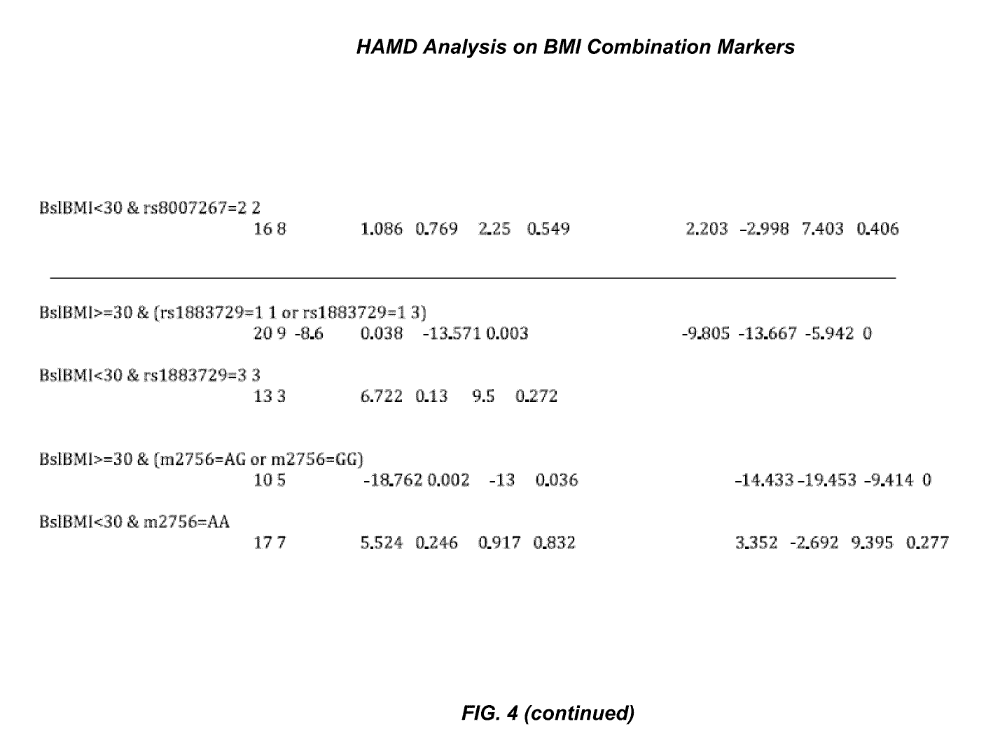

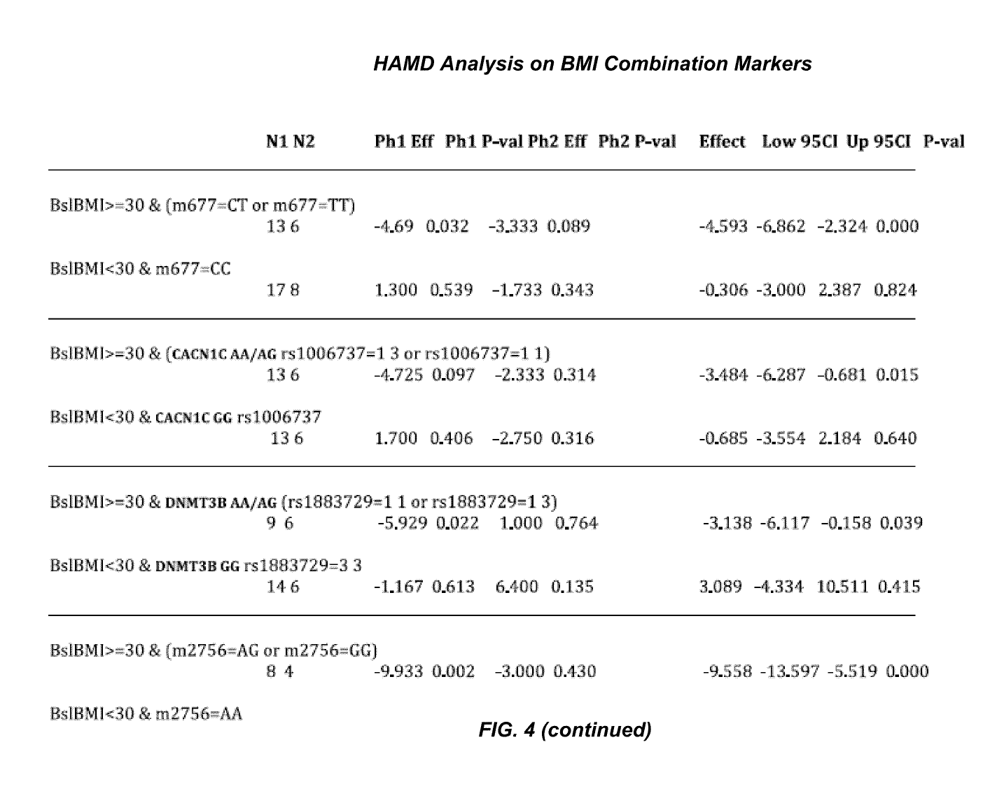

FIG. 4 is a set of result tables showing effects of the presence or absence of an indicated condition (a combination of 1 SNP marker and obesity indicator, e.g., BMI>30 kg/m²), in MDD patients on HAMD-28 or HAMD-7 value, when the patients were treated with a treatment regimen comprising a folate-comprising compound, e.g., as an adjunct to an SSRI.

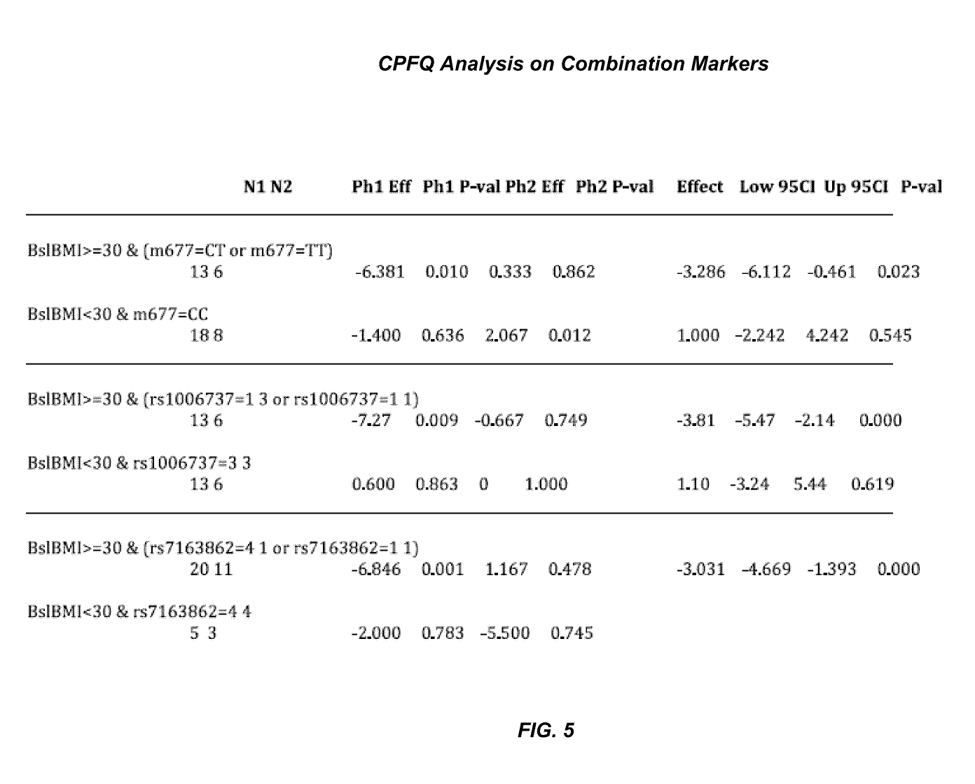

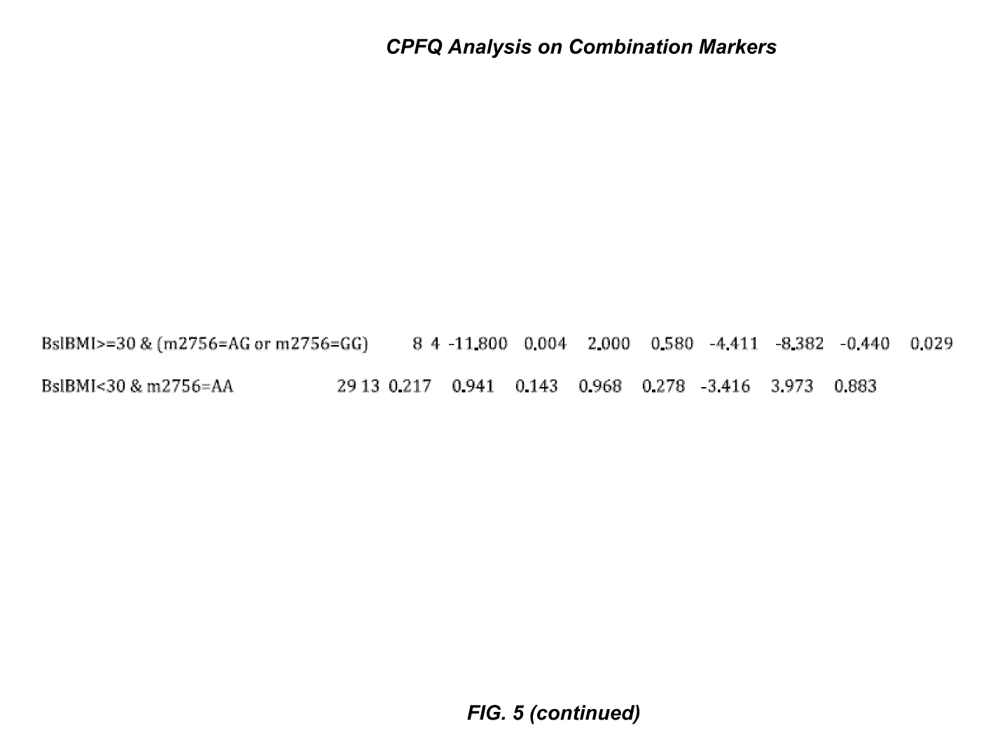

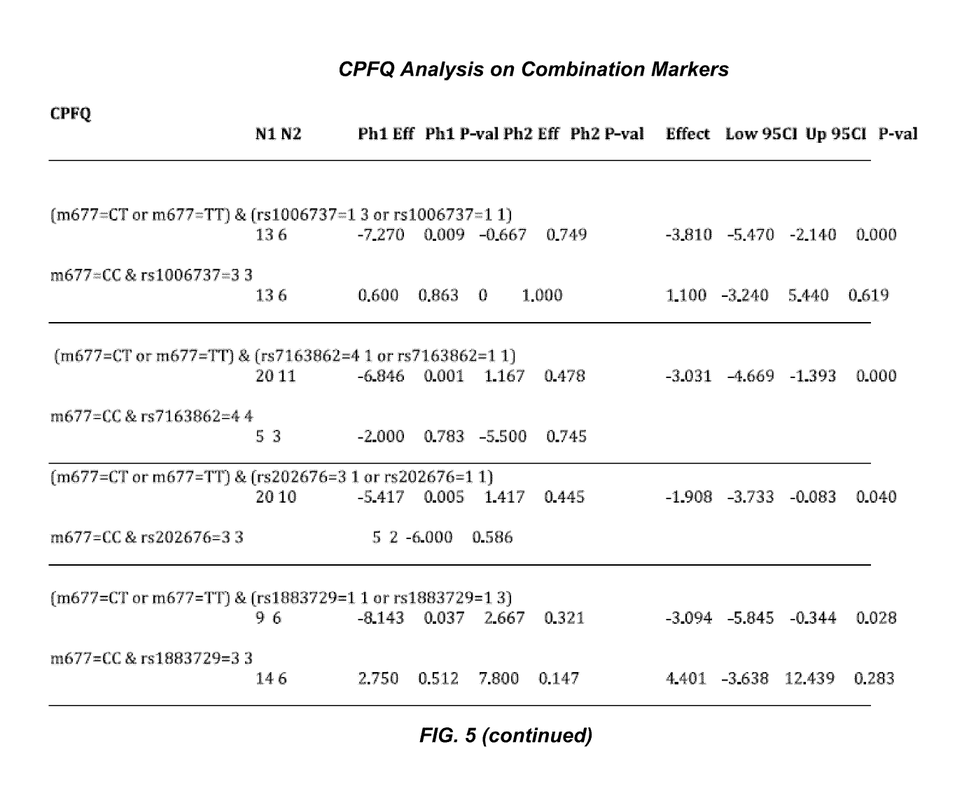

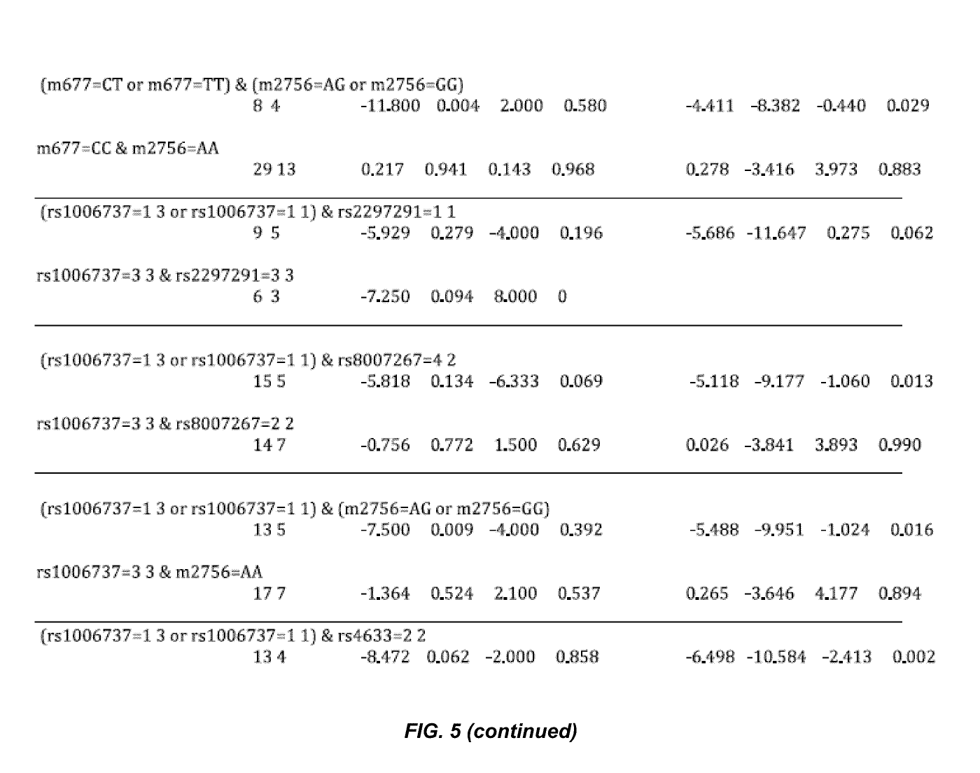

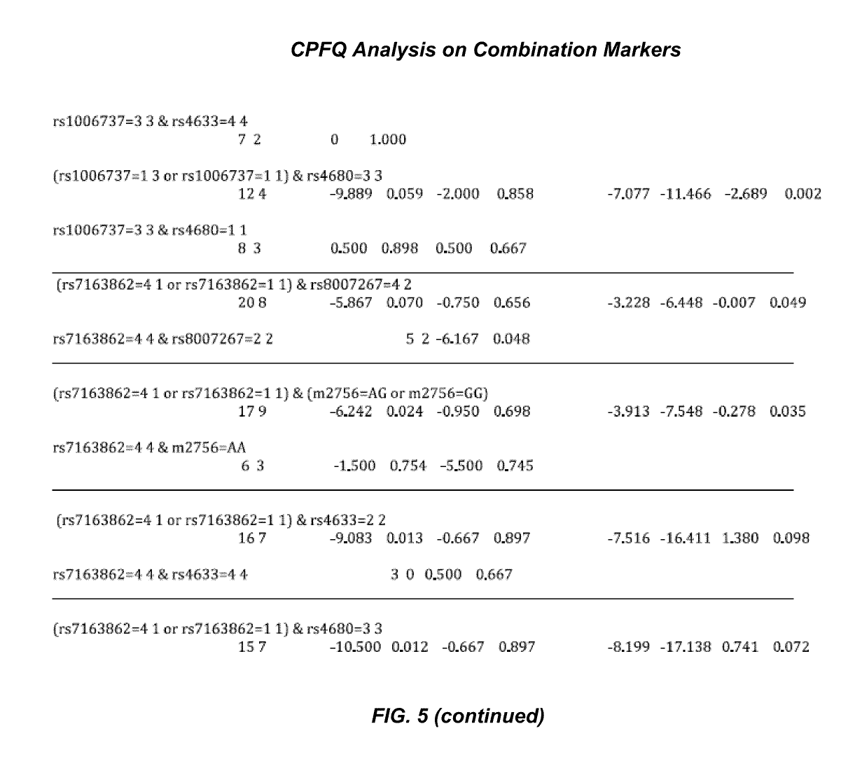

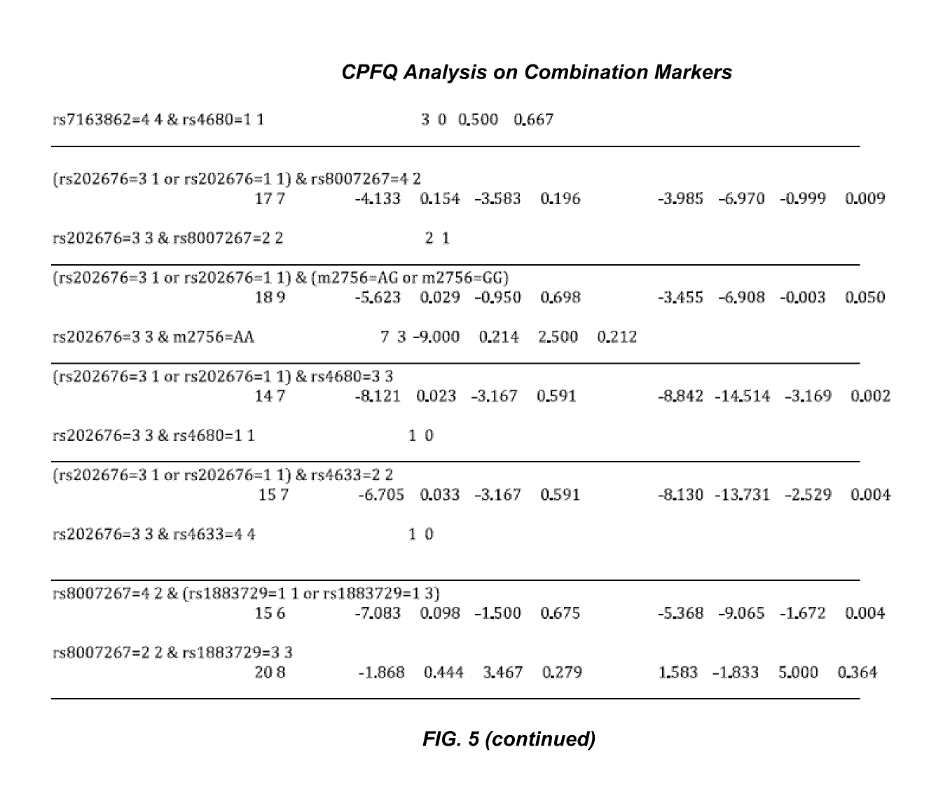

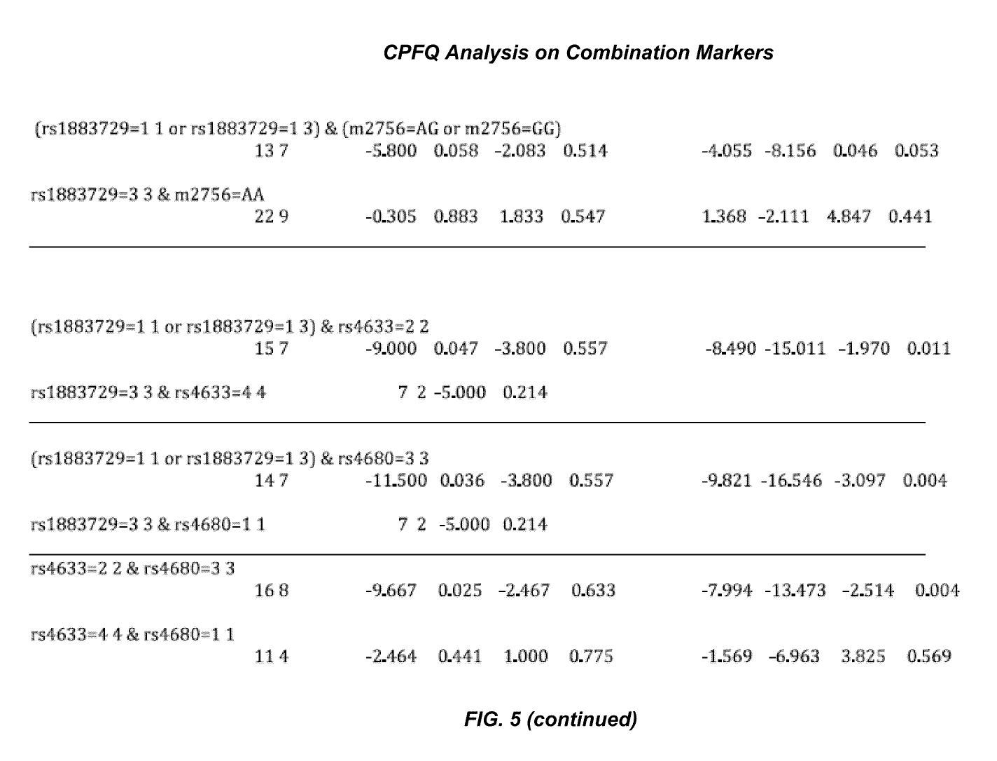

FIG. 5 is a set of result tables showing effects of the presence or absence of an indicated condition (a combination of 2 SNP markers, or a combination of 1 SNP marker and obesity indicator, e.g., BMI>30 kg/m²), in MDD patients on CPFQ value, when the patients were treated with a treatment regimen comprising a folate-comprising compound, e.g., as an adjunct to an SSRI.

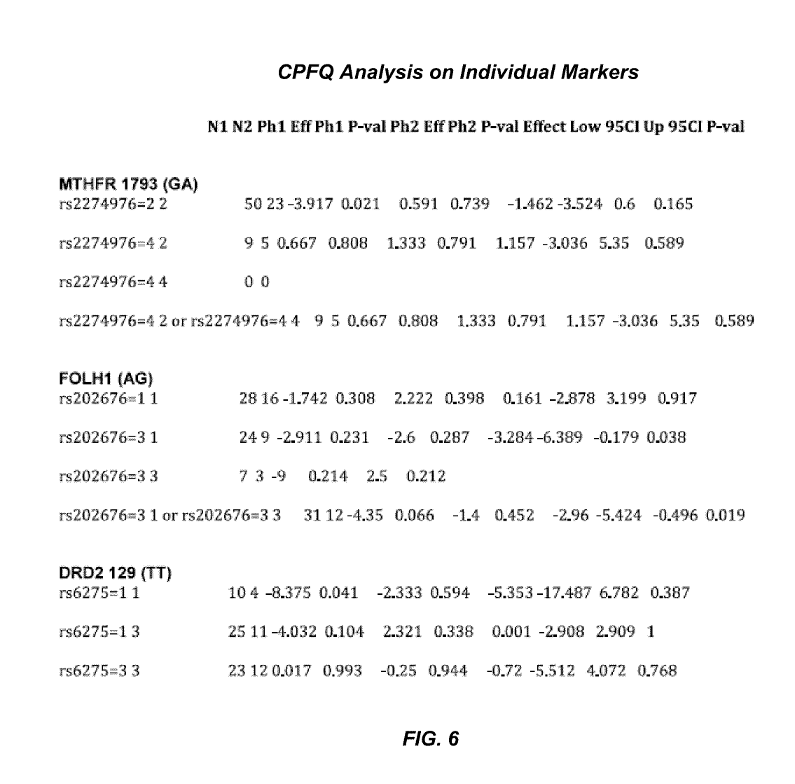

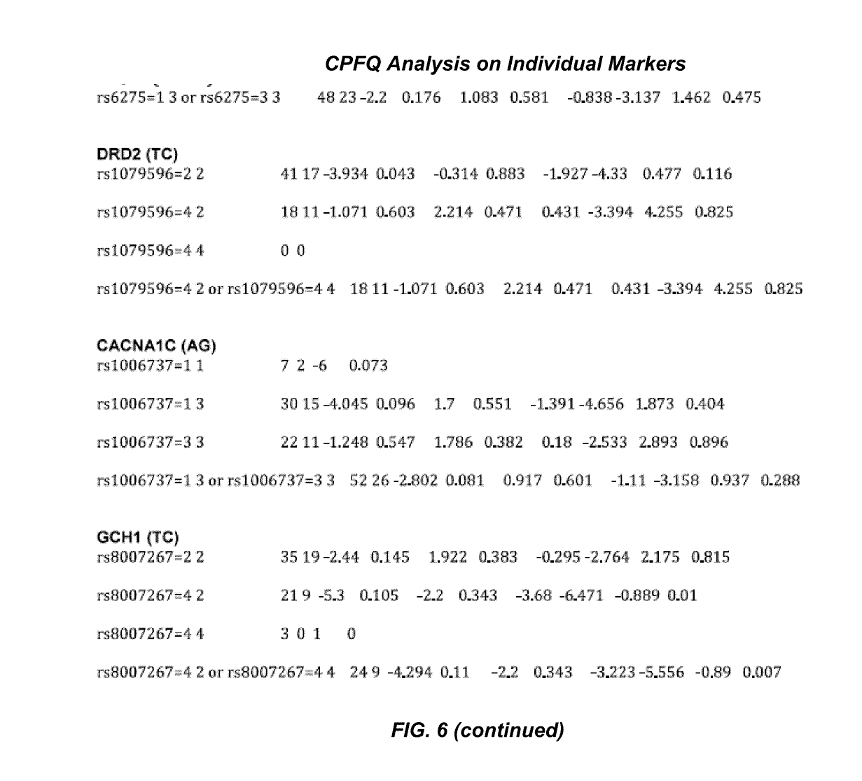

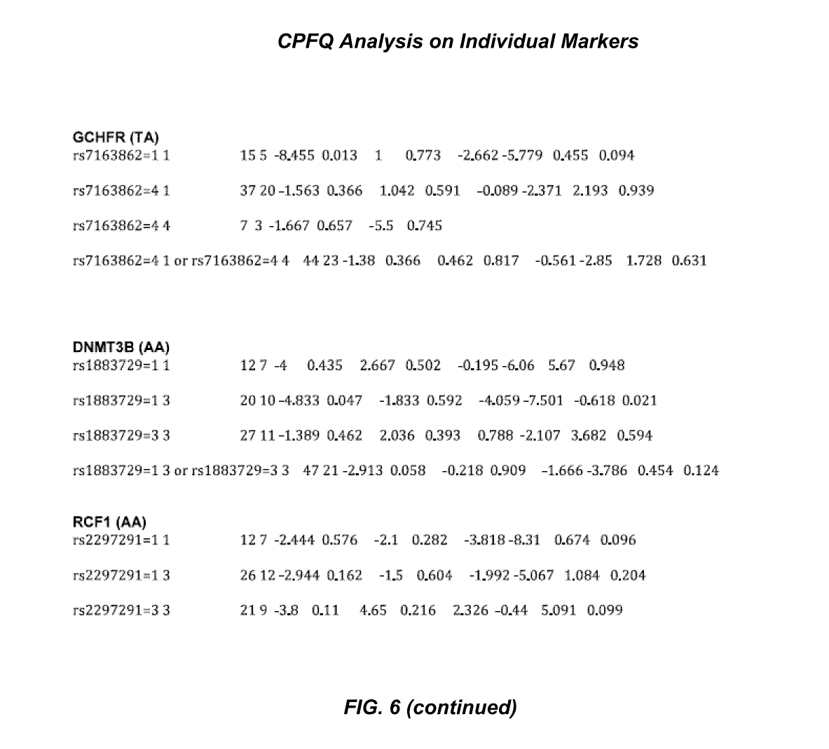

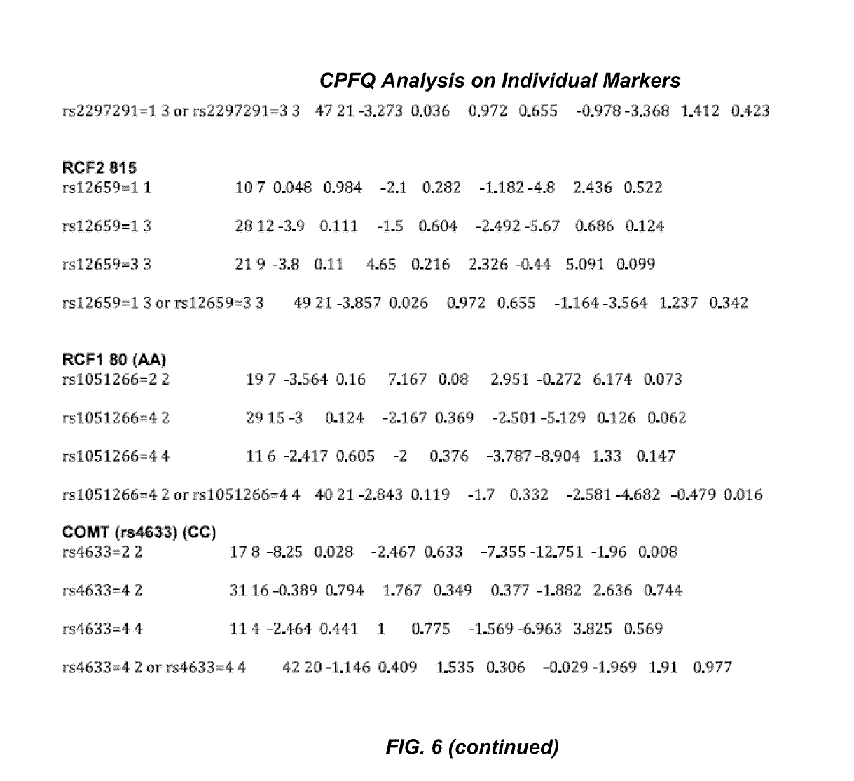

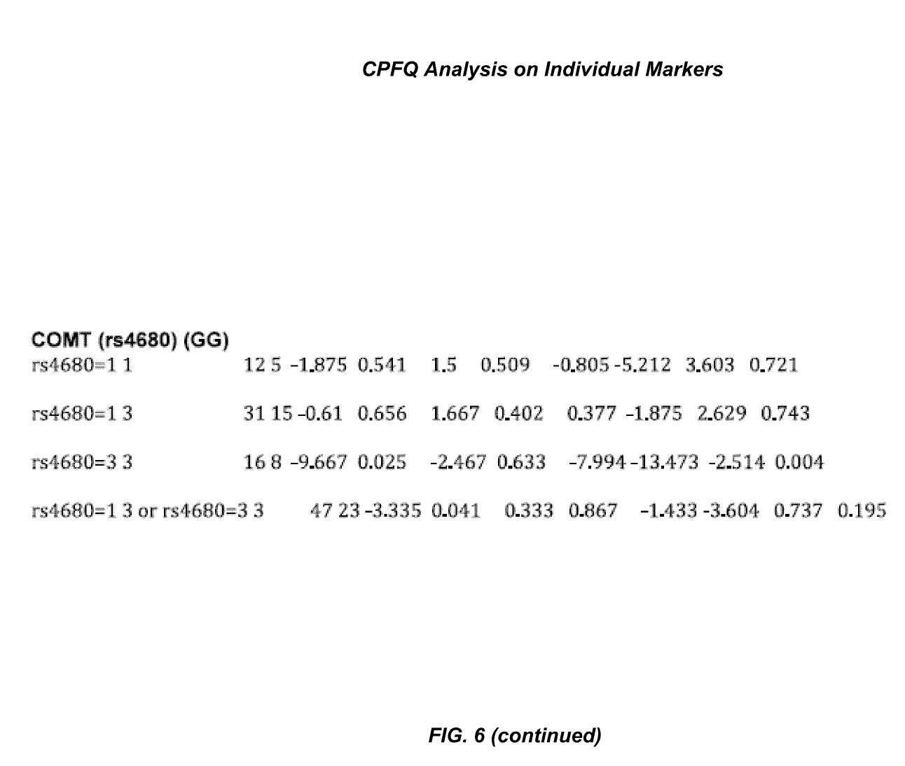

FIG. 6 is a set of result tables showing effects of the presence or absence of an indicated condition (a single SNP marker), in MDD patients on CPFQ value, when the patients were treated with a treatment regimen comprising a folate-comprising compound, e.g., as an adjunct to an SSRI.

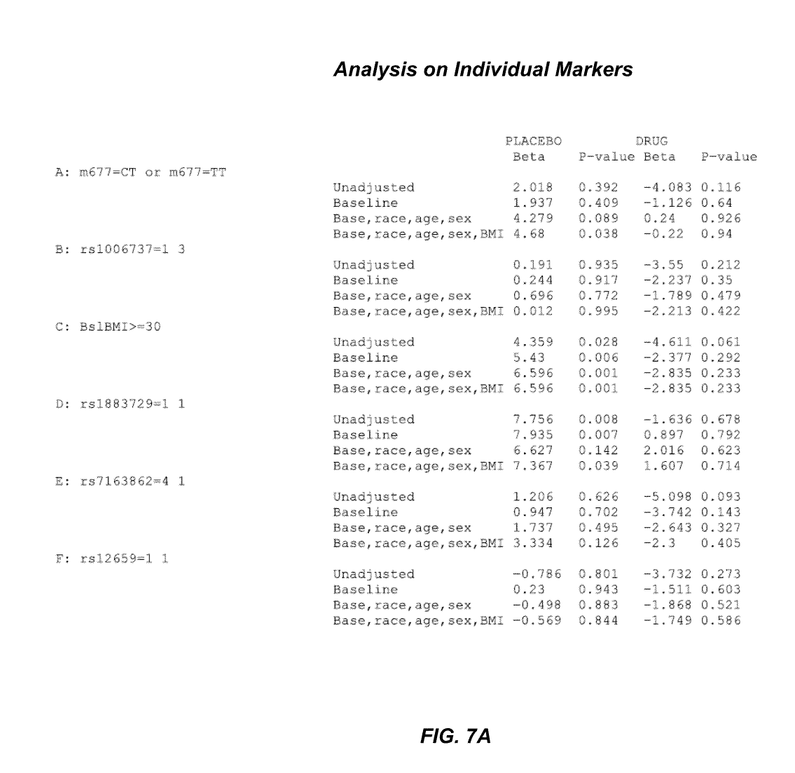

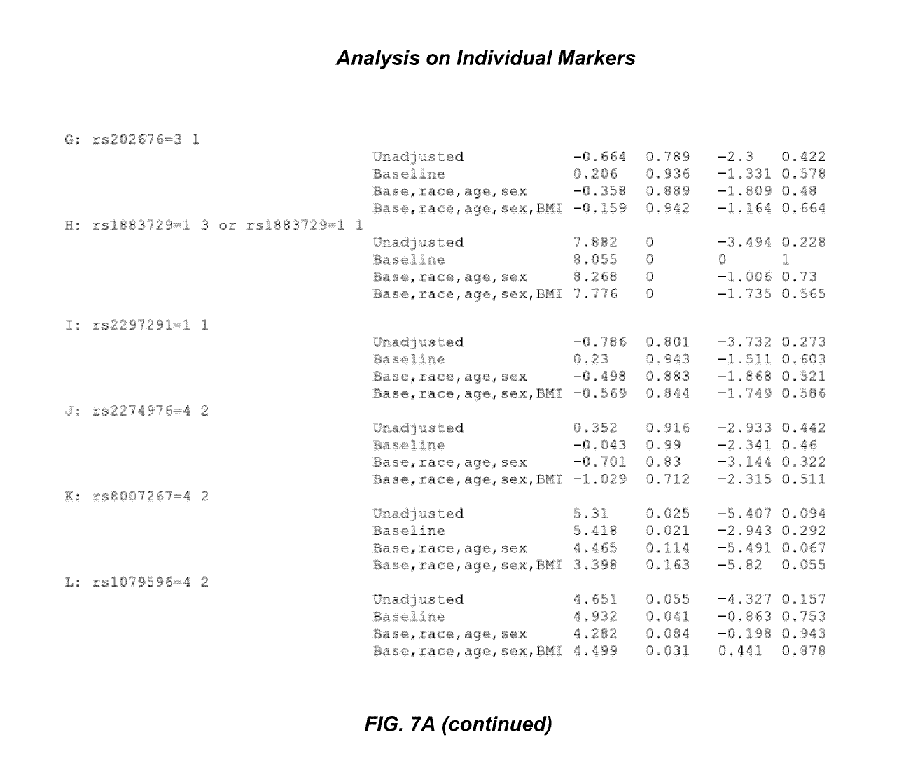

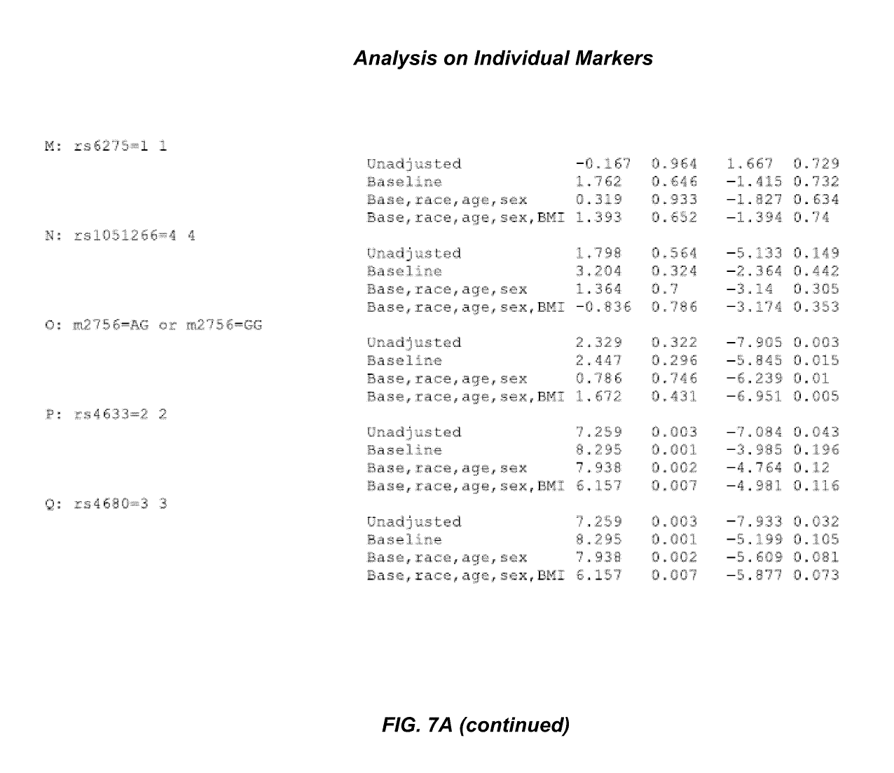

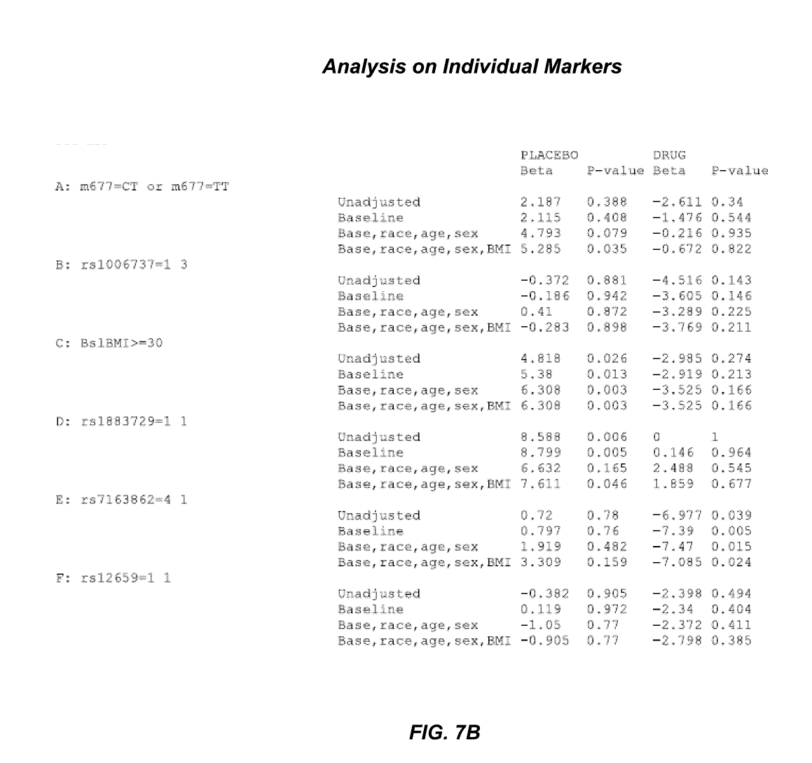

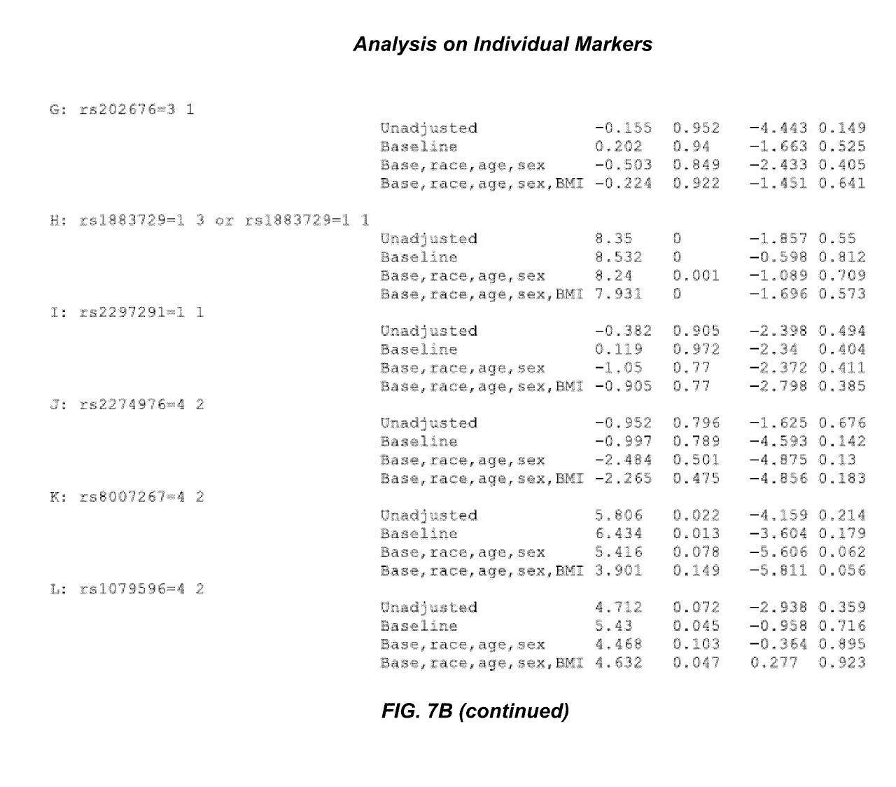

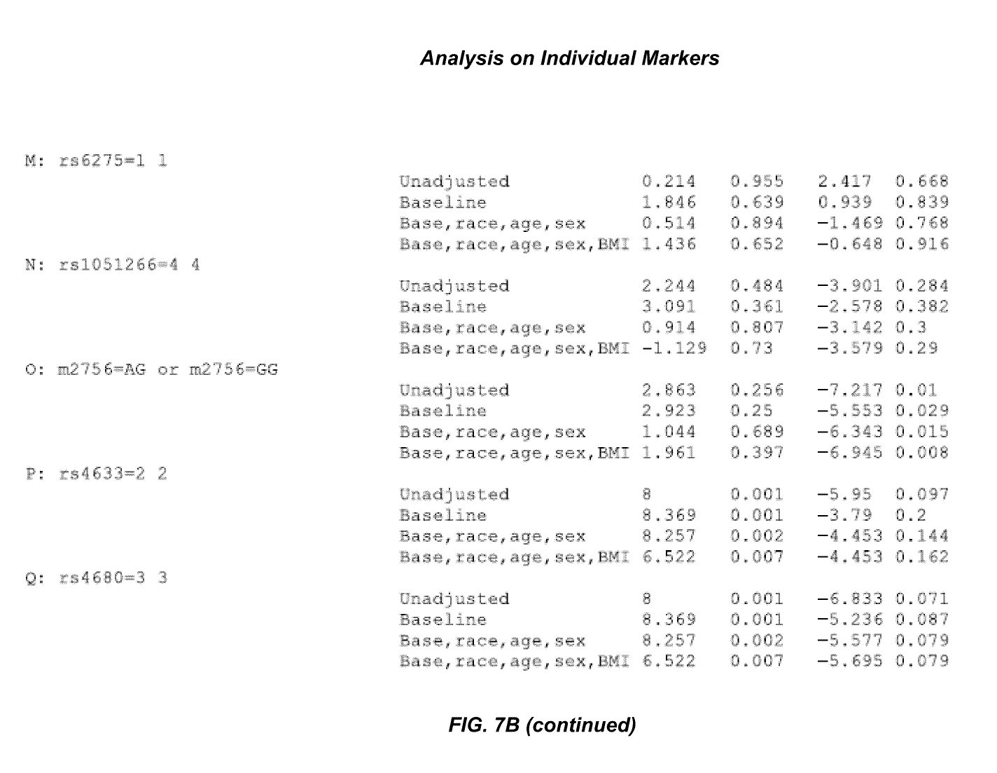

FIG. 7A-B is a set of result tables showing the statistical analysis on the effects of genetic moderators (e.g., race, age, sex, and BMI) comparing biomarker positive versus biomarker negative subjects within the placebo (an antidepressant administered without a folate-comprising compound) or folate-comprising treatment (a folate-comprising compound administered as an adjuvant to the antidepressant) arms of clinical studies. Results show statistically significant treatment effect within all of the indicated genetic modifiers (e.g., race, age, sex, and BMI) for subjects positive for the MTR 2756 AG or GG genotype [associated single-nucleotide polymorphism (SNP) rs1805087] compared to subjects negative for this SNP. Subjects positive for the COMT CC (rs4633) or GG (rs4680) SNP also show statistically significant treatment effect in at least one genetic moderator category.

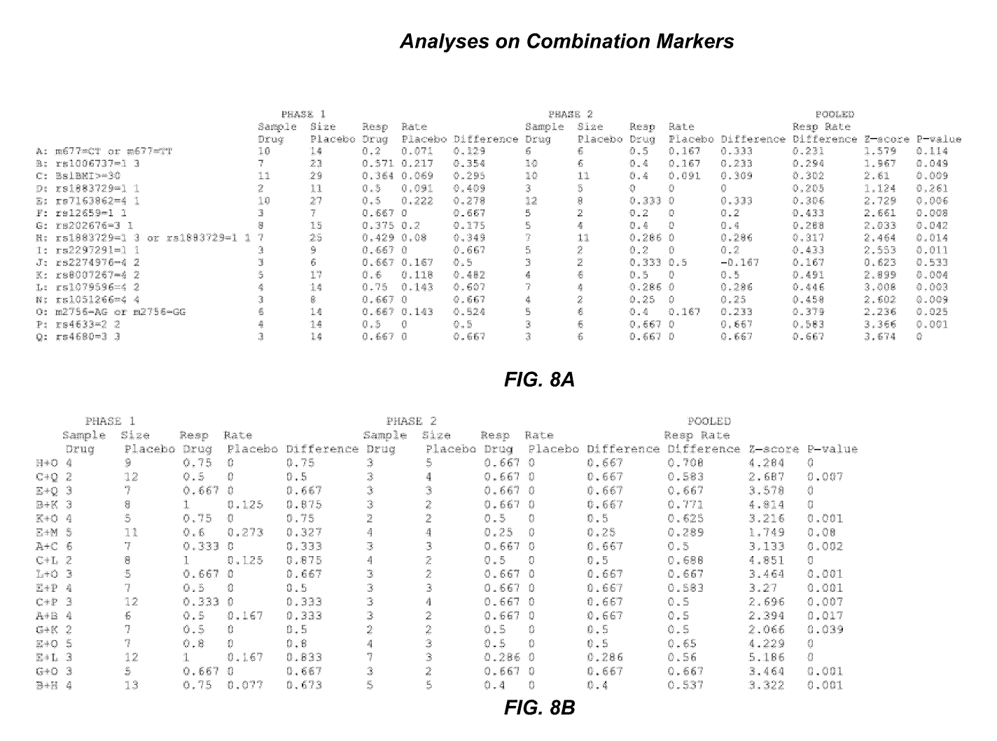

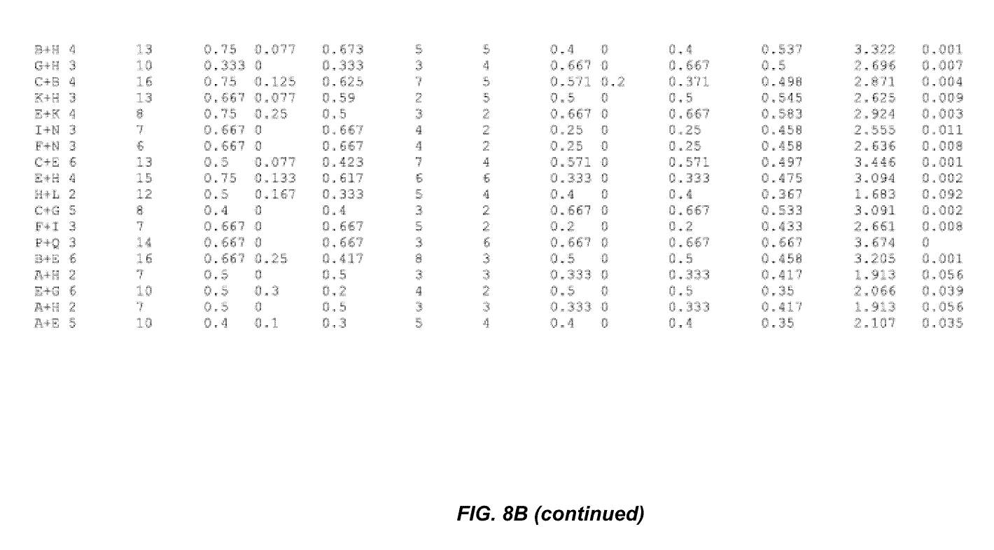

FIG. 8A-B is a set of result tables showing the response rates of biomarker positive subjects within the placebo and folate-comprising treatment arms of clinical studies. A responder is indicated by a reduction of at least about 50% in HAMD-28 over the evaluation period.

The result summary tables in FIG. 1B lists the combinations of two biomarkers in an order of their decreasing effects in MDD patients (i.e., a SNP marker with a greater reduction in HAMD-28 is listed earlier in the table). The combinations of two biomarkers that show a therapeutic response greater than the sum of the responses produced by each individual marker (as determined by change in HAMD-28) are highlighted in a darker shade, which are (A+0), (K+P), (K+Q), (B+Q), (C+0), (C+K), (B+0), (C+H), (A+G), and (A+C), as shown in FIG. 1B, wherein the alphabet identifiers for each corresponding SNP are listed in FIG. 1A. The combinations of two biomarkers that show a therapeutic response greater than the response produced by either marker alone (as determined by change in HAMD-28) are highlighted in a lighter shade, e.g., (0+P) as shown in FIG. 1B. The combinations of two biomarkers that show a therapeutic response comparable to or less than the response produced by either marker alone (as determined by change in HAMD-28) are not highlighted, e.g., (O+Q) as shown in FIG. 1B.

While the result tables in FIG. 1B lists the combinations of two biomarkers in an order of their decreasing effects in MDD patients, it should not be construed as, among the highlighted combinations (i.e., the ones marked by dark and light highlights), one dual-biomarker combination is a more reliable predictor than another one for use in the assays, methods, systems, and kits described herein, as all these highlighted dual-biomarker combinations have indicated a significant change in HAMD-28 when a MDD patient carrying a specific biomarker combination was treated with a folate-comprising compound, as compared to a MDD patient carrying normal alleles on the genes.

### Example 2

### Exemplary Methods of Selecting Patients for Folate-Containing Augmentation Therapy (with an SSRI) and Personalized Prognosis of MDD Patients Subjected to Such Therapy

Based upon the double-blind placebo controlled multi-site study on augmentation of MDD treatment with a folate-comprising compound, e.g., DEPLIN®15, in which 36 patients received a folate-comprising compound, e.g., DEPLIN®15, that had a reduction in their HAMD-28 from the baseline measurement, the patients diagnosed with at least one of the conditions as described in the Panel of Tests (PT) with the corresponding “Values” (as shown in detail later), generally responded in accordance with the expected HAMD-28-reduction as shown in the table below.

Accordingly, methods of treating a MMD patient and/or determining or improving the effectiveness of an antidepressant drug taken by the MMD patient are also provided herein. For example, in some embodiments, the method can include (1) screening for a treatment resistant MDD patient (see, e.g., Step 1 for details below); and (2) performing the Panel of Tests (PT) on a test sample of the MDD patient (see, e.g., Step 2 for details below). In some embodiments, if the PT results show at least one of the Code grouping as shown in Table 5 below, the patient can be recommended for a treatment regimen comprising an antidepressant drug and a folate-comprising compound (e.g., DEPLIN®15). In some embodiments, if the PT results show at least one of the Code grouping as shown in the table below, it can be expected that the corresponding reduction in HAMD-28 from the Baseline value (e.g., the value measured at the Baseline visit) would be achievable with a minimum of 4 weeks of treatment with a folate-comprising compound (e.g., DEPLIN®15) in combination with an antidepressant drug (e.g., an SSRI).

Step 1: Treatment-resistant MDD patients are screened to determine (a) if they meet DSM-IV criteria for MDD; and (b) if they are on an adequate dose of an SSRI and have not adequately responded to one or more courses of an SSRI. Should the patient meet both of the screen criteria (a) and (b) then it is recommended that the physician order the panel of test (PT) as described below.

Step 2: An example of a panel of test (PT) as shown below can be
performed.

| Code | Panel Of Tests | Sample | Value | | Decision | |
|---|---|---|---|---|---|---|
| A | BMI Calculation | Height & | ≧30 | kg/m² | Y | N |
| | | Weight | | | | |
| B | SAM/SAH Ratio | Plasma | <2.71 | | Y | N |
| | | (nmol/L) | | | | |
| C | 4-HNE | Plasma | ≧3.28 | μg/mL | Y | N |

| | | | | | |
|---|---|---|---|---|---|
| D | MTHFR 677 CT/TT | Whole Blood | Yes/No | Y | N |
| E | MTR 2756 AG/GG | Whole Blood | Yes/No | Y | N |
| F | MTRR 66 AG/GG | Whole Blood | Yes/No | Y | N |

The panel of test (PT) as shown above can be modified to delete or add at least one or any combinations of the biomarkers described herein, or include one or more of the 52 dual-biomarker combinations (see, Table 3) or one or more of the 10 synergistic dual-biomarker combinations (see, Table 4).

Step 3: Based on the test results of the conditions listed in PT of step (2), any of the PTs (items A through F) tested positive (i.e., with a decision Y) are identified, and then recorded in alphabetical order of the “Codes” the greatest number of Codes that are represented in the table below. Once the Code grouping has been selected for a given patient's PT, the corresponding “95% CI” (95% Confidence Intervals) for that Code grouping can be reviewed. In some embodiments, if the upper end of the CI is below zero, the HAMD-28 reduction from the Baseline value is likely to be significant. In other embodiments, if the upper end of the CI is above zero, then the HAMD-28 reduction from the Baseline value should be interpreted with caution.

Step 4: The expected reduction in HAMD-28 from Baseline can be determined, for example, as follows:
(a) If a patient has only a single hit of the PT (i.e., one condition is positive), then the HAMD-28 reduction from Baseline can be based upon the Code “All”; or
(b) If a patient has a double hit of the PT (i.e., two conditions are positive), then the HAMD-28 reduction from Baseline can be based upon the highest response (i.e., greatest change in HAM-D-28) obtained from either A, B, C, E or “ALL” as shown in Table 5. In some embodiments, if the double hit is “D+F,” then the reduction can be based upon Code “All”;
(c) If a patient has a triple hit of the PT (i.e., three conditions are positive), then the HAMD-28 reduction from Baseline can be based upon the highest response (i.e., greatest change in HAM-D-28) obtained from the best combination (i.e., the best of 2-code combinations) as shown in Table 5. By way of example only, if a patient has a triple hit on A, C and E of the PT, possible 2-code combinations are A+C, A+E and C+E. Among these combinations, as the combination “A+E” corresponds to the greatest HAMD-28 reduction as shown in Table 5, the combination “A+E” is considered as the best combination that corresponds to the greatest HAMD-28 reduction. However, if the triple hit contains “D+F,” then the HAM-D-28 reduction should be based upon the highest response obtained from the single Codes of A, B, C, E or “ALL”;
(d) The negative HAMDA numbers in Table 5 reflect the potential reduction from Trial 2 Baseline HAMD-28 (˜24.47). The HAMDA number represents the expected reduction in a HAMD-28 scale a patient can obtain in response to Deplin®5 augmentation therapy (with an SSRI) in as little as 4 weeks. In some embodiments, the actual reduction can fall anywhere within the 95% as shown in Table 5 below.

TABLE 5
The expected HAMD-28 reduction based upon Trial 2's
mean Baseline of 24.47 per various Code combinations.

| CODE | “N” | 95% CI | HAMDΔ |
|---|---|---|---|
| ALL | 36 | (−20.8, 7.2) | −6.8 |
| A | 21 | (−23.0, 8.2) | −7.4 |
| B | 16 | (−25.3, 6.7) | −9.3 |
| C | 21 | (−20.2, 7.5) | −6.4 |
| E | 11 | (−28.3, 7.6) | −10.3 |
| A + B | 13 | (−27.0, 7.8) | −9.6 |
| A + C | 12 | (−20.3, 7.2) | −6.6 |
| A + D | 9 | (−30.2, 6.9) | −11.7 |
| A + E | 5 | (−35.7, 0.5) | −17.6 |
| B + C | 11 | (−21.3, 5.4) | −7.9 |
| B + E | 6 | (−32.2, −2.1) | −17.2 |
| B + F | 11 | (−24.5, 3.0) | −10.8 |
| C + D | 8 | (−27.2, 9.4) | −8.9 |
| C + E | 9 | (−25.3, 9.3) | −8.0 |
| C + F | 15 | (−23.4, 8.4) | −7.5 |
| D + E | 4 | (−34.3, −10.6) | −22.5 |
| E + F | 8 | (−26.7, 9.2) | −8.7 |

### Example 3

### Effect of Adjunctive L-Methylfolate 15 mg in Depressed Patients Stratified by Biomarker Levels and Genotype: Results from a Randomized Clinical Trial

This example illustrates a method of improving the effectiveness of an antidepressant drug (e.g., a selective serotonin reuptake inhibitor) that is administered to a human patient diagnosed as having depression or is at risk of having depression. The method includes determining whether the patient carries at least one single nucleotide polymorphism (SNP), wherein the SNP is associated with increased effectiveness of the antidepressant drug when administered in combination with a folate-comprising compound, e.g., L-methylfolate. This example also provides a method for treating at least one symptom of depression (e.g., depressed mood, guilt, reduced work or interests, psychomotor retardation, agitation, psychic anxiety, somatic anxiety, general somatic symptoms, cognition impairment, or any combination thereof) in a human subject. The method includes administering a folate-comprising compound, e.g., L-methylfolate to the subject who 1) is diagnosed as having depression or is at risk of having depression and 2) carries a combination (e.g., plurality) of particular SNPs that is associated with a positive-symptom-reducing response to the folate-comprising compound.

The presence of specific genetic or biological markers may predict inadequate response to therapy for major depression. The objective of this analysis was to evaluate the effect of specific biological and genetic markers alone and in combination on the antidepressant efficacy of adjunctive L-methylfolate 15 mg vs. placebo from a trial of inadequate responders to SSRIs. This was a double blind, randomized, placebo-controlled trial using the sequential parallel comparison design (SPCD). Outpatients with SSRI-resistant depression received L-methylfolate 15 mg/day for 60 days, placebo for 30 days followed by L-methylfolate 15 mg/day for 30 days, or placebo for 60 days. The effects of baseline levels of select biological and genetic markers individually and combined on treatment response to L-methylfolate vs. placebo were evaluated. Seventy-five patients were enrolled. Patients with specific biological (body mass index [BMI]≧30 kg/m², high sensitivity C-reactive protein [hsCRP] or 4-hydroxy-2-nonenal[4-HNE], low S-adenosylmethionine/S-adenosylhomocysteine [SAM/SAH] ratio) and genetic markers at baseline had significantly (p≦0.05) greater pooled mean change from baseline on the HDRS-28 with L-methylfolate vs. placebo. Pooled mean change from baseline on the CGI-I was significantly (p<0.05) greater with L-methylfolate vs. placebo for most genetic markers. Most combinations of baseline biological and genetic markers predicted significantly (p≦0.05) greater reductions in pooled mean change from baseline for HDRS-28 scores with L-methylfolate vs. placebo. In summary, biomarkers associated with inflammation or metabolism or genomic markers associated with L-methylfolate synthesis and metabolism may identify patients with SSRI-resistant MDD who are responsive to adjunctive therapy with 15 mg L-methylfolate.

Despite the availability of numerous antidepressant drugs, over 60% of patients with major depressive disorder (MDD) fail to experience a complete remission of symptoms following their first antidepressant treatment, and the majority of those who do remit experience relapse or recurrence (Rush A J, Trivedi M H, Wisniewski S R, et al., Am J Psychiatry, 163:1905-1917 (2006); Papakostas G I, Int J Neuropsychopharmacol, 15:841-854 (2012)). This example describes a method of identifying specific patients with improved success rates to more efficacious treatment strategies by using clinical and biological markers.

Disturbances in metabolic systems have been implicated in the pathophysiology and course of MDD (McIntyre R S, Soczynska J K, Konarski J Z, et al., Ann Clin Psychiatry, 19:257-264 (2007); Vogelzangs N, Beekman A T, Boelhouwer I G, et al., J Clin Psychiatry, 72:598-604 (2011); Papakostas G I, Shelton R C, Kinrys G, et al., Mol Psychiatry, 18:332-339 (2013)). For instance, a prospective cohort study found that participants with depression and co-morbid metabolic syndrome had a higher risk of developing chronic, recurrent depression (Vogelzangs N, Beekman A T, Boelhouwer I G, et al., J Clin Psychiatry, 72:598-604 (2011)). In parallel, in recent years, an association has also been recognized between MDD and impaired cellular immunity and inflammation, characterized by elevated interleukin-6 (IL-6), tumor necrosis factor alpha (TNFα), and high sensitivity C-reactive protein (hsCRP) levels (Papakostas G I, Shelton R C, Kinrys G, et al., Mol Psychiatry, 18:332-339 (2013); Blume J, Douglas S D, Evans D L, Brain Behav Immun., 25:221-229 (2011); Miller A H, Maletic V, Raison C L, Biol Psychiatry, 65:732-741 (2009); Simon N M, McNamara K, Chow C W, et al., Eur Neuropsychopharmacol., 18:230-233 (2008)). Furthermore, activation of inflammatory pathways within the brain also may contribute to oxidative stress leading to the neuropathological characteristics of MDD (Miller A H, Malefic V, Raison C L, Biol Psychiatry, 65:732-741 (2009); Ng F, Berk M, Dean O, et al. Int J Neuropsychopharmacol., 11:851-876 (2008); Stafford L, Berk M., J Clin Psychiatry 72:1229-1235 (2011)). In a prospective study of patients hospitalized for cardiac intervention, use of statins, which have anti-inflammatory and antioxidative properties, was associated with a significant reduction in the risk of MDD at 9 months (Ng F, Berk M, Dean O, et al., Int J Neuropsychopharmacol., 11:851-876 (2008); Stafford L, Berk M., J Clin Psychiatry 72:1229-1235 (2011)).

An association has been observed between folate deficiency, metabolic dysregulation and inflammation (Vogelzangs N, Beekman A T, Boelhouwer I G, et al., J Clin Psychiatry, 72:598-604 (2011); Blume J, Douglas S D, Evans D L, Brain Behav Immun., 25:221-229 (2011); Miller A H, Maletic V, Raison C L, Biol Psychiatry, 65:732-741 (2009); Simon N M, McNamara K, Chow C W, et al., Eur Neuropsychopharmacol., 18:230-233 (2008)). The benefits of folic acid, and its biologically active form, L-methylfolate, for treating MDD have been recognized; also recently recognized are links between folate deficiency and an increased risk for MDD, reduced antidepressant effectiveness, and a more chronic course of illness (Vogelzangs N, Beekman A T, Boelhouwer I G, et al., J Clin Psychiatry, 72:598-604 (2011); Ginsberg L D, Oubre A, Daoud Y, Innov Clin Neurosci., 8:19-28 (2011); Fava M., J Clin Psychiatry, 68 Suppl 10:4-7 (2007); Fava M, Borus J S, Alpert J E, et al., Am J Psychiatry, 154:426-428 (1997); Papakostas G I, Petersen T, Mischoulon D, et al., J Clin Psychiatry, 65:1096-1098 (2004)). More recently, the results were published from a randomized, placebo-controlled trial in MDD patients not achieving an adequate response to selective serotonin reuptake inhibitors (SSRIs), which demonstrated greater efficacy for adjunctive treatment with 15 mg daily of L-methylfolate versus placebo using the sequential parallel comparison design (SPCD) (Papakostas G I, Shelton R C, Zajecka J M, et al., Am J Psychiatry, 169:1267-1274 (2012)).

This example describes the treatment effect of 15 mg of L-methylfolate versus placebo as a function of baseline biomarker levels or genotype focusing on markers of metabolic or inflammatory status. Specifically, it also illustrates the relationship between hypofolatemia and metabolic disturbances as well as inflammation, e.g., the interaction between metabolic or inflammatory status at baseline as defined using specific markers from these domains and treatment outcome with 15 mg daily of L-methylfolate versus placebo augmentation. In addition, the example shows the interaction in light of the role of L-methylfolate in enhancing tetrahydrobiopterin (BH₄)-dependent monoamine synthesis (Hyndman M E, Verma S, Rosenfeld R J, et al. Am J Physiol Heart Circ Physiol 282:H2167-2172 (2002)). Significant correlations have been observed between MDD and levels of red cell folate, monoamine neurotransmitters, and cerebral spinal fluid BH4. Furthermore, BH4 regulates the presynaptic release of neurotransmitters from nerve terminals (Bottiglieri T., Prog Neuropsychopharmacol Biol Psychiatry, 29:1103-12 (2005)). Finally, the example also shows the influence of markers associated with one-carbon cycle metabolism and treatment outcome.

### Methods

This report presents results from exploratory analyses from a multi-center, 60-day, randomized, double-blind trial of 15 mg L-methylfolate as adjunctive therapy for patients with SSRI-resistant MDD (Papakostas G I, Shelton R C, Zajecka J M, et al., Am J Psychiatry, 169:1267-1274 (2012)). The study was divided into two, 30-day phases (phases I and II), according to the sequential parallel comparison design (SPCD) of Fava M, Evins A E, Dorer D J, et al., Psychother Psychosom, 72:115-127 (2003). The study design and results were described previously (Papakostas G I, Shelton R C, Zajecka J M, et al., Am J Psychiatry, 169:1267-1274 (2012)) but are summarized briefly below. The study protocol was reviewed and approved by the following institutional review boards (IRB): Massachusetts General Hospital, Partners Human Research Office; Rush University Medical Center, Research and Clinical Trials Administration Office; Goodwyn IRB; University of California, San Diego, Human Research Protections Program; University of Cincinnati Medical Center, Institutional Review Board Office; Vanderbilt University, Institutional Review Board; and University of Pennsylvania, Office of Regulatory Affairs. Written informed consent was obtained from all study patients before any study procedures were conducted (ClinicalTrials.gov Registration Number NCT00955955).

### Patient Selection

Adults age 18-65 years and meeting DSM-IV criteria for a current episode of MDD were eligible if they had a Quick Inventory of Depressive Symptoms-Self Report (QIDS-SR) score ≧12 at screening and baseline visits. Patients must have been treated with an SSRI during the current episode of MDD for ≧8 weeks at adequate doses (defined as 20 mg/day or more of fluoxetine, citalopram, or paroxetine, 10 mg/day or more of escitalopram, and 50 mg/day or more of sertraline) as assessed using the Massachusetts General Hospital (MGH) Antidepressant Treatment Response Questionnaire (ATRQ) (Chandler G M, Iosifescu D V, Pollack M H, et al., CNS Neurosci Ther., 16:322-325 (2010)). Patients also must have been on a stable SSRI dose for the past 4 weeks. Patients were excluded if they had failed more than 2 adequate antidepressant trials during the current episode. Patients who demonstrate ≧25% decrease in depressive symptoms on the QIDS-SR total score from screening to baseline were excluded.

### Study Procedures

Eligibility was assessed during the screening and baseline visits, which occurred within 14 days of each other. Patients eligible during the baseline visit were enrolled in the study using the SPCD previously described (Fava M, Evins A E, Dorer D J, et al., Psychother Psychosom, 72:115-127 (2003)). Patients were randomized to one of three treatment groups where they received placebo-placebo, placebo-L-methylfolate 15 mg/day, or L-methylfolate-L-methylfolate 15 mg/day during phases I and II using a randomization code generated by the primary study center. Each phase was 30 days in duration. Study visits occurred every 10 days during which the concomitant SSRI doses remained constant, and patients unable to tolerate the study medications were withdrawn from the study. Patients and investigators were blinded to study assignment.

Patients were assessed at each study visit with the Hamilton Depression Rating Scale (HDRS). In addition, symptom response was evaluated with the HAMD-7 (McIntyre R, Kennedy S, Bagby R M, et al., J Psychiatry Neurosci, 27:235-239 (2002)), the Cognitive and Physical Function Questionnaire (CPFQ) (Fava M, Iosifescu D V, Pedrelli P, et al., Reliability and validity of the MGH Cognitive and Physical Functioning Questionnaire (CPFQ). Psychother Psychosom 78:91-97 (2009)), and the Clinical Global Impression Scale (CGI-S) (Guy W. Ecdeu. Assessment Manual for Psychopharmacology—Revised (DHEW Publ No ADM 76-338). Rockville, Md., U.S. Department of Health, Education, and Welfare, Public Health Service, Alcohol, Drug Abuse, and Mental Health Administration, NIMH Psychopharmacology Research Branch, Division of Extramural Research Programs (1976)). Height and weight were measured, and BMI was calculated in kg/m². Baseline blood samples were collected to assess baseline levels of plasma hsCRP, 4-hydroxy-2-nonenal[4-HNE], and low S-adenosylmethionine/S-adenosylhomocysteine [SAM/SAH] ratio). Also assessed were genetic polymorphisms for a) the C677T, 1298C, and G1793A genotype for methylenetetrahydrofolate reductase (MTHFR); b) the A66G genotype for methionine synthase reductase (MTRR); and c) the A2756G genotype for methionine synthase (MTR). For additional analyses, baseline samples were assessed for genetic polymorphisms for calcium channel, voltage-dependent, L type, alpha 1C subunit (CACNA1C); catechol-O-methyltransferase (COMT); DNA (cytosine-5-)-methyltransferase 3 beta (DNMT3B); dopamine receptor D₂ (DRD2); folate hydrolase 1 (FOLH1); GTP cyclohydrolase 1 (GCH1); GTP cyclohydrolase 1 feedback regulatory protein (GCHFR); and solute carrier family 19 (folate transporter), member 1 (also known as SLC19A1 or reduced folate carrier (RFC1))(Table 6).

TABLE 6
List of genetic markers examined in exploratory analyses

| Genetic Variant | RS Number | Genotype Comparisons |
|---|---|---|
| CACNA1C | rs1006737 | GG vs. AG/AA |
| COMT Val158 | rs4633 | TT vs. CC |
| COMT Val158Met | rs4680 | AA vs. GG |
| DNMT3B | rs1883729 | GG vs. AG/AA and GG vs. AA |
| DRD2 | rs1079596 | CC vs. TC/TT |
| DRD2 129 | rs6275 | CC vs. TT |
| FOLH1 | rs202676 | AA vs. AG/GG |
| GCH1 | rs8007267 | CC vs. TC/TT |
| GCHFR | rs7163862 | AA vs. TA/TT |
| MTHFR 677 | rs1801133 | CC vs. CT/TT |
| MTHFR 1298 | rs1801131 | AA vs. AC/CC |
| MTHFR 1793 | rs2274976 | AA vs. GA |
| MTR 2756 | rs1805087 | AA vs. AG/GG |
| MTRR 66 | rs1801394 | AA vs. AG/GG |
| RFC1 80 | rs1051266 | GG vs. AA |
| RFC1 | rs2297291 | GG vs. AA |
| RFC1 815 | rs12659 | CC vs. TT |

### Assay Methods

Serum hsCRP was measured by a commercially available kit latex particle enhanced immunoturbidimetric assay (Pointe Scientific, Inc., Canton, Mich.). The turbidity (absorbance) was read on an ACE Alera clinical chemistry analyzer (Alpha Wassermann, West Caldwell, N.J.). Plasma 4-HNE was measured by analysis of the amount of FINE-His protein adducts present in the sample using an enzyme immunoassay (OxiSelect HNE-His adduct ELISA kit, Cell Biolabs, Inc., San Diego, Calif.). Plasma SAM and SAH were determined by stable-isotope dilution liquid chromatography-electrospray ionization tandem mass spectrometry as previously described (Inoue-Choi M, Nelson M H, Robien K, et al., Int J Mol Epidemiol Genet, 3:160-173 (2012)). Determination of the presence of genetic polymorphisms was performed on DNA purified from whole blood using a DNeasy blood and tissue kit (Qiagen Inc, Valencia, Calif.). Genotyping was conducted using the MassArray platform (Sequenom, Inc., San Diego, Calif.).

### Statistical Analyses

For exploratory analyses, the pooled treatment effect was assessed by average differences in mean changes from baseline to endpoint for L-methylfolate and placebo groups, pooled across the two phases of the study, consistent with the SPCD of Fava M, Evins A E, Dorer D J, et al., Psychother Psychosom, 72:115-127 (2003) The effect of biomarkers on the response on the HDRS-28 with L-methylfolate compared to placebo was stratified by BMI (≧30 or <30 kg/m²), hsCRP level (median baseline value ≧ or <2.25 mg/L), SAM/SAH ratio (median baseline value ≧ or <2.71), and 4-HNE level (median baseline level ≧ or <3.28 μg/mL). Further, the presence of molecular polymorphisms of genotypes was measured. Elevated BMI, low ratio of SAM/SAH, elevated plasma levels of hsCRP, and 4-HNE, and molecular polymorphisms were evaluated as predictors of a greater pooled (phases I and II according to SPCD) drug/placebo difference.

A standard SPCD analysis approach was employed in order to analyze the study efficacy data. Specifically, an intent-to-treat/last observation carried forward (ITT/LOCF) approach was employed for patients treated with L-methylfolate during phase I. The phase II dataset of interest was limited to patients treated with placebo during phase I and who completed phase I, who did not experience a clinical response on the HDRS during phase I and entered phase II. The LOCF approach was applied to the dataset for phase II, with the final visit of phase I/first visit of phase II serving as the new baseline visit. The ITT/LOCF data comparing L-methylfolate and placebo during phase I were combined with the data comparing L-methylfolate and placebo in phase II according to the model for SPCD and were analyzed using the general approach outlined in Fava M, Evins A E, Dorer D J, et al., Psychother Psychosom, 72:115-127 (2003) using a weight (w=0.50) and a randomization fraction (a=0.333).

Dichotomous measures were analyzed according to the method for dichotomous outcomes (Fava M, Evins A E, Dorer D J, et al., Psychother Psychosom, 72:115-127 (2003)), while seemingly unrelated regression analysis, controlling for baseline scores, was employed for the comparison of continuous outcomes (Tamura R N, Huang X., Clin Trials, 4:309-317 (2007)). All tests were conducted as two-tailed, with alpha set at 0.05. Pooled mean changes from baseline to endpoint for L-methylfolate vs. placebo on the HDRS-28 were stratified for each biomarker and genetic marker. Treatment effect, effect size (difference between means divided by a standard deviation), and 95% confidence intervals (CI) were calculated for each biomarker. In addition, within group analyses, HDRS-28 response rate (at least 50% reduction from baseline), odds ratio, and number needed to treat were determined Within group analyses were conducted separately for individuals who received L-methylfolate (in phase I or as placebo non-responders in phase II) or placebo (in phase I or as placebo non-responders in phase II) with the biomarker or genetic marker status as exposure. Because individuals were not randomized based on their biomarkers status, the within-group analyses adjusted for potential confounders including age, sex, race, and BMI as well as baseline level of HDRS-28. Adjustment was made using linear regression for continuous HDRS-28 and through propensity score stratified analysis for binary outcomes (to decrease the number of predictors in the final model).

### Results

Overall, 74 patients provided data, and 61 (81.3%) completed the study. Detailed results from the primary analysis of the study (efficacy, safety, tolerability of 15 mg L-methylfolate versus placebo) have been published elsewhere (Papakostas G I, Shelton R C, Zajecka J M, et al., Am J Psychiatry, 169:1267-1274 (2012)). For all analyses, results from both phase I and phase II of the study were pooled according to the SPCD method (Fava M, Evins A E, Dorer D J, et al., Psychother Psychosom, 72:115-127 (2003)). Pooled (phases I and II) mean change from baseline was significantly greater with adjunctive L-methylfolate 15 mg/day than placebo for HDRS-28 (−6.8±7.2 vs. −3.7±6.5, p=0.017).

Pooled mean changes with L-methylfolate vs. placebo on the HDRS-28 were examined among subgroups of patients identified by the presence or absence of various biomarkers or their combinations. Pooled mean changes from baseline on the HDRS-28 for L-methylfolate vs. placebo were significantly (p≦0.05) greater among subgroups of patients with a plasma SAM/SAH ratio below the study median value, hsCRP or 4-HNE blood levels above the study median value, or a BMI≧30 kg/m² (consistent with obesity) (Table 7).

TABLE 7
Effect of L-methylfolate 15 mg/day vs. placebo on pooled mean change
from baseline for HDRS-28 stratified by baseline level of plasma marker

| | | Pooled* | | | |
|---|---|---|---|---|---|
| | | Mean | 95% | | Pooled* |
| | | Change vs. | Confidence | p- | Effect |
| Variable | N | Placebo | Interval | value | Size |
| SAM/SAH ≧2.71 | 36 | 0.07 | (−3.33, 3.48) | 0.966 | 0.01 |
| SAM/SAH <2.71 | 37 | −4.57 | (−7.73, −1.41) | 0.005 | −0.75 |
| hsCRP ≧2.25 mg/L | 37 | −3.61 | (−7.23, 0.002) | 0.050 | −0.50 |
| hsCRP <2.25 mg/L | 36 | −2.29 | (−5.47, 0.89) | 0.158 | −0.36 |
| 4-HNE ≧3.28 μg/ | 37 | −4.55 | (−7.61, −1.50) | 0.003 | −0.74 |
| mL | | | | | |
| 4-HNE <3.28 μg/ | 36 | −0.11 | (−3.67, 3.46) | 0.953 | 0.01 |
| mL | | | | | |

*Pooled across study phases with equal weights. A negative sign for pooled effect size indicates that the treatment effect favored the L-methylfolate group.

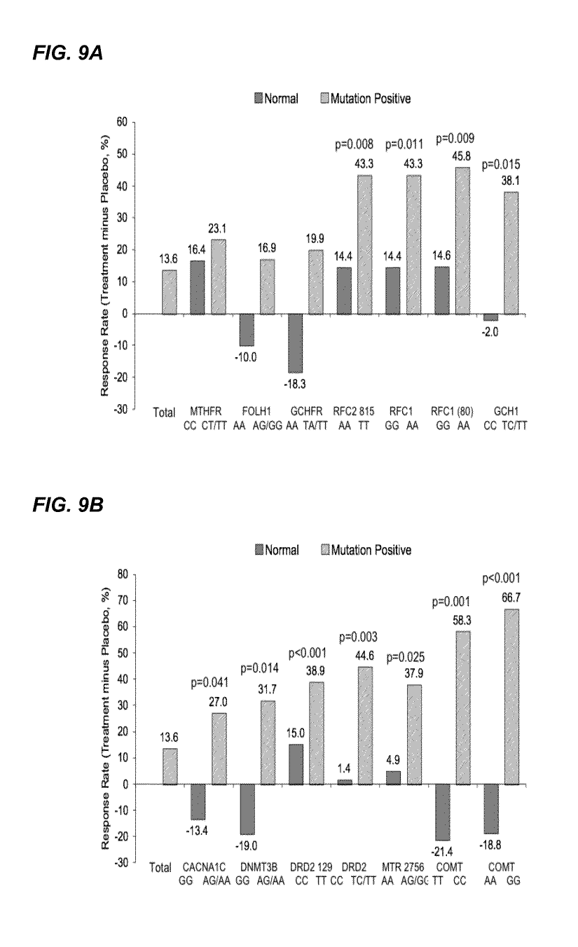

Exploratory analyses demonstrated significant (p≦0.05) differences for pooled mean change from baseline on the HDRS-28 for L-methylfolate vs. placebo based on the presence of most genetic markers at baseline (see, Table 8). Pooled mean change from baseline with L-methylfolate vs. placebo on the HDRS-28 was significantly (p<0.05) greater among subgroups of patients with the MTR 2756 AG/GG or MTRR 66 AG/GG genotype but not significant for the MTHFR 677 CT/TT or MTHFR 1298 AC/CC genotypes compared to the respective homozygous dominant genotypes (Table 8). For the HDRS-28, the pooled effect size ranged from −0.05 to −1.57 for significant mean changes from baseline across all genotypes. Similarly, HDRS-28 response rate (treatment minus placebo) was significantly (p<0.05) improved with L-methylfolate vs. placebo when stratified for baseline presence of most genetic markers. A comparison of the presence of normal and putative positive markers at baseline demonstrated marked differences in the HDRS-28 response rate, with significant (p<0.05) differences noted for most markers except for MTHFR 677CT/TT, FOLH1 AG/GG, and GCHFR TA/TT (FIG. 9A-B).

TABLE 8
Analysis of the effect of L-methylfolate stratified by baseline levels of individual markers (n = 59)

| | | | | | | Response | | | | |
|---|---|---|---|---|---|---|---|---|---|---|
| | | | | Within | Within | Rate | | | | |
| | | | | Treatment | Placebo | Treatment | Within | Pooled | | |
| | | Pooled* | Pooled* | Group | Group | minus | Treatment | Difference | | Number |
| | N | Mean | Effect | Mean | Mean | Placebo | Group | Treatment | Odds | Needed |
| Variable | (%) | Change | Size | Change^{#} | Change^{#} | (%)* | Difference | Group | Ratio | to Treat |
| COMT GG | 17 | −10.9 | −1.57 | −5.70 | 6.52 | 66.7 | 11.219 | 0.32 | NA | 1 |
| (rs4680) | 29% | p < 0.001 | | p = 0.079 | p = 0.007 | p < 0.001 | p = 0.084 | p = 0.045 | | |
| COMT CC | 18 | −9.2 | −1.40 | −4.45 | 6.52 | 58.3 | 11.219 | 0.248 | NA | 2 |
| (rs4633) | 31% | p < 0.001 | | p = 0.162 | p = 0.007 | p = 0.001 | p = 0.084 | p = 0.168 | | |
| MTR 2756 | 20 | −8.2 | −1.15 | −6.95 | 1.96 | 37.9 | 30.97 | 0.298 | 6.24 | 3 |
| AG/GG^{@} | 31% | p < 0.001 | | p = 0.008 | p = 0.397 | p = 0.025 | p = 0.025 | p = 0.037 | | |
| (rs1805087) | | | | | | | | | | |
| RFC1 (80) | 11 | −7.7 | −1.38 | −3.58 | −1.13 | 45.8 | 6.462 | 0.146 | NA | 2 |
| AA | 19% | p = 0.003 | | p = 0.29 | p = 0.73 | p = 0.009 | p = 0.241 | p = 0.314 | | |
| (rs1051266) | | | | | | | | | | |
| DRD2 129 | 10 | −7.6 | −0.70 | −0.65 | 1.44 | 38.9 | 0.000 | 0.09 | 8.00 | 3 |
| TT⁺ | 17% | p = 0.028 | | p = 0.916 | p = 0.652 | p < 0.001 | p = 1.000 | p = 0.693 | | |
| (rs6275) | | | | | | | | | | |
| DRD2 TC/TT | 18 | −6.9 | −0.98 | 0.28 | 4.63 | 44.6 | 2.005 | 0.126 | 13.96 | 2 |
| (rs1079596) | 31% | p = 0.002 | | p = 0.923 | p = 0.047 | p = 0.003 | p = 0.52 | 0.413 | | |
| RFC1 AA | 12 | −5.5 | −0.96 | −2.80 | −0.91 | 43.3 | 3.106 | 0.058 | NA | 2 |
| (rs2297291) | 20% | p = 0.02 | | p = 0.385 | p = 0.77 | p = 0.01 | p = 0.416 | p = 0.708 | | |
| DNMT3B | 32 | −5.4 | −0.84 | −1.70 | 7.93 | 31.7 | 5.977 | 0.113 | 13.33 | 3 |
| AG/AA | 54% | p < 0.001 | | p = 0.573 | p < 0.001 | p = 0.014 | p = 0.156 | p = 0.481 | | |
| (rs1883729) | | | | | | | | | | |
| GCH1 TC/TT | 24 | −5.1 | −0.78 | −4.37 | 1.54 | 38.1 | 119.10 | 0.268 | 9.53 | 3 |
| (rs8007267) | 41% | p = 0.01 | | p = 0.157 | p = 0.550 | p = 0.015 | p = 0.035 | p = 0.075 | | |
| RFC1 815 TT | 10 | −4.9 | −0.83 | −2.80 | −0.91 | 43.3 | 3.106 | NA | NA | 2 |
| (rs12659) | 17% | p = 0.036 | | p = 0.385 | p = 0.77 | p = 0.008 | p = 0.416 | | | |
| BMI ≧ 30 | 40 | −4.7 | −0.75 | −3.53 | 6.31 | 30.2 | 10.76 | 0.22 | 7.11 | 3 |
| kg/m^{2&} | 56% | p = 0.001 | | p = 0.166 | p = 0.003 | p = 0.009 | p = 0.067 | p = 0.117 | | |
| CACNA1C | 37 | −4.2 | −0.64 | −4.73 | 1.52 | 27.0 | >1000 | 0.193 | 4.08 | 4 |
| AG/AA | 63% | p = 0.005 | | p = 0.095 | p = 0.510 | p = 0.041 | p = 0.997 | p = 0.156 | | |
| (rs1006737) | | | | | | | | | | |
| MTHFR 677 | 24 | −3.8 | −0.53 | −0.67 | 5.29 | 23.1 | 0.36 | −0.06 | 3.99 | 4 |
| CT/TT^{@} | 37% | p = 0.087 | | p = 0.822 | p = 0.035 | p = 0.114 | p = 0.402 | p = 0.728 | | |
| (rs1801133) | | | | | | | | | | |
| FOLH1 | 30 | −3.2 | −0.40 | −2.99 | 0.83 | 16.9 | >1000 | −0.042 | 2.46 | 6 |
| AG/GG | 51% | p = 0.076 | | p = 0.497 | p = 0.850 | p = 0.119 | p = 0.997 | p = 0.828 | | |
| (rs202676) | | | | | | | | | | |
| GCHFR | 43 | −3.1 | −0.48 | −5.62 | 0.54 | 19.9 | >1000 | 0 | 2.92 | 5 |
| TA/TT | 73% | p = 0.037 | | p = 0.225 | p = 0.849 | p = 0.078 | p = 0.997 | p = 1.000 | | |
| (rs7163862) | | | | | | | | | | |
| MTRR66 | 49 | −2.9 | −0.45 | 1.27 | −4.12 | 22.6 | 1.47 | 0.001 | 3.38 | 4 |
| AG/GG^{@} | 75% | p = 0.041 | | p = 0.694 | p = 0.117 | p = 0.041 | p = 0.782 | p = 0.994 | | |
| (rs1801394) | | | | | | | | | | |
| MTHFR1298 | 30 | −0.5 | −0.05 | −0.08 | −5.58 | 19.1 | 2.069 | 0.123 | 2.47 | 5 |
| AC/CC^{@} | 46% | p = 0.807 | | p = 0.981 | p = 0.009 | p = 0.19 | p = 0.561 | p = 0.457 | | |
| (rs1801131) | | | | | | | | | | |

*HDRS-28, treatment minus placebo;
^{#}Adjusted for baseline, race, age, and BMI;
^{&}n = 72 (three patients were missing height value for calculating BMI;
^{@}n = 65 (10 patients did not consent to genetic testing);
⁺n = 58 (SNP results were unreadable in one patient);
for n = 59, samples for genetic testing were depleted;
NA = not available

Further exploratory analyses were conducted to determine the effect of L-methylfolate vs. placebo stratified by baseline levels of individual markers when response was assessed using HAMD-7, CGI-S, and CPFQ (Table 9). Significant (p<0.05) improvements were noted for pooled mean change from baseline with L-methylfolate vs. placebo for all genetic markers except for MTHFR 1298 AC/CC on the CGI-S and for many markers on the HAMD-7 and CPFQ.

TABLE 9
Analysis of the effect of L-methylfolate on HAMD-7, CGI-S, and CPFQ
stratified by baseline levels of individual genetic markers (n = 59)

| | | Pooled* | Pooled* | Pooled* |
|---|---|---|---|---|
| | | Mean | Mean | Mean |
| | | Change | Change | Change |
| Variable | N (%) | HAMD-7 | CGI-S | CPFQ |
| Total | 75 | −1.49 | −0.52 | −1.51 |
| | 100% | p = 0.018 | p = 0.002 | p = 0.068 |
| BMI ≧30 kg/m^{2 &} | 40 | −2.52 | 0.23 | −3.04 |
| | 56% | p = 0.002 | p < 0.001 | p = 0.032 |
| CACNA1C AG/AA | 37 | −2.43 | −0.83 | −1.92 |
| | 63% | p = 0.009 | p = 0.001 | p = 0.177 |
| COMT (Val158Met) GG | 17 | −5.17 | −1.71 | −7.99 |
| | 29% | p = 0.01 | p < 0.001 | p = 0.004 |
| COMT (Val158) CC | 18 | −5.06 | −1.53 | −7.36 |
| | 31% | p = 0.009 | p < 0.001 | p = 0.008 |
| DNMT3B AG/AA | 32 | −2.64 | −0.82 | −2.53 |
| | 54% | p = 0.003 | p = 0.001 | p = 0.079 |
| DRD2 TC/TT | 18 | −3.60 | −0.90 | 0.43 |
| | 31% | p = 0.007 | p = 0.013 | p = 0.825 |
| DRD2 129 TT⁺ | 10 | −1.31 | NA | −5.35 |
| | 17% | p = 0.489 | | p = 0.387 |
| FOLH1 AG/GG | 30 | −2.26 | −0.70 | −2.96 |
| | 51% | p = 0.017 | p = 0.013 | p = 0.019 |
| GCH1 TC/TT | 24 | −2.96 | −0.74 | −3.22 |
| | 41% | p = 0.004 | p-0.021 | p = 0.007 |
| GCHFR TA/TT | 43 | −1.75 | −0.61 | −0.56 |
| | 73% | p = 0.038 | p = 0.003 | p = 0.631 |
| MTHFR 677 CT/TT^{@} | 24 | −1.46 | −0.77 | −2.21 |
| | 37% | p = 0.176 | p = 0.024 | p = 0.02 |
| MTHFR 1298 AC/CC^{@} | 30 | −0.24 | −0.49 | −0.08 |
| | 46% | p = 0.819 | p = 0.055 | p = 0.960 |
| MTR 2756 AG/GG^{@} | 20 | −4.56 | −0.99 | −3.01 |
| | 31% | p < 0.001 | p = 0.003 | p = 0.05 |
| MTRR 66 AG/GG^{@} | 49 | −1.62 | −0.62 | −2.85 |
| | 75% | p = 0.052 | p = 0.004 | p = 0.026 |
| RFC1 AA | 12 | −2.91 | −1.07 | −3.82 |
| | 21% | p = 0.036 | p = 0.021 | p = 0.096 |
| RFC1 (80) AA | 11 | −3.65 | −1.31 | −3.79 |
| | 19% | p = 0.031 | p = 0.006 | p = 0.147 |
| RFC1 815 TT | 10 | −2.62 | −1.05 | −1.18 |
| | 17% | p = 0.053 | p = 0.015 | p = 0.522 |

*HDRS-28, treatment minus placebo;
^{#}Adjusted for baseline, race, age, and BMI;
^{&}n = 72 (three patients were missing height value for calculating BMI;
^{@}n = 65 (10 patients did not consent to genetic testing);
⁺n = 58 (SNP results were unreadable in one patient); for n = 59, samples for genetic testing were depleted;
NA = not available.

The effect on the pooled mean change from baseline with L-methylfolate vs. placebo for the HDRS-28 in patients with combinations of biological and genetic markers present at baseline also was examined Combinations of markers demonstrated pooled mean change from baseline for L-methylfolate vs. placebo that ranged from −3.6 to −23.3 and pooled effect size that ranged from −0.56 to −4.50. (Table 10). Combinations of MTHFR 677 CT/TT+MTR 2756 AG/GG, GCH1 TC/TT+COMT GG, and GCH1 TC/TT+COMT CC demonstrated the largest effect size (−23.3, −20.7, and −18.2, respectively) and pooled mean change for L-methylfolate vs. placebo was highly significant (p<0.001).

TABLE 10
Analysis of the effect of L-methylfolate 15 mg/day on pooled mean change from baseline vs. placebo
for HDRS-28 stratified by combinations of biomarker level status and genotype (n = 59)

| | | Pooled* | | | | |
|---|---|---|---|---|---|---|
| | | Mean | | Pooled* | Response Rate | Number |
| | | Change vs. | | Effect | Treatment | Needed |
| Variable | N (%) | Placebo | 95% CI | Size | Minus Placebo | to Treat |
| MTHFR 677 CT/TT + | 8 | −23.3 | (−32.09, −14.50) | −2.51 | 66.7% | 1 |
| MTR 2756 AG/GG | 14% | <0.001 | | | p = 0.002 | |
| GCH1 TC/TT + | 11 | −20.7 | (−29.99, −11.33) | NA | 75.0% | 1 |
| COMT (rs4680) GG | 19% | <0.001 | | | p < 0.001 | |
| GCH1 TC/TT + | 12 | −18.2 | (−24.70, −11.78) | NA | 66.7% | 1 |
| COMT (rs4633) CC | 20% | <0.001 | | | p < 0.001 | |
| CACNA1C AG/AA + | 13 | −16.2 | (−24.70, −7.77) | −2.93 | 83.3% | 1 |
| COMT (rs4680) GG | 22% | <0.001 | | | p < 0.001 | |
| MTR 2756 AG/GG + | 7 | −15.1 | (−24.17, −6.04) | −4.50 | 100% | 1 |
| COMT (rs4633) CC | 12% | 0.001 | | | p < 0.001 | |
| BMI ≧ 30 kg/m² + | 10 | −14.4 | (−19.45, −9.41) | −2.83 | 92.9% | 1 |
| MTR 2756 AG/GG | 17% | <0.001 | | | p < 0.001 | |
| CACNA1C AG/AA + | 13 | −13.5 | (−17.13, −9.91) | −2.90 | 83.8% | 1 |
| MTR 2756 AG/GG | 22% | <0.001 | | | p < 0.001 | |
| DNMT3B AG/AA + | 15 | −13.1 | (−18.49, −7.67) | −2.55 | 100% | 1 |
| COMT (rs4680) GG | 25% | <0.001 | | | p < 0.001 | |
| CACNA1C AG/AA + | 14 | −13.0 | (−19.27, −6.65) | −2.67 | 75.0% | 1 |
| COMT (rs4633) CC | 24% | <0.001 | | | p < 0.001 | |
| BMI ≧ 30 kg/m² + | 16 | −12.4 | (−16.95, −7.78) | −2.62 | 70.0% | 1 |
| GCH1 TC/TT | 27% | <0.001 | | | p < 0.001 | |
| DNMT3B AG/AA + | 13 | −12.0 | (−14.93, −9.08) | −2.38 | 70.8% | 1 |
| MTR 2756 AG/GG | 22% | <0.001 | | | p < 0.001 | |
| GCHFR TA/TT + | 12 | −12.0 | (−16.61, −7.48) | −2.25 | 65.0% | 2 |
| MTR 2756 AG/GG | 20% | <0.001 | | | p < 0.001 | |
| FOLH1 AG/GG + | 15 | −11.8 | (−18.33, −5.23) | −1.63 | 66.7% | 1 |
| COMT (rs4680) GG | 25% | 0.001 | | | p < 0.001 | |
| BMI ≧ 30 kg/m² + | 14 | −11.8 | (−20.94, −2.65) | −1.39 | 58.3 | 2 |
| COMT (rs4680) GG | 24% | 0.011 | | | p = 0.010 | |
| GCH1 TC/TT + | 7 | −11.4 | (−20.20, −2.51) | −2.02 | 75.0% | 1 |
| RFC1 80 AA | 12% | 0.012 | | | p < 0.001 | |
| DNMT3B AG/AA + | 16 | −10.9 | (−15.55, −6.24) | −2.24 | 83.3% | 1 |
| COMT (rs4633) CC | 27% | <0.001 | | | p < 0.001 | |
| RFC1 AA + | 7 | −10.5 | (−18.51, −2.56) | −2.66 | 75.0% | 1 |
| GCH1 TC/TT | 12% | 0.01 | | | p = 0.001 | |
| GCH1 TC/TT + | 9 | −10.4 | (−20.43, −0.46) | −1.36 | 62.5% | 2 |
| MTR 2756 AG/GG | 15% | 0.04 | | | p = 0.002 | |
| CACNA1C AG/AA + | 10 | −9.9 | (−16.32, −3.55) | −1.48 | 68.8% | 1 |
| DRD2 TC/TT | 17% | 0.002 | | | p < 0.001 | |
| MTHFR 677 CT/TT + | 13 | −9.9 | (−15.79, −3.97) | −1.45 | 50.0% | 2 |
| BMI ≧ 30 kg/m² | 22% | 0.001 | | | p = 0.003 | |
| BMI ≧ 30 kg/m² + | 20 | −9.8 | (−13.67, −5.94) | −1.98 | 66.7% | 1 |
| DNMT3B AG/AA | 34% | <0.001 | | | p < 0.001 | |
| FOLH1 AG/GG + | 16 | −9.8 | (−15.36, −4.29) | −1.47 | 58.3% | 2 |
| COMT (rs4633) CC | 27% | 0.001 | | | p = 0.001 | |
| DNMT3B AA + | 9 | −9.7 | (−15.23, −4.08) | −1.76 | 50.0% | 2 |
| FOLH1 AG/GG | 15% | 0.001 | | | p < 0.001 | |
| GCHFR TA/TT + | 15 | −9.7 | (−15.03, −4.40) | −1.91 | 56.0% | 2 |
| DRD2 TC/TT | 25% | <0.001 | | | p < 0.001 | |
| BMI ≧ 30 kg/m² + | 10 | −9.6 | (−16.40, −2.81) | −1.46 | 68.8% | 1 |
| DRD2 TC/TT | 17% | 0.006 | | | p < 0.001 | |
| DRD2 TC/TT + | 8 | −9.5 | (−14.95, −4.14) | −2.29 | 66.7% | 1 |
| MTR 2756 AG/GG | 14% | 0.001 | | | p = 0.001 | |
| DNMT3B AA + | 5 | −9.5 | (−17.42, −1.53) | −2.34 | 50.0% | 2 |
| MTR 2756 AG/GG | 8% | 0.019 | | | p < 0.001 | |
| CACNA1C AG/AA + | 17 | −9.4 | (−14.37, −4.52) | −1.48 | 59.2% | 2 |
| GCH1 TC/TT | 29% | <0.001 | | | p = 0.001 | |
| MTHFR 677 CT/TT + | 13 | −9.0 | (−13.59, −4.39) | −1.15 | 47.1% | 2 |
| CACNA1C AG/AA | 22% | <0.001 | | | p = 0.015 | |
| CACNA1C AG/AA + | 8 | −9.0 | (−17.07, −0.89) | −1.48 | 50.0% | 2 |
| DRD2 129 TT | 14% | 0.03 | | | p < 0.001 | |
| FOLH1 AG/GG + | 18 | −8.9 | (−13.30, −4.41) | −1.22 | 45.8% | 2 |
| MTR 2756 AG/GG | 31% | <0.001 | | | p = 0.010 | |
| CACNA1C AG/AA + | 21 | −8.8 | (−11.94, −5.73) | −1.64 | 54.6% | 2 |
| DNMT3B AG/AA | 36% | <0.001 | | | p = 0.001 | |
| FOLH1 AG/GG + | 8 | −8.1 | (−13.27, −2.96) | −1.68 | 33.3% | 3 |
| MTHFR 1793 GA | 14% | 0.002 | | | p = 0.176 | |
| FOLH1 AG/GG + | 9 | −8.1 | (−14.18, −1.99) | −1.36 | 50.0% | 2 |
| RFC1 80 AA | 15% | 0.009 | | | p = 0.012 | |
| GCHFR TA/TT + | 8 | −8.1 | (−13.27, −2.96) | −1.68 | 33.3% | 3 |
| MTHFR 1793 GA | 14% | 0.002 | | | p = 0.176 | |
| CACNA1C AG/AA + | 6 | −8.0 | (−15.94, −0.06) | −1.32 | 40.0% | 3 |
| MTHFR 1793 GA | 10% | 0.048 | | | p = 0.177 | |
| FOLH1 AG/GG + | 26 | −7.7 | (−10.85, −4.65) | −1.18 | 42.5% | 2 |
| DNMT3B AG/AA | 44% | <0.001 | | | p = 0.004 | |
| FOLH1 AG/GG + | 20 | −7.6 | (−11.66, −3.51) | −1.32 | 51.2% | 2 |
| GCH1 TC/TT | 34% | <0.001 | | | p = 0.002 | |
| GCHFR TA/TT + | 17 | −7.5 | (−13.24, −1.76) | −1.16 | 58.3% | 2 |
| COMT (rs4633) CC | 29% | 0.001 | | | p = 0.002 | |
| FOLH1 AG/GG + | 17 | −7.2 | (−11.53, −2.82) | −1.04 | 47.9% | 2 |
| DRD2 TC/TT | 29% | 0.001 | | | p = 0.001 | |
| GCHFR TA/TT + | 21 | −7.2 | (−10.58, −3.92) | −1.37 | 40.4% | 2 |
| DNMT3B AG/AA | 36% | <0.001 | | | p = 0.010 | |
| GCHFR TA/TT + | 7 | −7.1 | (−13.69, −0.50) | −2.63 | 50.0% | 2 |
| RFC1 80 AA | 12% | 0.035 | | | p = 0.016 | |
| BMI ≧ 30 kg/m² + | 26 | −7.1 | (−11.02, −3.13) | −1.05 | 40.2% | 2 |
| CACNA1C AG/AA | 44% | <0.001 | | | p = 0.014 | |
| FOLH1 AG/GG + | 9 | −6.9 | (−12.24, −1.51) | −1.31 | 50.0% | 2 |
| RFC1 AA | 15% | 0.012 | | | p = 0.020 | |
| RFC1 815 TT + | 8 | −6.2 | (−11.46, −0.84) | −1.19 | 50.0% | 2 |
| FOLH1 AG/GG | 14% | 0.023 | | | p = 0.015 | |
| GCH1 TC/TT + | 18 | −5.8 | (−12.86, 1.31) | −0.76 | 41.2% | 2 |
| DNMT3B AG/AA | 31% | 0.110 | | | p = 0.037 | |
| GCHFR TA/TT + | 8 | −5.6 | (−12.47, −1.24) | −0.85 | 45.8% | 2 |
| RFC1 AA | 14% | 0.109 | | | p = 0.014 | |
| GCHFR TA/TT + | 8 | −5.6 | (−12.47, −1.24) | −0.85 | 45.8% | 2 |
| RFC1 815 TT | 14% | 0.109 | | | p = 0.014 | |
| MTHFR 677 CT/TT + | 20 | −4.5 | (−9.69, −0.77) | −0.58 | 25.0% | 4 |
| FOLH1 AG/GG | 34% | 0.095 | | | p = 0.148 | |
| BMI ≧ 30 kg/m² + | 23 | −4.2 | (−8.19, −0.29) | −0.74 | 35.0% | 3 |
| GCHFR TA/TT | 39% | 0.035 | | | p = 0.025 | |
| GCHFR TA/TT + | 18 | −3.6 | (−8.02, −0.89) | −0.56 | 22.6% | 4 |
| DRD2 129 TT | 31% | 0.117 | | | p = 0.219 | |

*Pooled across study phases with equal weights;
NA = not available;
sample size differences were explained by n = 72 (three patients were missing height value for calculating BMI;
n = 65 (10 patients did not consent to genetic testing);
n = 59, samples for genetic testing were depleted;
n = 58 (SNP results were unreadable in one patient).

### Discussion

Results from the primary analyses demonstrated significant differential efficacy with 15 mg L-methylfolate versus placebo as adjunctive therapy among patients with an inadequate response to SSRIs (Papakostas G I, Shelton R C, Zajecka J M, et al., Am J Psychiatry, 169:1267-1274 (2012)). The overall effect size on the HDRS with L-methylfolate (0.41) was similar to the effect size (0.35 to 0.37) observed in other studies of adjunctive therapy in MDD (Marcus R N, McQuade R D, Carson W H, et al., J Clin Psychopharmacol., 28:156-165 (2008); Reimherr F W, Martin M L, Eudicone J M, et al., J Clin Psychopharmacol., 30:300-305 (2010)). The results provided herein revealed a greater differential treatment effect with L-methylfolate vs. placebo among patients stratified by the presence of baseline level biological and genetic biomarkers (moderators of outcome) that were associated with metabolic dysfunction, inflammation or variants of L-methylfolate metabolism (e.g., BMI, hsCRP, MTRR, MTR).

Provided herein are the associations between the presence of select biomarkers at baseline and the response to L-methylfolate. The treatment effect and effect size with 15 mg L-methylfolate versus placebo when stratified by the presence of specific biological plasma or genetic markers appears larger than those reported from conventional antidepressant-placebo trials (Fournier J C, DeRubeis R J, Hollon S D, et al., JAMA, 303:47-53 (2010); Nelson J C, Mankoski R, Baker R A, et al., J Affect Disord., 120:133-140 (2010)). Combinations of markers demonstrated an even greater treatment effect with 15 mg L-methylfolate with effect sizes exceeding 1.0 in most comparisons.

Several biomarkers have been identified that are associated with an increased risk or severity of MDD. Increased body weight and obesity are positively associated with an increased risk of MDD and a poorer response to antidepressant treatment (Kloiber S, Ising M, Reppermund S, et al., Biol Psychiatry, 62:321-326 (2007); Luppino F S, de Wit L M, Bouvy P F, et al., Arch Gen Psychiatry, 67:220-229 (2010); Faith M S, Butryn M, Wadden T A, et al., Obes Rev., 12:e438-453 (2011); Ma J, Xiao L., Obesity, 18:347-353 (2010)). Genetic markers related to folate metabolism have been investigated for their association with MDD (Peerbooms O L, van Os J, Drukker M, et al., Brain Behav Immun., 25:1530-1543 (2011); Slopien R, Jasniewicz K, Meczekalski B, et al., Maturitas, 61:252-255 (2008); Lewis S J, Lawlor D A, Davey Smith G, et al., Mol Psychiatry, 11:352-360 (2006); Lopez-Leon S, Janssens A C, Gonzalez-Zuloeta Ladd A M, et al., Mol Psychiatry, 13:772-785 (2008)). The results described herein provide support for the benefits of L-methylfolate as adjunctive treatment for patients not responding adequately to SSRIs and suggest additional avenues for identifying those individuals most likely to respond to this treatment. The methods described herein can be used to provide individualized treatment approaches for depressed patients unresponsive to initial antidepressant therapy.

The Hamilton Depression Rating Scale (HDRS) is widely used as the standard for assessing drug response in clinical trials of MDD. However, the HDRS has been criticized because it is multidimensional, lacks sensitivity to detect clinical change, and lacks discriminative power to define remission (Ballesteros J, Bobes J, Bulbena A, et al., J Affect Disord., 102:93-99 (2007)). For these analyses, the HDRS-28 rather than the HDRS-17 item score was used to compare symptom improvement with L-methylfolate vs. placebo because the longer version is more sensitive to changes in patients with symptoms of atypical or melancholic depression (Nemeroff C B, J Psychiatr Res., 41:189-206 (2007); Cusin C, Yang H, Yeung A, Fava M., In: Baer L, Blais M A (eds), Handbook of Clinical Rating Scales and Assessment in Psychiatry and Mental Health, Humana Press (2010)). Additionally, the 7-item Hamilton Depression Rating Scale (HAMD-7) and Cognitive and Physical Functioning Questionnaire (CPFQ) were used in the exploratory analyses because the HAMD-7 may be more sensitive to change in clinical trials of depression (McIntyre R, Kennedy S, Bagby R M, et al., J Psychiatry Neurosci, 27:235-239 (2002); McIntyre R S, Konarski J Z, Mancini D A, et al., CMAJ, 173:1327-1334 (2005)), and the CPFQ has been found to measure cognitive and physical symptoms of depression, which are predictive of residual symptoms (Fava M, Iosifescu D V, Pedrelli P, et al., Reliability and validity of the MGH Cognitive and Physical Functioning Questionnaire (CPFQ). Psychother Psychosom 78:91-97 (2009)).

In conclusion, greater efficacy was observed with L-methylfolate when used as an adjunct to SSRI treatment in inadequate responders. The present analyses indicate that the relative superiority of L-methylfolate versus placebo with respect to efficacy may be further enhanced among subsets of patients stratified by the presence of metabolic and genetic markers related to inflammation and disturbance of folate metabolism. These results indicate that the presence of certain surrogate markers can be used to identify patients with SSRI-resistant MDD who are particularly responsive to adjunctive therapy with 15 mg L-methylfolate.

### Example 4

### Predictive Biomarkers of the Antidepressant Response to Adjunctive L-Methylfolate in Patients with Major Depressive Disorder

This example illustrates the use of genetic biomarkers (e.g., single nucleotide polymorphisms (SNPs)) for predicting whether a patient with major depressive disorder will respond to combination therapy of L-methylfolate and an antidepressant drug (e.g., a selective serotonin reuptake inhibitor (SSRI)). In particular, the example shows that specific synergistic dual-marker combinations are indicative of a therapeutic response to adjunctive therapy of L-methylfolate and an antidepressant drug in patients diagnosed with MDD.

Furthermore, the therapeutic response to the adjunctive therapy was detected using various neuropsychological assessment scales such as HDRS, CGI-S and CPFQ.

### Abstract

Biological or genetic markers may be associated with an increased risk of MDD or inadequate response to therapy. The objective of the study analysis was to evaluate the effect of specific markers alone and in combination on the antidepressant effect of adjunctive L-methylfolate (15 mg) among patients with an inadequate response to SSRIs.

In the primary study, L-methylfolate was evaluated in 75 outpatients with SSRI-resistant MDD in a double-blind, randomized, placebo-controlled trial. Patients received either L-methylfolate at 15 mg/day for 60 days, placebo for 30 days followed by L-methylfolate at 15 mg/day for 30 days, or placebo for 60 days. Detailed descriptions of the clinical study are found in, e.g., Papakostas G I et al., Am J Psychiatry, 169:1267-74 (2012) and U.S. Patent App. Nos. US 2013/0172361 and US 2013/0267523 and International Patent. App. Pub. NO: WO 2013/074676, the disclosures of which are herein incorporated by reference in their entirety for all purposes.

In the analysis described below, the effect of baseline body mass index (BMI) and the presence of methylenetetrahydrofolate reductase (MTHFR 677 CT/TT; rs1801133), methionine synthase (MTR 2756 AG/GG; rs1805087), catechol-O-methyltransferase (COMT Val158Met GG; rs4680), and GTP cyclohydrolase 1 (GCH1 TC/TT; rs8007267) polymorphisms individually and in combination on treatment response to L-methylfolate was evaluated.

Patients with a BMI≧30 kg/m² experienced a significantly greater symptom reduction with L-methylfolate (the 28-item Hamilton Depression Rating Scale (HDRS-28) pooled mean change was −4.66; p=0.001), compared to those with a BMI<0 kg/m². Pooled mean changes from baseline for HDRS-28 with COMT Val158Met GG, GCH1 TC/TT, and MTR 2756 AG/GG were significantly (p≦0.05) greater with L-methylfolate vs. placebo with pooled effect sizes ranging from 0.75 to 1.57. Pooled mean changes from baseline for HDRS-28 with most combinations of these five markers (BMI, MTHFR, MTR, COMT and GCH1) also were significant (pooled mean changes range: −23.29, −9.88, respectively; p<0.05). These results were supported by significant differences for most comparisons using the outcome measures HDRS-7, the Clinical Global Impressions-Severity Scale (CGI-S) and the Cognitive and Physical Function Questionnaire (CPFQ).

Adjunctive therapy of L-methylfolate at 15 mg/day demonstrated a significant treatment effect in patients stratified by biomarkers, genotypes or their combination. These conditions either individually and in combination were predictive of an inadequate response to SSRI therapy. The presence of combinations of specific biomarkers and genotypes also appeared to predict a benefit from L-methylfolate augmentation while their absence indicated little to no benefit from such augmentation.

### Introduction

The 12-month prevalence of major depressive disorder (MDD) is reported to be 6.7% among U.S. adults (Kessler R C, Arch Gen Psychiatry., 62:617-27 (2005)) with a lifetime prevalence of 16.9% (Andrade L et al., Int J Methods Psychiatr Res., 12:3-21 (2003)). The occurrence of MDD is associated with a substantial burden to the patient and caregivers and markedly greater economic burden (Fostick L et al., Eur Neuropsychopharmacol., 20:671-5 (2010); Ivanova J I et al., Curr Med Res Opin., 26:2475-84 (2010); Knoth R L et al., Am J Manag Care, 16:e188-96 (2010)). The majority of patients experience a poor response rate to antidepressants (Rush A J et al., Am J Psychiatry, 163:1905-17 (2006)). There is no reliable predictor of treatment efficacy (Fabbri C et al., Am J Med Genet B Neuropsychiatr Genet., 162B:487-520 (2013)), and thus, antidepressant therapy often results in poor or inadequate drug response.

An inadequate response to initial antidepressant therapy continues to hinder effective management of MDD in up to two-thirds of patients (Thase, 2003; Rush A J et al., Am J Psychiatry, 163:1905-17 (2006)). While switching antidepressant drugs, augmentation strategies, and combination therapy are treatment options for patients who are not responding, another approach is to attempt to identify patients who will have an optimal response, i.e., by measuring levels of biomarkers or molecular markers that are associated with MDD risk and/or treatment response.

It has been hypothesized that genetic polymorphisms contribute to inter-individual variability of antidepressant drug response (Fabbri C et al., Am J Med Genet B Neuropsychiatr Genet., 162B:487-520 (2013)). Polymorphisms associated with antidepressant efficacy, tolerability, and safety have been identified. However, the studies have small sample sizes, poorly defined patient populations and confounding variables, which have limited the clinical applicability of the results (Fabbri C et al., Am J Med Genet B Neuropsychiatr Genet., 162B:487-520 (2013)). The complex neurobiology of MDD and pharmacogenetics pose a challenge for transitioning clinical trials results to clinical practice. An increased understanding of the molecular mechanisms that are associated with an antidepressant response, which should lead to improved outcomes in MDD is needed (O'Leary et al., Pharmacol Biochem Behay., [Epub ahead of print] PubMed PMID: 24161683 (2013 Oct. 24)).

Various genetic biomarkers have been identified that are associated with an increased risk of MDD, a greater severity of MDD or a poor response to antidepressant treatment (Vogelzangs N et al., J Clin Psychiatry, 71:391-9 (2010); Kloiber S, Biol Psychiatry., 62:321-6 (2007); Luppino F S et al., Arch Gen Psychiatry, 67:220-9 (2010)). For example, the genetic markers 5,10-methylenetetrahydrofolate reductase (MTHFR C677T) and catechol-O-methyltransferase (COMT) are recognized as predictive of a response to antidepressant treatment or an increased risk of suicide (Peerbooms O L et al., Brain Behav Immun., 25:1530-43 (2011); Lanctot et al., 2010; Slopien et al., 2008; Schosser A et al., Eur Neuropsychopharmacol., 22:259-66 (2012)). Thus, assessment of baseline levels of biomarkers or the presence or absence of genetic markers could be useful for identifying MDD patients who are more likely to respond to antidepressant treatment.

The role of L-methylfolate as a valuable adjunct to conventional antidepressant treatment of MDD is being increasingly recognized (Papakostas G I et al., Am J Psychiatry, 169:1267-74 (2012); Farah A., CNS Spectr., 14(1 Suppl 2):2-7 (2009); Ginsberg L D, Oubre A, Daoud Y., Innov Clin Neurosci., 8:19-28 (2011)). L-methylfolate modulates the synthesis of dopamine, norepinephrine, and serotonin via tetrahydrobiopterin (BH₄), which is critical for their synthesis by activating tyrosine hydroxylase and tryptophan hydroxylase (Bottiglieri, 2005; Miller A L, Altern Med Rev., 13:216-26 (2008)). Dysfunctional folate disposition can increase the risk for depression, interfere with antidepressant treatment effects, and result in decreased treatment response (Vogelzangs N, Beekman A T, Boelhouwer I G, et al., J Clin Psychiatry, 72:598-604 (2011)). Results from two randomized, placebo-controlled trials in MDD patients with an adequate response to SSRIs demonstrated improved response and remission rates with adjunctive L-methylfolate treatment compared with continued SSRI monotherapy (Papakostas G I et al., Am J Psychiatry, 169:1267-74 (2012)). The analysis of one trial, as presented herein, evaluated the predictive value of five genetic biomarkers alone and in combination on the treatment response to adjunctive L-methylfolate.

### Methods

A post-hoc analysis was conducted based on the results from a multicenter, 60-day, randomized, double-blind trial of L-methylfolate as adjunctive therapy for patients with MDD and previous inadequate responses to SSRI treatment. The study design and results were presented in full and are summarized in Papakostas G I et al., Am J Psychiatry, 169:1267-74 (2012). The study protocol, amendments, and informed consent forms were reviewed and approved by an Institutional Review Board. Written informed consent was obtained from all study patients before any study procedures were conducted (ClinicalTrials.gov Registration Number NCT00955955).

### Patient Selection

Patients satisfying Diagnostic and Statistical Manual IV (DSM-IV) criteria for a current episode of MDD and age 18-65 years were eligible if they had a Quick Inventory of Depressive Symptoms-Self Report (QIDS-SR) score ≧12 at screening and baseline visits. Patients were required to have a history of previous treatment with an SSRI during the current episode of MDD for ≧8 weeks at adequate doses (≧20 mg/day of fluoxetine, citalopram, or paroxetine, ≧10 mg/day of escitalopram or ≧50 mg/day of sertraline) that remained stable for the previous 4 weeks, as determined using the Massachusetts General Hospital (MGH) Antidepressant Treatment Response Questionnaire (ATRQ) (Chandler G M et al., CNS Neurosci Ther., 16:322-5 (2010)). Exclusion criteria included failure on ≧2 adequate antidepressant trials during the current episode and ≧25% decrease in depressive symptoms on the QIDS-SR total score from screening to baseline.

### Study Procedure

Screening and baseline visits occurred within 14 days of each other, and patients eligible during the baseline visit were enrolled in the study after providing written informed consent. Patients were randomized in a 2:3:3 ratio using the Sequential Parallel Comparison Design (SPCD) (Fava M et al., Psychother Psychosom., 72:115-27 (2003)) to treatment groups consisting of placebo-placebo, placebo-L-methylfolate at 15 mg/day or L-methylfolate-L-methylfolate at 15 mg/day during the 30-day treatment for Phase I and Phase II and were maintained at a stable dose on current SSRI treatment. Patients were withdrawn from the study if they were unable to tolerate study medications.

At each study visit response was evaluated with the Hamilton Depression Rating Scale (HDRS) (Hamilton M., J Neurol Neurosurg Psych., 23:56-62 (1960)) and the Clinical Global Impression Severity and scales (CGI-S) (Guy W., In: ECDEU assessment manual for psychopharmacology. Washington (DC): Superintendent of Documents, US Government Printing Office, US Department of Health, Education, and Welfare Publication NO: 76-338, 1976:218-22). In addition, symptom response was evaluated with the HDRS-7 (McIntyre R S, CMAJ, 173:1327-34 (2005)) and the Cognitive and Physical Function Questionnaire (CPFQ) (Fava M. et al., Psychother Psychosom., 78:91-7 (2009)). Response was defined as a ≧50% reduction from baseline on the HDRS-28. Height and weight were measured, and BMI was calculated in kg/m². Baseline blood samples were collected to assess the presence of genetic polymorphisms for the methylene tetrahydrofolate reductase (MTHFR) T677C allele, COMT Val158Met GG, GTP cyclohydrolase 1 (GCH1) TC/TT, and methionine synthase (MTR) A2756G.

### Assay Methods

Determination of genetic polymorphisms for MTHFR, MTR, COMT, and GCH1 was performed on DNA purified from whole blood using a DNeasy blood and tissue kit (Qiagen Inc, Valencia, Calif.). Genotyping was conducted using the MassArray platform (Sequenom, Inc., San Diego, Calif.).

### Statistical Analysis

For the analyses, the pooled treatment effect was assessed by differences in mean changes from baseline to endpoint for L-methylfolate and placebo groups, pooled across the two phases of the study. This is consistent with the SPCD of Fava M et al., Psychother Psychosom., 72:115-27 (2003)). Treatment response on the HDRS-28 with L-methylfolate compared to placebo was stratified by baseline BMI (≧30 kg/m² or <30 kg/m²). Further, the presence or absence of polymorphisms of MTHFR 677 TC/TT (rs1801133), MTR 2756 AG/GG (rs1805087), GCH1 TC/TT (rs8007267), and COMT Val158Met GG (rs4680) genotypes was determined and used as a dichotomous variable.

Efficacy data were analyzed using the SPCD and an intent-to-treat/last observation carried forward (ITT/LOCF) approach for patients treated with L-methylfolate during phase I. The phase II dataset was limited to patients who completed treatment with placebo during phase I and did not experience a clinical response on the HDRS and entered phase II. The final visit of phase I/first visit of phase II was considered the new baseline visit. Data comparing L-methylfolate and placebo from phase I and phase II were combined according to the SPCD model and were analyzed using the approach outlined in Fava M et al., Psychother Psychosom., 72:115-27 (2003) using a weight (w=0.50) and a randomization fraction (a=0.333). Dichotomous measures were analyzed according to the method for dichotomous outcomes (Fava M et al., Psychother Psychosom., 72:115-27 (2003)) while seemingly unrelated regression analysis, controlling for baseline scores, was employed for the comparison of continuous outcomes (Tamura R N, Huang X., Clin Trials, 4:309-17 (2007)). All tests were conducted as two-tailed, with alpha set at 0.05.

For the HDRS-28, pooled mean changes from baseline to endpoint for L-methylfolate vs. placebo were stratified for each biomarker and genetic marker. Treatment effect and effect size were calculated for each biomarker. Within group analyses, HDRS-28 response rate (≧50% reduction from baseline), odds ratio, and number needed to treat were calculated. For individuals who received L-methylfolate (in phase I or as placebo non-responders in phase II) or placebo (in phase I or as placebo non-responders in phase II), within-group analyses were conducted separately, with biomarker or genetic marker status factored as exposure. Within-group analyses were adjusted for potential confounders including age, sex, race, BMI, and baseline level of HDRS-28 to account for the fact that individual patients were not randomized by baseline marker status. To decrease the number of predictors in the final model, adjustments used linear regression for continuous HDRS-28 scores and propensity score stratified analysis for binary outcomes.

### Results

Data from 74 patients were available for analysis, and 61 (81.3%) patients completed the study. Results previously were published from the primary analysis of L-methylfolate versus placebo (Papakostas G I et al., Am J Psychiatry, 169:1267-74 (2012)). Overall, pooled mean (SD) baseline HDRS-28 score was 26.0 (5.0) for L-methylfolate and placebo groups combined. Pooled mean change from baseline vs. placebo was significantly greater with adjunctive L-methylfolate 15 mg/day for HDRS-28 (−2.74, p=0.017).

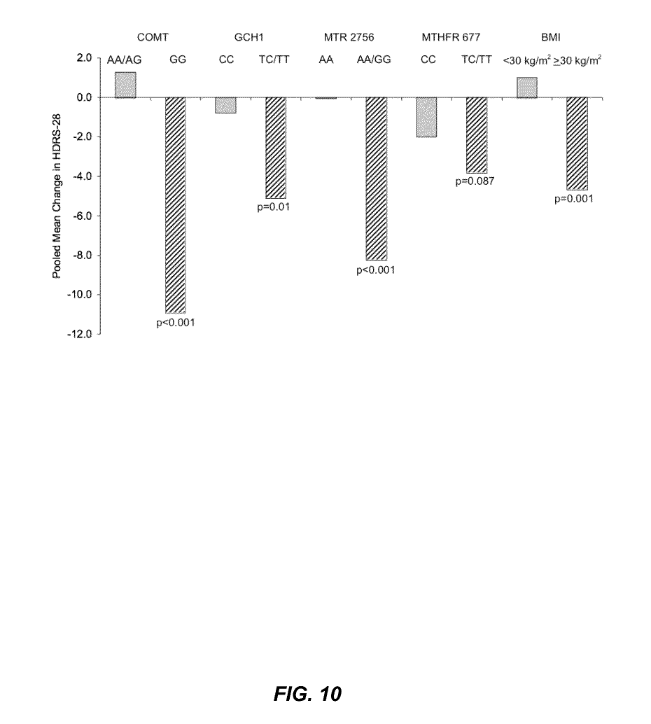

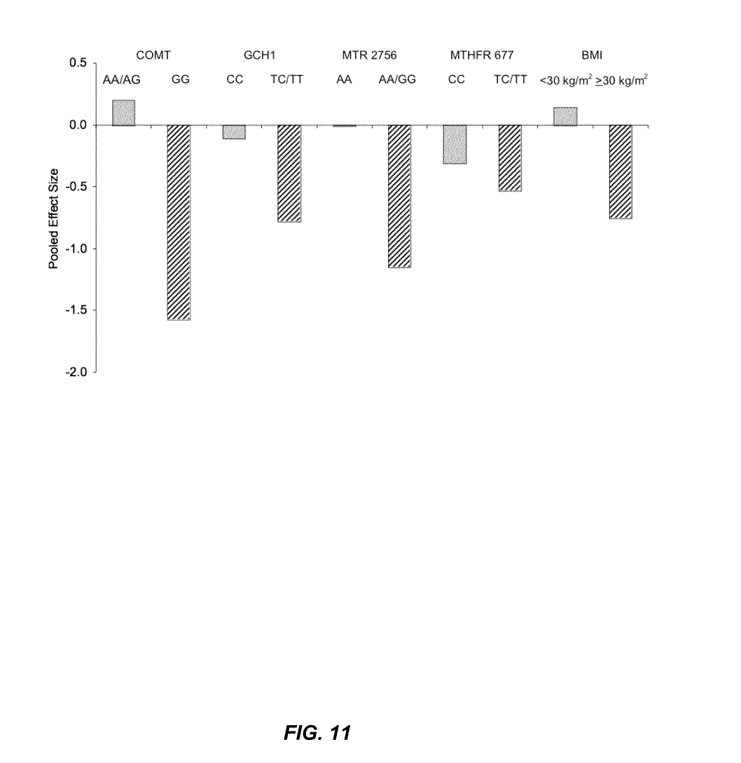

Pooled mean change from baseline with L-methylfolate vs. placebo on the HDRS-28 was significantly (p≦0.01) greater among subgroups of patients with COMT Val158Met GG, GCH1 TC/TT, and MTR 2756 AG/GG genotypes and elevated BMI, and marginally significant (p=0.087) for the MTHFR 677 TC/TT genotype (Table 11 and FIG. 10). Pooled effect size was greater than 0.50 for each of these markers (FIG. 11). Pooled response rate for treatment minus placebo ranged from 23.1% to 66.7% and was significantly (p<0.05) greater with L-methylfolate stratified by each marker except for MTHFR 677 TC/TT.

TABLE 11
Analyses of the effect of L- methylfolate stratified by individual molecular marker

| | | | | | | Response | | | | |
|---|---|---|---|---|---|---|---|---|---|---|
| | | | | Within | Within | Rate | | | | |
| | | | | Treatment | Placebo | Treatment | Within | Pooled | | |
| | | Pooled* | Pooled* | Group | Group | minus | Treatment | Difference | | Number |
| | N | Mean | Effect | Mean | Mean | Placebo | Group | Treatment | Odds | Needed |
| Variable | (%) | Change | Size | Change^{#} | Change^{#} | (%)* | Difference | Group | Ratio | to Treat |
| COMT | 42 | 1.27 | 0.20 | NA | NA | −6.0 | NA | NA | 0.72 | −17 |
| (Val158Met) | 71% | p = 0.437 | | | | p = 0.608 | | | | |
| AA/AG | | | | | | | | | | |
| COMT | 17 | −10.9 | −1.57 | −5.70 | 6.52 | 66.7 | 11.22 | 0.32 | NA | 1 |
| (Val158Met) | 29% | p < 0.001 | | p = 0.079 | p = 0.007 | p < 0.001 | p = 0.084 | p = 0.045 | | |
| GG | | | | | | | | | | |
| GCH1 CC | 35 | −0.76 | −0.11 | NA | NA | −2.0 | NA | NA | 0.89 | −50 |
| | 59% | p = 0.657 | | | | p = 0.874 | | | | |
| GCH1 TC/TT | 24 | −5.09 | −0.78 | −4.37 | 1.54 | 38.1 | 119.1 | 0.27 | 9.53 | 3 |
| | 41% | p = 0.01 | | p = 0.157 | p = 0.550 | p = 0.015 | p = 0.035 | p = 0.075 | | |
| MTR 2756 | 45 | −0.05 | −0.01 | NA | NA | 4.9 | NA | NA | 1.36 | 20 |
| AA | 69% | p = 0.975 | | | | p = 0.643 | | | | |
| MTR 2756 | 20 | −8.24 | −1.15 | −6.95 | 1.96 | 37.9 | 30.97 | 0.30 | 6.24 | 3 |
| AG/GG | 31% | p < 0.001 | | p = 0.008 | p = 0.397 | p = 0.025 | p = 0.025 | p = 0.037 | | |
| MTHFR 677 | 41 | −1.99 | −0.31 | NA | NA | 16.4 | NA | NA | 2.35 | 6 |
| CC | 63% | p = 0.198 | | | | p = 0.188 | | | | |
| MTHFR 677 | 24 | −3.75 | −0.53 | −0.67 | 5.29 | 23.1 | 0.36 | −0.06 | 3.99 | 4 |
| TC/TT^{#} | 37% | p = 0.087 | | p = 0.822 | p = 0.035 | p = 0.114 | p = 0.402 | p = 0.728 | | |
| BMI <30 | 32 | 0.99 | 0.14 | NA | NA | −19.0 | NA | NA | 0.24 | −5 |
| kg/m² | 44% | p = 0.648 | | | | p = 0.088 | | | | |
| BMI ≧ 30 | 40 | −4.66 | −0.75 | −3.53 | 6.31 | 30.2 | 10.76 | 0.22 | 7.11 | 3 |
| kg/m² | 56% | p = 0.001 | | p = 0.166 | p = 0.003 | p = 0.009 | p = 0.067 | p = 0.117 | | |

*HDRS-28, treatment minus placebo;
^{#}Adjusted for baseline, race, age, sex and BMI;
NA = not available;
sample size differences were explained by n = 72 (two patients were missing height value for calculating BMI;
n = 65 (10 patients did not consent to genetic testing);
n = 59, samples for genetic testing were depleted.

The effect on the pooled mean change from baseline with L-methylfolate vs. placebo for the HDRS-28 was examined in subgroups of patients using combinations of various biomarkers and genetic markers (Table 12). Significant (p<0.05) differences in pooled mean change from baseline for L-methylfolate vs. placebo exceeded −10 for all combinations except for MTHFR 677 TC/TT plus BMI≧30 kg/m². Pooled effect size ranged from −40.32 to −1.36 across all combinations. For combinations of three markers, numbers of patients were too small for statistical analysis of many combinations.

TABLE 12
Analyses of the effect of L- methylfolate stratified by combinations of molecular markers

| | | | | | | Response | | | |
|---|---|---|---|---|---|---|---|---|---|
| | | | | Within | Within | Rate | | | |
| | | | | Treatment | Placebo | Treatment | Within | Within | |
| | | Pooled* | Pooled* | Group | Group | minus | Treatment | Placebo | Number |
| | N | Mean | Effect | Mean | Mean | Placebo | Group | Group | Needed |
| Variable | (%) | Change | Size | Change^{#} | Change ^{#} | (%)* | Difference | Difference | to Treat |
| COMT (Val158Met) | 11 | −20.66 | −40.32 | −4.54 | 3.13 | 75.0 | NA | 0.11 | 1 |
| GG + GCH1 TC/TT | 19% | p < 0.001 | | p = 0.029 | p = 0.042 | p < 0.001 | | p = 0.270 | |
| COMT (Val158Met) | 7 | −15.11 | −4.50 | −5.37 | 3.62 | 100.0 | 11.35 | 0.21 | 1 |
| GG + MTR 2756 | 12% | p = 0.001 | | p = 0.003 | p = 0.021 | p < 0.001 | p = 0.037 | p = 0.258 | |
| AG/GG | | | | | | | | | |
| COMT (Val158Met) | 4 | NA | NA | −3.38 | 5.73 | NA | 2.33 | 0.01 | NA |
| GG + MTHFR 677 | 7% | | | p = 0.135 | p < 0.001 | | p = 0.423 | p = 0.100 | |
| TC/TT | | | | | | | | | |
| COMT (Val158Met) | 14 | −11.80 | −1.39 | −3.86 | 5.97 | 58.3 | 13.95 | 0.07 | 2 |
| GG + BMI ≧ 30 kg/m² | 24% | p = 0.011 | | p = 0.036 | p < 0.001 | p = 0.01 | p = 0.03 | p = 0.023 | |
| GCH1 TC/TT + | 9 | −10.45 | −1.36 | −4.79 | 1.75 | 62.5 | 68.89 | 0.40 | 2 |
| MTR 2756 AG/GG | 15% | p = 0.040 | | p = 0.007 | p = 0.316 | p = 0.002 | p = 0.061 | p = 0.476 | |
| GCH1 TC/TT + | 6 | NA | NA | −3.73 | 3.33 | 90.0 | 7.05 | 0.03 | 1 |
| MTHFR 677 TC/TT | 10% | | | p = 0.139 | p = 0.052 | p < 0.001 | p = 0.115 | p = 0.089 | |
| GCH1 TC/TT + | 16 | −12.36 | −2.62 | −3.88 | 4.38 | 70.0 | NA | 0.15 | 1 |
| BMI ≧ 30 kg/m² | 28% | p < 0.001 | | p = 0.04 | p = 0.008 | p < 0.001 | | p = 0.067 | |
| MTR 2756 AG/GG + | 8 | −23.29 | −2.51 | −6.09 | 2.69 | 66.7 | 15.24 | 0.31 | 1 |
| MTHFR 677 TC/TT | 12% | p < 0.001 | | p = 0.01 | p = 0.071 | p = 0.002 | p = 0.093 | p = 0.228 | |
| MTR 2756 AG/GG + | 10 | −14.43 | −2.83 | −6.34 | 4.53 | 92.9 | NA | 0.17 | 1 |
| BMI ≧ 30 kg/m² | 16% | p < 0.001 | | p = 0.001 | p = 0.008 | p < 0.001 | | p = 0.079 | |
| MTHFR 677 TC/TT + | 13 | −9.88 | −1.45 | −2.22 | 6.49 | 50.0 | 2.04 | 0.02 | 2 |
| BMI ≧ 30 kg/m² | 21% | p = 0.001 | | p = 0.24 | p < 0.001 | p = 0.003 | p = 0.375 | p = 0.028 | |

*HDRS-28, treatment minus placebo;
^{#}Adjusted for baseline, race, age, sex and BMI;
NA = not available.

All combinations that included BMI≧30 kg/m² demonstrated significant (p<0.05) within treatment group pooled mean changes with L-methylfolate vs. placebo (range: −5.29 to −2.85), except for GCH1 TC/TT plus MTHFR 677 TC/TT plus BMI≧30 kg/m², which was only trending (p=0.056) and COMT Val158Met GG plus MTHFR 677 TC/TT plus BMI≧30 kg/m² (p=0.071) (Table 13)

TABLE 13
Pooled mean change from baseline for L- methylfolate vs. placebo on the
HDRS-28 according to the presence of combinations of three markers

| | | Within | Within | | | |
|---|---|---|---|---|---|---|
| | | Treatment | Placebo | Within | Within | |
| | | Group | Group | Treatment | Placebo | Number |
| | N | Mean | Mean | Group | Group | Needed |
| Variable | (%) | Change * | Change * | Difference | Difference | to Treat |
| COMT (Val158Met) GG + | 4 | −4.09 | 2.59 | 13.90 | 0.22 | 1 |
| GCH1 TC/TT + MTR 2756 | 7% | p = 0.003 | p = 0.037 | p = 0.034 | p = 0.218 | |
| AG/GG | | | | | | |
| COMT (Val158Met) GG + | 2 | −4.23 | −3.50 | 17.63 | 0.03 | NA |
| GCH1 TC/TT + MTHFR 677 | 3% | p = 0.025 | p = 0.004 | p = 0.035 | p = 0.057 | |
| TC/TT | | | | | | |
| COMT (Val158Met) GG + | 9 | −3.60 | 3.95 | 42.35 | 0.11 | 2 |
| GCH1 TC/TT + | 16% | p = 0.012 | p = 0.001 | p = 0.021 | p = 0.043 | |
| BMI ≧ 30 kg/m² | | | | | | |
| COMT (Val158Met) GG + | 3 | −5.17 | 3.50 | 13.58 | 0.16 | NA |
| MTR 2756 AG/GG + | 5% | p = 0.002 | p = 0.003 | p = 0.062 | p = 0.088 | |
| MTHFR 677 TC/TT | | | | | | |
| COMT (Val158Met) GG + | 5 | −4.41 | 4.33 | 18.92 | 0.13 | 1 |
| MTR 2756 AG/GG + | 9% | p = 0.001 | p < 0.001 | p = 0.033 | p = 0.034 | |
| BMI ≧ 30 kg/m² | | | | | | |
| COMT (Val158Met) GG + | 4 | −2.85 | 5.48 | 3.33 | 0.01 | NA |
| MTHFR 677 TC/TT + | 7% | p = 0.071 | p < 0.001 | p = 0.128 | p = 0.050 | |
| BMI ≧ 30 kg/m² | | | | | | |
| GCH1 TC/TT + MTR 2756 | 3 | −5.53 | 2.43 | 62.82 | 0.22 | NA |
| AG/GG + MTHFR 677 | 5% | p = 0.002 | p = 0.057 | p = 0.053 | p = 0.141 | |
| TC/TT | | | | | | |
| GCH1 TC/TT + MTR 2756 | 6 | −4.38 | 3.30 | NA | 0.27 | 1 |
| AG/GG + BMI ≧ 30 kg/m² | 10% | p = 0.001 | p = 0.018 | | p = 0.109 | |
| GCH1 TC/TT + MTHFR 677 | 5 | −3.31 | 4.37 | 6.42 | 0.06 | 1 |
| TC/TT + BMI ≧ 30 kg/m² | 9% | p = 0.056 | p = 0.001 | p = 0.045 | p = 0.030 | |
| MTR 2756 AG/GG + | 5 | −5.29 | 4.05 | 20.08 | 0.16 | 1 |
| MTHFR 677 TC/TT + | 8% | p = 0.002 | p = 0.002 | p = 0.028 | p = 0.047 | |
| BMI ≧ 30 kg/m² | | | | | | |

* Adjusted for baseline, race, age, sex and BMI;
NA = not available.

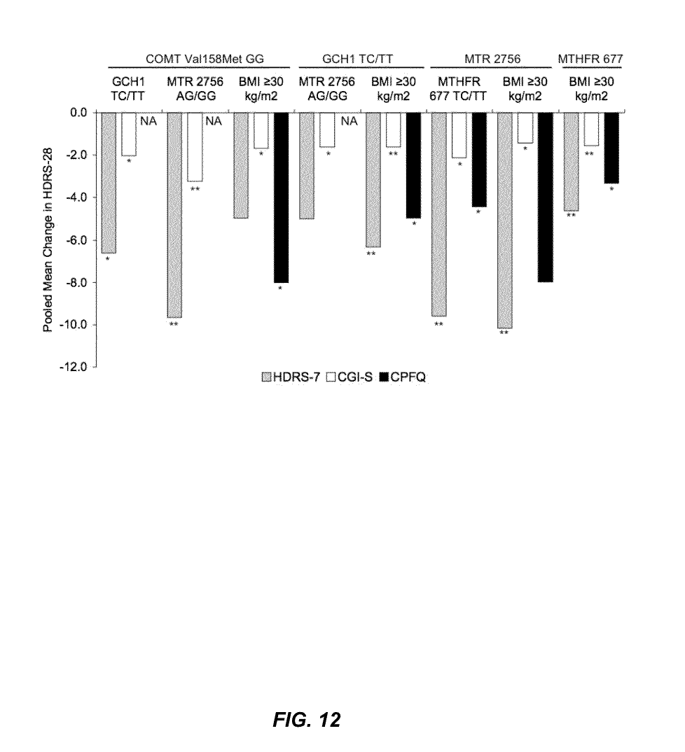

Pooled mean change for L-methylfolate vs. placebo was examined using the HDRS-7, CGI-S, and CPFQ scales to confirm the consistency of the findings from the HDRS-28 (Table 14). Significant (p<0.05) differences for L-methylfolate vs. placebo were observed in the overall population on the HDRS-7 and the CGI-S. For the three scales, significant (p<0.05) differences for L-methylfolate vs. placebo were observed for each of the comparisons of interest (COMT Val158Met GG, GCH1 TC/TT, and MTR 2756 AG/GG genotypes and BMI≧30 kg/m²), except for the MTHFR 677 TC/TT genotype, which was significant (p<0.05) on the CGI-S and CPFQ only. Combinations of markers demonstrated significant (p<0.05) differences for pooled mean change with L-methylfolate vs. placebo for most comparisons (FIG. 12).

TABLE 14
Pooled mean change from baseline for L-methylfolate vs. placebo
according to the presence of individual markers on the HDRS-7,
CGI-S and CPFQ

| Variable | N (%) | HDRS-7* | CGI-S* | CPFQ* |
|---|---|---|---|---|
| Total population | 74 | −1.49 | −0.52 | −1.51 |
| | 100% | p = 0.018 | p = 0.002 | p = 0.068 |
| COMT (Val158Met) | 42 | 0.41 | −0.06 | 0.08 |
| AA/AG | 71% | p = 0.634 | p = 0.807 | p = 0.934 |
| COMT (Val158Met) GG | 17 | −5.17 | −1.71 | −7.99 |
| | 29% | p = 0.010 | p < 0.001 | p = 0.004 |
| GCH1 CC | 35 | −0.41 | −0.42 | −0.30 |
| | 59% | p = 0.663 | p = 0.081 | p = 0.815 |
| GCH1 TC/TT | 24 | −2.96 | −0.74 | −3.22 |
| | 41% | p = 0.004 | p = 0.021 | p = 0.007 |
| MTR 2756 AA | 45 | 0.09 | −0.29 | −0.63 |
| | 69% | p = 0.912 | p = 0.190 | p = 0.620 |
| MTR 2756 AG/GG | 20 | −4.56 | −1.09 | −3.01 |
| | 31% | p < 0.001 | p = 0.001 | p = 0.050 |
| MTHFR 677 CC | 41 | −1.40 | −0.43 | −0.96 |
| | 63% | p = 0.101 | p = 0.063 | p = 0.511 |
| MTHFR 677 TC/TT^{#} | 24 | −1.46 | −0.77 | −2.21 |
| | 37% | p = 0.176 | p = 0.024 | p = 0.020 |
| BMI <30 kg/m² | 32 | 0.47 | 0.23 | 0.11 |
| | 44% | p = 0.665 | P = 0.391 | p = 0.909 |
| BMI ≧30 kg/m² | 40 | −2.52 | −0.85 | −3.04 |
| | 56% | p = 0.002 | p < 0.001 | p = 0.032 |

*Pooled mean change vs. placebo; sample size differences were explained by n = 72 (two patients were missing height value for calculating BMI;
^{#}adjusted for baseline, race, age, sex and BMI;
n = 65 (10 patients did not consent to genetic testing);
n = 59, samples for genetic testing were depleted.

### Discussion

The primary analysis of L-methylfolate vs. placebo as adjunctive therapy for patients with a documented inadequate response to SSRIs demonstrated significant improvement in the response rate with L-methylfolate (Papakostas G I et al., Am J Psychiatry, 169:1267-74 (2012)). Results from previous studies with L-methylfolate as monotherapy or adjunctive therapy among inadequate responders to SSRIs also have shown improved response (Farah A., CNS Spectr., 14(1 Suppl 2):2-7 (2009); Ginsberg L D, Oubre A, Daoud Y., Innov Clin Neurosci., 8:19-28 (2011)).

Results from this exploratory analysis demonstrated that the presence or absence of any of the five biomarkers provided prognostic information for the utility of L-methylfolate as adjunctive treatment to standard antidepressant therapy. Prognostic benefit was observed with individual markers, however, combinations of two or more markers surprisingly provided significantly improved predictive value. These results suggest that screening for these five biomarkers could be a valuable tool for identifying patients with a high unmet need for effective adjunctive therapy; for predicting treatment response or the lack of a response; and for identifying individual patients who may show a beneficial response to L-methylfolate. The presence of these biomarkers appears to suggest a moderate independent benefit of L-methylfolate augmentation and predict a more robust, synergistic L-methylfolate effect when certain combinations of biomarkers are present (e.g., the COMT (Val158Met) GG and GCH1 TC/TT pair and the MTR 2756 AG/GG+MTHFR 677 TC/TT pair). In the absence of these biomarkers, there appears to be little to no benefit from L-methylfolate augmentation

The effect sizes reported with individual markers or combinations of markers in this analysis were substantial compared with other studies of antidepressant therapy. A meta-analysis of placebo-controlled antidepressant trials reported an average effect size of 0.31 with active drug therapy (Turner E H, Rosenthal R., BMJ, 336:516-7 (2008)). A second meta-analysis of placebo-controlled trials with atypical antipsychotics (aripiprazole, quetiapine, risperidone, and olanzapine/fluoxetine) as adjunctive therapy for MDD also reported an effect size of 0.31 across all studies and a pooled difference in mean change vs. placebo on the MADRS of 2.69 points (Spielmans G I et al., PLoS Med., 10:e1001403 (2013)). In the analysis provided herein, the effect sizes and pooled mean changes were considerably higher with L-methylfolate when stratified on the basis of individual markers or combinations of these markers.

The presence of certain biomarkers tends to be associated with risk or severity of MDD, while other biomarkers are predictive factors for a response to antidepressant therapy. A sizeable body of literature demonstrates that increased body weight and obesity are associated with both a greater risk of MDD and a poorer response to standard antidepressant treatment (Oskooilar N, J Clin Psychiatry, 70:1609-10 (2009); Kloiber S, Biol Psychiatry., 62:321-6 (2007); Ma et al., 2010; Faith M S et al., Obes Rev., 12:e438-53 (2011); Luppino F S et al., Arch Gen Psychiatry, 67:220-9 (2010); Simon et al., 2008; McIntyre R S et al., Can J Psychiatry, 51:274-80 (2006)). A strong association has been reported between the presence of MTHFR 677 TC/TT polymorphism and the risk of MDD (Wu Y L et al., Prog Neuropsychopharmacol Biol Psychiatry, 46:78-85 (2013); Lopez-Leon S et al., Mol Psychiatry, 13:772-85 (2008); Peerbooms O L et al., Brain Behav Immun., 25:1530-43 (2011); Gilbody S, Lewis S, Lightfoot T., Am J Epidemiol., 165:1-13 (2007); Lewis S J et al., Mol Psychiatry, 11:352-60 (2006)). In addition, the COMT Val158Met polymorphism was associated with a worse response to standard antidepressant therapy or increased risk of suicide (Baune B T et al., Neuropsychopharmacology, 33:924-32 (2008); Benedetti et al., 2009; Schosser A et al., Eur Neuropsychopharmacol., 22:259-66 (2012)). Published data is much more limited with other genetic markers included in the analysis described herein and their association with MDD. In a study of Japanese patients with MDD, no association was found between the presence of GCH1 and MDD, but GCH1 was a predictor of response to SSRIs (Kishi T et al., J Affect Disord., 142:315-22 (2012)). Among postmenopausal women, the MTR 2756 GG genotype was associated with a 5-fold increased risk of MDD as was the presence of the MTHFR 677 TC/TT polymorphism (Slopien et al., 2008). Published literature is sparse on the impact of two or more markers on the risk of MDD and the response to treatment. Pan C C et al., Int J Geriatr Psychiatry, 24:847-55 (2009) used MRI to evaluate the association between COMT and MTHFR genes with putamen volumes and MDD in a geriatric population, and found that neither genotype alone demonstrated a role, but the presence of both polymorphisms was associated with MRI differences between depressed and non-depressed patients. Secher A et al., Int Clin Psychopharmacol., 24:199-203 (2009) reported an association between increased body weight and the presence of COMT polymorphisms among depressed patients on antidepressant therapy. In this study, mean changes for L-methylfolate vs. placebo on the HDRS-28 exceeded 10 points when stratified by the presence of specific marker combinations, and significant differences also were noted for most marker combinations with the CGI-S, HDRS-7, and CPFQ. The findings suggest specific combinations of biomarkers (e.g., (e.g., the COMT (Val158Met) GG and GCH1 TC/TT pair and the MTR 2756 AG/GG+MTHFR 677 TC/TT pair) that can be used to identify subjects diagnosed with MDD who are likely to respond to adjunctive L-methylfolate treatment regimen.

The HDRS is widely used as the standard for assessing drug response in clinical trials of depression. However, the HDRS has been criticized because it is multidimensional, lacks sensitivity to detect clinical change, and lacks discriminative power to define remission (Ballesteros J et al., J Affect Disord., 102:93-9 (2007)). Among the potential limitations of the HDRS is that in addition to core symptoms of MDD, it also assesses symptom responses that may reflect the adverse effects of conventional antidepressants, for example, sleep, insomnia, anxiety, and restlessness (Kennedy S H, Clin Neurosci., 10:271-7 (2008)). Other subscales of the HDRS have been extensively evaluated as more sensitive assessments of the core symptoms of MDD than the longer versions of the HDRS (Santen G et al., J Psychiatr Res., 42:1000-9 (2008); Kennedy S H, Clin Neurosci., 10:271-7 (2008); McIntyre R S, CMAJ, 173:1327-34 (2005)). For this reason, this study used the HDRS-7, CGI-S, and CPFQ, in addition to the HDRS-28, to validate the findings. Most of the significant differences identified for individual markers and combinations of markers on the HDRS-28 also were significant when measured with these additional scales, which adds strength to the findings. The results, even when stratified by the presence of various markers, demonstrated statistically significant, robust antidepressant response with L-methylfolate as adjunctive therapy during acute, short-term treatment of MDD.

The analysis provided herein demonstrated a robust response among subsets of patients stratified by the presence of bio- and genetic markers. The significant treatment effects of L-methylfolate were consistent regardless of whether analyses were based on the HDRS-28 or CGI-S, or when using other assessment scales. Thus, the results demonstrate the utility of individual markers or combinations of markers, such as the COMT (Val158Met) GG and GCH1 TC/TT pair and the MTR 2756 AG/GG+MTHFR 677 TC/TT pair, as predictive tools to identify subjects responsive to treatment with L-methylfolate.

### REFERENCES

1. V. Herbert, Experimental nutritional folate deficiency in man. Trans Assoc Am Phys 75 (1961), p. 307.
2. M. W. P. Carney, Serum folate values in 423 psychiatric patients. Br Med J 4 (1967), p. 512.
3. E. H. Reynolds, J. M. Preece, J. Bailey and A. Coppen, Folate deficiency in depressive illness. Br J Psychiatry 117 (1970), p. 287.
4. S. D. Shorvon, M. W. P. Carney, I. Chanarin and E. H. Reynolds, The neuropsychiatry of megaloblastic anaemia. Br Med J 281 (1980), p. 1036.
5. MRC Vitamin Study Research Group, Prevention of neural tube defects: results of the Medical Research Council Vitamin Study. Lancet 338 (1991), p. 131.
6. E. B. Rimm, W. C. Willett, F. B. Hu et al., Folate and vitamin B6 from diet and supplements in relation to risk of coronary heart disease among women. JAMA 279 (1998), p. 359.
7. D. Mischoulon, Herbal remedies for mental illness. Psychiatr Clin North Am Annu Drug Ther 6 (1999), p. 1.
8. A. Fugh-Berman and J. M. Cott, Dietary supplements and natural products as psychotherapeutic agents. Psychosom Med 61 (1999), p. 712.
9. American Psychiatric Association. Diagnostic and statistical manual of mental disorders, 4th ed. Washington, D.C.: American Psychiatric Association, 1994.
10. T. Bottiglieri, Folate, vitamin B12, and neuropsychiatric disorders. Nutr Rev 54 (1997), p. 382.
11. J. E. Alpert and M. Fava, Nutrition and depression: the role of folate. Nutr Rev 55 (1997), p. 145.
12. B. R. Hutto, Folate and cobalamin in psychiatric illness. Compr Psychiatry 38 (1997), p. 305.
13. M. I. Botez, T. Botez, J. Leveille, P. Bielmann and M. Cadotte, Neurological correlates of folic acid deficiency: facts and hypotheses. In: M. I. Botez and E. H. Reynolds, Editors, Folic acid in neurology, psychiatry and internal medicine, Raven Press, New York (1979), p. 435.
14. E. H. Reynolds, Anticonvulsant drugs, folate metabolism and mental symptoms. In: M. Dam, L. Gram and J. K. Penry, Editors, Advances in epileptology. XIIth Epilepsy International Symposium, Raven Press, New York (1981), p. 621.
15. J. Edeh and B. K. Toone, Antiepileptic therapy, folate deficiency, and psychiatric morbidity: a general practice survey. Epilepsia 26 (1985), p. 434.
16. S. D. Shorvon, M. W. P. Carney, I. Chanarin and E. H. Reynolds, The neuropsychiatry of megaloblastic anemia. Br Med J 281 (1980), p. 1036.
17. S. N. Young and M. Ghadirian, Folic acid and psychopathology. Prog Neuropsychopharmacol Biol Psychiatry 13 (1989), p. 841.
18. R. Crellin, T. Bottiglieri and E. H. Reynolds, Folates and psychiatric disorders: clinical potential. Drugs 45 (1993), p. 623.
19. A. M. Ghadirian, J. Anath and F. Engelsmann, Folic acid deficiency in depression. Psychosomatics 21 (1980), p. 926.
20. M. W. P. Carney, T. K. N. Chary, M. Laundy et al., Red cell folate concentrations in psychiatric patients. J Affect Dis 19 (1990), p. 207.
21. T. Bottiglieri, K. Hyland, M. Laundy et al., Folate deficiency, biopterin and monoamine metabolism in depression. Psychol Med 22 (1992), p. 871.
22. A. Wilkinson, D. Anderson, M. Abou-Saleh et al., 5-Methyltetrahydrofolate level in the serum of depressed subjects and its relationship to the outcome of ECT. J Affect Disord 32 (1994), p. 163.
23. M. T. Abou-Saleh and A. Coppen, Serum and red blood cell folate in depression. Acta Psychiatr Scand 80 (1989), p. 78.
24. E. H. Reynolds, J. M. Preece, J. Bailey and A. Coppen, Folate deficiency in depressive illness. Br J Psychiatry 117 (1970), p. 287.
25. V. A. Wesson, A. J. Levitt and R. T. Joffe, Change in folate status with antidepressant treatment. Psychiatry Res 53 (1994), p. 313.
26. A. Levitt and R. Joffee, Folate, B12, and life course of depressive illness. Biol Psychiatry 25 (1989), p. 867.
27. I. R. Bell, J. S. Edman, D. W. Marby et al., Vitamin B12 and folate status in acute geropsychiatric inpatients: affective and cognitive characteristics of a vitamin nondeficient population. Biol Psychiatry 27 (1990), p. 125.
28. M. Fava, J. S. Borus, J. E. Alpert et al., Folate, B12, and homocysteine in major depressive disorder. Am J Psychiatry 154 (1997), p. 426.
29. Alpert M, Silva R, Pouget E. Folate as a predictor of response to sertraline or nortriptyline in geriatric depression. Presented at the 36th Annual Meeting of the NCDEU. Boca Raton, Fla., May 28-31, 1996.
30. M. Fava and K. G. Davidson, Definition and epidemiology of treatment-resistant depression. Psychiatr Clin North Am 119 (1996), p. 176.
31. P. S. A. Godfrey, B. K. Toone, M. W. P. Carney et al., Enhancement of recovery from psychiatric illness by methylfolate. Lancet 336 (1990), p. 392.
32. M. W. P. Carney and B. F. Sheffield, Associations of subnormal serum folate and vitamin B12 values and effects of replacement therapy. J Nery Ment Dis 150 (1970), p. 404.
33. S. Lee, Y. K. Wing and S. Fong, A controlled study of folate levels in Chinese inpatients with major depression in Hong Kong. J Affect Disord 49 (1998), p. 73.
34. P. F. Jacques, J. Selhub, A. G. Bostom, P. W. F. Wilson and I. H. Rosenberg, The effect of folic acid fortification on plasma folate and total homocysteine concentrations. N Engl J Med 340 (1999), p. 1449.
35. M. T. Abou-Saleh and A. Coppen, The biology of folate in depression: implications for nutritional hypotheses of psychoses. J Psychiatr Res 20 (1986), p. 91.
36. W. E. Thorton and B. P. Thornton, Folic acid, mental function and dietary habits. J Clin Psychiatry 39 (1978), p. 315.
37. A. Coppen, S. Chaudry and C. Swade, Folic acid enhances lithium prophylaxis. J Affect Disord 10 (1986), p. 9.
38. M. Passeri, S. Ventura, G. Abate, D. Cucinotta and P. LaGreca, Oral 5-methyltetrahydrofolate (MTHF) in depression association with Senile Organic Mental Disorders (OMDs): a double-blind, multicenter study vs. trazodone (TRZ). Eur J Clin Invest 21 (1993), p. 24.
39. G. Guaraldi, M. Fava, F. Mazzi and P. LaGreca, An open trial of methyltetrahydrofolate (MTHF) in elderly depressed patients. Ann Clin Psychiatry 5 (1993), p. 101.
40. Alpert J E, Mischoulon D, Rubenstein G E, Bottonari K, Nierenberg A A, Fava M. Folinic acid (Leucovorin) as an adjunctive treatment for SSRI-refractory depression. Ann Clin Psychiatry. 2002 March; 14(1):33-8.
41. M. Spillman and M. Fava, S-adenosyl-methionine in psychiatric disorders: historical perspective and current status. CNS Drugs 6 (1996), p. 416.
42. A. Ruck, S. Kramer, J. Metz and M. Brennan, Methyltetrahydrofolate is a potent and selective agonist for kainic acid receptors. Nature 287 (1980), p. 852.
43. R. A. Schatz, T. E. Wilens and O. Z. Sellinger, Decreased in vivo protein and phospholipid methylation after in vivo elevation of brain S-adenosylhomocysteine. Biochem Biophys Res Commun 98 (1981), p. 1097.
44. P. J. Shaw, Excitatory amino acid receptors, excitotoxicity and the human nervous system. Curr Opin Neurol Neurosurg 6 (1993), p. 414.
45. M. B. Bowers, Jr and E. H. Reynolds, Cerebrospinal fluid folate and acid monoamine metabolites. Lancet 2 (1972), p. 137.
46. M. I. Botez and S. N. Young, Effects of anticonvulsants and low levels of folate and thiamine on amine metabolites in cerebrospinal fluid. Brain 114 (1991), p. 333.
47. M. I. Botez, S. N. Young, J. Bachevalier and S. Gauthier, Folate deficiency and decreased brain 5-hydroxytryptamine synthesis in man and rat. Nature 278 (1979), p. 182.
48. R. Surtees, S. Heales and A. Bowron, Association of cerebrospinal fluid deficiency of 5-methyltetrahydrofolate, but not S-adenosylmethionine, with reduced concentrations of the acid metabolites of 5-hydroxytryptamine and dopamine. Clin Sci 986 (1994), p. 697.
49. Alpert J E, Mischoulon D, Nierenberg A A, Fava M. Nutrition and depression: focus on folate. Nutrition. 2000 July-August; 16(7-8):544-6
50. Fava M, Davidson K G. Definition and epidemiology of treatment-resistant depression. Psychiatr Clin North Am. 1996 June; 19(2):179-200.
51. Fava M, Evins A E, Dorer D J, Schoenfeld D A. The problem of the placebo response in clinical trials for psychiatric disorders: culprits, possible remedies, and a novel study design approach. Psychother Psychosom. 2003 May-June; 72(3):115-27.
52. Trivedi M H, Rush A J, Ibrahim H M et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychol Med 2004; 34(1):73-82.
53. Fava M. Diagnosis and definition of treatment-resistant depression. Biol Psychiatry. 2003 Apr. 15; 53(8):649-59.
54. Levine J, Schooler N R. General versus specific inquiry with SAFTEE. J Clin Psychopharmacol 1992; 12(6):448.
55. Hamilton M. A rating scale for depression. J Neurol Neurosurg Psychiatry. 1960 February; 23:56-62.
56. Fava M, Rankin M A, Alpert J E, Nierenberg A A, Worthington J J. An open trial of oral sildenafil in antidepressant-induced sexual dysfunction. Psychother Psychosom. 1998; 67(6):328-31.
57. DeLoach U, Higgins M S, Caplan A B, Stiff J L. The visual analog scale in the immediate postoperative period: intrasubject variability and correlation with a numeric scale. Anesth Analg. 1998; 86(1):102-6.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

```
INFORMAL SEQUENCE LISTING
----human MTHFR (NM_005957.4 CDS plus 12 nucleotides in the
beginning of the sequence) C677T and A1298C
SEQ ID NO: 1
      1 aggaacccag ccatggtgaa cgaagccaga ggaaacagca gcctcaaccc ctgcttggag
    61 ggcagtgcca gcagtggcag tgagagctcc aaagatagtt cgagatgttc caccccgggc
  121 ctggaccccg agcggcatga gagactccgg gagaagatga ggcggcgatt ggaatctggt
  181 gacaagtggt tctccctgga attcttccct cctcgaactg ctgagggagc tgtcaatctc
  241 atctcaaggt ttgaccggat ggcagcaggt ggccccctct acatagacgt gacctggcac
  301 ccagcaggtg accctggctc agacaaggag acctcctcca tgatgatcgc cagcaccgcc
  361 gtgaactact gtggcctgga gaccatcctg cacatgacct gctgccgtca gcgcctggag
  421 gagatcacgg gccatctgca caaagctaag cagctgggcc tgaagaacat catggcgctg
  481 cggggagacc caataggtga ccagtgggaa gaggaggagg gaggcttcaa ctacgcagtg
  541 gacctggtga agcacatccg aagtgagttt ggtgactact ttgacatctg tgtggcaggt
  601 taccccaaag gccaccccga agcagggagc tttgaggctg acctgaagca cttgaaggag
  661 aaggtgtctg cgggagccga tttcatcatc acgcagcttt tctttgaggc tgacacattc
  721 ttccgctttg tgaaggcatg caccgacatg ggcatcactt gccccatcgt ccccgggatc
  781 tttcccatcc agggctacca ctcccttcgg cagcttgtga agctgtccaa gctggaggtg
  841 ccacaggaga tcaaggacgt gattgagcca atcaaagaca acgatgctgc catccgcaac
  901 tatggcatcg agctggccgt gagcctgtgc caggagcttc tggccagtgg cttggtgcca
  961 ggcctccact tctacaccct caaccgcgag atggctacca cagaggtgct gaagcgcctg
1021 gggatgtgga ctgaggaccc caggcgtccc ctaccctggg ctctcagcgc ccaccccaag
1081 cgccgagagg aagatgtacg tcccatcttc tgggcctcca gaccaaagag ttacatctac
1141 cgtacccagg agtgggacga gttccctaac ggccgctggg gcaattcctc ttcccctgcc
1201 tttggggagc tgaaggacta ctacctcttc tacctgaaga gcaagtcccc caaggaggag
1261 ctgctgaaga tgtgggggga ggagctgacc agtgaagaaa gtgtctttga agtcttcgtt
1321 ctttacctct cgggagaacc aaaccggaat ggtcacaaag tgacttgcct gccctggaac
1381 gatgagcccc tggcggctga gaccagcctg ctgaaggagg agctgctgcg ggtgaaccgc
1441 cagggcatcc tcaccatcaa ctcacagccc aacatcaacg ggaagccgtc ctccgacccc
1501 atcgtgggct ggggccccag cgggggctat gtcttccaga aggcctactt agagtttttc
1561 acttcccgcg agacagcgga agcacttctg caagtgctga agaagtacga gctccgggtt
1621 aattaccacc ttgtcaatgt gaagggtgaa aacatcacca atgcccctga actgcagccg
1681 aatgctgtca cttggggcat cttccctggg cgagagatca tccagcccac cgtagtggat
1741 cccgtcagct tcatgttctg gaaggacgag gcctttgccc tgtggattga gcggtgggga
1801 aagctgtatg aggaggagtc cccgtcccgc accatcatcc agtacatcca cgacaactac
1861 ttcctggtca acctggtgga caatgacttc ccactggaca actgcctctg gcaggtggtg
1921 gaagacacat tggagcttct caacaggccc acccagaatg cgagagaaac ggaggctcca
1981 tga
SEQ ID NO 2: ----human MTR (NM_000254.2) CDS A2756G
      1 atgtcacccg cgctccaaga cctgtcgcaa cccgaaggtc tgaagaaaac cctgcgggat
    61 gagatcaatg ccattctgca gaagaggatt atggtgctgg atggagggat ggggaccatg
  121 atccagcggg agaagctaaa cgaagaacac ttccgaggtc aggaatttaa agatcatgcc
  181 aggccgctga aaggcaacaa tgacatttta agtataactc agcctgatgt catttaccaa
  241 atccataagg aatacttgct ggctggggca gatatcattg aaacaaatac ttttagcagc
  301 actagtattg cccaagctga ctatggcctt gaacacttgg cctaccggat gaacatgtgc
  361 tctgcaggag tggccagaaa agctgccgag gaggtaactc tccagacagg aattaagagg
  421 tttgtggcag gggctctggg tccgactaat aagacactct ctgtgtcccc atctgtggaa
  481 aggccggatt ataggaacat cacatttgat gagcttgttg aagcatacca agagcaggcc
  541 aaaggacttc tggatggcgg ggttgatatc ttactcattg aaactatttt tgatactgcc
  601 aatgccaagg cagccttgtt tgcactccaa aatctttttg aggagaaata tgctccccgg
  661 cctatcttta tttcagggac gatcgttgat aaaagtgggc ggactctttc cggacagaca
  721 ggagagggat ttgtcatcag cgtgtctcat ggagaaccac tctgcattgg attaaattgt
  781 gctttgggtg cagctgaaat gagacctttt attgaaataa ttggaaaatg tacaacagcc
  841 tatgtcctct gttatcccaa tgcaggtctt cccaacacct ttggtgacta tgatgaaacg
  901 ccttctatga tggccaagca cctaaaggat tttgctatgg atggcttggt caatatagtt
  961 ggaggatgct gtgggtcaac accagatcat atcagggaaa ttgctgaagc tgtgaaaaat
1021 tgtaagccta gagttccacc tgccactgct tttgaaggac atatgttact gtctggtcta
1081 gagcccttca ggattggacc gtacaccaac tttgttaaca ttggagagcg ctgtaatgtt
1141 gcaggatcaa ggaagtttgc taaactcatc atggcaggaa actatgaaga agccttgtgt
1201 gttgccaaag tgcaggtgga aatgggagcc caggtgttgg atgtcaacat ggatgatggc
1261 atgctagatg gtccaagtgc aatgaccaga ttttgcaact taattgcttc cgagccagac
1321 atcgcaaagg tacctttgtg catcgactcc tccaattttg ctgtgattga agctgggtta
1381 aagtgctgcc aagggaagtg cattgtcaat agcattagtc tgaaggaagg agaggacgac
1441 ttcttggaga aggccaggaa gattaaaaag tatggagctg ctatggtggt catggctttt
1501 gatgaagaag gacaggcaac agaaacagac acaaaaatca gagtgtgcac ccgggcctac
1561 catctgcttg tgaaaaaact gggctttaat ccaaatgaca ttatttttga ccctaatatc
1621 ctaaccattg ggactggaat ggaggaacac aacttgtatg ccattaattt tatccatgca
1681 acaaaagtca ttaaagaaac attacctgga gccagaataa gtggaggtct ttccaacttg
1741 tccttctcct tccgaggaat ggaagccatt cgagaagcaa tgcatggggt tttcctttac
1801 catgcaatca agtctggcat ggacatgggg atagtgaatg ctggaaacct ccctgtgtat
1861 gatgatatcc ataaggaact tctgcagctc tgtgaagatc tcatctggaa taaagaccct
1921 gaggccactg agaagctctt acgttatgcc cagactcaag gcacaggagg gaagaaagtc
1981 attcagactg atgagtggag aaatggccct gtcgaagaac gccttgagta tgcccttgtg
2041 aagggcattg aaaaacatat tattgaggat actgaggaag ccaggttaaa ccaaaaaaaa
2101 tatccccgac ctctcaatat aattgaagga cccctgatga atggaatgaa aattgttggt
2161 gatctttttg gagctggaaa aatgtttcta cctcaggtta taaagtcagc ccgggttatg
2221 aagaaggctg ttggccacct tatccctttc atggaaaaag aaagagaaga aaccagagtg
2281 cttaacggca cagtagaaga agaggaccct taccagggca ccatcgtgct ggccactgtt
2341 aaaggcgacg tgcacgacat aggcaagaac atagttggag tagtccttgg ctgcaataat
2401 ttccgagtta ttgatttagg agtcatgact ccatgtgata agatactgaa agctgctctt
2461 gaccacaaag cagatataat tggcctgtca ggactcatca ctccttccct ggatgaaatg
2521 atttttgttg ccaaggaaat ggagagatta gctataagga ttccattgtt gattggagga
2581 gcaaccactt caaaaaccca cacagcagtt aaaatagctc cgagatacag tgcacctgta
2641 atccatgtcc tggacgcgtc caagagtgtg gtggtgtgtt cccagctgtt agatgaaaat
2701 ctaaaggatg aatactttga ggaaatcatg gaagaatatg aagatattag acaggaccat
2761 tatgagtctc tcaaggagag gagatactta cccttaagtc aagccagaaa aagtggtttc
2821 caaatggatt ggctgtctga acctcaccca gtgaagccca cgtttattgg gacccaggtc
2881 tttgaagact atgacctgca gaagctggtg gactacattg actggaagcc tttctttgat
2941 gtctggcagc tccggggcaa gtacccgaat cgaggctttc ccaagatatt taacgacaaa
3001 acagtaggtg gagaggccag gaaggtctac gatgatgccc acaatatgct gaacacactg
3061 attagtcaaa agaaactccg ggcccggggt gtggttgggt tctggccagc acagagtatc
3121 caagacgaca ttcacctgta cgcagaggct gctgtgcccc aggctgcaga gcccatagcc
3181 accttctatg ggttaaggca acaggctgag aaggactctg ccagcacgga gccatactac
3241 tgcctctcag acttcatcgc tcccttgcat tctggcatcc gtgactacct gggcctgttt
3301 gccgttgcct gctttggggt agaagagctg agcaaggcct atgaggatga tggtgacgac
3361 tacagcagca tcatggtcaa ggcgctgggg gaccggctgg cagaggcctt tgcagaagag
3421 ctccatgaaa gagttcgccg agaactgtgg gcctactgtg gcagtgagca gctggacgtc
3481 gcagacctgc gcaggctgcg gtacaagggc atccgcccgg ctcctggcta ccccagccag
3541 cccgaccaca ccgagaagct caccatgtgg agactcgcag acatcgagca gtctacaggc
3601 attaggttaa cagaatcatt agcaatggca cctgcttcag cagtctcagg cctctacttc
3661 tccaatttga agtccaaata ttttgctgtg gggaagattt ccaaggatca ggttgaggat
3721 tatgcattga ggaagaacat atctgtggct gaggttgaga aatggcttgg acccattttg
3781 ggatatgata cagactaa
-----human MTRR (NM_002454.2) A66G
SEQ ID NO: 3
      1 atgaggaggt ttctgttact atatgctaca cagcagggac aggcaaaggc catcgcagaa
    61 gaaatatgtg agcaagctgt ggtacatgga ttttctgcag atcttcactg tattagtgaa
  121 tccgataagt atgacctaaa aaccgaaaca gctcctcttg ttgttgtggt ttctaccacg
  181 ggcaccggag acccacccga cacagcccgc aagtttgtta aggaaataca gaaccaaaca
  241 ctgccggttg atttctttgc tcacctgcgg tatgggttac tgggtctcgg tgattcagaa
  301 tacacctact tttgcaatgg ggggaagata attgataaac gacttcaaga gcttggagcc
  361 cggcatttct atgacactgg acatgcagat gactgtgtag gtttagaact tgtggttgag
  421 ccgtggattg ctggactctg gccagccctc agaaagcatt ttaggtcaag cagaggacaa
  481 gaggagataa gtggcgcact cccggtggca tcacctgcat cctcgaggac agaccttgtg
  541 aagtcagagc tgctacacat tgaatctcaa gtcgagcttc tgagattcga tgattcagga
  601 agaaaggatt ctgaggtttt gaagcaaaat gcagtgaaca gcaaccaatc caatgttgta
  661 attgaagact ttgagtcctc acttacccgt tcggtacccc cactctcaca agcctctctg
  721 aatattcctg gtttaccccc agaatattta caggtacatc tgcaggagtc tcttggccag
  781 gaggaaagcc aagtatctgt gacttcagca gatccagttt ttcaagtgcc aatttcaaag
  841 gcagttcaac ttactacgaa tgatgccata aaaaccactc tgctggtaga attggacatt
  901 tcaaatacag acttttccta tcagcctgga gatgccttca gcgtgatctg ccctaacagt
  961 gattctgagg tacaaagcct actccaaaga ctgcagcttg aagataaaag agagcactgc
1021 gtccttttga aaataaaggc agacacaaag aagaaaggag ctaccttacc ccagcatata
1081 cctgcgggat gttctctcca gttcattttt acctggtgtc ttgaaatccg agcaattcct
1141 aaaaaggcat ttttgcgagc ccttgtggac tataccagtg acagtgctga aaagcgcagg
1201 ctacaggagc tgtgcagtaa acaaggggca gccgattata gccgctttgt acgagatgcc
1261 tgtgcctgct tgttggatct cctcctcgct ttcccttctt gccagccacc actcagtctc
1321 ctgctcgaac atcttcctaa acttcaaccc agaccatatt cgtgtgcaag ctcaagttta
1381 tttcacccag gaaagctcca ttttgtcttc aacattgtgg aatttctgtc tactgccaca
1441 acagaggttc tgcggaaggg agtatgtaca ggctggctgg ccttgttggt tgcttcagtt
1501 cttcagccaa acatacatgc atcccatgaa gacagcggga aagccctggc tcctaagata
1561 tccatctctc ctcgaacaac aaattctttc cacttaccag atgacccctc aatccccatc
1621 ataatggtgg gtccaggaac cggcatagcc ccgtttattg ggttcctaca acatagagag
1681 aaactccaag aacaacaccc agatggaaat tttggagcaa tgtggttgtt ttttggctgc
1741 aggcataagg atagggatta tctattcaga aaagagctca gacatttcct taagcatggg
1801 atcttaactc atctaaaggt ttccttctca agagatgctc ctgttgggga ggaggaagcc
1861 ccagcaaagt atgtgcaaga caacatccag cttcatggcc agcaggtggc gagaatcctc
1921 ctccaggaga acggccatat ttatgtgtgt ggagatgcaa agaatatggc caaggatgta
1981 catgatgccc ttgtgcaaat aataagcaaa gaggttggag ttgaaaaact agaagcaatg
2041 aaaaccctgg ccactttaaa agaagaaaaa cgctaccttc aggatatttg gtcataa
----MTHFR human amino acid sequence (NP_005948.3) A222V &
E429A
SEQ ID NO: 4
      1 mvneargnss lnpclegsas sgsesskdss rcstpgldpe rherlrekmr rrlesgdkwf
    61 sleffpprta egavnlisrf drmaaggply idvtwhpagd pgsdketssm miastavnyc
  121 gletilhmtc crqrleeitg hlhkakqlgl knimalrgdp igdqweeeeg gfnyavdlvk
  181 hirsefgdyf dicvagypkg hpeagsfead lkhlkekvsa gadfiitqlf feadtffrfv
  241 kactdmgitc pivpgifpiq gyhslrqlvk lsklevpqei kdviepikdn daairnygie
  301 layslcqell asglvpglhf ytlnrematt evlkrlgmwt edprrplpwa lsahpkrree
  361 dvrpifwasr pksyiyrtqe wdefpngrwg nssspafgel kdyylfylks kspkeellkm
  421 wgeeltsees vfevfvlyls gepnrnghkv tclpwndepl aaetsllkee llrvnrqgil
  481 tinsqpning kpssdpivgw gpsggyvfqk aylefftsre taeallqvlk kyelrvnyhl
  541 vnvkgenitn apelqpnavt wgifpgreii qptvvdpvsf mfwkdeafal wierwgklye
  601 eespsrtiiq yihdnyflvn lvdndfpldn clwqvvedtl ellnrptqna reteap
----MTR human amino acid sequence (NP_000245.2) D919G
SEQ ID NO: 5
      1 mspalqdlsq peglkktlrd einailqkri mvldggmgtm iqreklneeh frgqefkdha
    61 rplkgnndil sitqpdviyq ihkeyllaga diietntfss tsiaqadygl ehlayrmnmc
  121 sagvarkaae evtlqtgikr fvagalgptn ktlsyspsve rpdyrnitfd elveayqeqa
  181 kglldggvdi llietifdta nakaalfalq nlfeekyapr pifisgtivd ksgrtlsgqt
  241 gegfvisysh geplciglnc algaaemrpf ieiigkctta yvlcypnagl pntfgdydet
  301 psmmakhlkd famdglvniv ggccgstpdh ireiaeavkn ckprvppata feghmllsgl
  361 epfrigpytn fvnigercnv agsrkfakli magnyeealc vakvqvemga qvldvnmddg
  421 mldgpsamtr fcnliasepd iakvplcids snfavieagl kccqgkcivn sislkegedd
  481 flekarkikk ygaamvvmaf deegqatetd tkirvctray hllvkklgfn pndiifdpni
  541 ltigtgmeeh nlyainfiha tkviketlpg arisgglsnl sfsfrgmeai reamhgvfly
  601 haiksgmdmg ivnagnlpvy ddihkellql cedliwnkdp eatekllrya qtqgtggkkv
  661 iqtdewrngp veerleyalv kgiekhiied teearinqkk yprpiniieg plmngmkivg
  721 dlfgagkmfl pqviksarvm kkavghlipf mekereetry lngtveeedp yqgtivlatv
  781 kgdvhdigkn ivgvvlgcnn frvidlgvmt pcdkilkaal dhkadiigls glitpsldem
  841 ifvakemerl airiplligg attskthtav kiaprysapv ihvldasksv vvcsqllden
  901 lkdeyfeeim eeyedirqdh yeslkerryl plsqarksgf qmdwlsephp vkptfigtqv
  961 fedydlqklv dyidwkpffd vwqlrgkypn rgfpkifndk tvggearkvy ddahnmlntl
1021 isqkklrarg vvgfwpaqsi qddihlyaea avpqaaepia tfyglrqqae kdsastepyy
1081 clsdfiaplh sgirdylglf avacfgveel skayeddgdd yssimvkalg drlaeafaee
1141 lhervrrelw aycgseqldv adlrrlrykg irpapgypsq pdhtekltmw rladieqstg
1201 irlteslama pasavsglyf snlkskyfav gkiskdqved yalrknisva evekwlgpil
1261 gydtd
----MTRR human amino acid sequence (NP_002445.2) I22M
SEQ ID NO: 6
      1 mrrflllyat qqgqakaiae eiceqavvhg fsadlhcise sdkydlktet aplvvvvstt
    61 gtgdppdtar kfvkeqanqt lpvdffahlr ygllglgdse ytyfcnggki idkrlqelga
  121 rhfydtghad dcvglelvve pwiaglwpal rkhfrssrgq eeisgalpva spassrtdlv
  181 ksellhiesq vellrfddsg rkdsevlkqn avnsnqsnvv iedfessltr svpplsgasl
  241 nipglppeyl qvhlqeslgq eesqvsvtsa dpvfqvpisk avqlttndai kttllveldi
  301 sntdfsyqpg dafsvicpns dsevqsllqr lqledkrehc vllkikadtk kkgatlpqhi
  361 pagcslqfif twcleiraip kkaflralvd ytsdsaekrr lqelcskqga adysrfvrda
  421 caclldllla fpscqpplsl llehlpklqp rpyscasssl fhpgklhfvf niveflstat
  481 tevlrkgvct gwlallvasv lqpnihashe dsgkalapki sisprttnsf hlpddpsipi
  541 imvgpgtgia pfigflqhre klqeqhpdgn fgamwlffgc rhkdrdylfr kelrhflkhg
  601 ilthlkvsfs rdapvgeeea pakyvqdniq lhgqqvaril lqenghiyvc gdaknmakdv
  661 hdalvqiisk evgvekleam ktlatlkeek rylqdiws
rs1801133
SEQ ID NO: 7
CTTGAAGGAGAAGGTGTCTGCGGGAG[C/T]CGATTTCATCATCACGCAGCTTTTC
rs2274976
SEQ ID NO: 8
CGAGGCCTTTGCCCTGTGGATTGAGC[A/G]GTGGGGAAAGCTGTATGAGGAGGAG
rs1805087
SEQ ID NO: 9
GGAAGAATATGAAGATATTAGACAGG[A/G]CCATTATGAGTCTCTCAAGGTAAGT
rs1801394
SEQ ID NO: 10
CAGGCAAAGGCCATCGCAGAAGAAAT [A/G) TGTGAGCAAGCTGTGGTACATGGAT
rs1006737
SEQ ID NO: 11
TAAGTTCCATTCCATCTCAGCCCGAA [A/G) TGTTTTCAGAGCCGGAGACCTCACA
rs1883729
SEQ ID NO: 12
CTGCTGCTGGTATCAGCCTGGAGGAA(A/G]TGAGTGACATCAGTTCTCAGCATTA
rs7163862
SEQ ID NO: 13
AACCAATCACAACAAGGCAGATAAAG[A/T]AGGATGAGTTGTCAGATTTTGATAA
rs12659
SEQ ID NO: 14
GCTTCGGAGCTGGAGCGCATGAATCC[C/T]GGCCCAGGCGGGAAGCTGGGACACG
rs202676
SEQ ID NO: 15
AAGCTGAGAACATCAAGAAGTTCTTA[C/T]AGTAAGTACATCCTCGAAAGTTTAT
rs2297291
SEQ ID NO: 16
GGGAGGGCACCCGCAGAGGCCTGCGC[A/G]CTGACACTGCTGAGTGGCTCTGCTC
rs1051266
SEQ ID NO: 17
TGACCCCGAGCTCCGGTCCTGGCGGC[A/G]CCTCGTGTGCTACCTTTGCTTCTAC
rs8007267
SEQ ID NO: 18
CAATAGGAGCGTGTGTTTGAACAGTA[C/T]ACGCCAAACTTCAGTCATTCAAGTA
rs7639752
SEQ ID NO: 19
GGCCTAATCAATCCTTCTCATCTTTT[A/G]TACCCACCTTTTGCAGGAAACCTGT
rs6275
SEQ ID NO: 20
CTGACTCTCCCCGACCCGTCCCACCA[C/T]GGTCTCCACAGCACTCCCGACAGCC
rs1079596
SEQ ID NO: 21
GTCCCTGCAGTTTAATTATCCTCAAC[A/G]TTACTGCCATACCCTACATTTTTGG
rs11240594
SEQ ID NO: 22
CTCACAGTTTGTGGTTGAGACTAAGT[A/G]TGACAACAGTGGCACTTTGTGGTCC
rs4633
SEQ ID NO: 23
ACCAAGGAGCAGCGCATCCTGAACCA[C/T]GTGCTGCAGCATGCGGAGCCCGGGA
rs4680
SEQ ID NO: 24
CCCAGCGGATGGTGGATTTCGCTGGC[A/G]TGAAGGACAAGGTGTGCATGCCTGA
rs250682
SEQ ID NO: 25
TAATATGGCCACCCCAACTTTCGTAT[C/G]ATTACTGTTTGTGTGGTATTATCTT
rs2277820
SEQ ID NO: 26
ATCAGCCCTAGATGCTTGACCAGCTC[C/T]TCGGGCCTCACCTCCTGGTTCTTCC
rs2236225
SEQ ID NO: 27
CTGGGCCAACAAGCTTGAGTGCGATC[C/T]GGTCTGCAATGATGGAGGAATTGCC
---COMT human amino acid sequence (NP_000745.1) V158M
SEQ ID NO: 28
      1 mpeappllla avllglvllv vlllllrhwg wglcligwne filqpihnll mgdtkeqril
    61 nhvlqhaepg naqsvleaid tyceqkewam nvgdkkgkiv daviqehqps vllelgaycg
  121 ysavrmarll spgarlitie inpdcaaitq rmvdfagvkd kvtivvgasq diipqlkkky
  181 dvdtldmvfl dhwkdrylpd tllleecgll rkgtvlladn vicpgapdf1 ahvrgsscfe
  241 cthyqsfley revvdgleka iykgpgseag p
```

What is claimed is:

1. An assay for selecting a treatment regimen for a human subject diagnosed as having depression or having a risk for depression, the assay comprising:
(a) analyzing a sample from the subject to determine the genotype of at least two genetic biomarkers selected from the group consisting of methylenetetrahydrofolate reductase (MTHFR), methionine synthase (MTR), GTP cyclohydrolase 1 (GCH1), catechol-O-methyltransferase (COMT), and a combination thereof:
(b) detecting by genotyping for the presence or absence of a single nucleotide polymorphism (SNP) in each of the at least two genetic biomarkers, wherein the presence of the SNP is set forth as the following:
(i) a SNP at position 677 of SEQ ID NO. 1 or position 27 of SEQ ID NO: 7 as identified as rs1801133 comprising at least one thymine “T” allele for MTFHR;
(ii) a SNP at position 2756 of SEQ ID NO: 2 or position 27 of SEQ ID NO: 9 as identified as rs1805087 comprising at least one guanine “G” allele for MTR;
(iii) a SNP at position 27 of SEQ ID NO: 18 as identified as rs8007267 comprising at least one thymine “T” allele for GCH1;
(iv) a SNP at position 27 of SEQ ID NO: 24 as identified as rs4680 comprising two guanine “G” alleles for COMT; and
(c) selecting the treatment regimen comprising an effective amount of a folate-comprising compound based on the presence of said SNPs.

2. The assay of claim 1, further comprising administering said treatment regimen.

3. The assay of claim 1, wherein the at least two genetic biomarkers are the MTHFR and MTR pair.

4. The assay of claim 1, wherein the at least two genetic biomarkers are the GCH1 and COMT pair.

5. The assay of claim 1, wherein step (a) further comprises determining at least one additional parameter selected from the group consisting of obesity, SAM/SAH ratio, level of 4-HNE, level of hsCRP, and a combination thereof.

6. The assay of claim 5, wherein step (b) further comprises detecting at least one of the following conditions:
(i) an expression level ratio of SAM to SAH smaller than a pre-determined reference ratio;
(ii) an expression level of 4-HNE greater than a first pre-determined reference value; and
(iii) an expression level of hsCRP greater than a second pre-determined reference value.

7. The assay of claim 5, wherein obesity is determined, if any of the following conditions are present in the subject:
a BMI value is 30 kg/m² or greater,
a waist circumference is greater than 40 inches in men or greater than 35 inches in women,
a waist-hip ratio is about 0.95 for men or above 0.8 for women, or
a body fat percentage of at least about 25% in men or at least about 32% in women.

8. The assay of claim 6, wherein the pre-determined reference ratio of SAM/SAH is about 3.0 if measured in a plasma sample from a normal, healthy subject.

9. The assay of claim 6, wherein the first pre-determined reference value of 4HNE is about 0.24 mole per liter or about 0.04 mg per liter if measured in a serum sample from a normal, healthy subject.

10. The assay of claim 6, wherein the first pre-determined reference value of 4-HNE is about 3.0 mg per liter if measured in a plasma sample from a normal, healthy subject.

11. The assay of claim 6, wherein the second pre-determined reference value of hsCRP is from about 0.5 mg per liter to about 4.5 mg per liter if measured in a serum sample from a normal, healthy subject.

12. The assay of claim 1, wherein depression is major depressive disorder.

13. The assay of claim 1, wherein the effective amount of the folate-comprising compound is about 15 mg/day to about 50 mg/day.

14. The assay of claim 1, wherein said treatment regimen further comprises an anti-depressant drug.

15. A folate-comprising composition for use in the treatment of depression in a human subject who is diagnosed as having depression or having a risk for depression and carries at least two of the following SNPs selected from the group consisting of:
(i) the SNP at position 677 of SEQ ID NO. 1 or position 27 of SEQ ID NO: 7 as identified as rs1801133 comprising at least one thymine “T” allele for MTFHR;
(ii) the SNP at position 2756 of SEQ ID NO: 2 or position 27 of SEQ ID NO: 9 as identified as rs1805087 comprising at least one guanine “G” allele for MTR;
(iii) the SNP at position 27 of SEQ ID NO: 18 as identified as rs8007267 comprising at least one thymine “T” allele for GCH1; and
(iv) the SNP at position 27 of SEQ ID NO: 24 as identified as rs4680 comprising two guanine “G” alleles for COMT.

16. The composition of claim 15, wherein the at least two SNPs are the SNP at position 677 of SEQ ID NO. 1 or position 27 of SEQ ID NO: 7 as identified as rs1801133 comprising at least one thymine “T” allele for MTFHR and the SNP at position 2756 of SEQ ID NO: 2 or position 27 of SEQ ID NO: 9 as identified as rs1805087 comprising at least one guanine “G” allele for MTR.

17. The composition of claim 15, wherein the at least two SNPs are the SNP at position 27 of SEQ ID NO: 18 as identified as rs8007267 comprising at least one thymine “T” allele for GCH1 and the SNP at position 27 of SEQ ID NO: 24 as identified as rs4680 comprising two guanine “G” alleles for COMT.

18. The composition of claim 15, wherein the depression is major depressive disorder.

19. The composition of claim 15, wherein the subject is receiving at least one antidepressant drug.

20. A kit for use in selecting a treatment regimen for a human subject diagnosed as having depression or having a risk for depression comprising
at least one reagent for determining the presence or absence of at least two of the following single nucleotide polymorphisms (SNPs) in a sample taken from the subject:
(i) a SNP at position 677 of SEQ ID NO. 1 or position 27 of SEQ ID NO: 7 as identified as rs1801133 comprising at least one thymine “T” allele for MTFHR;
(ii) a SNP at position 2756 of SEQ ID NO: 2 or position 27 of SEQ ID NO: 9 as identified as rs1805087 comprising at least one guanine “G” allele for MTR;
(iii) a SNP at position 27 of SEQ ID NO: 18 as identified as rs8007267 comprising at least one thymine “T” allele for GCH1;
(iv) a SNP at position 27 of SEQ ID NO: 24 as identified as rs4680 comprising two guanine “G” alleles for COMT; and
instructions for use of said kit.
